SEQUENCE LISTING (351 sequences)

SEQ ID NO: 1 - DNA, 2664 bp, Homo sapiens
  misc_feature (2652)..(2652): n = g or t
gtgagacacctgacgtctgccccgcgctgccggcggtaacatcccagaagcgggtttgaacgtgcctagccgtgcccccagcctcttcccctgagcggagcttgagccccagacctctagtcctcccggtctttatctgagttcagcttagagatgaacggggagccgccctcctgtgctgggcttggggctggaggctgcatcttcccgtttctagggtttcctttccccttttgatcgacgcagtgctcagtcctggccgggacccgagccacctctcctgctcctgcaggacgcacatggctgggtctgaatccctggggtgaggagcaccgtggcctgagagggggcccctgggccagctctgaaatctgaatgtctcaatcacaaagacccccttaggccaggccaggggtgactgtctctggtctttgtccctggttgctggcacatagcacccgaaacccttggaaaccgagtgatgagagagccttttgctcatgaggtgactgatgaccggggacaccaggtggcttcaggatggaagcagatggccagaaagaccaaggcctgatgacgggttgggatggaaaaggggtgaggggctggagattgagtgaatcaccagtggcttagtcaaccatgcctgcacaatggaaccccgtaagaaaccacagggatcagagggcttcccgccgggttgtggaacacaccaaggcactggagggtggtgcgagcagagagcacagcatcactgcccccacctcacaccaggccctacgcatctcttccatacggctgtctgagttttatcctttgtaataaaccagcaactgtaagaaacgcactttcctgagttctgtgaccctgaagagggagtcctgggaacctctgaatttataactagttgatcgaaagtacaagtgacaacctgggatttgccattggcctctgaagtgaaggcagtgttgtgggactgagcccttaacctgtggagtctgtgctgactccaggtagtgtcaagattgaattgaattgtaggacacccagccgtgtccagaaagttgcagaattgatgggtgtgagaaaaaccctacacatttaatgtcagaagtgtgggtaaaatgtttcaccctccagcccagagagccctaatttaccagtggcccacggtggaacaccacgtccggccgggggcagagcgttcccagccaagccttctgtaacatgacatgacaggtcagactccctcgggccctgagttcacttcttcctggtatgtgaccagctcccagtaccagagaaggttgcacagtcctctgctccaaggagcttcactggccaggggctgctttctgaaatccttgcctgcctctgctccaaggcccgttcctcagagacgcagacccctctgatggctgactttggtttgaggacctctctgcatccctcccccatggccttgctcctaggacaccttcttcctcctttccctggggtcagacttgcctaggtgcggtggctctcccagccttccccacgccctccccatggtgtattacacacaccaaagggactcccctattgaaatccatgcatattgaatcgcatgtgggttccggctgctcctgggaggagccaggctaatagaatgtttgccataaaatattaatgtacagagaagcgaaacaaaggtcgttggtacttgttaaccttaccagcagaataatgaaagcgaacccccatatctcatctgcacgcgacatccttgttgtgtctgtacccgaggctccaggtgcagccactgttacagagactgtgtttcttccccatgtacctcgggggccgggaggggttctgatctgcaaagtcgccagaggttaagtcctttctctcttgtggctttgccacccctggagtgtcaccctcagctgcggtgcccaggattccccactgtggtatgtccgtgcaccagtcaataggaaagggagcaaggaaaggtactgggtccccctaaggacatacgagttgccagaatcacttccgctgacacccagtggaccaagccgcacctttatgcagaagtggggctcccagccaggcgtggtcactcctgaaatcccagcacttcggaaggccaaggggggtggatcacttgagctcaggagttcgagaccagcctgggtaacatggcaaaatcccgtctctacaaaaatacagaaaattagctgggtgcggtggtgtgtgcctacagtcccagctactcaggaggctgaagtgggaggattgcttgagtctgggaggtggaggttgcagtgagccaggatctcaccacagcactctggcccaggcgacagctgtttggcctgtttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccccacctgcctgggtgctgcagtgccagccgcggttgatgtctcagagctgctttgagagccccgtgagtgccgcccctcccgcctccctgctgagcccgcttncttctcccgcag

SEQ ID NO: 2 - DNA, 578 bp, Homo sapiens
gcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacg

SEQ ID NO: 3 - DNA, 616 bp, Homo sapiens
gtgggcagggcaggggcgggggcggcggccagggcagagggtgcgcgtggacatcgacacccacgcacctcacaagggtggggtgcatgttgcaccactgtgtgctgggcccttgctgggagcggaggtgtgagcagacaatggcagcgcccctcggggagcagtggggacaccacggtgacaggtactccagaaggcagggctcggggctcattcatctttatgaaaaggtgggtcaggtagagtagggctgccagaggttgcgaatgaaaacaggatgcccagtaaacccgaattgcagataccccaggcatgactttgtttttttgtgtaaggatgcaaaatttgggatgtatttatactagaaaagctgcttgttgtttatctgaaattcagagttatcaggtgttctgtattttacctccatcctgggggaggcgtcctcctcctggctctgcagatgagggagccgaggctcagagaggctgaatgtgctgcccatggtcccacatccatgtgtggctgcaccaggacctgacctgtccttggcgtgcgggttgttctctggagagtaaggtggctgtggggaacatcaataaacccccatctcttctag

SEQ ID NO: 4 - DNA, 25 bp, Artificial Sequence
  Antisense oligomer
gccctggtctgctggctccctgctg

SEQ ID NO: 5 - DNA, 25 bp, Artificial Sequence
  Antisense oligomer
ccctggtctgctggctccctgctgg

SEQ ID NO: 6 - DNA, 25 bp, Artificial Sequence
  Antisense oligomer
cctggtctgctggctccctgctggt

SEQ ID NO: 7 - DNA, 25 bp, Artificial Sequence
  Antisense oligomer
ctggtctgctggctccctgctggtg

SEQ ID NO: 8 - DNA, 25 bp, Artificial Sequence
  Antisense oligomer
tggtctgctggctccctgctggtga

SEQ ID NO: 9 - DNA, 25 bp, Artificial Sequence
  Antisense oligomer
ggtctgctggctccctgctggtgag

SEQ ID NO: 10 - DNA, 25 bp, Artificial Sequence
  Antisense oligomer
gtctgctggctccctgctggtgagc

SEQ ID NO: 11 - DNA, 25 bp, Artificial Sequence
  Antisense oligomer
tctgctggctccctgctggtgagct

SEQ ID NO: 12 - DNA, 25 bp, Artificial Sequence
  Antisense oligomer
gggccctggtctgctggctccctgc

SEQ ID NO: 13 - DNA, 25 bp, Artificial Sequence
  Antisense oligomer
ggggccctggtctgctggctccctg

SEQ ID NO: 14 - DNA, 25 bp, Artificial Sequence
  Antisense oligomer
cggggccctggtctgctggctccct

SEQ ID NO: 15 - DNA, 25 bp, Artificial Sequence
  Antisense oligomer
ccggggccctggtctgctggctccc

SEQ ID NO: 16 - DNA, 25 bp, Artificial Sequence
  Antisense oligomer
cccggggccctggtctgctggctcc

SEQ ID NO: 17 - DNA, 25 bp, Artificial Sequence
  Antisense oligomer
tcccggggccctggtctgctggctc

SEQ ID NO: 18 - DNA, 25 bp, Artificial Sequence
  Antisense oligomer
atcccggggccctggtctgctggct

SEQ ID NO: 19 - DNA, 25 bp, Artificial Sequence
  Antisense oligomer
catcccggggccctggtctgctggc

SEQ ID NO: 20 - DNA, 27 bp, Artificial Sequence
  Antisense oligomer
tctgccctggccgccgcccccgcccct

SEQ ID NO: 21 - DNA, 25 bp, Artificial Sequence
  Antisense oligomer
tgaggtgcgtgggtgtcgatgtcca

SEQ ID NO: 22 - DNA, 25 bp, Artificial Sequence
  Antisense oligomer
gaggtgcgtgggtgtcgatgtccac

SEQ ID NO: 23 - DNA, 25 bp, Artificial Sequence
  Antisense oligomer
aggtgcgtgggtgtcgatgtccacg

SEQ ID NO: 24 - DNA, 25 bp, Artificial Sequence
  Antisense oligomer
gcgcgtggacatcgacacccacgca

SEQ ID NO: 25 - DNA, 25 bp, Artificial Sequence
  Antisense oligomer
tgtgagggcgcgtggacatcgacac

SEQ ID NO: 26 - DNA, 25 bp, Artificial Sequence
  Antisense oligomer
ttgtgagggcgcgtggacatcgaca

SEQ ID NO: 27 - DNA, 24 bp, Artificial Sequence
  Antisense oligomer
ctgtgagggcgcgtggacatcgac

SEQ ID NO: 28 - DNA, 25 bp, Artificial Sequence
  Antisense oligomer
  misc_feature (1)..(25): n = t or u
ggcccnggncngcnggcncccngcn

SEQ ID NO: 29 - DNA, 20 bp, Artificial Sequence
  Antisense oligomer
tggccgccgcccccgcccct

SEQ ID NO: 30 - DNA, 20 bp, Artificial Sequence
  Antisense oligomer
gtgaggtgcgtgggtgtcga

SEQ ID NO: 31 - protein, 6 aa, Artificial Sequence
  synthetic
RRRRRR

SEQ ID NO: 32 - protein, 7 aa, Artificial Sequence
  synthetic
RRRRRRG

SEQ ID NO: 33 - DNA, 17 bp, Artificial Sequence
  RT-PCR primer FWD124
cgttgttcagcgaggga

SEQ ID NO: 34 - DNA, 21 bp, Artificial Sequence
  RT-PCR primer FWD645
ctcctctgaaatgggctacac

SEQ ID NO: 35 - DNA, 18 bp, Artificial Sequence
  RT-PCR primer REV780
acctcgtagcgcctgtta

SEQ ID NO: 36 - DNA, 20 bp, Artificial Sequence
  Antisense oligomer
gaaggaagggcgagaaaagc

SEQ ID NO: 37 - DNA, 20 bp, Artificial Sequence
  Antisense oligomer
gcagaaaagctccagcaggg

SEQ ID NO: 38 - DNA, 20 bp, Artificial Sequence
  Antisense oligomer
gctcagcagggaggcgggag

SEQ ID NO: 39 - DNA, 20 bp, Artificial Sequence
  Antisense oligomer
ggctctcaaagcagctctga

SEQ ID NO: 40 - DNA, 25 bp, Artificial Sequence
  Antisense oligomer
gacatcaaccgcggctggcactgca

SEQ ID NO: 41 - DNA, 25 bp, Artificial Sequence
  Antisense oligomer
gggtaaggtggccagggtgggtgtt

SEQ ID NO: 42 - DNA, 19 bp, Artificial Sequence
  Antisense oligomer
gccctgctgtctagactgg

SEQ ID NO: 43 - DNA, 20 bp, Artificial Sequence
  Antisense oligomer
gagagggccagaaggaaggg

SEQ ID NO: 44 - DNA, 17 bp, Artificial Sequence
  Antisense oligomer
cccgcccctgccctgcc

SEQ ID NO: 45 - DNA, 18 bp, Artificial Sequence
  Antisense oligomer
tggccgccgcccccgccc

SEQ ID NO: 46 - DNA, 20 bp, Artificial Sequence
  Antisense oligomer
tgtccacgcgcaccctctgc

SEQ ID NO: 47 - DNA, 20 bp, Artificial Sequence
  Antisense oligomer
gcaacatgcaccccaccctt

SEQ ID NO: 48 - DNA, 20 bp, Artificial Sequence
  Antisense oligomer
agggcccagcacacagtggt

SEQ ID NO: 49 - DNA, 20 bp, Artificial Sequence
  Antisense oligomer
tcacacctccgctcccagca

SEQ ID NO: 50 - DNA, 18 bp, Artificial Sequence
  Antisense oligomer
ggcgctgccattgtctgc

SEQ ID NO: 51 - DNA, 20 bp, Artificial Sequence
  Antisense oligomer
gtgtccccactgctccccga

SEQ ID NO: 52 - DNA, 20 bp, Artificial Sequence
  Antisense oligomer
ctggagtacctgtcaccgtg

SEQ ID NO: 53 - DNA, 20 bp, Artificial Sequence
  Antisense oligomer
tgagccccgagccctgcctt

SEQ ID NO: 54 - DNA, 25 bp, Artificial Sequence
  Antisense oligomer
tgacccaccttttcataaagatgaa

SEQ ID NO: 55 - DNA, 25 bp, Artificial Sequence
  Antisense oligomer
ctctggcagccctactctacctgac

SEQ ID NO: 56 - DNA, 27 bp, Artificial Sequence
  Antisense oligomer
ctagtataaatacatcccaaattttgc

SEQ ID NO: 57 - DNA, 25 bp, Artificial Sequence
  Antisense oligomer
ggccctggtctgctggctccctgct

SEQ ID NO: 58 - DNA, 25 bp, Artificial Sequence
  Antisense oligomer
gctccctgcagcccctgctttgcag

SEQ ID NO: 59 - DNA, 20 bp, Artificial Sequence
  Antisense oligomer
gcggggcagacgtcaggtgt

SEQ ID NO: 60 - DNA, 20 bp, Artificial Sequence
  Antisense oligomer
cagcgcggggcagacgtcag

SEQ ID NO: 61 - DNA, 20 bp, Artificial Sequence
  Antisense oligomer
ccggcagcgcggggcagacg

SEQ ID NO: 62 - DNA, 20 bp, Artificial Sequence
  Antisense oligomer
ccgccggcagcgcggggcag

SEQ ID NO: 63 - DNA, 20 bp, Artificial Sequence
  Antisense oligomer
gatgttaccgccggcagcgc

SEQ ID NO: 64 - DNA, 20 bp, Artificial Sequence
  Antisense oligomer
ctgggatgttaccgccggca

SEQ ID NO: 65 - DNA, 20 bp, Artificial Sequence
  Antisense oligomer
gcttctgggatgttaccgcc

SEQ ID NO: 66 - DNA, 20 bp, Artificial Sequence
  Antisense oligomer
tggcaactcgtatgtcctta

SEQ ID NO: 67 - DNA, 20 bp, Artificial Sequence
  Antisense oligomer
attctggcaactcgtatgtc

SEQ ID NO: 68 - DNA, 20 bp, Artificial Sequence
  Antisense oligomer
aagtgattctggcaactcgt

SEQ ID NO: 69 - DNA, 20 bp, Artificial Sequence
  Antisense oligomer
tgggtgtcagcggaagtgat

SEQ ID NO: 70 - DNA, 20 bp, Artificial Sequence
  Antisense oligomer
gtccactgggtgtcagcgga

SEQ ID NO: 71 - DNA, 20 bp, Artificial Sequence
  Antisense oligomer
gcttggtccactgggtgtca

SEQ ID NO: 72 - DNA, 20 bp, Artificial Sequence
  Antisense oligomer
ccccacttctgcataaaggt

SEQ ID NO: 73 - DNA, 20 bp, Artificial Sequence
  Antisense oligomer
ggagccccacttctgcataa

SEQ ID NO: 74 - DNA, 20 bp, Artificial Sequence
  Antisense oligomer
gctgggagccccacttctgc

SEQ ID NO: 75 - DNA, 20 bp, Artificial Sequence
  Antisense oligomer
ccacgcctggctgggagccc

SEQ ID NO: 76 - DNA, 20 bp, Artificial Sequence
  Antisense oligomer
tccgaagtgctgggatttca

SEQ ID NO: 77 - DNA, 20 bp, Artificial Sequence
  Antisense oligomer
tccaccccccttggccttcc

SEQ ID NO: 78 - DNA, 20 bp, Artificial Sequence
  Antisense oligomer
tgatccaccccccttggcct

SEQ ID NO: 79 - DNA, 20 bp, Artificial Sequence
  Antisense oligomer
tcaagtgatccacccccctt

SEQ ID NO: 80 - DNA, 20 bp, Artificial Sequence
  Antisense oligomer
gaactcctgagctcaagtga

SEQ ID NO: 81 - DNA, 20 bp, Artificial Sequence
  Antisense oligomer
tctcgaactcctgagctcaa

SEQ ID NO: 82 - DNA, 20 bp, Artificial Sequence
  Antisense oligomer
ccaggctggtctcgaactcc

SEQ ID NO: 83 - DNA, 20 bp, Artificial Sequence
  Antisense oligomer
tttgccatgttacccaggct

SEQ ID NO: 84 - DNA, 20 bp, Artificial Sequence
  Antisense oligomer
acgggattttgccatgttac

SEQ ID NO: 85 - DNA, 20 bp, Artificial Sequence
  Antisense oligomer
tagagacgggattttgccat

SEQ ID NO: 86 - DNA, 20 bp, Artificial Sequence
  Antisense oligomer
ttttgtagagacgggatttt

SEQ ID NO: 87 - DNA, 20 bp, Artificial Sequence
  Antisense oligomer
tctgtatttttgtagagacg

SEQ ID NO: 88 - DNA, 20 bp, Artificial Sequence
  Antisense oligomer
attttctgtatttttgtaga

SEQ ID NO: 89 - DNA, 20 bp, Artificial Sequence
  Antisense oligomer
gctaattttctgtatttttg

SEQ ID NO: 90 - DNA, 20 bp, Artificial Sequence
  Antisense oligomer
ccgccgcccccgcccctgcc

SEQ ID NO: 91 - DNA, 20 bp, Artificial Sequence
  Antisense oligomer
ctgccctggccgccgccccc

SEQ ID NO: 92 - DNA, 20 bp, Artificial Sequence
  Antisense oligomer
caccctctgccctggccgcc

SEQ ID NO: 93 - DNA, 20 bp, Artificial Sequence
  Antisense oligomer
gcgcaccctctgccctggcc

SEQ ID NO: 94 - DNA, 20 bp, Artificial Sequence
  Antisense oligomer
tgtcgatgtccacgcgcacc

SEQ ID NO: 95 - DNA, 20 bp, Artificial Sequence
  Antisense oligomer
tgcgtgggtgtcgatgtcca

SEQ ID NO: 96 - DNA, 20 bp, Artificial Sequence
  Antisense oligomer
gcaccccacccttgtgaggt

SEQ ID NO: 97 - DNA, 20 bp, Artificial Sequence
  Antisense oligomer
aacatgcaccccacccttgt

SEQ ID NO: 98 - DNA, 20 bp, Artificial Sequence
  Antisense oligomer
aggaggaggacgcctccccc

SEQ ID NO: 99 - DNA, 20 bp, Artificial Sequence
  Antisense oligomer
ctcatctgcagagccaggag

SEQ ID NO: 100 - DNA, 20 bp, Artificial Sequence
  Antisense oligomer
gctccctcatctgcagagcc

SEQ ID NO: 101 - DNA, 20 bp, Artificial Sequence
  Antisense oligomer
tcggctccctcatctgcaga

SEQ ID NO: 102 - DNA, 20 bp, Artificial Sequence
  Antisense oligomer
gcctcggctccctcatctgc

SEQ ID NO: 103 - DNA, 20 bp, Artificial Sequence
  Antisense oligomer
ttctgggatgttaccgccgg

SEQ ID NO: 104 - DNA, 20 bp, Artificial Sequence
  Antisense oligomer
cttctgggatgttaccgccg

SEQ ID NO: 105 - DNA, 20 bp, Artificial Sequence
  Antisense oligomer
cgcttctgggatgttaccgc

SEQ ID NO: 106 - DNA, 20 bp, Artificial Sequence
  Antisense oligomer
ccgcttctgggatgttaccg

SEQ ID NO: 107 - DNA, 20 bp, Artificial Sequence
  Antisense oligomer
acccgcttctgggatgttac

SEQ ID NO: 108 - DNA, 20 bp, Artificial Sequence
  Antisense oligomer
tcaaacccgcttctgggatg

SEQ ID NO: 109 - DNA, 20 bp, Artificial Sequence
  Antisense oligomer
acgttcaaacccgcttctgg

SEQ ID NO: 110 - DNA, 20 bp, Artificial Sequence
  Antisense oligomer
gggctctcaaagcagctctg

SEQ ID NO: 111 - DNA, 20 bp, Artificial Sequence
  Antisense oligomer
ggggctctcaaagcagctct

SEQ ID NO: 112 - DNA, 20 bp, Artificial Sequence
  Antisense oligomer
acggggctctcaaagcagct

SEQ ID NO: 113 - DNA, 20 bp, Artificial Sequence
  Antisense oligomer
tcacggggctctcaaagcag

SEQ ID NO: 114 - DNA, 20 bp, Artificial Sequence
  Antisense oligomer
gctctcaaagcagctctgag

SEQ ID NO: 115 - DNA, 20 bp, Artificial Sequence
  Antisense oligomer
ctctcaaagcagctctgaga

SEQ ID NO: 116 - DNA, 20 bp, Artificial Sequence
  Antisense oligomer
ctcaaagcagctctgagaca

SEQ ID NO: 117 - DNA, 20 bp, Artificial Sequence
  Antisense oligomer
caaagcagctctgagacatc

SEQ ID NO: 118 - DNA, 20 bp, Artificial Sequence
  Antisense oligomer
aagcagctctgagacatcaa

SEQ ID NO: 119 - DNA, 18 bp, Artificial Sequence
  Antisense oligomer
gagagggccagaaggaag

SEQ ID NO: 120 - DNA, 18 bp, Artificial Sequence
  Antisense oligomer
gagggccagaaggaaggg

SEQ ID NO: 121 - DNA, 18 bp, Artificial Sequence
  Antisense oligomer
gggccagaaggaagggcg

SEQ ID NO: 122 - DNA, 18 bp, Artificial Sequence
  Antisense oligomer
gggagagggccagaagga

SEQ ID NO: 123 - DNA, 20 bp, Artificial Sequence
  Antisense oligomer
agagggccagaaggaagggc

SEQ ID NO: 124 - DNA, 20 bp, Artificial Sequence
  Antisense oligomer
gagggccagaaggaagggcg

SEQ ID NO: 125 - DNA, 20 bp, Artificial Sequence
  Antisense oligomer
agggccagaaggaagggcga

SEQ ID NO: 126 - DNA, 20 bp, Artificial Sequence
  Antisense oligomer
gggccagaaggaagggcgag

SEQ ID NO: 127 - DNA, 20 bp, Artificial Sequence
  Antisense oligomer
ggccagaaggaagggcgaga

SEQ ID NO: 128 - DNA, 20 bp, Artificial Sequence
  Antisense oligomer
gccagaaggaagggcgagaa

SEQ ID NO: 129 - DNA, 22 bp, Artificial Sequence
  Antisense oligomer
gggagagggccagaaggaaggg

SEQ ID NO: 130 - DNA, 25 bp, Artificial Sequence
  Antisense oligomer
ctggggagagggccagaaggaaggg

SEQ ID NO: 131 - DNA, 22 bp, Artificial Sequence
  Antisense oligomer
gagagggccagaaggaagggcg

SEQ ID NO: 132 - DNA, 25 bp, Artificial Sequence
  Antisense oligomer
gagagggccagaaggaagggcgaga

SEQ ID NO: 133 - DNA, 20 bp, Artificial Sequence
  Antisense oligomer
  misc_feature (1)..(20): n = t or u
gcncagcagggaggcgggag

SEQ ID NO: 134 - DNA, 20 bp, Artificial Sequence
  Antisense oligomer
  misc_feature (1)..(20): n = t or u
ggcncncaaagcagcncnga

SEQ ID NO: 135 - DNA, 25 bp, Artificial Sequence
  Antisense oligomer
  misc_feature (1)..(25): n = t or u
gacancaaccgcggcnggcacngca

SEQ ID NO: 136 - DNA, 25 bp, Artificial Sequence
  Antisense oligomer
  misc_feature (1)..(25): n = t or u
gggnaaggnggccagggngggngnn

SEQ ID NO: 137 - DNA, 19 bp, Artificial Sequence
  Antisense oligomer
  misc_feature (1)..(19): n = t or u
gcccngcngncnagacngg

SEQ ID NO: 138 - DNA, 20 bp, Artificial Sequence
  Antisense oligomer
gagagggccagaaggaaggg

SEQ ID NO: 139 - DNA, 17 bp, Artificial Sequence
  Antisense oligomer
  misc_feature (1)..(17): n = t or u
cccgccccngcccngcc

SEQ ID NO: 140 - DNA, 18 bp, Artificial Sequence
  Antisense oligomer
  misc_feature (1)..(18): n = t or u
nggccgccgcccccgccc

SEQ ID NO: 141 - DNA, 20 bp, Artificial Sequence
  Antisense oligomer
  misc_feature (1)..(20): n = t or u
ngnccacgcgcacccncngc

SEQ ID NO: 142 - DNA, 20 bp, Artificial Sequence
  Antisense oligomer
  misc_feature (1)..(20): n = t or u
gngaggngcgngggngncga

SEQ ID NO: 143 - DNA, 20 bp, Artificial Sequence
  Antisense oligomer
  misc_feature (1)..(20): n = t or u
gcaacangcaccccacccnn

SEQ ID NO: 144 - DNA, 20 bp, Artificial Sequence
  Antisense oligomer
  misc_feature (1)..(20): n = t or u
agggcccagcacacagnggn

SEQ ID NO: 145 - DNA, 20 bp, Artificial Sequence
  Antisense oligomer
  misc_feature (1)..(20): n = t or u
ncacaccnccgcncccagca

SEQ ID NO: 146 - DNA, 18 bp, Artificial Sequence
  Antisense oligomer
  misc_feature (1)..(18): n = t or u
ggcgcngccanngncngc

SEQ ID NO: 147 - DNA, 20 bp, Artificial Sequence
  Antisense oligomer
  misc_feature (1)..(20): n = t or u
gngnccccacngcnccccga

SEQ ID NO: 148 - DNA, 20 bp, Artificial Sequence
  Antisense oligomer
  misc_feature (1)..(20): n = t or u
cnggagnaccngncaccgng

SEQ ID NO: 149 - DNA, 20 bp, Artificial Sequence
  Antisense oligomer
  misc_feature (1)..(20): n = t or u
ngagccccgagcccngccnn

SEQ ID NO: 150 - DNA, 25 bp, Artificial Sequence
  Antisense oligomer
  misc_feature (1)..(25): n = t or u
ngacccaccnnnncanaaagangaa

SEQ ID NO: 151 - DNA, 25 bp, Artificial Sequence
  Antisense oligomer
  misc_feature (1)..(25): n = t or u
cncnggcagcccnacncnaccngac

SEQ ID NO: 152 - DNA, 27 bp, Artificial Sequence
  Antisense oligomer
  misc_feature (1)..(27): n = t or u
cnagnanaaanacancccaaannnngc

SEQ ID NO: 153 - DNA, 25 bp, Artificial Sequence
  Antisense oligomer
  misc_feature (1)..(25): n = t or u
ggcccnggncngcnggcncccngcn

SEQ ID NO: 154 - DNA, 25 bp, Artificial Sequence
  Antisense oligomer
  misc_feature (1)..(25): n = t or u
gcncccngcagccccngcnnngcag

SEQ ID NO: 155 - DNA, 20 bp, Artificial Sequence
  Antisense oligomer
  misc_feature (1)..(20): n = t or u
gcggggcagacgncaggngn

SEQ ID NO: 156 - DNA, 20 bp, Artificial Sequence
  Antisense oligomer
  misc_feature (1)..(20): n = t or u
cagcgcggggcagacgncag

SEQ ID NO: 157 - DNA, 20 bp, Artificial Sequence
  Antisense oligomer
ccggcagcgcggggcagacg

SEQ ID NO: 158 - DNA, 20 bp, Artificial Sequence
  Antisense oligomer
ccgccggcagcgcggggcag

SEQ ID NO: 159 - DNA, 20 bp, Artificial Sequence
  Antisense oligomer
  misc_feature (1)..(20): n = t or u
gangnnaccgccggcagcgc

SEQ ID NO: 160 - DNA, 20 bp, Artificial Sequence
  Antisense oligomer
  misc_feature (1)..(20): n = t or u
cngggangnnaccgccggca

SEQ ID NO: 161 - DNA, 20 bp, Artificial Sequence
  Antisense oligomer
  misc_feature (1)..(20): n = t or u
gcnncngggangnnaccgcc

SEQ ID NO: 162 - DNA, 20 bp, Artificial Sequence
  Antisense oligomer
  misc_feature (1)..(20): n = t or u
nggcaacncgnangnccnna

SEQ ID NO: 163 - DNA, 20 bp, Artificial Sequence
  Antisense oligomer
  misc_feature (1)..(20): n = t or u
anncnggcaacncgnangnc

SEQ ID NO: 164 - DNA, 20 bp, Artificial Sequence
  Antisense oligomer
  misc_feature (1)..(20): n = t or u
aagnganncnggcaacncgn

SEQ ID NO: 165 - DNA, 20 bp, Artificial Sequence
  Antisense oligomer
  misc_feature (1)..(20): n = t or u
ngggngncagcggaagngan

SEQ ID NO: 166 - DNA, 20 bp, Artificial Sequence
  Antisense oligomer
  misc_feature (1)..(20): n = t or u
gnccacngggngncagcgga

SEQ ID NO: 167 - DNA, 20 bp, Artificial Sequence
  Antisense oligomer
  misc_feature (1)..(20): n = t or u
gcnnggnccacngggngnca

SEQ ID NO: 168 - DNA, 20 bp, Artificial Sequence
  Antisense oligomer
  misc_feature (1)..(20): n = t or u
ccccacnncngcanaaaggn

SEQ ID NO: 169 - DNA, 20 bp, Artificial Sequence
  Antisense oligomer
  misc_feature (1)..(20): n = t or u
ggagccccacnncngcanaa

SEQ ID NO: 170 - DNA, 20 bp, Artificial Sequence
  Antisense oligomer
  misc_feature (1)..(20): n = t or u
gcngggagccccacnncngc

SEQ ID NO: 171 - DNA, 20 bp, Artificial Sequence
  Antisense oligomer
  misc_feature (1)..(20): n = t or u
ccacgccnggcngggagccc

SEQ ID NO: 172 - DNA, 20 bp, Artificial Sequence
  Antisense oligomer
  misc_feature (1)..(20): n = t or u
nccgaagngcngggannnca

SEQ ID NO: 173 - DNA, 20 bp, Artificial Sequence
  Antisense oligomer
  misc_feature (1)..(20): n = t or u
nccaccccccnnggccnncc

SEQ ID NO: 174 - DNA, 20 bp, Artificial Sequence
  Antisense oligomer
  misc_feature (1)..(20): n = t or u
nganccaccccccnnggccn

SEQ ID NO: 175 - DNA, 20 bp, Artificial Sequence
  Antisense oligomer
  misc_feature (1)..(20): n = t or u
ncaagnganccaccccccnn

SEQ ID NO: 176 - DNA, 20 bp, Artificial Sequence
  Antisense oligomer
  misc_feature (1)..(20): n = t or u
gaacnccngagcncaagnga

SEQ ID NO: 177 - DNA, 20 bp, Artificial Sequence
  Antisense oligomer
  misc_feature (1)..(20): n = t or u
ncncgaacnccngagcncaa

SEQ ID NO: 178 - DNA, 20 bp, Artificial Sequence
  Antisense oligomer
  misc_feature (1)..(20): n = t or u
ccaggcnggncncgaacncc

SEQ ID NO: 179 - DNA, 20 bp, Artificial Sequence
  Antisense oligomer
  misc_feature (1)..(20): n = t or u
nnngccangnnacccaggcn

SEQ ID NO: 180 - DNA, 20 bp, Artificial Sequence
  Antisense oligomer
  misc_feature (1)..(20): n = t or u
acgggannnngccangnnac

SEQ ID NO: 181 - DNA, 20 bp, Artificial Sequence
  Antisense oligomer
  misc_feature (1)..(20): n = t or u
nagagacgggannnngccan

SEQ ID NO: 182 - DNA, 20 bp, Artificial Sequence
  Antisense oligomer
  misc_feature (1)..(20): n = t or u
nnnngnagagacgggannnn

SEQ ID NO: 183 - DNA, 20 bp, Artificial Sequence
  Antisense oligomer
  misc_feature (1)..(20): n = t or u
ncngnannnnngnagagacg

SEQ ID NO: 184 - DNA, 20 bp, Artificial Sequence
  Antisense oligomer
  misc_feature (1)..(20): n = t or u
annnncngnannnnngnaga

SEQ ID NO: 185 - DNA, 20 bp, Artificial Sequence
  Antisense oligomer
  misc_feature (1)..(20): n = t or u
gcnaannnncngnannnnng

SEQ ID NO: 186 - DNA, 20 bp, Artificial Sequence
  Antisense oligomer
  misc_feature (10)..(20): n = t or u
ccgccgcccccgccccngcc

SEQ ID NO: 187 - DNA, 20 bp, Artificial Sequence
  Antisense oligomer
  misc_feature (1)..(20): n = t or u
nggccgccgcccccgccccn

SEQ ID NO: 188 - DNA, 20 bp, Artificial Sequence
  Antisense oligomer
  misc_feature (1)..(20): n = t or u
cngcccnggccgccgccccc

SEQ ID NO: 189 - DNA, 20 bp, Artificial Sequence
  Antisense oligomer
  misc_feature (1)..(20): n = t or u
cacccncngcccnggccgcc

SEQ ID NO: 190 - DNA, 20 bp, Artificial Sequence
  Antisense oligomer
  misc_feature (1)..(20): n = t or u
gcgcacccncngcccnggcc

SEQ ID NO: 191 - DNA, 20 bp, Artificial Sequence
  Antisense oligomer
  misc_feature (1)..(20): n = t or u
ngncgangnccacgcgcacc

SEQ ID NO: 192 - DNA, 20 bp, Artificial Sequence
  Antisense oligomer
  misc_feature (1)..(20): n = t or u
ngcgngggngncgangncca

SEQ ID NO: 193 - DNA, 20 bp, Artificial Sequence
  Antisense oligomer
  misc_feature (1)..(20): n = t or u
gcaccccacccnngngaggn

SEQ ID NO: 194 - DNA, 20 bp, Artificial Sequence
  Antisense oligomer
  misc_feature (1)..(20): n = t or u
aacangcaccccacccnngn

SEQ ID NO: 195 - DNA, 20 bp, Artificial Sequence
  Antisense oligomer
  misc_feature (1)..(20): n = t or u
aggaggaggacgccnccccc

SEQ ID NO: 196 - DNA, 20 bp, Artificial Sequence
  Antisense oligomer
  misc_feature (1)..(20): n = t or u
cncancngcagagccaggag

SEQ ID NO: 197 - DNA, 20 bp, Artificial Sequence
  Antisense oligomer
  misc_feature (1)..(20): n = t or u
gcncccncancngcagagcc

SEQ ID NO: 198 - DNA, 20 bp, Artificial Sequence
  Antisense oligomer
  misc_feature (1)..(20): n = t or u
ncggcncccncancngcaga

SEQ ID NO: 199 - DNA, 20 bp, Artificial Sequence
  Antisense oligomer
  misc_feature (1)..(20): n = t or u
gccncggcncccncancngc

SEQ ID NO: 200 - DNA, 20 bp, Artificial Sequence
  Antisense oligomer
  misc_feature (1)..(20): n = t or u
nncngggangnnaccgccgg

SEQ ID NO: 201 - DNA, 20 bp, Artificial Sequence
  Antisense oligomer
  misc_feature (1)..(20): n = t or u
cnncngggangnnaccgccg

SEQ ID NO: 202 - DNA, 20 bp, Artificial Sequence
  Antisense oligomer
  misc_feature (1)..(20): n = t or u
cgcnncngggangnnaccgc

SEQ ID NO: 203 - DNA, 20 bp, Artificial Sequence
  Antisense oligomer
  misc_feature (1)..(20): n = t or u
ccgcnncngggangnnaccg

SEQ ID NO: 204 - DNA, 20 bp, Artificial Sequence
  Antisense oligomer
  misc_feature (1)..(20): n = t or u
acccgcnncngggangnnac

SEQ ID NO: 205 - DNA, 20 bp, Artificial Sequence
  Antisense oligomer
  misc_feature (1)..(20): n = t or u
ncaaacccgcnncngggang

SEQ ID NO: 206 - DNA, 20 bp, Artificial Sequence
  Antisense oligomer
  misc_feature (1)..(20): n = t or u
acgnncaaacccgcnncngg

SEQ ID NO: 207 - DNA, 20 bp, Artificial Sequence
  Antisense oligomer
  misc_feature (1)..(20): n = t or u
gggcncncaaagcagcncng

SEQ ID NO: 208 - DNA, 20 bp, Artificial Sequence
  Antisense oligomer
  misc_feature (1)..(20): n = t or u
ggggcncncaaagcagcncn

SEQ ID NO: 209 - DNA, 20 bp, Artificial Sequence
  Antisense oligomer
  misc_feature (1)..(20): n = t or u
acggggcncncaaagcagcn

SEQ ID NO: 210 - DNA, 20 bp, Artificial Sequence
  Antisense oligomer
  misc_feature (1)..(20): n = t or u
ncacggggcncncaaagcag

SEQ ID NO: 211 - DNA, 20 bp, Artificial Sequence
  Antisense oligomer
  misc_feature (1)..(20): n = t or u
gcncncaaagcagcncngag

SEQ ID NO: 212 - DNA, 20 bp, Artificial Sequence
  Antisense oligomer
  misc_feature (1)..(20): n = t or u
cncncaaagcagcncngaga

SEQ ID NO: 213 - DNA, 20 bp, Artificial Sequence
  Antisense oligomer
  misc_feature (1)..(20): n = t or u
cncaaagcagcncngagaca

SEQ ID NO: 214 - DNA, 20 bp, Artificial Sequence
  Antisense oligomer
  misc_feature (1)..(20): n = t or u
caaagcagcncngagacanc

SEQ ID NO: 215 - DNA, 20 bp, Artificial Sequence
  Antisense oligomer
  misc_feature (1)..(20): n = t or u
aagcagcncngagacancaa

SEQ ID NO: 216 - DNA, 25 bp, Artificial Sequence
  Antisense oligomer
  misc_feature (1)..(25): n = t or u
gcccnggncngcnggcncccngcng

SEQ ID NO: 217 - DNA, 25 bp, Artificial Sequence
  Antisense oligomer
  misc_feature (1)..(25): n = t or u
cccnggncngcnggcncccngcngg

SEQ ID NO: 218 - DNA, 25 bp, Artificial Sequence
  Antisense oligomer
  misc_feature (1)..(25): n = t or u
ccnggncngcnggcncccngcnggn

SEQ ID NO: 219 - DNA, 25 bp, Artificial Sequence
  Antisense oligomer
  misc_feature (1)..(25): n = t or u
cnggncngcnggcncccngcnggng

SEQ ID NO: 220 - DNA, 25 bp, Artificial Sequence
  Antisense oligomer
  misc_feature (1)..(25): n = t or u
nggncngcnggcncccngcnggnga

SEQ ID NO: 221 - DNA, 25 bp, Artificial Sequence
  Antisense oligomer
  misc_feature (1)..(25): n = t or u
ggncngcnggcncccngcnggngag

SEQ ID NO: 222 - DNA, 25 bp, Artificial Sequence
  Antisense oligomer
  misc_feature (1)..(25): n = t or u
gncngcnggcncccngcnggngagc

SEQ ID NO: 223 - DNA, 25 bp, Artificial Sequence
  Antisense oligomer
  misc_feature (1)..(25): n = t or u
ncngcnggcncccngcnggngagcn

SEQ ID NO: 224 - DNA, 25 bp, Artificial Sequence
  Antisense oligomer
  misc_feature (1)..(25): n = t or u
gggcccnggncngcnggcncccngc

SEQ ID NO: 225 - DNA, 25 bp, Artificial Sequence
  Antisense oligomer
  misc_feature (1)..(25): n = t or u
ggggcccnggncngcnggcncccng

SEQ ID NO: 226 - DNA, 25 bp, Artificial Sequence
  Antisense oligomer
  misc_feature (1)..(25): n = t or u
cggggcccnggncngcnggcncccn

SEQ ID NO: 227 - DNA, 25 bp, Artificial Sequence
  Antisense oligomer
  misc_feature (1)..(25): n = t or u
ccggggcccnggncngcnggcnccc

SEQ ID NO: 228 - DNA, 25 bp, Artificial Sequence
  Antisense oligomer
  misc_feature (1)..(25): n = t or u
cccggggcccnggncngcnggcncc

SEQ ID NO: 229 - DNA, 25 bp, Artificial Sequence
  Antisense oligomer
  misc_feature (1)..(25): n = t or u
ncccggggcccnggncngcnggcnc

SEQ ID NO: 230 - DNA, 25 bp, Artificial Sequence
  Antisense oligomer
  misc_feature (1)..(25): n = t or u
ancccggggcccnggncngcnggcn

SEQ ID NO: 231 - DNA, 25 bp, Artificial Sequence
  Antisense oligomer
  misc_feature (1)..(25): n = t or u
cancccggggcccnggncngcnggc

SEQ ID NO: 232 - DNA, 27 bp, Artificial Sequence
  Antisense oligomer
  misc_feature (1)..(27): n = t or u
ncngcccnggccgccgcccccgccccn

SEQ ID NO: 233 - DNA, 25 bp, Artificial Sequence
  Antisense oligomer
  misc_feature (1)..(25): n = t or u
ngaggngcgngggngncgangncca

SEQ ID NO: 234 - DNA, 25 bp, Artificial Sequence
  Antisense oligomer
  misc_feature (1)..(25): n = t or u
gaggngcgngggngncgangnccac

SEQ ID NO: 235 - DNA, 25 bp, Artificial Sequence
  Antisense oligomer
  misc_feature (1)..(25): n = t or u
aggngcgngggngncgangnccacg

SEQ ID NO: 236 - DNA, 25 bp, Artificial Sequence
  Antisense oligomer
  misc_feature (1)..(25): n = t or u
gcgcgnggacancgacacccacgca

SEQ ID NO: 237 - DNA, 25 bp, Artificial Sequence
  Antisense oligomer
  misc_feature (1)..(25): n = t or u
ngngagggcgcgnggacancgacac

SEQ ID NO: 238 - DNA, 25 bp, Artificial Sequence
  Antisense oligomer
  misc_feature (1)..(25): n = t or u
nngngagggcgcgnggacancgaca

SEQ ID NO: 239 - DNA, 25 bp, Artificial Sequence
  Antisense oligomer
  misc_feature (1)..(25): n = t or u
cnngngagggcgcgnggacancgac

SEQ ID NO: 240 - DNA, 18 bp, Artificial Sequence
  Antisense oligomer
gagagggccagaaggaag

SEQ ID NO: 241 - DNA, 18 bp, Artificial Sequence
  Antisense oligomer
gagggccagaaggaaggg

SEQ ID NO: 242 - DNA, 18 bp, Artificial Sequence
  Antisense oligomer
gggccagaaggaagggcg

SEQ ID NO: 243 - DNA, 18 bp, Artificial Sequence
  Antisense oligomer
gggagagggccagaagga

SEQ ID NO: 244 - DNA, 20 bp, Artificial Sequence
  Antisense oligomer
agagggccagaaggaagggc

SEQ ID NO: 245 - DNA, 20 bp, Artificial Sequence
  Antisense oligomer
gagggccagaaggaagggcg

SEQ ID NO: 246 - DNA, 20 bp, Artificial Sequence
  Antisense oligomer
agggccagaaggaagggcga

SEQ ID NO: 247 - DNA, 20 bp, Artificial Sequence
  Antisense oligomer
gggccagaaggaagggcgag

SEQ ID NO: 248 - DNA, 20 bp, Artificial Sequence
  Antisense oligomer
ggccagaaggaagggcgaga

SEQ ID NO: 249 - DNA, 20 bp, Artificial Sequence
  Antisense oligomer
gccagaaggaagggcgagaa

SEQ ID NO: 250 - DNA, 22 bp, Artificial Sequence
  Antisense oligomer
gggagagggccagaaggaaggg

SEQ ID NO: 251 - DNA, 25 bp, Artificial Sequence
  Antisense oligomer
  misc_feature (1)..(25): n = t or u
cnggggagagggccagaaggaaggg

SEQ ID NO: 252 - DNA, 22 bp, Artificial Sequence
  Antisense oligomer
gagagggccagaaggaagggcg

SEQ ID NO: 253 - DNA, 25 bp, Artificial Sequence
  Antisense oligomer
gagagggccagaaggaagggcgaga

SEQ ID NO: 254 - DNA, 20 bp, Artificial Sequence
  Antisense oligomer
gaaggaagggcgagaaaagc

SEQ ID NO: 255 - DNA, 20 bp, Artificial Sequence
  Antisense oligomer
  misc_feature (1)..(20): n = t or u
gcagaaaagcnccagcaggg

SEQ ID NO: 256 - DNA, 25 bp, Artificial Sequence
  Antisense oligomer
aagctccagcaggggagtgcagagc

SEQ ID NO: 257 - DNA, 25 bp, Artificial Sequence
  Antisense oligomer
aaaagctccagcaggggagtgcaga

SEQ ID NO: 258 - DNA, 25 bp, Artificial Sequence
  Antisense oligomer
agaaaagctccagcaggggagtgca

SEQ ID NO: 259 - DNA, 25 bp, Artificial Sequence
  Antisense oligomer
cgagaaaagctccagcaggggagtg

SEQ ID NO: 260 - DNA, 25 bp, Artificial Sequence
  Antisense oligomer
ggcgagaaaagctccagcaggggag

SEQ ID NO: 261 - DNA, 25 bp, Artificial Sequence
  Antisense oligomer
agggcgagaaaagctccagcagggg

SEQ ID NO: 262 - DNA, 25 bp, Artificial Sequence
  Antisense oligomer
gaagggcgagaaaagctccagcagg

SEQ ID NO: 263 - DNA, 25 bp, Artificial Sequence
  Antisense oligomer
aggaagggcgagaaaagctccagca

SEQ ID NO: 264 - DNA, 25 bp, Artificial Sequence
  Antisense oligomer
gaaggaagggcgagaaaagctccag

SEQ ID NO: 265 - DNA, 25 bp, Artificial Sequence
  Antisense oligomer
cagaaggaagggcgagaaaagctcc

SEQ ID NO: 266 - DNA, 25 bp, Artificial Sequence
  Antisense oligomer
gccagaaggaagggcgagaaaagct

SEQ ID NO: 267 - DNA, 25 bp, Artificial Sequence
  Antisense oligomer
gggccagaaggaagggcgagaaaag

SEQ ID NO: 268 - DNA, 25 bp, Artificial Sequence
  Antisense oligomer
gagggccagaaggaagggcgagaaa

SEQ ID NO: 269 - DNA, 25 bp, Artificial Sequence
  Antisense oligomer
gagagggccagaaggaagggcgaga

SEQ ID NO: 270 - DNA, 20 bp, Artificial Sequence
  Antisense oligomer
agggccagaaggaagggcga

SEQ ID NO: 271 - DNA, 20 bp, Artificial Sequence
  Antisense oligomer
gagagggccagaaggaaggg

SEQ ID NO: 272 - DNA, 25 bp, Artificial Sequence
  Antisense oligomer
ctggggagagggccagaaggaaggg

SEQ ID NO: 273 - DNA, 18 bp, Artificial Sequence
  Antisense oligomer
gagagggccagaaggaag

SEQ ID NO: 274 - DNA, 18 bp, Artificial Sequence
  Antisense oligomer
gggagagggccagaagga

SEQ ID NO: 275 - DNA, 25 bp, Artificial Sequence
  Antisense oligomer
ggtctgctggctccctgctggtgag

SEQ ID NO: 276 - DNA, 25 bp, Artificial Sequence
  Antisense oligomer
cactcacggggctctcaaagcagct

SEQ ID NO: 277 - DNA, 25 bp, Artificial Sequence
  Antisense oligomer
tctgggatgttaccgccggcagcgc

SEQ ID NO: 278 - DNA, 20 bp, Artificial Sequence
  Antisense oligomer
tttgccatgttacccaggct

SEQ ID NO: 279 - DNA, 25 bp, Artificial Sequence
  Antisense oligomer
actcacggggctctcaaagcagctc

SEQ ID NO: 280 - DNA, 25 bp, Artificial Sequence
  Antisense oligomer
gcactcacggggctctcaaagcagc

SEQ ID NO: 281 - DNA, 25 bp, Artificial Sequence
  Antisense oligomer
cggggctctcaaagcagctctgaga

SEQ ID NO: 282 - DNA, 25 bp, Artificial Sequence
  Antisense oligomer
acggggctctcaaagcagctctgag

SEQ ID NO: 283 - DNA, 25 bp, Artificial Sequence
  Antisense oligomer
cacggggctctcaaagcagctctga

SEQ ID NO: 284 - DNA, 25 bp, Artificial Sequence
  Antisense oligomer
tcacggggctctcaaagcagctctg

SEQ ID NO: 285 - DNA, 25 bp, Artificial Sequence
  Antisense oligomer
ctcacggggctctcaaagcagctct

SEQ ID NO: 286 - DNA, 25 bp, Artificial Sequence
  Antisense oligomer
ggcactcacggggctctcaaagcag

SEQ ID NO: 287 - DNA, 25 bp, Artificial Sequence
  Antisense oligomer
cggcactcacggggctctcaaagca

SEQ ID NO: 288 - DNA, 25 bp, Artificial Sequence
  Antisense oligomer
gcggcactcacggggctctcaaagc

SEQ ID NO: 289 - skipped

SEQ ID NO: 290 - DNA, 25 bp, Artificial Sequence
  Antisense oligomer
ggcggcactcacggggctctcaaag

SEQ ID NO: 291 - DNA, 25 bp, Artificial Sequence
  Antisense oligomer
gggcggcactcacggggctctcaaa

SEQ ID NO: 292 - DNA, 25 bp, Artificial Sequence
  Antisense oligomer
ggggcggcactcacggggctctcaa

SEQ ID NO: 293 - DNA, 25 bp, Artificial Sequence
  Antisense oligomer
aggggcggcactcacggggctctca

SEQ ID NO: 294 - DNA, 25 bp, Artificial Sequence
  Antisense oligomer
gaggggcggcactcacggggctctc

SEQ ID NO: 295 - DNA, 20 bp, Artificial Sequence
  Antisense oligomer
ggctctcaaagcagctctga

SEQ ID NO: 296 - DNA, 25 bp, Artificial Sequence
  Antisense oligomer
  misc_feature (1)..(25): n = t or u
aagcnccagcaggggagngcagagc

SEQ ID NO: 297 - DNA, 25 bp, Artificial Sequence
  Antisense oligomer
  misc_feature (1)..(25): n = t or u
aaaagcnccagcaggggagngcaga

SEQ ID NO: 298 - DNA, 25 bp, Artificial Sequence
  Antisense oligomer
  misc_feature (1)..(25): n = t or u
agaaaagcnccagcaggggagngca

SEQ ID NO: 299 - DNA, 25 bp, Artificial Sequence
  Antisense oligomer
  misc_feature (1)..(25): n = t or u
cgagaaaagcnccagcaggggagng

SEQ ID NO: 300 - DNA, 25 bp, Artificial Sequence
  Antisense oligomer
  misc_feature (1)..(25): n = t or u
ggcgagaaaagcnccagcaggggag

SEQ ID NO: 301 - DNA, 25 bp, Artificial Sequence
  Antisense oligomer
  misc_feature (1)..(25): n = t or u
agggcgagaaaagcnccagcagggg

SEQ ID NO: 302 - DNA, 25 bp, Artificial Sequence
  Antisense oligomer
  misc_feature (1)..(25): n = t or u
gaagggcgagaaaagcnccagcagg

SEQ ID NO: 303 - DNA, 25 bp, Artificial Sequence
  Antisense oligomer
  misc_feature (1)..(25): n = t or u
aggaagggcgagaaaagcnccagca

SEQ ID NO: 304 - DNA, 25 bp, Artificial Sequence
  Antisense oligomer
  misc_feature (1)..(25): n = t or u
gaaggaagggcgagaaaagcnccag

SEQ ID NO: 305 - DNA, 25 bp, Artificial Sequence
  Antisense oligomer
  misc_feature (1)..(25): n = t or u
cagaaggaagggcgagaaaagcncc

SEQ ID NO: 306 - DNA, 25 bp, Artificial Sequence
  Antisense oligomer
  misc_feature (1)..(25): n = t or u
gccagaaggaagggcgagaaaagcn

SEQ ID NO: 307 - DNA, 25 bp, Artificial Sequence
  Antisense oligomer
gggccagaaggaagggcgagaaaag

SEQ ID NO: 308 - DNA, 25 bp, Artificial Sequence
  Antisense oligomer
gagggccagaaggaagggcgagaaa

SEQ ID NO: 309 - DNA, 25 bp, Artificial Sequence
  Antisense oligomer
gagagggccagaaggaagggcgaga

SEQ ID NO: 310 - DNA, 20 bp, Artificial Sequence
  Antisense oligomer
agggccagaaggaagggcga

SEQ ID NO: 311 - DNA, 20 bp, Artificial Sequence
  Antisense oligomer
gagagggccagaaggaaggg

SEQ ID NO: 312 - DNA, 25 bp, Artificial Sequence
  Antisense oligomer
  misc_feature (1)..(25): n = t or u
cnggggagagggccagaaggaaggg

SEQ ID NO: 313 - DNA, 18 bp, Artificial Sequence
  Antisense oligomer
gagagggccagaaggaag

SEQ ID NO: 314 - DNA, 18 bp, Artificial Sequence
  Antisense oligomer
gggagagggccagaagga

SEQ ID NO: 315 - DNA, 25 bp, Artificial Sequence
  Antisense oligomer
  misc_feature (1)..(25): n = t or u
ggncngcnggcncccngcnggngag

SEQ ID NO: 316 - DNA, 25 bp, Artificial Sequence
  Antisense oligomer
  misc_feature (1)..(25): n = t or u
cacncacggggcncncaaagcagcn

SEQ ID NO: 317 - DNA, 25 bp, Artificial Sequence
  Antisense oligomer
  misc_feature (1)..(25): n = t or u
ncngggangnnaccgccggcagcgc

SEQ ID NO: 318 - DNA, 20 bp, Artificial Sequence
  Antisense oligomer
  misc_feature (1)..(20): n = t or u
nnngccangnnacccaggcn

SEQ ID NO: 319 - DNA, 25 bp, Artificial Sequence
  Antisense oligomer
  misc_feature (1)..(25): n = t or u
acncacggggcncncaaagcagcnc

SEQ ID NO: 320 - DNA, 25 bp, Artificial Sequence
  Antisense oligomer
  misc_feature (1)..(25): n = t or u
gcacncacggggcncncaaagcagc

SEQ ID NO: 321 - DNA, 25 bp, Artificial Sequence
  Antisense oligomer
  misc_feature (1)..(25): n = t or u
cggggcncncaaagcagcncngaga

SEQ ID NO: 322 - DNA, 25 bp, Artificial Sequence
  Antisense oligomer
  misc_feature (1)..(25): n = t or u
acggggcncncaaagcagcncngag

SEQ ID NO: 323 - DNA, 25 bp, Artificial Sequence
  Antisense oligomer
  misc_feature (1)..(25): n = t or u
cacggggcncncaaagcagcncnga

SEQ ID NO: 324 - DNA, 25 bp, Artificial Sequence
  Antisense oligomer
  misc_feature (1)..(25): n = t or u
ncacggggcncncaaagcagcncng

SEQ ID NO: 325 - DNA, 25 bp, Artificial Sequence
  Antisense oligomer
  misc_feature (1)..(25): n = t or u
cncacggggcncncaaagcagcncn

SEQ ID NO: 326 - DNA, 25 bp, Artificial Sequence
  Antisense oligomer
  misc_feature (1)..(25): n = t or u
ggcacncacggggcncncaaagcag

SEQ ID NO: 327 - DNA, 25 bp, Artificial Sequence
  Antisense oligomer
  misc_feature (1)..(25): n = t or u
cggcacncacggggcncncaaagca

SEQ ID NO: 328 - DNA, 25 bp, Artificial Sequence
  Antisense oligomer
  misc_feature (1)..(25): n = t or u
gcggcacncacggggcncncaaagc

SEQ ID NO: 329 - DNA, 25 bp, Artificial Sequence
  Antisense oligomer
  misc_feature (1)..(25): n = t or u
ggcggcacncacggggcncncaaag

SEQ ID NO: 330 - DNA, 25 bp, Artificial Sequence
  Antisense oligomer
  misc_feature (1)..(25): n = t or u
gggcggcacncacggggcncncaaa

SEQ ID NO: 331 - DNA, 25 bp, Artificial Sequence
  Antisense oligomer
  misc_feature (1)..(25): n = t or u
ggggcggcacncacggggcncncaa

SEQ ID NO: 332 - DNA, 25 bp, Artificial Sequence
  Antisense oligomer
  misc_feature (1)..(25): n = t or u
aggggcggcacncacggggcncnca

SEQ ID NO: 333 - DNA, 25 bp, Artificial Sequence
  Antisense oligomer
  misc_feature (1)..(25): n = t or u
gaggggcggcacncacggggcncnc

SEQ ID NO: 334 - DNA, 20 bp, Artificial Sequence
  Antisense oligomer
  misc_feature (1)..(20): n = t or u
ggcncncaaagcagcncnga

SEQ ID NO: 335 - DNA, 25 bp, Artificial Sequence
  Antisense oligomer
  misc_feature (1)..(25): n = t or u
nncngggangnnaccgccggcagcg

SEQ ID NO: 336 - DNA, 25 bp, Artificial Sequence
  Antisense oligomer
  misc_feature (1)..(25): n = t or u
cnncngggangnnaccgccggcagc

SEQ ID NO: 337 - DNA, 25 bp, Artificial Sequence
  Antisense oligomer
  misc_feature (1)..(25): n = t or u
gcnncngggangnnaccgccggcag

SEQ ID NO: 338 - DNA, 25 bp, Artificial Sequence
  Antisense oligomer
  misc_feature (1)..(25): n = t or u
cgcnncngggangnnaccgccggca

SEQ ID NO: 339 - DNA, 25 bp, Artificial Sequence
  Antisense oligomer
  misc_feature (1)..(25): n = t or u
ccgcnncngggangnnaccgccggc

SEQ ID NO: 340 - DNA, 25 bp, Artificial Sequence
  Antisense oligomer
  misc_feature (1)..(25): n = t or u
cccgcnncngggangnnaccgccgg

SEQ ID NO: 341 - DNA, 25 bp, Artificial Sequence
  Antisense oligomer
  misc_feature (1)..(25): n = t or u
acccgcnncngggangnnaccgccg

SEQ ID NO: 342 - DNA, 25 bp, Artificial Sequence
  Antisense oligomer
  misc_feature (1)..(25): n = t or u
aacccgcnncngggangnnaccgcc

SEQ ID NO: 343 - DNA, 25 bp, Artificial Sequence
  Antisense oligomer
ttctgggatgttaccgccggcagcg

SEQ ID NO: 344 - DNA, 25 bp, Artificial Sequence
  Antisense oligomer
cttctgggatgttaccgccggcagc

SEQ ID NO: 345 - DNA, 25 bp, Artificial Sequence
  Antisense oligomer
gcttctgggatgttaccgccggcag

SEQ ID NO: 346 - DNA, 25 bp, Artificial Sequence
  Antisense oligomer
cgcttctgggatgttaccgccggca

SEQ ID NO: 347 - DNA, 25 bp, Artificial Sequence
  Antisense oligomer
ccgcttctgggatgttaccgccggc

SEQ ID NO: 348 - DNA, 25 bp, Artificial Sequence
  Antisense oligomer
cccgcttctgggatgttaccgccgg

SEQ ID NO: 349 - DNA, 25 bp, Artificial Sequence
  Antisense oligomer
acccgcttctgggatgttaccgccg

SEQ ID NO: 350 - DNA, 25 bp, Artificial Sequence
  Antisense oligomer
aacccgcttctgggatgttaccgcc

SEQ ID NO: 351 - DNA, 25 bp, Artificial Sequence
  Antisense oligomer
  misc_feature (1)..(25): n = t or u
cnngngagggcgcgnggacancgac

(19) United States
(12) Patent Application Publication
(10) Pub. No.: US 2018/0216111 A1
(43) Pub. Date: Aug. 2, 2018

(54) ANTISENSE-INDUCED EXON2 INCLUSION IN ACID ALPHA-GLUCOSIDASE

(71) Applicants: Sarepta Therapeutics, Inc., Cambridge, MA (US); Murdoch University, Murdoch (AU)

(72) Inventors: Stephen Donald WILTON, Murdoch (AU); Sue FLETCHER, Murdoch (AU); Gunnar James HANSON, Cambridge, MA (US); Richard Keith BESTWICK, Cambridge, MA (US); Frederick J. SCHNELL, Corvallis, OR (US)

(21) Appl. No.: 15/553,911

(22) PCT Filed: Feb. 29, 2016
(86) PCT No.: PCT/US2016/020127

§ 371 Date: Aug. 25, 2017

Related U.S. Application Data
(60) Provisional application No. 62/300,635, filed on Feb. 26, 2016.
(60) Provisional application No. 62/234,263, filed on Sep. 29, 2015.
(60) Provisional application No. 62/126,346, filed on Feb. 27, 2015.

(51) Int. Cl. C12N 15/113

(52) U.S. Cl. CPC C12N 15/1137; C12Y 302/0102; C12N 2320/33; C12N 2310/3233; C12N 2310/11

(57) ABSTRACT

The present disclosure relates to antisense oligomers and related compositions and methods for inducing exon inclusion as a treatment for glycogen storage disease type II (GSD-II) (also known as Pompe disease, glycogenosis II, acid maltase deficiency (AMD), acid alpha-glucosidase deficiency, and lysosomal alpha-glucosidase deficiency), and more specifically relates to inducing inclusion of exon 2 and thereby restoring levels of enzymatically active acid alpha-glucosidase (GAA) protein encoded by the GAA gene.

### BACKGROUND

### Field of the Disclosure

The present disclosure relates to antisense oligomers and related compositions and methods for inducing exon inclusion as a treatment for glycogen storage disease type II (GSD-II) (also known as Pompe disease, glycogenosis II, acid maltase deficiency (AMD), acid alpha-glucosidase deficiency, and lysosomal alpha-glucosidase deficiency), and more specifically relates to inducing inclusion of exon 2 and thereby restoring levels of enzymatically active acid alpha-glucosidase (GAA) protein encoded by the GAA gene.

### Description of the Related Art

Alternative splicing increases the coding potential of the human genome by producing multiple proteins from a single gene. Inappropriate alternative splicing is also associated with a growing number of human diseases.

GSD-II is an inherited autosomal recessive lysosomal storage disorder caused by deficiency of an enzyme called acid alpha-glucosidase (GAA). The role of GAA within the body is to break down glycogen. Reduced or absent levels of GAA activity leads to the accumulation of glycogen in the affected tissues, including the heart, skeletal muscles (including those involved with breathing), liver, and nervous system. This accumulation of glycogen is believed to cause progressive muscle weakness and respiratory insufficiency in individuals with GSD-II. GSD-II can occur in infants, toddlers, or adults, and the prognosis varies according to the time of onset and severity of symptoms. Clinically, GSD-II may manifest with a broad and continuous spectrum of severity ranging from severe (infantile) to milder late onset adult form. The patients eventually die due to respiratory insufficiency. There is a good correlation between the severity of the disease and the residual acid alpha-glucosidase activity, the activity being 10-20% of normal in late onset and less than 2% in early onset forms of the disease. It is estimated that GSD-II affects approximately 5,000 to 10,000 people worldwide.

The most common mutation associated with the adult onset form of disease is IVS1-13T>G. Found in over two thirds of adult onset GSD-II patients, this mutation may confer a selective advantage in heterozygous individuals or is a very old mutation. The wide ethnic variation of adult onset GSD-II individuals with this mutation argues against a common founder.

The GAA gene consists of 20 exons spanning some 20 kb. The 3.4 kb mRNA encodes a protein with a molecular weight of approximately 105 kD. The IVS1-13T>G mutation leads to the loss of exon 2 (577 bases) which contains the initiation AUG codon.

Treatment for GSD-II has involved drug treatment strategies, dietary manipulations, and bone marrow transplantation without significant success. In recent years, enzyme replacement therapy (ERT) has provided new hope for GSD-II patients. For example, Myozyme®, a recombinant GAA protein drug, received approval for use in patients with GSD-II disease in 2006 in both the U.S. and Europe. Myozyme® depends on mannose-6-phosphates (M6P) on the surface of the GAA protein for delivery to lysosomes.

Antisense technology, used mostly for RNA down regulation, recently has been adapted to alter the splicing process. Processing the primary gene transcripts (pre-mRNA) of many genes involves the removal of introns and the precise splicing of exons where a donor splice site is joined to an acceptor splice site. Splicing is a precise process, involving the coordinated recognition of donor and acceptor splice sites, and the branch point (upstream of the acceptor splice site) with a balance of positive exon splice enhancers (predominantly located within the exon) and negative splice motifs (splice silencers are located predominantly in the introns).

Effective agents that can alter splicing of GAA pre-mRNAs are likely to be useful therapeutically for improved treatment of GSD-II.

### SUMMARY

Embodiments of present disclosure relate to antisense oligomers and related compositions and methods for increasing the levels of exon 2-containing GAA-coding mRNA in a cell, comprising contacting the cell with an antisense oligomer of sufficient length and complementarity to specifically hybridize to a region within the pre-mRNA of the GAA gene, wherein binding of the antisense oligomer to the region increases the levels of exon 2-containing GAA-coding mRNA in the cell.

Accordingly, in some embodiments, the instant disclosure relates to an antisense oligomer of 10 to 40 nucleotides or nucleotide analogs, comprising a targeting sequence of sufficient length and complementarity to specifically hybridize to a region within intron 1 (SEQ ID NO: 1), exon 2 (SEQ ID NO:2), or intron 2 (SEQ ID NO:3) of the pre-mRNA of the human acid alpha-glucosidase (GAA) gene.

In certain embodiments, the instant disclosure relates to an antisense oligomer compound, comprising:
at least one modification selected from (i) a backbone modification between at least two contiguous sugar moieties, (ii) a modified sugar moiety, or (iii) a combination of the foregoing; and a targeting sequence complementary to 10 or more contiguous nucleotides in a target region within intron 1 (SEQ ID. NO: 1), intron 2 (SEQ ID. NO: 2), or exon 2 (SEQ ID. NO: 3) of a pre-mRNA of the human acid alpha-glucosidase (GAA) gene.

In some embodiments, the antisense oligomer specifically hybridizes to a region within the intron 1, exon 2, and/or intron 2 GAA sequence(s) set forth in Table 1. In some embodiments, the antisense oligomer specifically hybridizes to an intronic splice silencer element or an exonic splice silencer element. In certain embodiments, the antisense oligomer comprises a targeting sequence set forth in Tables 2A, 2B, or 2C, a fragment of at least 10 contiguous nucleotides of a targeting sequence in Tables 2A, 2B, or 2C, or variant having at least 80% sequence identity to a targeting sequence in Tables 2A, 2B, or 2C. In specific embodiments, the antisense oligomer consists or consists essentially of a targeting sequence set forth in Tables 2A, 2B, or 2C.

In certain embodiments, the disclosure relates to an antisense oligomer compound comprising: (a) at least one modification selected from (i) one or more backbone modifications between at least two contiguous sugar moieties, (ii) one or more modified sugar moieties, or (iii) any combination of the foregoing; and (b) a targeting sequence comprising a sequence selected from the group consisting of SEQ ID Nos:4-30, 133-255, and 296-334, where X is selected from uracil (U) or thymine (T).

In certain embodiments, the disclosure relates to an antisense oligomer compound comprising: (a) at least one modification selected from (i) one or more backbone modifications between at least two contiguous sugar moieties, (ii) one or more modified sugar moieties, or (iii) any combination of the foregoing; and (b) a targeting sequence comprising a sequence selected from the group consisting essentially of SEQ ID Nos:4-30, 133-255, and 296-334, where X is selected from uracil (U) or thymine (T).

In certain embodiments, the disclosure relates to an antisense oligomer compound comprising: (a) one or more modifications selected from (i) one or more backbone modifications between at least two contiguous sugar moieties, (ii) one or more modified sugar moieties, or (iii) any combination of the foregoing; and (b) a targeting sequence comprising a sequence selected from the group consisting of SEQ ID Nos:4-30, 133-255, and 296-334, where X is selected from uracil (U) or thymine (T).

In certain embodiments, the modification is selected from one or more of phosphoramidate morpholino, phosphorodiamidate morpholino, phosphorothioate, 2′ O-methyl, peptide nucleic acid, locked nucleic acid, phosphorothioate, 2′ O-MOE, 2′-fluoro, 2′O,4′C-ethylene-bridged nucleic acid, tricyclo-DNA, tricyclo-DNA phosphorothioate nucleotide, 2′-O-[2-(N-methylcarbamoyl)ethyl], morpholino, peptide-conjugated phosphoramidate morpholino, phosphorodiamidate morpholino having a phosphorous atom with (i) a covalent bond to the nitrogen atom of a morpholino ring, and (ii) a second covalent bond to a (1,4-piperazin)-1-yl substituent or to a substituted (1,4-piperazin)-1-yl, and phosphorodiamidate morpholino having a phosphorus atom with (i) a covalent bond to the nitrogen atom of a morpholino ring and (ii) a second covalent bond to the ring nitrogen of a 4-aminopiperdin-1-yl or a derivative of 4-aminopiperdin-1-yl chemistries, or any combination of the foregoing.

In some embodiments, the antisense oligomer contains about, at least about, or no more than about 1, 2, 3, 4, 5, 6, 7, 8, 9, or 10 cationic internucleoside linkages. In certain embodiments, the antisense oligomer contains about or at least about 10%, 15%, 20%, 25%, 30%, 35%, 40%, 45%, 50%, 55%, 60%, 65%, 70%, 75%, 80%, 85%, 90%, 95%, or 100% cationic internucleoside linkages. In certain embodiments, the antisense oligomer contains about, at least about, or no more than about 1, 2, 3, 4, 5, 6, 7, 8, 9, or 10 internucleoside linkages that exhibits a pKa between about 4.5 and about 12. In some embodiments, the antisense oligomer contains about or at least about 10%, 15%, 20%, 25%, 30%, 35%, 40%, 45%, 50%, 55%, 60%, 65%, 70%, 75%, 80%, 85%, 90%, 95%, or 100% internucleoside linkages that exhibit a pKa between about 4.5 and about 12. In some embodiments, the antisense oligomer has an internucleoside linkage containing both a basic nitrogen and an alkyl, aryl, or aralkyl group. In some embodiments, the antisense oligomer comprises a morpholino.

In certain embodiments, the antisense oligomer of the disclosure is a compound of formula (I):

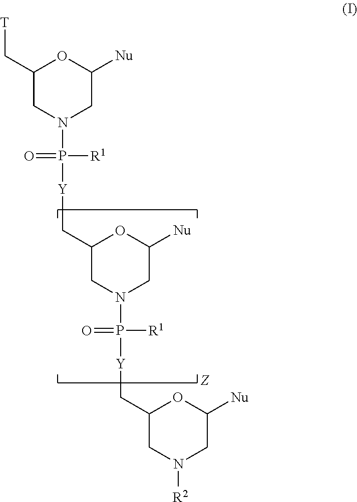

or a pharmaceutically acceptable salt thereof, wherein:
each Nu is a nucleobase which taken together form a targeting sequence;
Z is an integer from 8 to 38;
each Y is independently selected from O and —NR⁴, wherein each R⁴ is independently selected from H, C₁-C₆ alkyl, aralkyl, —C(═NH)NH₂, —C(O)(CH₂)ₙNR⁵C(═NH)NH₂, —C(O)(CH₂)₂NHC(O)(CH₂)₅NR⁵C(═NH)NH₂, and G, wherein R⁵ is selected from H and C₁-C₆ alkyl and n is an integer from 1 to 5;
T is selected from OH and a moiety of the formula:

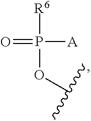

wherein:
A is selected from —OH, —N(R⁷)₂, and R¹ wherein each R⁷ is independently selected from H and C₁-C₆ alkyl, and
R⁶ is selected from OH, —N(R⁹)CH₂C(O)NH₂, and a moiety of the formula:

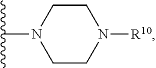

wherein:
   R⁹ is selected from H and C₁-C₆ alkyl; and
   R¹⁰ is selected from G, —C(O)—R¹¹OH, acyl, trityl, 4-methoxytrityl, —C(═NH)NH₂, —C(O)(CH₂)ₘNR²C(═NH)NH₂, and —C(O)(CH₂)₂NHC(O)(CH₂)₅NR¹²C(═NH)NH₂, wherein:
      m is an integer from 1 to 5,
      R¹¹ is of the formula —(O-alkyl)_{y}- wherein y is an integer from 3 to 10 and each of the y alkyl groups is independently selected from C₂-C₆ alkyl; and
      R¹² is selected from H and C₁-C₆ alkyl;
each instance of R¹ is independently selected from:
   —N(R¹³)₂, wherein each R³ is independently selected from H and C₁-C₆ alkyl; a moiety of formula (II):

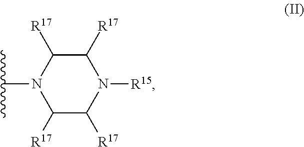

wherein:
   R¹⁵ is selected from H, G, C₁-C₆ alkyl, —C(═NH)NH₂, —C(O)(CH₂)_{q}NR¹⁸C(═NH)NH₂, and —C(O)(CH₂)₂NHC(O)(CH₂)₅NR¹⁸C(═NH)NH₂, wherein:
   R¹⁸ is selected from H and C₁-C₆ alkyl; and
   q is an integer from 1 to 5, and
   each R¹⁷ is independently selected from H and methyl; and
a moiety of formula (III):

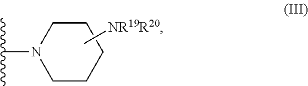

wherein:
   R¹⁹ is selected from H, C₁-C₆ alkyl, —C(═NH)NH₂, —C(O)(CH₂)ᵣNR²²C(═NH)NH₂, —C(O)CH(NH₂)(CH₂)₃NHC(═NH)NH₂, —C(O)(CH₂)₂NHC(O)(CH₂)₅NR²²C(═NH)NH₂, —C(O)CH(NH₂)(CH₂)₄NH₂ and G, wherein:
   R²² is selected from H and C₁-C₆ alkyl; and
   r is an integer from 1 to 5, and
   R²⁰ is selected from H and C₁-C₆ alkyl; and
R² is selected from H, G, acyl, trityl, 4-methoxytrityl, C₁-C₆ alkyl, —C(═NH)NH₂, —C(O)—R²³, —C(O)(CH₂)ₛNR²⁴C(═NH)NH₂, —C(O)(CH₂)₂NHC(O)(CH₂)NR²⁴C(═NH)NH₂, —C(O)CH(NH₂)(CH₂)₃NHC(═NH)NH₂, and a moiety of the formula:

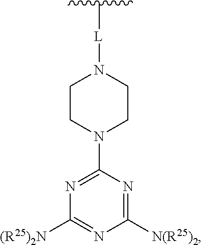

wherein,
   R²³ is of the formula —(O-alkyl)ᵥ-OH wherein v is an integer from 3 to 10 and each of the v alkyl groups is independently selected from C₂-C₆ alkyl; and
   R²⁴ is selected from H and C₁-C₆ alkyl;
   s is an integer from 1 to 5;
   L is selected from —C(O)(CH₂)₆C(O)— and —C(O)(CH₂)₂S₂(CH₂)₂C(O)—; and each R²⁵ is of the formula —(CH₂)₂₀C(O)N(R²⁶)₂ wherein each R²⁶ is of the formula —(CH₂)₆NHC(═NH)NH₂,

wherein G is a cell penetrating peptide (“CPP”) and linker moiety selected from —C(O)(CH₂)₅NH-CPP, —C(O)(CH₂)₂NH-CPP, —C(O)(CH₂)₂NHC(O)(CH₂)ₛNH-CPP, and —C(O)CH₂NH—CPP, or G is of the formula:

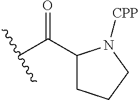

wherein the CPP is attached to the linker moiety by an amide bond at the CPP carboxy terminus, with the proviso that up to one instance of G is present, and

wherein the targeting sequence is complementary to 10 or more contiguous nucleotides in a target region within intron 1 (SEQ ID. NO: 1), intron 2 (SEQ ID. NO: 2), or exon 2 (SEQ ID. NO: 3) of a pre-mRNA of the human acid alpha-glucosidase (GAA) gene.

In certain embodiments, the antisense oligomer further comprises a peptide moiety which enhances cellular uptake. For example, in certain embodiments, the antisense oligomer of the disclosure is a compound of formula (IVb):

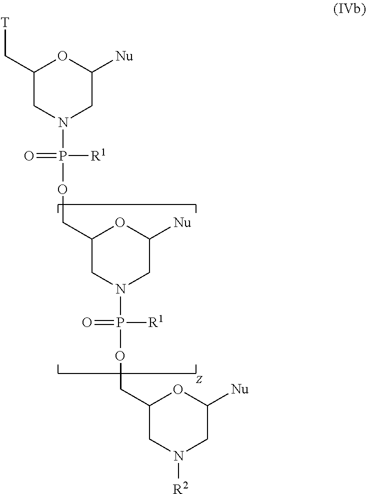

or a pharmaceutically acceptable salt thereof, where:
each Nu is a nucleobase which taken together forms a targeting sequence;
Z is an integer from 8 to 38;
T is selected from a moiety of the formula:

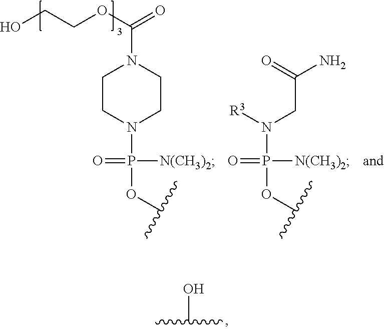

wherein R³ is selected from H and C₁-C₆ alkyl;
each instance of R¹ is independently —N(R⁴)₂, wherein each R⁴ is independently selected from H and C₁-C₆ alkyl; and
R² is selected from H, acyl, trityl, 4-methoxytrityl, and C₁-C₆ alkyl,

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene.

Also included within the scope of the disclosure are antisense oligomers, such as any of those of the formula above, comprising a targeting sequence of sufficient length and complementarity to specifically hybridize to a region within intron 1 (SEQ ID NO: 1), exon 2 (SEQ ID NO:2), or intron 2 (SEQ ID NO:3) of the pre-mRNA of the human acid alpha-glucosidase (GAA) gene, as set forth in Tables 2A, 2B, or 2C. In some embodiments, the targeting sequence comprises 10 or more (e.g., 11, 12, 13, 14, 15, 16, 17, 18, 19, 20, 21, 22, 23, 24, or 25 or more) contiguous nucleotides of a targeting sequence in Tables 2A, 2B, or 2C, e.g., a targeting sequence selected from SEQ ID. NOS: 4 to 30, 133 to 255, or 296 to 342, wherein X is selected from uracil (U) or thymine (T). In certain embodiments, the targeting sequence comprises 80% sequence identity to a targeting sequence selected from SEQ ID. NOS: 4 to 30, 133 to 255, or 296 to 342, wherein X is selected from uracil (U) or thymine (T).

In some embodiments of any of the methods or compositions described herein, Z is an integer from 8 to 28, from 15 to 38, 15 to 28, 8 to 25, from 15 to 25, from 10 to 38, from 10 to 25, from 12 to 38, from 12 to 25, from 14 to 38, or from 14 to 25. In some embodiments of any of the methods or compositions described herein, Z is 12, 13, 14, 15, 16, 17, 18, 19, 20, 21, 22, 23, 24, 25, 26, 27, 28, 29, 30, 31, 32, 33, 34, 35, 36, 37, or 38. In some embodiments of any of the methods or compositions described herein, Z is 12, 13, 14, 15, 16, 17, 18, 19, 20, 21, 22, 23, 24, 25, 26, 27, or 28. In some embodiments of any of the methods or compositions described herein, Z is 15, 16, 17, 18, 19, 20, 21, 22, 23, 24, or 25.

In particular embodiments, the antisense oligomer is a phosphoramidate morpholino, phosphorodiamidate morpholino, phosphorothioate, 2′ O-methyl, peptide nucleic acid, locked nucleic acid, phosphorothioate, 2′ O-MOE, 2′-fluoro, 2′O,4′C-ethylene-bridged nucleic acid, tricyclo-DNA, tricyclo-DNA phosphorothioate nucleotide, 2′-O-[2-(N-methylcarbamoyl)ethyl], morpholino, peptide-conjugated phosphoramidate morpholino, phosphorodiamidate morpholino having a phosphorous atom with (i) a covalent bond to the nitrogen atom of a morpholino ring, and (ii) a second covalent bond to a (1,4-piperazin)-1-yl substituent or to a substituted (1,4-piperazin)-1-yl, and phosphorodiamidate morpholino having a phosphorus atom with (i) a covalent bond to the nitrogen atom of a morpholino ring and (ii) a second covalent bond to the ring nitrogen of a 4-aminopiperdin-1-yl or a derivative of 4-aminopiperdin-1-yl chemistries, or any combination of the foregoing.

In some embodiments, the antisense oligomer or compound suppress an ISS and/or ESS element in the GAA pre-mRNA. In some embodiments, he antisense oligomer or compound increases, enhances, or promotes exon 2 retention in the mature GAA mRNA, optionally by at least about 10%, 15%, 20%, 25%, 30%, 35%, 40%, 45%, 50%, 55%, 60%, or 65% or more relative to a control, according to at least one of the examples or methods described herein. In some embodiments, the antisense oligomer or compouns increases, enhances, or promotes GAA protein expression in a cell (e.g., a cell from a patient having a IVS1-13T>G mutation), optionally by at least about 10%, 15%, 20%, 25%, 30%, 35%, 40%, 45%, 50%, 55%, 60%, or 65% or more relative to a control, according to at least one of the examples or methods described here. In some embodiments, the antisense oligomer or compound increases, enhances, or promotes GAA enzymatic activity in a cell (e.g., a cell from a patient having a IVS1-13T>G mutation), optionally by at least about 10%, 15%, 20%, 25%, 30%, 35%, 40%, 45%, 50%, 55%, 60%, or 65% or more relative to a control, according to at least one of the examples or methods described herein).

In some embodiments, the antisense oligomer or compound induces at least about a 2 (e.g., 2.5, 3, 3.5, 4, 4.5, 5, 5.5, 6, 6.5, 7, 7.5, 8, 8.5, 9, 9.5, 10, or greater) fold increase in GAA enzyme activity in a cell (e.g., a cell from a patient having a IVS1-13T>G mutation), relative to the GAA activity in the cell not contacted with the oligomers or compounds, according to at least one of the examples or methods described herein. In some embodiments, the antisense oligomer or compound induces at least about a 2 (e.g., 2.5, 3, 3.5, 4, 4.5, 5, 5.5, 6, 6.5, 7, 7.5, 8, 8.5, 9, 9.5, 10, or greater) fold increase in GAA enzyme activity in a cell (e.g., a cell from a patient having a IVS1-13T>G mutation) at, e.g., 0.4 μM or 0.2 μM, relative to the GAA activity in the cell not contacted with the oligomers or compounds, according to at least one of the examples or methods described herein.

In some embodiments, the antisense oligomer or compound comprises a targeting sequence comprising, consisting of, or consisting essentially of, a targeting sequence set forth in any one of Tables 4A, 5, or 6.

Also included are pharmaceutical compositions, comprising a physiologically-acceptable carrier and an antisense oligomer described herein.

Certain embodiments also include methods of increasing the level of exon 2-containing acid alpha-glucosidase (GAA) mRNA in a cell, comprising contacting the cell with an antisense oligomer of sufficient length and complementarity to specifically hybridize to a region within the pre-mRNA of the GAA gene, wherein binding of the antisense oligomer to the region increases the level of exon 2-containing GAA mRNA in the cell.

In some embodiments, the level of exon 2-containing GAA mRNA in the cell is increased by at least about 10% relative to a control. In certain embodiments, the level of functional GAA protein in the cell is increased by at least about 10% relative to a control. In certain embodiments, the cell has an IVS1-13T>G mutation in one or more alleles of its genome which (in the absence of antisense treatment) causes reduced expression of exon 2-containing GAA mRNA.

In some embodiments, the cell is in a subject in need thereof, and the method comprises administering the antisense oligomer to the subject. In some embodiments, the subject has or is at risk for having glycogen storage disease type II (GSD-II). Some embodiments of the disclosure relate to methods of treating glycogen storage disease type II (GSD-II; Pompe disease) in a subject in need thereof, comprising administering to the subject an effective amount of an antisense oligomer of the disclosure. While certain embodiments relate to antisense oligomers for use in the preparation of a medicament for the treatment of glycogen storage disease type II (GSD-II; Pompe disease).

In certain embodiments, the subject has or is at risk for having infantile GSD-II. In particular embodiments, the subject has or is at risk for having late onset GSD-II. In certain embodiments, the method comprises reducing the glycogen levels in one or more tissues of the subject by at least about 10% relative to a control.

In addition, the instant disclosure also includes a method of detecting exon 2 inclusion in a human acid alpha-glucosidase (GAA) gene mRNA, the method comprising:

amplifying the GAA mRNA with at least one polymerase chain reaction primer comprising a base sequence selected from the group consisting of SEQ ID NOS: 33, 34, or 35.

These and other aspects of the present disclosure will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 illustrates one mechanism by which steric-blocking antisense oligomers can enhance the level of exon 2-containing GAA mRNA relative to exon-deleted GAA mRNA.

FIGS. 2-4 are bar graphs depicting the protein expression levels (Wes) and GAA enzyme activity (Enzyme Assay) in cells treated with various PMO compounds. The Y axis represents fold increase in protein expression or GAA enzyme activity relative to cells that were not treated with PMO. “N” refers to the number of replicates evaluated in each study.

FIGS. 5-7 are bar graphs depicting the GAA enzyme activity (Enzyme Assay) in cells treated with various PMO compounds. The Y axis represents fold increase in GAA enzyme activity relative to cells that were not treated with PMO. “N” refers to the number of replicates evaluated in each study.

FIGS. 8 and 9 are bar graphs depicting the GAA enzyme activity (Enzyme Assay) in cells treated with PMO compounds at several concentrations as indicated. The Y axis represents fold increase GAA enzyme activity relative to cells that were not treated with PMO.

FIG. 10 is a bar graph depicting the GAA enzyme activity (Enzyme Assay) in cells treated with various PMO compounds. The Y axis represents fold increase in GAA enzyme activity relative to cells that were not treated with PMO. The horizontal hashed line signifies the level of GAA activity in untreated cells. Individual compounds were dosed at 20 μM.

FIG. 11 is a bar graph depicting the GAA enzyme activity (Enzyme Assay) in cells treated with various PMO compounds. The Y axis represents fold increase in GAA enzyme activity relative to cells that were not treated with PMO. The horizontal hashed line signifies the level of GAA activity in untreated cells. Individual compounds were dosed at 5 μM, 1 μM, and 0.2 μM.

FIG. 12 is a bar graph depicting the GAA enzyme activity (Enzyme Assay) in cells treated with various PMO compounds. The Y axis represents fold increase in GAA enzyme activity relative to cells that were not treated with PMO. The horizontal hashed line signifies the level of GAA activity in untreated cells. Individual compounds were dosed at 5 μM, 1 μM, 0.2 μM, and 0.04 μM.

FIG. 13 is a bar graph depicting the GAA enzyme activity (Enzyme Assay) in cells treated with various PMO compounds. The Y axis represents fold increase in GAA enzyme activity relative to cells that were not treated with PMO. The horizontal hashed line signifies the level of GAA activity in untreated cells. Individual compounds were dosed at 20 μM.

FIG. 14 is a bar graph depicting the GAA enzyme activity (Enzyme Assay) in cells treated with various PMO compounds. The Y axis represents fold increase in GAA enzyme activity relative to cells that were not treated with PMO. The horizontal hashed line signifies the level of GAA activity in untreated cells. Individual compounds were dosed at 20 μM.

FIG. 15 is a bar graph depicting the GAA enzyme activity (Enzyme Assay) in cells treated with various PMO compounds. The Y axis represents fold increase in GAA enzyme activity relative to cells that were not treated with PMO. The horizontal hashed line signifies the level of GAA activity in untreated cells. Individual compounds were dosed at 20 μM.

FIG. 16 is a bar graph depicting the GAA enzyme activity (Enzyme Assay) in cells treated with various PMO compounds. The Y axis represents fold increase in GAA enzyme activity relative to cells that were not treated with PMO. The horizontal hashed line signifies the level of GAA activity in untreated cells. Individual compounds were dosed at 20 μM.

### DETAILED DESCRIPTION

### I. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the subject matter of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of:” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “contacting a cell”, “introducing” or “delivering” include delivery of the oligomers of the disclosure into a cell by methods routine in the art, e.g., transfection (e.g., liposome, calcium-phosphate, polyethyleneimine), electroporation (e.g., nucleofection), microinjection).

As used herein, the term “alkyl” is intended to include linear (i.e., unbranched or acyclic), branched, cyclic, or polycyclic non aromatic hydrocarbon groups, which are optionally substituted with one or more functional groups. Unless otherwise specified, “alkyl” groups contain one to eight, and preferably one to six carbon atoms. C₁-C₆ alkyl, is intended to include C₁, C₂, C₃, C₄, C₅, and C₆ alkyl groups. Lower alkyl refers to alkyl groups containing 1 to 6 carbon atoms. Examples of Alkyl include, but are not limited to, methyl, ethyl, n-propyl, isopropyl, cyclopropyl, butyl, isobutyl, sec-butyl, tert-butyl, cyclobutyl, pentyl, isopentyl tert-pentyl, cyclopentyl, hexyl, isohexyl, cyclohexyl, etc. Alkyl may be substituted or unsubstituted. Illustrative substituted alkyl groups include, but are not limited to, fluoromethyl, difluoromethyl, trifluoromethyl, 2-fluoroethyl, 3-fluoropropyl, hydroxymethyl, 2-hydroxyethyl, 3-hydroxypropyl, benzyl, substituted benzyl, phenethyl, substituted phenethyl, etc.

As used herein, the term “Alkoxy” means a subset of alkyl in which an alkyl group as defined above with the indicated number of carbons attached through an oxygen bridge. For example, “alkoxy” refers to groups —O-alkyl, wherein the alkyl group contains 1 to 8 carbons atoms of a linear, branched, cyclic configuration. Examples of “alkoxy” include, but are not limited to, methoxy, ethoxy, n-propoxy, i-propoxy, t-butoxy, n-butoxy, s-pentoxy and the like.

As used herein, the term “aryl” used alone or as part of a larger moiety as in “aralkyl”, “aralkoxy”, or “aryloxy-alkyl”, refers to aromatic ring groups having six to fourteen ring atoms, such as phenyl, 1-naphthyl, 2-naphthyl, 1-anthracyl and 2-anthracyl. An “aryl” ring may contain one or more substituents. The term “aryl” may be used interchangeably with the term “aryl ring”. “Aryl” also includes fused polycyclic aromatic ring systems in which an aromatic ring is fused to one or more rings. Non-limiting examples of useful aryl ring groups include phenyl, hydroxyphenyl, halophenyl, alkoxyphenyl, dialkoxyphenyl, trialkoxyphenyl, alkylenedioxyphenyl, naphthyl, phenanthryl, anthryl, phenanthro and the like, as well as 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in a indanyl, phenanthridinyl, or tetrahydronaphthyl, where the radical or point of attachment is on the aromatic ring.

The term “acyl” means a C(O)R group (in which R signifies H, alkyl or aryl as defined above). Examples of acyl groups include formyl, acetyl, benzoyl, phenylacetyl and similar groups.

The term “homolog” as used herein means compounds differing regularly by the successive addition of the same chemical group. For example, a homolog of a compound may differ by the addition of one or more —CH₂— groups, amino acid residues, nucleotides, or nucleotide analogs.

The terms “cell penetrating peptide” (CPP) or “a peptide moiety which enhances cellular uptake” are used interchangeably and refer to cationic cell penetrating peptides, also called “transport peptides”, “carrier peptides”, or “peptide transduction domains”. The peptides, as shown herein, have the capability of inducing cell penetration within about or at least about 30%, 40%, 50%, 60%, 70%, 80%, 90%, or 100% of cells of a given cell culture population and allow macromolecular translocation within multiple tissues in vivo upon systemic administration. In some embodiments, the CPPs are of the formula —[(C(O)CHR′NH)ₘ]R″ wherein R′ is a side chain of a naturally occurring amino acid or a one- or two-carbon homolog thereof, R″ is selected from Hydrogen or acyl, and m is an integer up to 50. Additional CPPs are well-known in the art and are disclosed, for example, in U.S. Application No. 2010/0016215, which is incorporated by reference in its entirety. In other embodiments, m is an integer selected from 1 to 50 where, when m is 1, the moiety is a single amino acid or derivative thereof.

As used herein, “amino acid” refers to a compound consisting of a carbon atom to which are attached a primary amino group, a carboxylic acid group, a side chain, and a hydrogen atom. For example, the term “amino acid” includes, but is not limited to, Glycine, Alanine, Valine, Leucine, Isoleucine, Asparagine, Glutamine, Lysine and Arginine. Additionally, as used herein, “amino acid” also includes derivatives of amino acids such as esters, and amides, and salts, as well as other derivatives, including derivatives having pharmacoproperties upon metabolism to an active form. Accordingly, the term “amino acid” is understood to include naturally occurring and non-naturally occurring amino acids.

“An electron pair” refers to a valence pair of electrons that are not bonded or shared with other atoms.

“Homology” refers to the percentage number of amino acids that are identical or constitute conservative substitutions. Homology may be determined using sequence comparison programs such as GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

By “isolated” is meant material that is substantially or essentially free from components that normally accompany it in its native state. For example, an “isolated polynucleotide,” “isolated oligonucleotide,” or “isolated oligomer” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

The terms “modulate” includes to “increase” or “decrease” one or more quantifiable parameters, optionally by a defined and/or statistically significant amount. By “increase” or “increasing,” “enhance” or “enhancing,” or “stimulate” or “stimulating,” refers generally to the ability of one or more antisense compounds or compositions to produce or cause a greater physiological response (i.e., downstream effects) in a cell or a subject relative to the response caused by either no antisense compound or a control compound. Relevant physiological or cellular responses (in vivo or in vitro) will be apparent to persons skilled in the art, and may include increases in the inclusion of exon 2 in a GAA-coding pre-mRNA, or increases in the expression of functional GAA enzyme in a cell, tissue, or subject in need thereof. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times), including all integers and decimal points in between and above 1 (e.g., 1.5, 1.6, 1.7. 1.8), the amount produced by no antisense compound (the absence of an agent) or a control compound. The term “reduce” or “inhibit” may relate generally to the ability of one or more antisense compounds or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a glycogen storage disease such as Pompe disease, for example, a decrease in the accumulation of glycogen in one or more tissues. A “decrease” in a response may be “statistically significant” as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

As used herein, an “antisense oligonucleotide,” “antisense oligomer” or “oligonucleotide” refers to a linear sequence of nucleotides, or nucleotide analogs, which allows the nucleobase to hybridize to a target sequence in an RNA by Watson-Crick base pairing, to form an oligomer:RNA heteroduplex within the target sequence. The terms “antisense oligonucleotide”, “antisense oligomer”, “oligomer” and “compound” may be used interchangeably to refer to an oligomer. The cyclic subunits may be based on ribose or another pentose sugar or, in certain embodiments, a morpholino group (see description of morpholino oligomers below). Also contemplated are peptide nucleic acids (PNAs), locked nucleic acids (LNAs), tricyclo-DNA oligomers, tricyclo-phosphorothioate oligomers, and 2′-O-Methyl oligomers, among other antisense agents known in the art.

Included are non-naturally-occurring oligomers, or “oligonucleotide analogs,” including oligomers having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligomer analogs support bases capable of hydrogen bonding by Watson-Crick base pairing to standard polynucleotide bases, where the analog backbone presents the bases in a manner to permit such hydrogen bonding in a sequence-specific fashion between the oligomer analog molecule and bases in a standard polynucleotide (e.g., single-stranded RNA or single-stranded DNA). Preferred analogs are those having a substantially uncharged, phosphorus containing backbone.

A “nuclease-resistant” oligomer refers to one whose backbone is substantially resistant to nuclease cleavage, in non-hybridized or hybridized form; by common extracellular and intracellular nucleases in the body (for example, by exonucleases such as 3′-exonucleases, endonucleases, RNase H); that is, the oligomer shows little or no nuclease cleavage under normal nuclease conditions in the body to which the oligomer is exposed. A “nuclease-resistant heteroduplex” refers to a heteroduplex formed by the binding of an antisense oligomer to its complementary target, such that the heteroduplex is substantially resistant to in vivo degradation by intracellular and extracellular nucleases, which are capable of cutting double-stranded RNA/RNA or RNA/DNA complexes. A “heteroduplex” refers to a duplex between an antisense oligomer and the complementary portion of a target RNA.

As used herein, “nucleobase” (Nu), “base pairing moiety” or “base” are used interchangeably to refer to a purine or pyrimidine base found in native DNA or RNA (uracil, thymine, adenine, cytosine, and guanine), as well as analogs of the naturally occurring purines and pyrimidines, that confer improved properties, such as binding affinity to the oligomer. Exemplary analogs include hypoxanthine (the base component of the nucleoside inosine); 2, 6-diaminopurine; 5-methyl cytosine; C5-propynyl-modified pyrimidines; 9-(aminoethoxy)phenoxazine (G-clamp) and the like.

Further examples of base pairing moieties include, but are not limited to, uracil, thymine, adenine, cytosine, guanine and hypoxanthine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313, are also contemplated.

Further examples of base pairing moieties include, but are not limited to, expanded-size nucleobases in which one or more benzene rings has been added. Nucleic base replacements described in the Glen Research catalog (www.glenresearch.com); Krueger A T et al, Acc. Chem. Res., 2007, 40, 141-150; Kool, E T, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, are contemplated as useful for the synthesis of the oligomers described herein. Examples of expanded-size nucleobases are shown below:

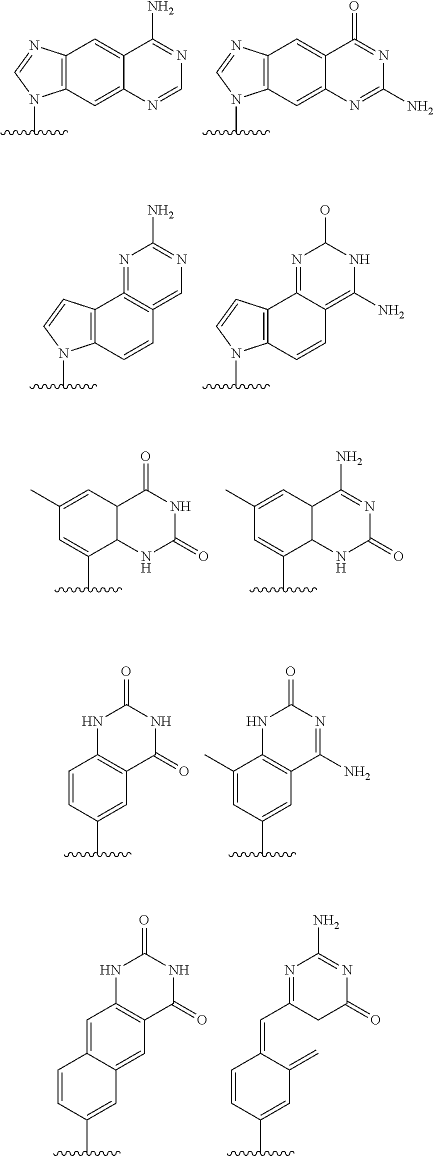

A nucleobase covalently linked to a ribose, sugar analog or morpholino comprises a nucleoside. “Nucleotides” are composed of a nucleoside together with one phosphate group. The phosphate groups covalently link adjacent nucleotides to one another to form an oligomer.

An oligomer “specifically hybridizes” to a target polynucleotide if the oligomer hybridizes to the target under physiological conditions, with a Tm substantially greater than 40° C. or 45° C., preferably at least 50° C., and typically 60° C.-80° C. or higher. Such hybridization preferably corresponds to stringent hybridization conditions. At a given ionic strength and pH, the Tm is the temperature at which 50% of a target sequence hybridizes to a complementary polynucleotide. Such hybridization may occur with “near” or “substantial” complementarity of the antisense oligomer to the target sequence, as well as with exact complementarity.

As used herein, “sufficient length” refers to an antisense oligomer or a targeting sequence thereof that is complementary to at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 or more, such as 8-40, contiguous nucleobases in a region of GAA intron 1, exon 2, or intron 2, or a region spanning any of the foregoing. An antisense oligomer of sufficient length has at least a minimal number of nucleotides to be capable of specifically hybridizing to a region of the GAA pre-mRNA repeat in the mutant RNA. Preferably an oligomer of sufficient length is from 8 to 30 nucleotides in length. More preferably, an oligomer of sufficient length is from 9 to 27 nucleotides in length.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

A “subject” or a “subject in need thereof” includes a mammalian subject such as a human subject. Exemplary mammalian subjects have or are at risk for having GSD-II (or Pompe disease). As used herein, the term “GSD-II” refers to glycogen storage disease type II (GSD-II or Pompe disease), a human autosomal recessive disease that is often characterized by under expression of GAA protein in affected individuals. In certain embodiments, a subject has reduced expression and/or activity of GAA protein in one or more tissues, for example, heart, skeletal muscle, liver, and nervous system tissues. In some embodiments, the subject has increased accumulation of glycogen in one or more tissues, for example, heart, skeletal muscle, liver, and nervous system tissues. In specific embodiments, the subject has a IVS1-13T>G mutation or other mutation that leads to reduced expression of functional GAA protein (see, e.g., Zampieri et al., European J. Human Genetics. 19:422-431, 2011).

As used herein, the term “target” refers to a RNA region, and specifically, to a region identified by the GAA gene. In a particular embodiment the target is a region within intron 1 or intron 2 of the GAA-coding pre-mRNA, which is responsible for suppression of a signal that promotes exon 2 inclusion. In another embodiment the target region is a region of the mRNA of GAA exon 2.

The term “target sequence” refers to a portion of the target RNA against which the oligomer analog is directed, that is, the sequence to which the oligomer analog will hybridize by Watson-Crick base pairing of a complementary sequence.

The term “targeting sequence” is the sequence in the oligomer or oligomer analog that is complementary (meaning, in addition, substantially complementary) to the “target sequence” in the RNA genome. The entire sequence, or only a portion, of the antisense oligomer may be complementary to the target sequence. For example, in an oligomer having 20-30 bases, about 6, 7, 8, 9, 10, 11, 12, 13, 14, 15, 16, 17, 18, 19, 20, 21, 22, 23, 24, 25, 26, 27, 28, or 29 may be targeting sequences that are complementary to the target region. Typically, the targeting sequence is formed of contiguous bases in the oligomer, but may alternatively be formed of non-contiguous sequences that when placed together, e.g., from opposite ends of the oligomer, constitute sequence that spans the target sequence.

A “targeting sequence” may have “near” or “substantial” complementarity to the target sequence and still function for the purpose of the present disclosure, that is, still be “complementary.” Preferably, the oligomer analog compounds employed in the present disclosure have at most one mismatch with the target sequence out of 10 nucleotides, and preferably at most one mismatch out of 20. Alternatively, the antisense oligomers employed have at least 90% sequence homology, and preferably at least 95% sequence homology, with the exemplary targeting sequences as designated herein.

The terms “TEG” or “triethylene glycol tail” refer to triethylene glycol moieties conjugated to the oligonucleotide, e.g., at its 3′- or 5′-end. For example, in some embodiments, “TEG” includes wherein, for example, T of the compound of formula (I), (VI), or (VII) is of the formula:

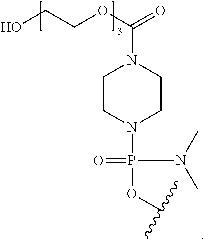

As used herein, the term “quantifying”, “quantification” or other related words refer to determining the quantity, mass, or concentration in a unit volume, of a nucleic acid, polynucleotide, oligomer, peptide, polypeptide, or protein.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent.

Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

### II. Sequences for Splice Modulation of GAA

Certain embodiments relate to methods for enhancing the level of exon 2-containing GAA-coding mRNA relative to exon-2 deleted GAA mRNA in a cell, comprising contacting the cell with an antisense oligomer of sufficient length and complementarity to specifically hybridize to a region within the GAA gene, such that the level of exon 2-containing GAA mRNA relative to exon-2 deleted GAA mRNA in the cell is enhanced. In some embodiments, the cell is in a subject, and the method comprises administering to the antisense oligomer to the subject.

An antisense oligomer can be designed to block or inhibit or modulate translation of mRNA or to inhibit or modulate pre-mRNA splice processing, or induce degradation of targeted mRNAs, and may be said to be “directed to” or “targeted against” a target sequence with which it hybridizes. In certain embodiments, the target sequence includes a region including a 3′ or 5′ splice site of a pre-processed mRNA, a branch point, or other sequence involved in the regulation of splicing. The target sequence may be within an exon or within an intron or spanning an intron/exon junction.

In certain embodiments, the antisense oligomer has sufficient sequence complementarity to a target RNA (i.e., the RNA for which splice site selection is modulated) to block a region of a target RNA (e.g., pre-mRNA) in an effective manner. In exemplary embodiments, such blocking of GAA pre-mRNA serves to modulate splicing, either by masking a binding site for a native protein that would otherwise modulate splicing and/or by altering the structure of the targeted RNA. In some embodiments, the target RNA is target pre-mRNA (e.g., GAA gene pre-mRNA).

An antisense oligomer having a sufficient sequence complementarity to a target RNA sequence to modulate splicing of the target RNA means that the antisense agent has a sequence sufficient to trigger the masking of a binding site for a native protein that would otherwise modulate splicing and/or alters the three-dimensional structure of the targeted RNA. Likewise, an oligomer reagent having a sufficient sequence complementary to a target RNA sequence to modulate splicing of the target RNA means that the oligomer reagent has a sequence sufficient to trigger the masking of a binding site for a native protein that would otherwise modulate splicing and/or alters the three-dimensional structure of the targeted RNA.

In certain embodiments, the antisense oligomer has sufficient length and complementarity to a sequence in intron 1 of the human GAA pre-mRNA, exon 2 of the human GAA pre-mRNA, or intron 2 of the human GAA pre-mRNA. Also included are antisense oligomers which are complementary to a region that spans intron 1/exon 2 of the human GAA pre-mRNA, or a region that spans exon 2/intron 2 of the human GAA pre-mRNA. The intron 1 (SEQ ID NO: 1), exon 2 (SEQ ID NO:2), and intron 2 (SEQ ID NO:3) sequences for human the GAA gene are shown in Table 1 below (The highlighted T/G near the 3′ end of SEQ ID NO: 1 is the IVS1-13T>G mutation described above; the nucleotide at this position is either T or G).

TABLE 1
Target sequences for GAA-targeted oligomers (from NG_009822)

| | | SEQ |
|---|---|---|
| Name | Sequence (5′-3′) | ID NO |
| GAA- | GTGAGACACCTGACGTCTGCCCCGCGCTGCCGGCGGTAACATCCCAGA | 1 |
| IVS1 | AGCGGGTTTGAACGTGCCTAGCCGTGCCCCCAGCCTCTTCCCCTGAGC | |
| | GGAGCTTGAGCCCCAGACCTCTAGTCCTCCCGGTCTTTATCTGAGTTCA | |
| | GCTTAGAGATGAACGGGGAGCCGCCCTCCTGTGCTGGGCTTGGGGCTG | |
| | GAGGCTGCATCTTCCCGTTTCTAGGGTTTCCTTTCCCCTTTTGATCGAC | |
| | GCAGTGCTCAGTCCTGGCCGGGACCCGAGCCACCTCTCCTGCTCCTGC | |
| | AGGACGCACATGGCTGGGTCTGAATCCCTGGGGTGAGGAGCACCGTG | |
| | GCCTGAGAGGGGGCCCCTGGGCCAGCTCTGAAATCTGAATGTCTCAAT | |
| | CACAAAGACCCCCTTAGGCCAGGCCAGGGGTGACTGTCTCTGGTCTTT | |
| | GTCCCTGGTTGCTGGCACATAGCACCCGAAACCCTTGGAAACCGAGTG | |
| | ATGAGAGAGCCTTTTGCTCATGAGGTGACTGATGACCGGGGACACCA | |
| | GGTGGCTTCAGGATGGAAGCAGATGGCCAGAAAGACCAAGGCCTGAT | |
| | GACGGGTTGGGATGGAAAAGGGGTGAGGGGCTGGAGATTGAGTGAAT | |
| | CACCAGTGGCTTAGTCAACCATGCCTGCACAATGGAACCCCGTAAGAA | |
| | ACCACAGGGATCAGAGGGCTTCCCGCCGGGTTGTGGAACACACCAAG | |
| | GCACTGGAGGGTGGTGCGAGCAGAGAGCACAGCATCACTGCCCCCAC | |
| | CTCACACCAGGCCCTACGCATCTCTTCCATACGGCTGTCTGAGTTTTAT | |
| | CCTTTGTAATAAACCAGCAACTGTAAGAAACGCACTTTCCTGAGTTCT | |
| | GTGACCCTGAAGAGGGAGTCCTGGGAACCTCTGAATTTATAACTAGTT | |
| | GATCGAAAGTACAAGTGACAACCTGGGATTTGCCATTGGCCTCTGAAG | |
| | TGAAGGCAGTGTTGTGGGACTGAGCCCTTAACCTGTGGAGTCTGTGCT | |
| | GACTCCAGGTAGTGTCAAGATTGAATTGAATTGTAGGACACCCAGCCG | |
| | TGTCCAGAAAGTTGCAGAATTGATGGGTGTGAGAAAAACCCTACACA | |
| | TTTAATGTCAGAAGTGTGGGTAAAATGTTTCACCCTCCAGCCCAGAGA | |
| | GCCCTAATTTACCAGTGGCCCACGGTGGAACACCACGTCCGGCCGGGG | |
| | GCAGAGCGTTCCCAGCCAAGCCTTCTGTAACATGACATGACAGGTCAG | |
| | ACTCCCTCGGGCCCTGAGTTCACTTCTTCCTGGTATGTGACCAGCTCCC | |
| | AGTACCAGAGAAGGTTGCACAGTCCTCTGCTCCAAGGAGCTTCACTGG | |
| | CCAGGGGCTGCTTTCTGAAATCCTTGCCTGCCTCTGCTCCAAGGCCCGT | |
| | TCCTCAGAGACGCAGACCCCTCTGATGGCTGACTTTGGTTTGAGGACC | |
| | TCTCTGCATCCCTCCCCCATGGCCTTGCTCCTAGGACACCTTCTTCCTC | |
| | CTTTCCCTGGGGTCAGACTTGCCTAGGTGCGGTGGCTCTCCCAGCCTTC | |
| | CCCACGCCCTCCCCATGGTGTATTACACACACCAAAGGGACTCCCCTA | |
| | TTGAAATCCATGCATATTGAATCGCATGTGGGTTCCGGCTGCTCCTGG | |
| | GAGGAGCCAGGCTAATAGAATGTTTGCCATAAAATATTAATGTACAG | |
| | AGAAGCGAAACAAAGGTCGTTGGTACTTGTTAACCTTACCAGCAGAAT | |
| | AATGAAAGCGAACCCCCATATCTCATCTGCACGCGACATCCTTGTTGT | |
| | GTCTGTACCCGAGGCTCCAGGTGCAGCCACTGTTACAGAGACTGTGTT | |
| | TCTTCCCCATGTACCTCGGGGGCCGGGAGGGGTTCTGATCTGCAAAGT | |
| | CGCCAGAGGTTAAGTCCTTTCTCTCTTGTGGCTTTGCCACCCCTGGAGT | |
| | GTCACCCTCAGCTGCGGTGCCCAGGATTCCCCACTGTGGTATGTCCGT | |
| | GCACCAGTCAATAGGAAAGGGAGCAAGGAAAGGTACTGGGTCCCCCT | |
| | AAGGACATACGAGTTGCCAGAATCACTTCCGCTGACACCCAGTGGACC | |
| | AAGCCGCACCTTTATGCAGAAGTGGGGCTCCCAGCCAGGCGTGGTCAC | |
| | TCCTGAAATCCCAGCACTTCGGAAGGCCAAGGGGGGTGGATCACTTG | |
| | AGCTCAGGAGTTCGAGACCAGCCTGGGTAACATGGCAAAATCCCGTCT | |
| | CTACAAAAATACAGAAAATTAGCTGGGTGCGGTGGTGTGTGCCTACA | |
| | GTCCCAGCTACTCAGGAGGCTGAAGTGGGAGGATTGCTTGAGTCTGGG | |
| | AGGTGGAGGTTGCAGTGAGCCAGGATCTCACCACAGCACTCTGGCCC | |
| | AGGCGACAGCTGTTTGGCCTGTTTCAAGTGTCTACCTGCCTTGCTGGTC | |
| | TTCCTGGGGACATTCTAAGCGTGTTTGATTTGTAACATTTTAGCAGACT | |
| | GTGCAAGTGCTCTGCACTCCCCTGCTGGAGCTTTTCTCGCCCTTCCTTC | |
| | TGGCCCTCTCCCCAGTCTAGACAGCAGGGCAACACCCACCCTGGCCAC | |
| | CTTACCCCACCTGCCTGGGTGCTGCAGTGCCAGCCGCGGTTGATGTCT | |
| | CAGAGCTGCTTTGAGAGCCCCGTGAGTGCCGCCCCTCCCGCCTCCCTG | |
| | CTGAGCCCGCTTT/GCTTCTCCCGCAG | |
| GAA- | GCCTGTAGGAGCTGTCCAGGCCATCTCCAACCATGGGAGTGAGGCACC | 2 |
| exon2 | CGCCCTGCTCCCACCGGCTCCTGGCCGTCTGCGCCCTCGTGTCCTTGGC | |
| | AACCGCTGCACTCCTGGGGCACATCCTACTCCATGATTTCCTGCTGGTT | |
| | CCCCGAGAGCTGAGTGGCTCCTCCCCAGTCCTGGAGGAGACTCACCCA | |
| | GCTCACCAGCAGGGAGCCAGCAGACCAGGGCCCCGGGATGCCCAGGC | |
| | ACACCCCGGCCGTCCCAGAGCAGTGCCCACACAGTGCGACGTCCCCCC | |
| | CAACAGCCGCTTCGATTGCGCCCCTGACAAGGCCATCACCCAGGAACA | |
| | GTGCGAGGCCCGCGGCTGTTGCTACATCCCTGCAAAGCAGGGGCTGCA | |
| | GGGAGCCCAGATGGGGCAGCCCTGGTGCTTCTTCCCACCCAGCTACCC | |
| | CAGCTACAAGCTGGAGAACCTGAGCTCCTCTGAAATGGGCTACACGG | |
| | CCACCCTGACCCGTACCACCCCCACCTTCTTCCCCAAGGACATCCTGA | |
| | CCCTGCGGCTGGACGTGATGATGGAGACTGAGAACCGCCTCCACTTCA | |
| | CG | |
| GAA- | GTGGGCAGGGCAGGGGCGGGGGCGGCGGCCAGGGCAGAGGGTGCGC | 3 |
| IVS2 | GTGGACATCGACACCCACGCACCTCACAAGGGTGGGGTGCATGTTGC | |
| | ACCACTGTGTGCTGGGCCCTTGCTGGGAGCGGAGGTGTGAGCAGACA | |
| | ATGGCAGCGCCCCTCGGGGAGCAGTGGGGACACCACGGTGACAGGTA | |
| | CTCCAGAAGGCAGGGCTCGGGGCTCATTCATCTTTATGAAAAGGTGGG | |
| | TCAGGTAGAGTAGGGCTGCCAGAGGTTGCGAATGAAAACAGGATGCC | |
| | CAGTAAACCCGAATTGCAGATACCCCAGGCATGACTTTGTTTTTTTGT | |
| | GTAAGGATGCAAAATTTGGGATGTATTTATACTAGAAAAGCTGCTTGT | |
| | TGTTTATCTGAAATTCAGAGTTATCAGGTGTTCTGTATTTTACCTCCAT | |
| | CCTGGGGGAGGCGTCCTCCTCCTGGCTCTGCAGATGAGGGAGCCGAG | |
| | GCTCAGAGAGGCTGAATGTGCTGCCCATGGTCCCACATCCATGTGTGG | |
| | CTGCACCAGGACCTGACCTGTCCTTGGCGTGCGGGTTGTTCTCTGGAG | |
| | AGTAAGGTGGCTGTGGGGAACATCAATAAACCCCCATCTCTTCTAG | |

In certain embodiments, antisense targeting sequences are designed to hybridize to a region of one or more of the target sequences listed in Table 1. Selected antisense targeting sequences can be made shorter, e.g., about 12 bases, or longer, e.g., about 40 bases, and include a small number of mismatches, as long as the sequence is sufficiently complementary to effect splice modulation upon hybridization to the target sequence, and optionally forms with the RNA a heteroduplex having a Tm of 45° C. or greater.

In certain embodiments, the degree of complementarity between the target sequence and antisense targeting sequence is sufficient to form a stable duplex. The region of complementarity of the antisense oligomers with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligomer of about 14-15 bases is generally long enough to have a unique complementary sequence. In certain embodiments, a minimum length of complementary bases may be required to achieve the requisite binding Tm, as discussed herein.

In certain embodiments, oligomers as long as 40 bases may be suitable, where at least a minimum number of bases, e.g., 10-12 bases, are complementary to the target sequence. In some embodiments, facilitated or active uptake in cells is optimized at oligomer lengths of less than about 30 bases. For PMO oligomers, described further herein, an optimum balance of binding stability and uptake generally occurs at lengths of 18-25 bases. Included in the disclosure are antisense oligomers (e.g., PMOs, PMO-X, PNAs, LNAs, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases, in which at least about 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous or non-contiguous bases are complementary to the target sequences of Table 1 (e.g., SEQ ID NOS: 1-3, a sequence that spans SEQ ID NOS:1/2 or SEQ ID NOS:2/3).

The antisense oligomers typically comprises a base sequence which is sufficiently complementary to a sequence or region within or adjacent to intron 1, exon 2, or intron 2 of the pre-mRNA sequence of the human GAA gene. Ideally, an antisense oligomer is able to effectively modulate aberrant splicing of the GAA pre-mRNA, and thereby increase expression of active GAA protein. This requirement is optionally met when the oligomer compound has the ability to be actively taken up by mammalian cells, and once taken up, form a stable duplex (or heteroduplex) with the target mRNA, optionally with a Tm greater than about 40° C. or 45° C.

In certain embodiments, antisense oligomers may be 100% complementary to the target sequence, or may include mismatches, e.g., to accommodate variants, as long as a heteroduplex formed between the oligomer and target sequence is sufficiently stable to withstand the action of cellular nucleases and other modes of degradation which may occur in vivo. Hence, certain oligomers may have substantial complementarity, meaning, about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligomer and the target sequence. Oligomer backbones that are less susceptible to cleavage by nucleases are discussed herein. Mismatches, if present, are typically less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the v target sequence, it is effective to stably and specifically bind to the target sequence, such that splicing of the target pre-RNA is modulated.

The stability of the duplex formed between an oligomer and a target sequence is a function of the binding Tm and the susceptibility of the duplex to cellular enzymatic cleavage. The Tm of an oligomer with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligomer Hybridization Techniques, Methods Enzymol. Vol. 154 pp. 94-107. In certain embodiments, antisense oligomers may have a binding Tm, with respect to a complementary-sequence RNA, of greater than body temperature and preferably greater than about 45° C. or 50° C. Tm's in the range 60-80° C. or greater are also included. According to well-known principles, the Tm of an oligomer, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tm (45-50° C. or greater) at a length of 25 bases or less are generally preferred over those requiring greater than 25 bases for high Tm values.

Tables 2A, 2B, and 2C below show exemplary targeting sequences (in a 5′-to-3′ orientation) complementary to pre-mRNA sequences of the human GAA gene.

TABLE 2A
Antisense oligomer sequences for GAA-targeted oligomers

| | | SEQ |
|---|---|---|
| Name | Sequence (5′-3′) | ID NO |
| GAAEx2A(+201+225) | GCC CXG GXC XGC XGG CXC CCX GCX G | 4 |
| GAAEx2A(+200+224) | CCC XGG XCT GCT GGC TCC CTG CTG G | 5 |
| GAAEx2A(+199+223) | CCX GGX CXG CXG GCX CCC XGC XGG X | 6 |
| GAAEx2A(+198+222) | CXG GXC XGC XGG CXC CCX GCX GGX G | 7 |
| GAAEx2A(+197+221) | XGG XCX GCX GGC XCC CXG CXG GXG A | 8 |
| GAAEx2A(+196+220) | GGX CXG CXG GCX CCC XGC XGG XGA G | 9 |
| GAAEx2A(+195+219) | GXC XGC XGG CXC CCX GCX GGX GAG C | 10 |
| GAAEx2A(+194+218) | XCX GCX GGC XCC CXG CXG GXG AGC X | 11 |
| GAAEx2A(+203+227) | GGG CCC XGG XCX GCX GGC XCC CXG C | 12 |
| GAAEx2A(+204+228) | GGG GCC CXG GXC XGC XGG CXC CCX G | 13 |
| GAAEx2A(+205+229) | CGG GGC CCX GGX CXG CXG GCX CCC X | 14 |
| GAAEx2A(+206+230) | CCG GGG CCC XGG XCX GCX GGC XCC C | 15 |
| GAAEx2A(+207+231) | CCC GGG GCC CXG GXC XGC XGG CXC C | 16 |
| GAAEx2A(+208+232) | XCC CGG GGC CCX GGX CXG CXG GCX C | 17 |
| GAAEx2A(+209+233) | AXC CCG GGG CCC XGG XCX GCX GGC X | 18 |
| GAAEx2A(+210+234) | CAX CCC GGG GCC CXG GXC XGC XGG C | 19 |
| GAAEx2D(-12-38) | XCX GCC CXG GCC GCC GCC CCC GCC CCX | 20 |
| GAAEx2D(-54-78) | XGA GGX GCG XGG GXG XCG AXG XCC A | 21 |
| GAAEx2D(-55-79) | GAG GXG CGX GGG XGX CGA XGX CCA C | 22 |
| GAAEx2D(-56-80) | AGG XGC GXG GGX GXC GAX GXC CAC G | 23 |
| GAAEx2D(-59-83) | GCG CGX GGA CAX CGA CAC CCA CGC A | 24 |
| GAAEx2D(-52-76) | XGX GAG GGC GCG XGG ACA XCG ACA C | 25 |
| GAAEx2D(-51-75) | XXG XGA GGG CGC GXG GAC AXC GAC A | 26 |
| GAAEx2D(-50-74) | CX GXG AGG GCG CGX GGA CAX CGA C | 27 |
| GAAEx2A(+202+226) | GGC CCX GGX CXG CXG GCX CCC XGC X | 28 |
| GAA-IVS2.12.20 | XGG CCG CCG CCC CCG CCC CX | 29 |
| GAA-IVS2(53-72) | GXG AGG XGC GXG GGX GXC GA | 30 |

For any of the sequences in Table 2A, each X is independently selected from thymine (T) or uracil (U)

TABLE 2B
Antisense oligomer sequences for GAA-targeted oligomers

| | | SEQ |
|---|---|---|
| Name | Sequence (5′-3′) | ID NO |
| GAA-IVS1(-39-20) | GCX CAG CAG GGA GGC GGG AG | 133 |
| GAA-IVS1(-74-55) | GGC XCX CAA AGC AGC XCX GA | 134 |
| GAA-IVS1(-99-75) | GAC AXC AAC CGC GGC XGG CAC XGC A | 135 |
| GAA-IVS1(-139-115) | GGG XAA GGX GGC CAG GGX GGG XGX X | 136 |
| GAA-IVS1(-158-140) | GCC CXG CXG XCX AGA CXG G | 137 |
| GAA-IVS1(-179-160) | GAG AGG GCC AGA AGG AAG GG | 138 |
| GAA-IVS2(-9-20) | CCC GCC CCX GCC CXG CC | 139 |
| GAA-IVS2(-14-30) | XGG CCG CCG CCC CCG CCC | 140 |
| GAA-IVS2(-33-52) | XGX CCA CGC GCA CCC XCX GC | 141 |
| GAA-IVS2(-53-72) | GXG AGG XGC GXG GGX GXC GA | 142 |
| GAA-IVS2(-73-92) | GCA ACA XGC ACC CCA CCC XX | 143 |
| GAA-IVS2(-93-112) | AGG GCC CAG CAC ACA GXG GX | 144 |
| GAA-IVS2(-113-132) | XCA CAC CXC CGC XCC CAG CA | 145 |
| GAA-IVS2(-133-150) | GGC GCX GCC AXX GXC XGC | 146 |
| GAA-IVS2(-153-172) | GXG XCC CCA CXG CXC CCC GA | 147 |
| GAA-IVS2(-173-192) | CXG GAG XAC CXG XCA CCG XG | 148 |
| GAA-IVS2(-193-212) | XGA GCC CCG AGC CCX GCC XX | 149 |
| GAA-IVS2(-213-237) | XGA CCC ACC XXX XCA XAA AGA XGA A | 150 |
| GAA-IVS2(-234-258) | CXC XGG CAG CCC XAC XCX ACC XGA C | 151 |
| GAA-IVS2(-338-364) | CXA GXA XAA AXA CAX CCC AAA XXX XGC | 152 |
| GAAEx2A(+202+226) | GGC CCX GGX CXG CXG GCX CCC XGC X | 153 |
| GAAEx2A(+367+391) | GCX CCC XGC AGC CCC XGC XXX GCA G | 154 |
| GAA-IVS1.6.20 | GCG GGG CAG ACG XCA GGX GX | 155 |
| GAA-IVS1.10.20 | CAG CGC GGG GCA GAC GXC AG | 156 |
| GAA-IVS1.14.20 | CCG GCA GCG CGG GGC AGA CG | 157 |
| GAA-IVS1.17.20 | CCG CCG GCA GCG CGG GGC AG | 158 |
| GAA-IVS1.24.20 | GAX GXX ACC GCC GGC AGC GC | 159 |
| GAA-IVS1.28.20 | CXG GGA XGX XAC CGC CGG CA | 160 |
| GAA-IVS1.32.20 | GCX XCX GGG AXG XXA CCG CC | 161 |
| GAA-IVS1.2015.20 | XGG CAA CXC GXA XGX CCX XA | 162 |
| GAA-IVS1.2019.20 | AXX CXG GCA ACX CGX AXG XC | 163 |
| GAA-IVS1.2024.20 | AAG XGA XXC XGG CAA CXC GX | 164 |
| GAA-IVS1.2037.20 | XGG GXG XCA GCG GAA GXG AX | 165 |
| GAA-IVS1.2043.20 | GXC CAC XGG GXG XCA GCG GA | 166 |
| GAA-IVS1.2048.20 | GCX XGG XCC ACX GGG XGX CA | 167 |
| GAA-IVS1.2071.20 | CCC CAC XXC XGC AXA AAG GX | 168 |
| GAA-IVS1.2075.20 | GGA GCC CCA CXX CXG CAX AA | 169 |
| GAA-IVS1.2079.20 | GCX GGG AGC CCC ACX XCX GC | 170 |
| GAA-IVS1.2088.20 | CCA CGC CXG GCX GGG AGC CC | 171 |
| GAA-IVS1.2115.20 | XCC GAA GXG CXG GGA XXX CA | 172 |
| GAA-IVS1.2132.20 | XCC ACC CCC CXX GGC CXX CC | 173 |
| GAA-IVS1.2135.20 | XGA XCC ACC CCC CXX GGC CX | 174 |
| GAA-IVS1.2140.20 | XCA AGX GAX CCA CCC CCC XX | 175 |
| GAA-IVS1.2152.20 | GAA CXC CXG AGC XCA AGX GA | 176 |
| GAA-IVS1.2156.20 | XCX CGA ACX CCX GAG CXC AA | 177 |
| GAA-IVS1.2165.20 | CCA GGC XGG XCX CGA ACX CC | 178 |
| GAA-IVS1.2178.20 | XXX GCC AXG XXA CCC AGG CX | 179 |
| GAA-IVS1.2185.20 | ACG GGA XXX XGC CAX GXX AC | 180 |
| GAA-IVS1.2190.20 | XAG AGA CGG GAX XXX GCC AX | 181 |
| GAA-IVS1.2195.20 | XXX XGX AGA GAC GGG AXX XX | 182 |
| GAA-IVS1.2202.20 | XCX GXA XXX XXG XAG AGA CG | 183 |
| GAA-IVS1.2206.20 | AXX XXC XGX AXX XXX GXA GA | 184 |
| GAA-IVS1.2210.20 | GCX AAX XXX CXG XAX XXX XG | 185 |
| GAA-IVS2.9.20 | CCG CCG CCC CCG CCC CXG CC | 186 |
| GAA-IVS2.12.20 | XGG CCG CCG CCC CCG CCC CX | 187 |
| GAA-IVS2.18.20 | CXG CCC XGG CCG CCG CCC CC | 188 |
| GAA-IVS2.24.20 | CAC CCX CXG CCC XGG CCG CC | 189 |
| GAA-IVS2.27.20 | GCG CAC CCX CXG CCC XGG CC | 190 |
| GAA-IVS2.40.20 | XGX CGA XGX CCA CGC GCA CC | 191 |
| GAA-IVS2.48.20 | XGC GXG GGX GXC GAX GXC CA | 192 |
| GAA-IVS2.67.20 | GCA CCC CAC CCX XGX GAG GX | 193 |
| GAA-IVS2.72.20 | AAC AXG CAC CCC ACC CXX GX | 194 |
| GAA-IVS2.431.20 | AGG AGG AGG ACG CCX CCC CC | 195 |
| GAA-IVS2.446.20 | CXC AXC XGC AGA GCC AGG AG | 196 |
| GAA-IVS2.451.20 | GCX CCC XCA XCX GCA GAG CC | 197 |
| GAA-IVS2.454.20 | XCG GCX CCC XCA XCX GCA GA | 198 |
| GAA-IVS2.457.20 | GCC XCG GCX CCC XCA XCX GC | 199 |
| GAA-IVS1.30.20 | XXC XGG GAX GXX ACC GCC GG | 200 |
| GAA-IVS1.31.20 | CXX CXG GGA XGX XAC CGC CG | 201 |
| GAA-IVS1.33.20 | CGC XXC XGG GAX GXX ACC GC | 202 |
| GAA-IVS1.34.20 | CCG CXX CXG GGA XGX XAC CG | 203 |
| GAA-IVS1.36.20 | ACC CGC XXC XGG GAX GXX AC | 204 |
| GAA-IVS1.40.20 | XCA AAC CCG CXX CXG GGA XG | 205 |
| GAA-IVS1.44.20 | ACG XXC AAA CCC GCX XCX GG | 206 |
| GAA-IVS1(-73-54) | GGG CXC XCA AAG CAG CXC XG | 207 |
| GAA-IVS1(-72-53) | GGG GCX CXC AAA GCA GCX CX | 208 |
| GAA-IVS1(-70-51) | ACG GGG CXC XCA AAG CAG CX | 209 |
| GAA-IVS1(-68-49) | XCA CGG GGC XCX CAA AGC AG | 210 |
| GAA-IVS1(-75-56) | GCX CXC AAA GCA GCX CXG AG | 211 |
| GAA-IVS1(-76-57) | CXC XCA AAG CAG CXC XGA GA | 212 |
| GAA-IVS1(-78-59) | CXC AAA GCA GCX CXG AGA CA | 213 |
| GAA-IVS1(-80-61) | CAA AGC AGC XCX GAG ACA XC | 214 |
| GAA-IVS1(-82-63) | AAG CAG CXC XGA GAC AXC AA | 215 |
| GAAEx2A(+201+225) | GCC CXG GXC XGC XGG CXC CCX GCX G | 216 |
| GAAEx2A(+200+224) | CCC XGG XCX GCX GGC XCC CXG CXG G | 217 |
| GAAEx2A(+199+223) | CCX GGX CXG CXG GCX CCC XGC XGG X | 218 |
| GAAEx2A(+198+222) | CXG GXC XGC XGG CXC CCX GCX GGX G | 219 |
| GAAEx2A(+197+221) | XGG XCX GCX GGC XCC CXG CXG GXG A | 220 |
| GAAEx2A(+196+220) | GGX CXG CXG GCX CCC XGC XGG XGA G | 221 |
| GAAEx2A(+195+219) | GXC XGC XGG CXC CCX GCX GGX GAG C | 222 |
| GAAEx2A(+194+218) | XCX GCX GGC XCC CXG CXG GXG AGC X | 223 |
| GAAEx2A(+203+227) | GGG CCC XGG XCX GCX GGC XCC CXG C | 224 |
| GAAEx2A(+204+228) | GGG GCC CXG GXC XGC XGG CXC CCX G | 225 |
| GAAEx2A(+205+229) | CGG GGC CCX GGX CXG CXG GCX CCC X | 226 |
| GAAEx2A(+206+230) | CCG GGG CCC XGG XCX GCX GGC XCC C | 227 |
| GAAEx2A(+207+231) | CCC GGG GCC CXG GXC XGC XGG CXC C | 228 |
| GAAEx2A(+208+232) | XCC CGG GGC CCX GGX CXG CXG GCX C | 229 |
| GAAEx2A(+209+233) | AXC CCG GGG CCC XGG XCX GCX GGC X | 230 |
| GAAEx2A(+210+234) | CAX CCC GGG GCC CXG GXC XGC XGG C | 231 |
| GAAEx2D(-12-38) | XCX GCC CXG GCC GCC GCC CCC GCC CCX | 232 |
| GAAEx2D(-54-78) | XGA GGX GCG XGG GXG XCG AXG XCC A | 233 |
| GAAEx2D(-55-79) | GAG GXG CGX GGG XGX CGA XGX CCA C | 234 |
| GAAEx2D(-56-80) | AGG XGC GXG GGX GXC GAX GXC CAC G | 235 |
| GAAEx2D(-59-83) | GCG CGX GGA CAX CGA CAC CCA CGC A | 236 |
| GAAEx2D(-52-76) | XGX GAG GGC GCG XGG ACA XCG ACA C | 237 |
| GAAEx2D(-51-75) | XXG XGA GGG CGC GXG GAC AXC GAC A | 238 |
| GAAEx2D(-50-74) | CXX GXG AGG GCG CGX GGA CAX CGA C | 239 |
| GAA-IVS1(-177-160) | GAG AGG GCC AGA AGG AAG | 240 |
| GAA-IVS1(-179-162) | GAG GGC CAG AAG GAA GGG | 241 |
| GAA-IVS1(-181-164) | GGG CCA GAA GGA AGG GCG | 242 |
| GAA-IVS1(-175-158) | GGG AGA GGG CCA GAA GGA | 243 |
| GAA-IVS1(-180-161) | AGA GGG CCA GAA GGA AGG GC | 244 |
| GAA-IVS1(-181-162) | GAG GGC CAG AAG GAA GGG CG | 245 |
| GAA-IVS1(-182-163) | AGG GCC AGA AGG AAG GGC GA | 246 |
| GAA-IVS1(-182-164) | GGG CCA GAA GGA AGG GCG AG | 247 |
| GAA-IVS1(-184-165) | GGC CAG AAG GAA GGG CGA GA | 248 |
| GAA-IVS1(-185-166) | GCC AGA AGG AAG GGC GAG AA | 249 |
| GAA-IVS1(-179-158) | GGG AGA GGG CCA GAA GGA AGG G | 250 |
| GAA-IVS1(-179-155) | CXG GGG AGA GGG CCA GAA GGA AGG G | 251 |
| GAA-IVS1(-181-160) | GAG AGG GCC AGA AGG AAG GGC G | 252 |
| GAA-IVS1(-184-160) | GAG AGG GCC AGA AGG AAG GGC GAG A | 253 |
| GAA-IVS1(-189-170) | GAA GGA AGG GCG AGA AAA GC | 254 |
| GAA-IVS1(-209-190) | GCA GAA AAG CXC CAG CAG GG | 255 |

For any of the sequences in Table 2B, each X is independently selected from thymine (T) or uracil (U)

TABLE 2C
Antisense oligomer sequences for GAA-targeted oligomers

| | | SEQ |
|---|---|---|
| Name | Sequence (5′-3′) | ID NO |
| GAA-IVS1.SA.(-210,-186) | AAG CXC CAG CAG GGG AGX GCA GAG C | 296 |
| GAA-IVS1.SA.(-208,-184) | AAA AGC XCC AGC AGG GGA GXG CAG A | 297 |
| GAA-IVS1.SA.(-206,-182) | AGA AAA GCX CCA GCA GGG GAG XGC A | 298 |
| GAA-IVS1.SA.(-204,-180) | CGA GAA AAG CXC CAG CAG GGG AGX G | 299 |
| GAA-IVS1.SA.(-202,-178) | GGC GAG AAA AGC XCC AGC AGG GGA G | 300 |
| GAA-IVS1.SA.(-200,-176) | AGG GCG AGA AAA GCX CCA GCA GGG G | 301 |
| GAA-IVS1.SA.(-198,-174) | GAA GGG CGA GAA AAG CXC CAG CAG G | 302 |
| GAA-IVS1.SA.(-196,-172) | AGG AAG GGC GAG AAA AGC XCC AGC A | 303 |
| GAA-IVS1.SA.(-194,-170) | GAA GGA AGG GCG AGA AAA GCX CCA G | 304 |
| GAA-IVS1.SA.(-192,-168) | CAG AAG GAA GGG CGA GAA AAG CXC C | 305 |
| GAA-IVS1.SA.(-190,-166) | GCC AGA AGG AAG GGC GAG AAA AGC X | 306 |
| GAA-IVS1.SA.(-188,-164) | GGG CCA GAA GGA AGG GCG AGA AAA G | 307 |
| GAA-IVS1.SA.(-186,-162) | GAG GGC CAG AAG GAA GGG CGA GAA A | 308 |
| GAA-IVS1(-184-160) | GAG AGG GCC AGA AGG AAG GGC GAG A | 309 |
| GAA-IVS1(-182-163) | AGG GCC AGA AGG AAG GGC GA | 310 |
| GAA-IVS1(-179-160) | GAG AGG GCC AGA AGG AAG GG | 311 |
| GAA-IVS1(-179-155) | CXG GGG AGA GGG CCA GAA GGA AGG G | 312 |
| GAA-IVS1(-177-160) | GAG AGG GCC AGA AGG AAG | 313 |
| GAA-IVS1(-175-158) | GGG AGA GGG CCA GAA GGA | 314 |
| GAAEx2A(+196+220) | GGX CXG CXG GCX CCC XGC XGG XGA G | 315 |
| GAA-IVS1(-70-46) | CAC XCA CGG GGC XCX CAA AGC AGC X | 316 |
| GAA-IVS1.24.25 | XCX GGG AXG XXA CCG CCG GCA GCG C | 317 |
| GAA-IVS1.2178.20 | XXX GCC AXG XXA CCC AGG CX | 318 |
| GAA-IVS1(-71-47) | ACX CAC GGG GCX CXC AAA GCA GCX C | 319 |
| GAA-IVS1(-69-45) | GCA CXC ACG GGG CXC XCA AAG CAG C | 320 |
| GAA-IVS1(-76-52) | CGG GGC XCX CAA AGC AGC XCX GAG A | 321 |
| GAA-IVS1(-75-51) | ACG GGG CXC XCA AAG CAG CXC XGA G | 322 |
| GAA-IVS1(-74-50) | CAC GGG GCX CXC AAA GCA GCX CXG A | 323 |
| GAA-IVS1(-73-49) | XCA CGG GGC XCX CAA AGC AGC XCX G | 324 |
| GAA-IVS1(-72-48) | CXC ACG GGG CXC XCA AAG CAG CXC X | 325 |
| GAA-IVS1(-68-44) | GGC ACX CAC GGG GCX CXC AAA GCA G | 326 |
| GAA-IVS1(-67-43) | CGG CAC XCA CGG GGC XCX CAA AGC A | 327 |
| GAA-IVS1(-66-42) | GCG GCA CXC ACG GGG CXC XCA AAG C | 328 |
| GAA-IVS1(-65-41) | GGC GGC ACX CAC GGG GCX CXC AAA G | 329 |
| GAA-IVS1(-64-40) | GGG CGG CAC XCA CGG GGC XCX CAA A | 330 |
| GAA-IVS1(-63-39) | GGG GCG GCA CXC ACG GGG CXC XCA A | 331 |
| GAA-IVS1(-62-38) | AGG GGC GGC ACX CAC GGG GCX CXC A | 332 |
| GAA-IVS1(-61-37) | GAG GGG CGG CAC XCA CGG GGC XCX C | 333 |
| GAA-IVS1(-74-55) | GGC XCX CAA AGC AGC XCX GA | 334 |
| GAA-IVS1.25.25 | XXC XGG GAX GXX ACC GCC GGC AGC G | 335 |
| GAA-IVS1.26.25 | CXX CXG GGA XGX XAC CGC CGG CAG C | 336 |
| GAA-IVS1.27.25 | GCX XCX GGG AXG XXA CCG CCG GCA G | 337 |
| GAA-IVS1.28.25 | CGC XXC XGG GAX GXX ACC GCC GGC A | 338 |
| GAA-IVS1.29.25 | CCG CXX CXG GGA XGX XAC CGC CGG C | 339 |
| GAA-IVS1.30.25 | CCC GCX XCX GGG AXG XXA CCG CCG G | 340 |
| GAA-IVS1.31.25 | ACC CGC XXC XGG GAX GXX ACC GCC G | 341 |
| GAA-IVS1.32.25 | AAC CCG CXX CXG GGA XGX XAC CGC C | 342 |

For any of the sequences in Table 2C, each X is independently selected from thymine (T) or uracil (U)

Certain antisense oligomers thus comprise, consist, or consist essentially of a sequence in Table 2A (e.g., SEQ ID NOS:4-30) or a variant or contiguous or non-contiguous portion(s) thereof. For instance, certain antisense oligomers comprise about or at least about 6, 7, 8, 9, 10, 11, 12, 13, 14, 15, 16, 17, 18, 19, 20, 21, 22, 23, 24, 25, 26, or 27 contiguous or non-contiguous nucleotides of any of SEQ ID NOS:4-30. For non-contiguous portions, intervening nucleotides can be deleted or substituted with a different nucleotide, or intervening nucleotides can be added. Additional examples of variants include oligomers having about or at least about 70% sequence identity or homology, e.g., 70%, 71%, 72%, 73%, 74%, 75%, 76%, 77%, 78%, 79%, 80% 1,%, 81%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS:4-30. In some embodiments, any of the antisense oligomers or compounds comprising, consisting of, or consisting essentially of such variant sequences suppress an ISS and/or ESS element in the GAA pre-mRNA. In some embodiments, the antisense oligomer or compound with a targeting sequence that comprises, consists of, or consists essentially of such a variant sequence suppresses an ISS and/or ESS element in the GAA pre-mRNA. In some embodiments, the antisense oligomer or compound with a targeting sequence that comprises, consists of, or consists essentially of such a variant sequence increases, enhances, or promotes exon 2 retention in the mature GAA mRNA, optionally, by at least about 10%, 15%, 20%, 25%, 30%, 35%, 40%, 45%, 50%, 55%, 60%, or 65% or more relative to a control, according to at least one of the examples or methods described herein. In some embodiments, the antisense oligomer or compound with a targeting sequence that comprises, consists of, or consists essentially of such a variant sequence increases, enhances, or promotes GAA protein expression in a cell, optionally, by at least about 10%, 15%, 20%, 25%, 30%, 35%, 40%, 45%, 50%, 55%, 60%, or 65% or more relative to a control, according to at least one of the examples or methods described herein. In some embodiments, the antisense oligomer or compound comprising, consisting of, or consisting essentially of such a variant sequence increases, enhances, or promotes GAA enzymatic activity in a cell, optionally, by at least about 10%, 15%, 20%, 25%, 30%, 35%, 40%, 45%, 50%, 55%, 60%, or 65% or more relative to a control, according to at least one of the examples or methods described herein. As exemplified in the working examples, the cell (e.g., a fibroblast cell) can be obtained from a patient having a IVS1-13T>G mutation.

In some embodiments, certain antisense oligomers comprise, consist, or consist essentially of a sequence in Table 2B (e.g., SEQ ID NOS: 133-255) or a variant or contiguous or non-contiguous portion(s) thereof. For instance, certain antisense oligomers comprise about or at least about 6, 7, 8, 9, 10, 11, 12, 13, 14, 15, 16, 17, 18, 19, 20, 21, 22, 23, 24, 25, 26, or 27 contiguous or non-contiguous nucleotides of any of SEQ ID NOS:133-255. For non-contiguous portions, intervening nucleotides can be deleted or substituted with a different nucleotide, or intervening nucleotides can be added. Additional examples of variants include oligomers having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS:133-255. In some embodiments, the antisense oligomer or compound with a targeting sequence that comprises, consists of, or consists essentially of such a variant sequence suppresses an ISS and/or ESS element in the GAA pre-mRNA. In some embodiments, the antisense oligomer or compound with a targeting sequence that comprises, consists of, or consists essentially of such a variant sequence increases, enhances, or promotes exon 2 retention in the mature GAA mRNA, optionally, by at least about 10%, 15%, 20%, 25%, 30%, 35%, 40%, 45%, 50%, 55%, 60%, or 65% or more relative to a control, according to at least one of the examples or methods described herein. In some embodiments, the antisense oligomer or compound with a targeting sequence that comprises, consists of, or consists essentially of such a variant sequence increases, enhances, or promotes GAA protein expression in a cell, optionally, by at least about 10%, 15%, 20%, 25%, 30%, 35%, 40%, 45%, 50%, 55%, 60%, or 65% or more relative to a control, according to at least one of the examples or methods described herein. In some embodiments, the antisense oligomer or compound comprising, consisting of, or consisting essentially of such a variant sequence increases, enhances, or promotes GAA enzymatic activity in a cell, optionally, by at least about 10%, 15%, 20%, 25%, 30%, 35%, 40%, 45%, 50%, 55%, 60%, or 65% or more relative to a control, according to at least one of the examples or methods described herein. As exemplified in the working examples, the cell (e.g., a fibroblast cell) can be obtained from a patient having a IVS1-13T>G mutation.

In some embodiments, certain antisense oligomers comprise, consist, or consist essentially of a sequence in Table 2C (e.g., SEQ ID NOS:296-342) or a variant or contiguous or non-contiguous portion(s) thereof. For instance, certain antisense oligomers comprise about or at least about 6, 7, 8, 9, 10, 11, 12, 13, 14, 15, 16, 17, 18, 19, 20, 21, 22, 23, 24, 25, 26, or 27 contiguous or non-contiguous nucleotides of any of SEQ ID NOS:296-342. For non-contiguous portions, intervening nucleotides can be deleted or substituted with a different nucleotide, or intervening nucleotides can be added. Additional examples of variants include oligomers having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS:296-342. In some embodiments, the antisense oligomer or compound with a targeting sequence that comprises, consists of, or consists essentially of such a variant sequence suppresses an ISS and/or ESS element in the GAA pre-mRNA. In some embodiments, the antisense oligomer or compound with a targeting sequence that comprises, consists of, or consists essentially of such a variant sequence increases, enhances, or promotes exon 2 retention in the mature GAA mRNA, optionally, by at least about 10%, 15%, 20%, 25%, 30%, 35%, 40%, 45%, 50%, 55%, 60%, or 65% or more relative to a control, according to at least one of the examples or methods described herein. In some embodiments, the antisense oligomer or compound with a targeting sequence that comprises, consists of, or consists essentially of such a variant sequence increases, enhances, or promotes GAA protein expression in a cell, optionally, by at least about 10%, 15%, 20%, 25%, 30%, 35%, 40%, 45%, 50%, 55%, 60%, or 65% or more relative to a control, according to at least one of the examples or methods described herein. In some embodiments, the antisense oligomer or compound comprising, consisting of, or consisting essentially of such a variant sequence increases, enhances, or promotes GAA enzymatic activity in a cell, optionally, by at least about 10%, 15%, 20%, 25%, 30%, 35%, 40%, 45%, 50%, 55%, 60%, or 65% or more relative to a control, according to at least one of the examples or methods described herein. As exemplified in the working examples, the cell (e.g., a fibroblast cell) can be obtained from a patient having a IVS1-13T>G mutation.

In various aspects an antisense oligomer or compound is provided, comprising a targeting sequence that is complementary (e.g., at least 80, 81, 82, 83, 84, 85, 86, 87, 88, 89, 90, 91, 92, 93, 94, 95, 96, 97, 98, 99, or 100% complementary) to a target region of the human GAA pre-mRNA, optionally where the targeting sequences is as set forth in Table 2A, 2B, or 2C. In another aspect, an antisense oligomer or compound is provided, comprising a variant targeting sequence, such as any of those described herein, wherein the variant targeting sequence binds to a target region of the human pre-mRNA that is complementary (e.g., 80%-100% complementary) to one or more of the targeting sequences set forth in Table 2A, 2B, or 2C. In some embodiments, the antisense oligomer or compound binds to a target sequence comprising at least 10 (e.g., at least 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40) consecutive bases of the human GAA pre-mRNA (e.g., any of SEQ ID Nos: 1, 2, or 3 or a sequence that spans a GAA pre-mRNA splice junction defined by SEQ ID NO:1/2 or SEQ ID NO:2/3). In some embodiments, the target sequence is complementary (e.g., at least 80, 81, 82, 83, 84, 85, 86, 87, 88, 89, 90, 91, 92, 93, 94, 95, 96, 97, 98, 99, or 100% complementary) to one or more of the targeting sequences set forth in Table 2A, 2B, or 2C. In some embodiments, the target sequence is complementary (e.g., at least 80, 81, 82, 83, 84, 85, 86, 87, 88, 89, 90, 91, 92, 93, 94, 95, 96, 97, 98, 99, or 100% complementary) to at least 10 (e.g., at least 11, 12, 13, 14, 15, 16, 17, 18, 19, 20, 21, 22, 23, 24, 25, 26, 27, or 28) consecutive bases of one or more of the targeting sequences set forth in Table 2A, 2B, or 2C. In some embodiments, the target sequence is defined by an annealing site (e.g., GAAEx2A(+201+225)) as set forth in any one of Tables 2A, 2B, or 2C.

The activity of antisense oligomers and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e., a transcribed polynucleotide) from a cell, tissue or organism, and by hybridizing the mRNA/cDNA with a reference polynucleotide that is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

### III. Antisense Oligomer Chemistries

A. General Characteristics

Certain antisense oligomers of the instant disclosure specifically hybridize to an intronic splice silencer element or an exonic splice silencer element. Some antisense oligomers comprise a targeting sequence set forth in Tables 2A-2C, a fragment of at least 10 contiguous nucleotides of a targeting sequence in Tables 2A-2C, or variant having at least 80% sequence identity to a targeting sequence in Tables 2A-2C. Specific antisense oligomers consist or consist essentially of a targeting sequence set forth in Tables 2A-2C. In some embodiments, the oligomer is nuclease-resistant.

In certain embodiments, the antisense oligomer comprises a non-natural chemical backbone selected from a phosphoramidate or phosphorodiamidate morpholino oligomer (PMO), a peptide nucleic acid (PNA), a locked nucleic acid (LNA), a phosphorothioate oligomer, a tricyclo-DNA oligomer, a tricyclo-phosphorothioate oligomer, a 2′O-Me-modified oligomer, or any combination of the foregoing, and a targeting sequence complementary to a region within intron 1 (SEQ ID. NO: 1), intron 2 (SEQ ID. NO: 2), or exon 2 (SEQ ID. NO: 3) of a pre-mRNA of the human acid alpha-glucosidase (GAA) gene. For example, in some embodiments, the targeting sequence is selected from SEQ ID NOS: 4 to 30, 133 to 255, or 296 to 342, wherein X is selected from uracil (U) or thymine (T).

Antisense oligomers of the disclosure generally comprise a plurality of nucleotide subunits each bearing a nucleobase which taken together form or comprise a targeting sequence, for example, as discussed above. Accordingly, in some embodiments, the antisense oligomers range in length from about 10 to about 40 subunits, more preferably about 10 to 30 subunits, and typically 15-25 subunits. For example, antisense compounds of the disclosure may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 subunits in length, or range from 10 subunits to 40 subunits, 10 subunits to 30 subunits, 14 subunits to 25 subunits, 15 subunits to 30 subunits, 17 subunits to 30 subunits, 17 subunits to 27 subunits, 10 subunits to 27 subunits, 10 subunits to 25 subunits, and 10 subunits to 20 subunits. In certain embodiments, the antisense oligomer is about 10 to about 40 or about 5 to about 30 nucleotides in length. In some embodiments, the antisense oligomer is about 14 to about 25 or about 17 to about 27 nucleotides in length.

In various embodiments, an antisense oligomer may comprise a completely modified backbone, for example, 100% of the backbone is modified (for example, a 25 mer antisense oligomer comprises its entire backbone modified with any combination of the backbone modifications as described herein). In various embodiments, an antisense oligomer may comprise about 100% to 2.5% of its backbone modified. In various embodiments, an antisense oligomer may comprise about 99%, 95%, 90%, 85%, 80%, 75%, 70%, 65%, 60%, 55%, 50%, 45%, 40%, 35%, 30%, 25%, 20%, 15%, 10%, 5%, or 2.5% of its backbone modified, and iterations in between. In other embodiments, an antisense oligomer may comprise any combination of backbone modifications as described herein.

In various embodiments, an antisense oligomer may comprise, consist of, or consist essentially of phosphoramidate morpholino oligomers and phosphorodiamidate morpholino oligomers (PMO), phosphorothioate modified oligomers, 2′ O-methyl modified oligomers, peptide nucleic acid (PNA), locked nucleic acid (LNA), phosphorothioate oligomers, 2′ O-MOE modified oligomers, 2′-fluoro-modified oligomer, 2′O,4′C-ethylene-bridged nucleic acids (ENAs), tricyclo-DNAs, tricyclo-DNA phosphorothioate nucleotides, 2′-O-[2-(N-methylcarbamoyl)ethyl] modified oligomers, morpholino oligomers, peptide-conjugated phosphoramidate morpholino oligomers (PPMO), phosphorodiamidate morpholino oligomers having a phosphorous atom with (i) a covalent bonds to the nitrogen atom of a morpholino ring, and (ii) a second covalent bond to a (1,4-piperazin)-1-yl substituent or to a substituted (1,4-piperazin)-1-yl (PMOplus), and phosphorodiamidate morpholino oligomers having a phosphorus atom with (i) a covalent bond to the nitrogen atom of a morpholino ring and (ii) a second covalent bond to the ring nitrogen of a 4-aminopiperdin-1-yl (i.e., APN) or a derivative of 4-aminopiperdin-1-yl (PMO-X) chemistries, including combinations of any of the foregoing.

In some embodiments, the backbone of the antisense oligomer is substantially uncharged, and is optionally recognized as a substrate for active or facilitated transport across the cell membrane. In some embodiments, all the internucleoside linkages are uncharged. The ability of the oligomer to form a stable duplex with the target RNA may also relate to other features of the backbone, including the length and degree of complementarity of the antisense oligomer with respect to the target, the ratio of G:C to A:T base matches, and the positions of any mismatched bases. The ability of the antisense oligomer to resist cellular nucleases may promote survival and ultimate delivery of the agent to the cell cytoplasm. Exemplary antisense oligomer targeting sequences are listed in Tables 2A, 2B, and 2C (supra).

In certain embodiments, the antisense oligomer has at least one internucleoside linkage that is positively charged or cationic at physiological pH. In some embodiments, the antisense oligomer has at least one internucleoside linkage that exhibits a pKa between about 5.5 and about 12. In further embodiments, the antisense oligomer contains about, at least about, or no more than about 1, 2, 3, 4, 5, 6, 7, 8, 9, or 10 internucleoside linkages that exhibits a pKa between about 4.5 and about 12. In some embodiments, the antisense oligomer contains about or at least about 10%, 15%, 20%, 25%, 30%, 35%, 40%, 45%, 50%, 55%, 60%, 65%, 70%, 75%, 80%, 85%, 90%, 95%, or 100% internucleoside linkages that exhibit a pKa between about 4.5 and about 12. Optionally, the antisense oligomer has at least one internucleoside linkage with both a basic nitrogen and an alkyl, aryl, or aralkyl group. In particular embodiments, the cationic internucleoside linkage or linkages comprise a 4-aminopiperdin-1-yl (APN) group, or a derivative thereof. While not being bound by any one theory, it is believed that the presence of a cationic linkage or linkages (e.g., APN group or APN derivative) in the oligomer facilitates binding to the negatively charged phosphates in the target nucleotide. Thus, the formation of a heteroduplex between mutant RNA and the cationic linkage-containing oligomer may be held together by both an ionic attractive force and Watson-Crick base pairing.

In some embodiments, the number of cationic linkages is at least 2 and no more than about half the total internucleoside linkages, e.g., about or no more than about 1, 2, 3, 4, 5, 6, 7, 8, 9, 10, 11, 12, 13, 14, 15, 16, 17, 18, 19, 20 cationic linkages. In some embodiments, however, up to all of the internucleoside linkages are cationic linkages, e.g., about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 of the total internucleoside linkages are cationic linkages. In specific embodiments, an oligomer of about 19-20 subunits may have 2-10, e.g., 4-8, cationic linkages, and the remainder uncharged linkages. In other specific embodiments, an oligomer of 14-15 subunits may have 2-7, e.g., 2, 3, 4, 5, 6, or 7 cationic linkages and the remainder uncharged linkages. The total number of cationic linkages in the oligomer can thus vary from about 1 to 10 to 15 to 20 to 30 or more (including all integers in between), and can be interspersed throughout the oligomer.

In some embodiments, an antisense oligomer may have about or up to about 1 cationic linkage per every 2-5 or 2, 3, 4, or 5 uncharged linkages, such as about 4-5 or 4 or 5 per every 10 uncharged linkages.

Certain embodiments include antisense oligomers that contain about 10%, 15%, 20%, 25%, 30%, 35%, 40%, 45%, 50%, 55%, 60%, 65%, 70%, 75%, 80%, 85%, 90%, 95%, or 100% cationic linkages. In certain embodiments, optimal improvement in antisense activity may be seen if about 25% of the backbone linkages are cationic. In certain embodiments, enhancement may be seen with a small number e.g., 10-20% cationic linkages, or where the number of cationic linkages are in the range 50-80%, such as about 60%.

In some embodiments, the cationic linkages are interspersed along the backbone. Such oligomers optionally contain at least two consecutive uncharged linkages; that is, the oligomer optionally does not have a strictly alternating pattern along its entire length. In specific instances, each one or two cationic linkage(s) is/are separated along the backbone by at least 1, 2, 3, 4, or 5 uncharged linkages.

Also included are oligomers having blocks of cationic linkages and blocks of uncharged linkages. For example, a central block of uncharged linkages may be flanked by blocks of cationic linkages, or vice versa. In some embodiments, the oligomer has approximately equal-length 5′, 3′ and center regions, and the percentage of cationic linkages in the center region is greater than about 50%, 60%, 70%, or 80% of the total number of cationic linkages.

In certain antisense oligomers, the bulk of the cationic linkages (e.g., 70, 75%, 80%, 90% of the cationic linkages) are distributed close to the “center-region” backbone linkages, e.g., the 6, 7, 8, 9, 10, 11, 12, 13, 14, or 15 centermost linkages. For example, a 16, 17, 18, 19, 20, 21, 22, 23, or 24-mer oligomer with may have at least 50%, 60%, 70%, or 80% of the total cationic linkages localized to the 8, 9, 10, 11, or 12 centermost linkages.

B. Backbone Chemistry Features

The antisense oligomers can employ a variety of antisense chemistries. Examples of oligomer chemistries include, without limitation, phosphoramidate morpholino oligomers and phosphorodiamidate morpholino oligomers (PMO), phosphorothioate modified oligomers, 2′ O-methyl modified oligomers, peptide nucleic acid (PNA), locked nucleic acid (LNA), phosphorothioate oligomers, 2′ O-MOE modified oligomers, 2′-fluoro-modified oligomer, 2′O,4′C-ethylene-bridged nucleic acids (ENAs), tricyclo-DNAs, tricyclo-DNA phosphorothioate nucleotides, 2′-O-[2-(N-methylcarbamoyl)ethyl] modified oligomers, morpholino oligomers, peptide-conjugated phosphoramidate morpholino oligomers (PPMO), phosphorodiamidate morpholino oligomers having a phosphorous atom with (i) a covalent bonds to the nitrogen atom of a morpholino ring, and (ii) a second covalent bond to a (1,4-piperazin)-1-yl substituent or to a substituted (1,4-piperazin)-1-yl (PMOplus), and phosphorodiamidate morpholino oligomers having a phosphorus atom with (i) a covalent bond to the nitrogen atom of a morpholino ring and (ii) a second covalent bond to the ring nitrogen of a 4-aminopiperdin-1-yl (i.e., APN) or a derivative of 4-aminopiperdin-1-yl (PMO-X) chemistries, including combinations of any of the foregoing. In general, PNA and LNA chemistries can utilize shorter targeting sequences because of their relatively high target binding strength relative to PMO and 2′O-Me modified oligomers. Phosphorothioate and 2′O-Me-modified chemistries can be combined to generate a 2′O-Me-phosphorothioate backbone. See, e.g., PCT Publication Nos. WO/2013/112053 and WO/2009/008725, which are hereby incorporated by reference in their entireties.

In some instances, antisense oligomers such as PMOs can be conjugated to cell penetrating peptides (CPPs) to facilitate intracellular delivery. Peptide-conjugated PMOs are called PPMOs and certain embodiments include those described in PCT Publication No. WO/2012/150960, incorporated herein by reference in its entirety. In some embodiments, an arginine-rich peptide sequence conjugated or linked to, for example, the 3′ terminal end of an antisense oligomer as described herein may be used. In certain embodiments, an arginine-rich peptide sequence conjugated or linked to, for example, the 5′ terminal end of an antisense oligomer as described herein may be used.

1. Peptide Nucleic Acids (PNAs)

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligomers obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases. A non-limiting example of a PNA is depicted below:

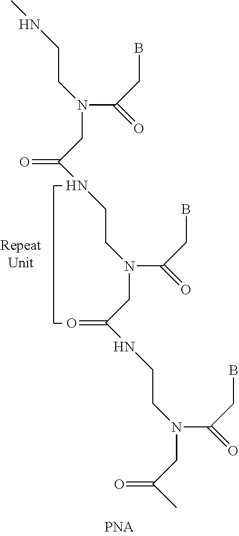

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

2. Locked Nucleic Acids (LNAs)

Antisense oligomer compounds may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230, which are hereby incorporated by reference in their entirety. A non-limiting example of an LNA is depicted below:

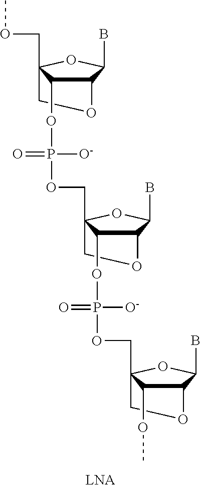

Compounds of the disclosure may incorporate one or more LNAs; in some cases, the compounds may be entirely composed of LNAs. Methods for the synthesis of individual LNA nucleoside subunits and their incorporation into oligomers are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. Further embodiments include an LNA containing compound where each LNA subunit is separated by a DNA subunit. Certain compounds are composed of alternating LNA and DNA subunits where the intersubunit linker is phosphorothioate.

2′O,4′C-ethylene-bridged nucleic acids (ENAs) are another member of the class of BNAs. A non-limiting example is depicted below:

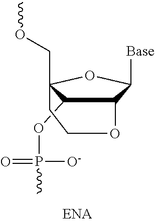

ENA oligomers and their preparation are described in Obika et al., Tetrahedron Ltt 38(50): 8735, which is hereby incorporated by reference in its entirety. Compounds of the disclosure may incorporate one or more ENA subunits.

3. Phosphorothioates

“Phosphorothioates” (or S-oligos) are a variant of normal DNA in which one of the nonbridging oxygens is replaced by a sulfur. A non-limiting example of a phosphorothioate is depicted below:

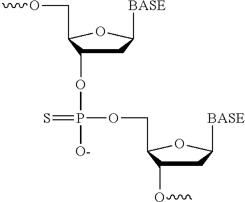

The sulfurization of the internucleotide bond reduces the action of endo- and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990, which are hereby incorporated by reference in their entirety). The latter methods avoid the problem of elemental sulfur's insolubility in most organic solvents and the toxicity of carbon disulfide. The TETD and BDTD methods also yield higher purity phosphorothioates.

4. Triclyclo-DNAs and Tricyclo-Phosphorothioate Nucleotides

Tricyclo-DNAs (tc-DNA) are a class of constrained DNA analogs in which each nucleotide is modified by the introduction of a cyclopropane ring to restrict conformational flexibility of the backbone and to optimize the backbone geometry of the torsion angle γ. Homobasic adenine- and thymine-containing tc-DNAs form extraordinarily stable A-T base pairs with complementary RNAs. Tricyclo-DNAs and their synthesis are described in International Patent Application Publication No. WO 2010/115993, which are hereby incorporated by reference in their entirety. Compounds of the disclosure may incorporate one or more tricycle-DNA nucleotides; in some cases, the compounds may be entirely composed of tricycle-DNA nucleotides.

Tricyclo-phosphorothioate nucleotides are tricyclo-DNA nucleotides with phosphorothioate intersubunit linkages. Tricyclo-phosphorothioate nucleotides and their synthesis are described in International Patent Application Publication No. WO 2013/053928, which are hereby incorporated by reference in their entirety. Compounds of the disclosure may incorporate one or more tricycle-DNA nucleotides; in some cases, the compounds may be entirely composed of tricycle-DNA nucleotides. A non-limiting example of a tricycle-DNA/tricycle-phosphothioate nucleotide is depicted below:

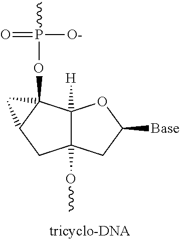

5. 2′ O-Methyl, 2′ O-MOE, and 2′-F Oligomers

“2′O-Me oligomer” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligomers (PTOs) for further stabilization. 2′O-Me oligomers (phosphodiester or phosphothioate) can be synthesized according to routine techniques in the art (see, e.g., Yoo et al., Nucleic Acids Res. 32:2008-16, 2004, which is hereby incorporated by reference in its entirety). A non-limiting example of a 2′ O-Me oligomer is depicted below:

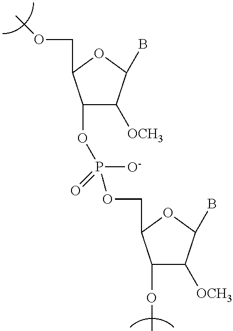

2′ O-Me oligomers may also comprise a phosphorothioate linkage (2′ O-Me phosphorothioate oligomers). 2′ O-Methoxyethyl Oligomers (2′-0 MOE), like 2′ O-Me oligomers, carry a methoxyethyl group at the 2′-OH residue of the ribose molecule and are discussed in Martin et al., Helv. Chim. Acta, 78, 486-504, 1995, which are hereby incorporated by reference in their entirety. A non-limiting example of a 2′ O-MOE nucleotide is depicted below:

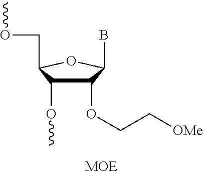

In contrast to the preceding alkylated 2′OH ribose derivatives, 2′-fluoro oligomers have a fluoro radical in at the 2′ position in place of the 2′OH. A non-limiting example of a 2′-F oligomer is depicted below:

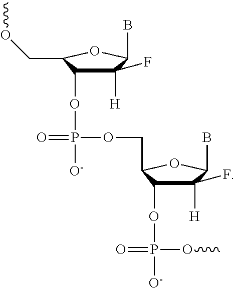

2′-fluoro oligomers are further described in WO 2004/043977, which is hereby incorporated by reference in its entirety. Compounds of the disclosure may incorporate one or more 2′O-Methyl, 2′ O-MOE, and 2′-F subunits and may utilize any of the intersubunit linkages described here. In some instances, a compound of the disclosure could be composed of entirely 2′O-Methyl, 2′ O-MOE, or 2′-F subunits. One embodiment of a compound of the disclosure is composed entirely of 2′O-methyl subunits.

6. 2′-O-[2-(N-methylcarbamoyl)ethyl] Oligonucleotides (MCEs)

MCEs are another example of 2′O modified ribonucleosides useful in the compounds of the disclosure. Here, the 2′OH is derivatized to a 2-(N-methylcarbamoyl)ethyl moiety to increase nuclease resistance. A non-limiting example of an MCE oligomer is depicted below:

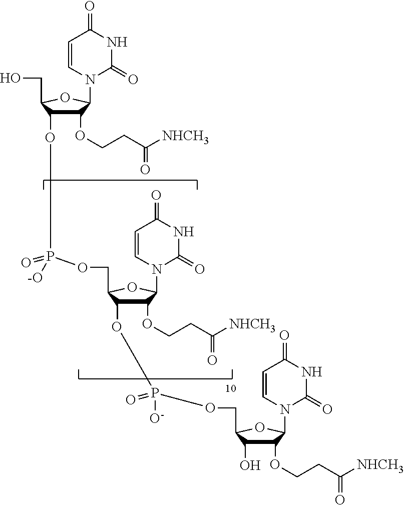

MCEs and their synthesis are described in Yamada et al., J. Org. Chem., 76(9):3042-53, which is hereby incorporated by reference in its entirety. Compounds of the disclosure may incorporate one or more MCE subunits.

7. Morpholino-based Oligomers

Morpholino-based oligomers refer to an oligomer comprising morpholino subunits supporting a nucleobase and, instead of a ribose, contains a morpholine ring. Exemplary internucleoside linkages include, for example, phosphoramidate or phosphorodiamidate internucleoside linkages joining the morpholine ring nitrogen of one morpholino subunit to the 4′ exocyclic carbon of an adjacent morpholino subunit. Each morpholino subunit comprises a purine or pyrimidine nucleobase effective to bind, by base-specific hydrogen bonding, to a base in an oligonucleotide.

Morpholino-based oligomers (including antisense oligomers) are detailed, for example, in U.S. Pat. Nos. 5,698,685; 5,217,866; 5,142,047; 5,034,506; 5,166,315; 5,185,444; 5,521,063; 5,506,337 and pending U.S. patent application Ser. Nos. 12/271,036; 12/271,040; and PCT Publication No. WO/2009/064471 and WO/2012/043730 and Summerton et al. 1997, Antisense and Nucleic Acid Drug Development, 7, 187-195, which are hereby incorporated by reference in their entirety. Within the oligomer structure, the phosphate groups are commonly referred to as forming the “internucleoside linkages” of the oligomer. The naturally occurring internucleoside linkage of RNA and DNA is a 3′ to 5′ phosphodiester linkage. A “phosphoramidate” group comprises phosphorus having three attached oxygen atoms and one attached nitrogen atom, while a “phosphorodiamidate” group comprises phosphorus having two attached oxygen atoms and two attached nitrogen atoms. In the uncharged or the cationic intersubunit linkages of morpholino-based oligomers described herein, one nitrogen is always pendant to the backbone chain. The second nitrogen, in a phosphorodiamidate linkage, is typically the ring nitrogen in a morpholine ring structure.

“PMO-X” refers to phosphorodiamidate morpholino-based oligomers having a phosphorus atom with (i) a covalent bond to the nitrogen atom of a morpholine ring and (ii) a second covalent bond to the ring nitrogen of a 4-aminopiperdin-1-yl (i.e., APN) or a derivative of 4-aminopiperdin-1-yl. Exemplary PMO-X oligomers are disclosed in PCT Application No. PCT/US2011/38459 and PCT Publication No. WO 2013/074834, which are hereby incorporated by reference in their entirety. PMO-X includes “PMO-apn” or “APN,” which refers to a PMO-X oligomer which comprises at least one internucleoside linkage where a phosphorus atom is linked to a morpholino group and to the ring nitrogen of a 4-aminopiperdin-1-yl (i.e., APN). In specific embodiments, an antisense oligomer comprising a targeting sequence as set forth in Tables 2A, 2B, or 2C comprises at least one APN-containing linkage or APN derivative-containing linkage. Various embodiments include morpholino-based oligomers that have about 10%, 15%, 20%, 25%, 30%, 35%, 40%, 45%, 50%, 55%, 60%, 65%, 70%, 75%, 80%, 85%, 90%, 95%, or 100% APN/APN derivative-containing linkages, where the remaining linkages (if less than 100%) are uncharged linkages, e.g., about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 of the total internucleoside linkages are APN/APN derivative-containing linkages.

In some embodiments, the antisense oligomer is a compound of formula (I):

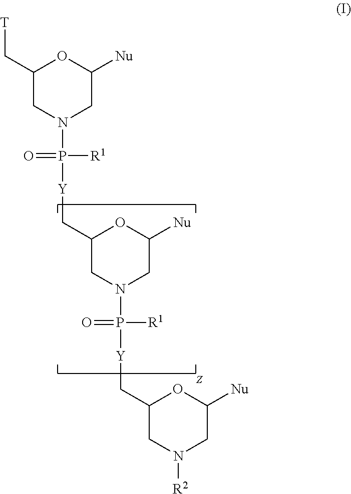

or a pharmaceutically acceptable salt thereof, wherein:
each Nu is a nucleobase which taken together form a targeting sequence;
Z is an integer from 8 to 38;
each Y is independently selected from O and —NR⁴, wherein each R⁴ is independently selected from H, C₁-C₆ alkyl, aralkyl, —C(═NH)NH₂, —C(O)(CH₂)NR⁵C(═NH)NH₂, —C(O)(CH₂)₂NHC(O)(CH₂)₅NR⁵C(═NH)NH₂, and G, wherein R⁵ is selected from H and C₁-C₆ alkyl and n is an integer from 1 to 5;
T is selected from OH and a moiety of the formula:

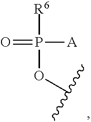

wherein:
A is selected from —OH, —N(R⁷)₂, and R¹ wherein each R⁷ is independently selected from H and C₁-C₆ alkyl, and
R⁶ is selected from OH, —N(R⁹)CH₂C(O)NH₂, and a moiety of the formula:

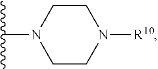

wherein:
   R⁹ is selected from H and C₁-C₆ alkyl; and
   R¹⁰ is selected from G, —C(O)—R¹¹OH, acyl, trityl, 4-methoxytrityl, —C(═NH)NH₂, —C(O)(CH₂)ₘNR¹²C(═NH)NH₂, and —C(O)(CH₂)₂NHC(O)(CH₂)₅NR¹²C(═NH)NH₂, wherein:
      m is an integer from 1 to 5,
      R¹¹ is of the formula —(O-alkyl)_{y}- wherein y is an integer from 3 to 10 and each of the y alkyl groups is independently selected from C₂-C₆ alkyl; and
      R¹² is selected from H and C₁-C₆ alkyl;
each instance of R¹ is independently selected from:
   —N(R¹³)₂, wherein each R³ is independently selected from H and C₁-C₆ alkyl; a moiety of formula (II):

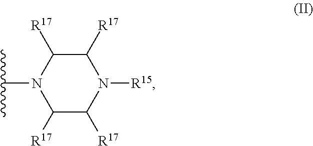

wherein:
   R¹⁵ is selected from H, G, C₁-C₆ alkyl, —C(═NH)NH₂, —C(O)(CH₂)_{q}NR¹⁸C(═NH)NH₂, and —C(O)(CH₂)₂NHC(O)(CH₂)₅NR⁸C(═NH)NH₂, wherein:
   R¹⁸ is selected from H and C₁-C₆ alkyl; and
   q is an integer from 1 to 5, and
   each R¹⁷ is independently selected from H and methyl; and a moiety of formula (III):

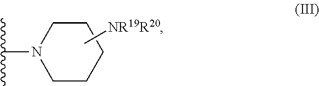

wherein:
   R¹⁹ is selected from H, C₁-C₆ alkyl, —C(═NH)NH₂, —C(O)(CH₂)ᵣNR²²C(═NH)NH₂, —C(O)CH(NH₂)(CH₂)₃NHC(═NH)NH₂, —C(O)(CH₂)₂NHC(O)(CH₂)₅NR²²C(═NH)NH₂, —C(O)CH(NH₂)(CH₂)₄NH₂ and G, wherein:
   R²² is selected from H and C₁-C₆ alkyl; and
   r is an integer from 1 to 5,
   R²⁰ is selected from H and C₁-C₆ alkyl; and
R² is selected from H, G, acyl, trityl, 4-methoxytrityl, C₁-C₆ alkyl, —C(═NH)NH₂, —C(O)—R²³, —C(O)(CH₂)ₛNR²⁴C(═NH)NH₂, —C(O)(CH₂)₂NHC(O)(CH₂)NR²⁴C(═NH)NH₂, —C(O)CH(NH₂)(CH₂)₃NHC(═NH)NH₂, and a moiety of the formula:

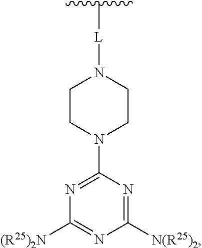

wherein,
   R²³ is of the formula —(O-alkyl)ᵥ-OH wherein v is an integer from 3 to 10 and each of the v alkyl groups is independently selected from C₂-C₆ alkyl; and
   R²⁴ is selected from H and C₁-C₆ alkyl;
   s is an integer from 1 to 5;
   L is selected from —C(O)(CH₂)₆C(O)— and —C(O)(CH₂)₂S₂(CH₂)₂C(O)—; and each R²⁵ is of the formula —(CH₂)₂₀C(O)N(R²⁶)₂ wherein each R²⁶ is of the formula —(CH₂)₆NHC(═NH)NH₂,

wherein G is a cell penetrating peptide (“CPP”) and linker moiety selected from —C(O)(CH₂)₅NH-CPP, —C(O)(CH₂)₂NH-CPP, —C(O)(CH₂)₂NHC(O)(CH₂)₅NH-CPP, and —C(O)CH₂NH—CPP, or G is of the formula:

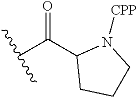

wherein the CPP is attached to the linker moiety by an amide bond at the CPP carboxy terminus, with the proviso that up to one instance of G is present, and

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ. ID NOS: 4 to 30, 133 to 255, or 296 to 342, is a fragment of at least 10 contiguous nucleotides of a sequence selected from SEQ. ID NOS: 4 to 30, 133 to 255, or 296 to 342, or is variant having at least 80% sequence identity to a sequence selected from SEQ. ID NOS: 4-30 or 133-255, where X is selected from uracil (U) or thymine (T). In some embodiments, the targeting sequence is selected from SEQ ID NOS: 4 to 30, 133 to 255, or 296 to 342, wherein X is selected from uracil (U) or thymine (T).

In some embodiments, R² is a moiety of the formula:

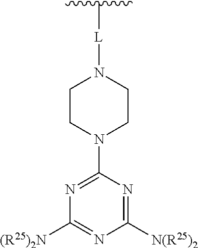

where L is selected from —C(O)(CH₂)₆C(O)— or —C(O)(CH₂)₂S₂(CH₂)₂C(O)—, and

and each R²⁵ is of the formula —(CH₂)₂₀C(O)N(R²⁶)₂ wherein each R²⁶ is of the formula —(CH₂)₆NHC(═NH)NH₂. Such moieties are further described in U.S. Pat. No. 7,935,816 incorporated herein by reference in its entirety.

In certain embodiments, R² may comprise either moiety depicted below:

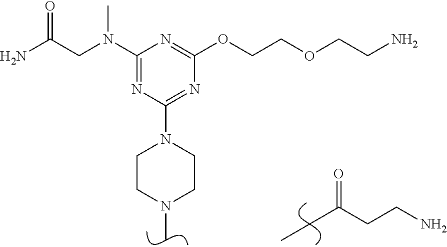

In certain embodiments, each R¹ is —N(CH₃)₂. In some embodiments, about 50-90% of the R₁ groups are dimethylamino (i.e. —N(CH₃)₂). In certain embodiments, about 66% of the R₁ groups are dimethylamino.

In some embodiments, the targeting sequence is selected from SEQ. ID NOS: 4 to 30, wherein X is selected from uracil (U) or thymine (T). In some embodiments, each R¹ is —N(CH₃)₂ and the targeting sequence is selected from SEQ. ID NOS: 4 to 30, wherein X is selected from uracil (U) or thymine (T).

In some embodiments of the disclosure, R₁ may be selected from:

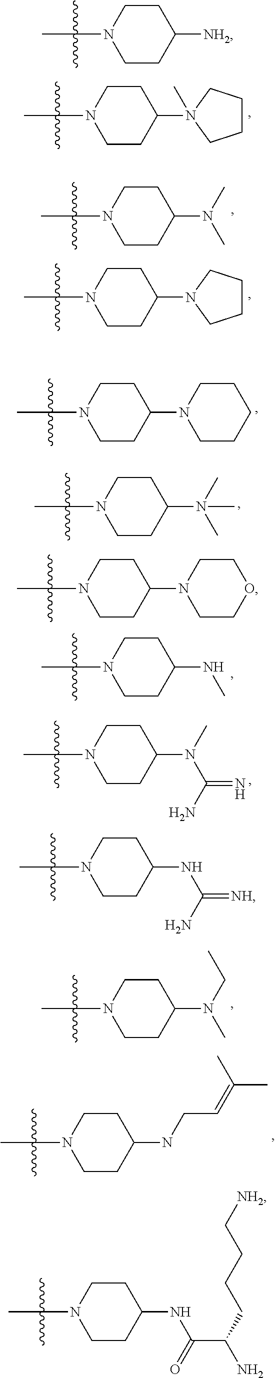

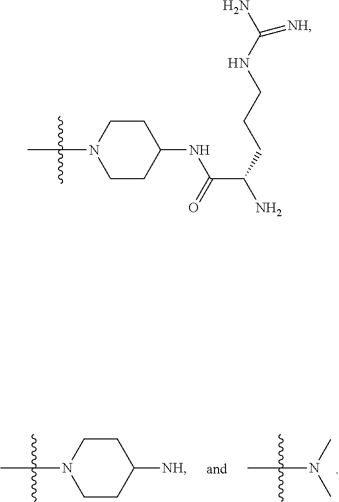

In some embodiments, at least one R¹ is:

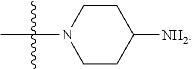 [SMILES: *N1CCC(N)CC1]

In certain embodiments, T is selected from:

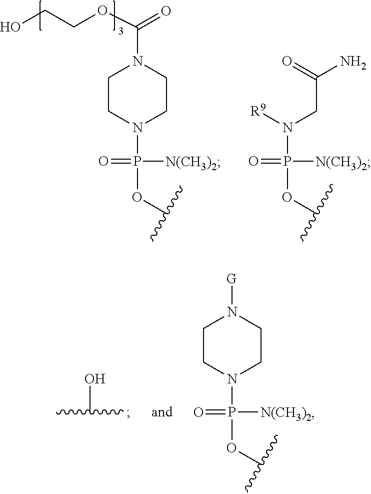

Y is O at each occurrence. In some embodiments, R² is selected from H, G, acyl, trityl, 4-methoxytrityl, benzoyl, and stearoyl.

In various embodiments, T is selected from:

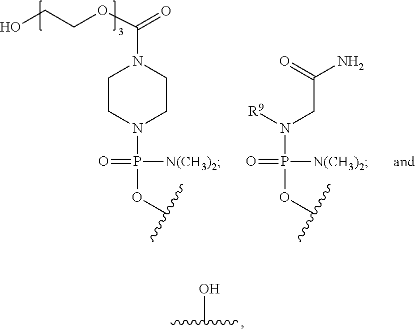

Y is O at each occurrence and R is G.

In some embodiments, T is of the formula:

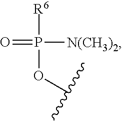

R⁶ is of the formula:

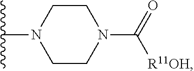

Y is O at each occurrence and R² is G.

In certain embodiments, T is of the formula:

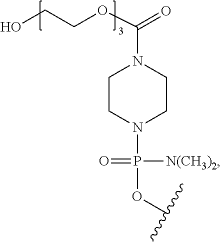

Y is O at each occurrence and R² is G.

In certain embodiments, T is of the formula:

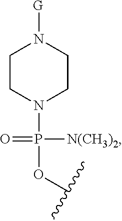

and Y is O at each occurrence.

In certain embodiments, T is of the formula:

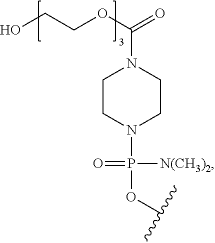

Y is O at each occurrence, each R¹ is —N(CH₃)₂, and R² is H.

In some embodiments, R² is selected from H, acyl, trityl, 4-methoxytrityl, benzoyl, and stearoyl. In various embodiments, R² is selected from H or G. In a particular embodiment, R² is G. In some embodiments, R² is H or acyl. In some embodiments, each R¹ is —N(CH₃)₂. In some embodiments, at least one instance of R¹ is —N(CH₃)₂. In certain embodiments, each instance of R¹ is —N(CH₃)₂.

In some embodiments, G is of the formula:

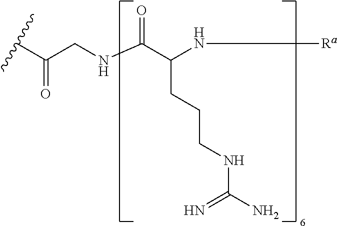

wherein R^{a} is selected from H acetyl, benzoyl, and stearoyl.

In certain embodiments, the CPP is of the formula:

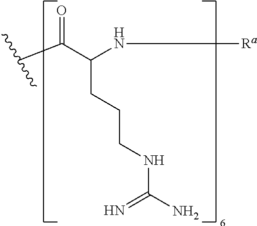

wherein R^{a} is selected from H, acetyl, benzoyl, and stearoyl.

In certain embodiments, the antisense oligomer of the disclosure is a compound of formula (IVa):

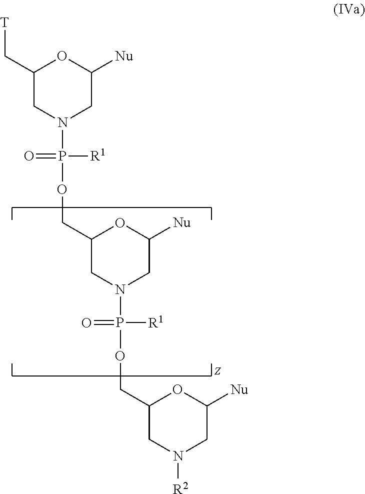

or a pharmaceutically acceptable salt thereof, where:
each Nu is a nucleobase which taken together forms a targeting sequence;
Z is an integer from 8 to 38;
T is selected from OH and a moiety of the formula:

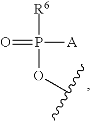

wherein:
A is selected from —OH, —N(R⁷)₂R⁸, and R¹ wherein:
   each R⁷ is independently selected from H and C₁-C₆ alkyl, and
   R⁸ is selected from an electron pair and H, and
R⁶ is selected from OH, —N(R⁹)CH₂C(O)NH₂, and a moiety of the formula:

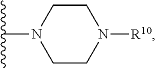

wherein:
   R⁹ is selected from H and C₁-C₆ alkyl; and
   R¹⁰ is selected from —C(O)—R¹¹OH, acyl, trityl, 4-methoxytrityl, —C(═NH)NH₂, —C(O)(CH₂)ₘNR²C(═NH)NH₂, and —C(O)(CH₂)₂NHC(O)(CH₂)₅NR¹²C(═NH)NH₂, wherein:
      m is an integer from 1 to 5,
      R¹¹ is of the formula —(O-alkyl)_{y}- wherein y is an integer from 3 to 10 and each of the y alkyl groups is independently selected from C₂-C₆ alkyl; and
   R¹² is selected from H and C₁-C₆ alkyl;
each instance of R¹ is independently —N(R¹³)₂R¹⁴, wherein each R¹³ is independently selected from H and C₁-C₆ alkyl, and R¹⁴ is selected from an electron pair and H; and
R² is selected from H, acyl, trityl, 4-methoxytrityl, benzoyl, stearoyl, and C₁-C₆ alkyl,

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ. ID NOS: 4 to 30, 133 to 255, or 296 to 342, is a fragment of at least 10 contiguous nucleotides of a sequence selected from SEQ. ID NOS: 4 to 30, 133 to 255, or 296 to 342, or is variant having at least 80% sequence identity to a sequence selected from SEQ. ID NOS: 4 to 30, 133 to 255, or 296 to 342, where X is selected from uracil (U) or thymine (T). In some embodiments, the targeting sequence is selected from SEQ ID NOS: 4 to 30, 133 to 255, or 296 to 342, wherein X is selected from uracil (U) or thymine (T).

In certain embodiments, T is selected from:

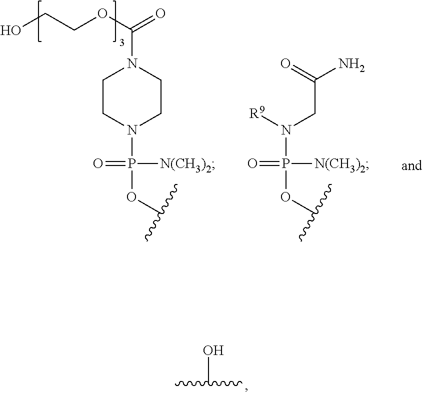

and

Y is O at each occurrence. In some embodiments, R² is selected from H, acyl, trityl, 4-methoxytrityl, benzoyl, and stearoyl.

In various embodiments, T is selected from:

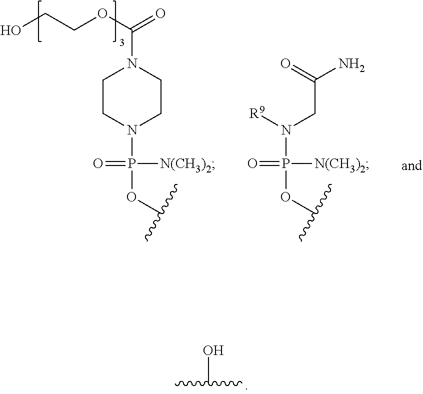

In some embodiments, T is of the formula:

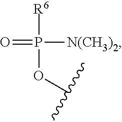

and

R⁶ is of the formula:

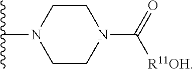

In certain embodiments, T is of the formula:

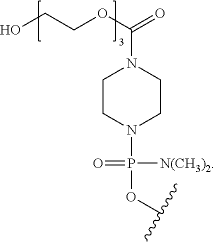

In some embodiments, R² is H, trityl, or acyl. In some embodiments, at least one instance of R¹ is —N(CH₃)₂. In some embodiments, each R¹ is —N(CH₃)₂.

In certain embodiments, the antisense oligomer of the disclosure is a compound of formula (IVb):

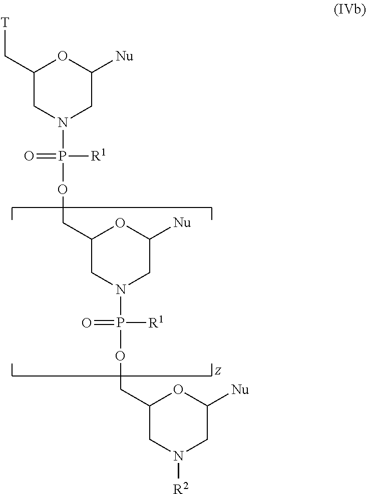

or a pharmaceutically acceptable salt thereof, where:
each Nu is a nucleobase which taken together forms a targeting sequence;
Z is an integer from 8 to 38;
T is selected from a moiety of the formula:

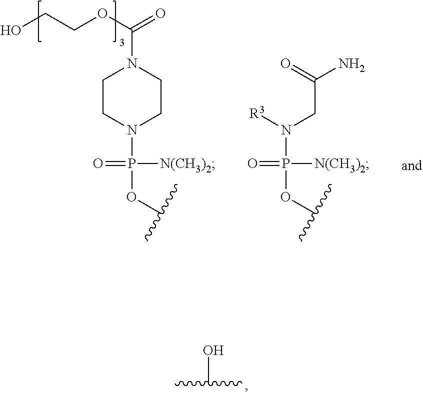

wherein R³ is selected from H and C₁-C₆ alkyl;
each instance of R¹ is independently —N(R⁴)₂, wherein each R⁴ is independently selected from H and C₁-C₆ alkyl; and
R² is selected from H, acyl, trityl, 4-methoxytrityl, and C₁-C₆ alkyl,

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ. ID NOS: 4 to 30, 133 to 255, or 296 to 342, is a fragment of at least 10 contiguous nucleotides of a sequence selected from SEQ. ID NOS: 4 to 30, 133 to 255, or 296 to 342, or is variant having at least 80% sequence identity to a sequence selected from SEQ. ID NOS: 4 to 30, 133 to 255, or 296 to 342, where X is selected from uracil (U) or thymine (T). In some embodiments, the targeting sequence is selected from SEQ ID NOS: 4 to 30, 133 to 255, or 296 to 342, wherein X is selected from uracil (U) or thymine (T).

In various embodiments, R² is selected from H or acyl. In some embodiments, R² is H.

In certain embodiments, T is of the formula:

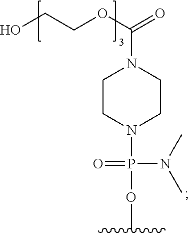

and

R² is hydrogen.

In certain embodiments, the antisense oligomer of the disclosure is a compound of formula (IVc):

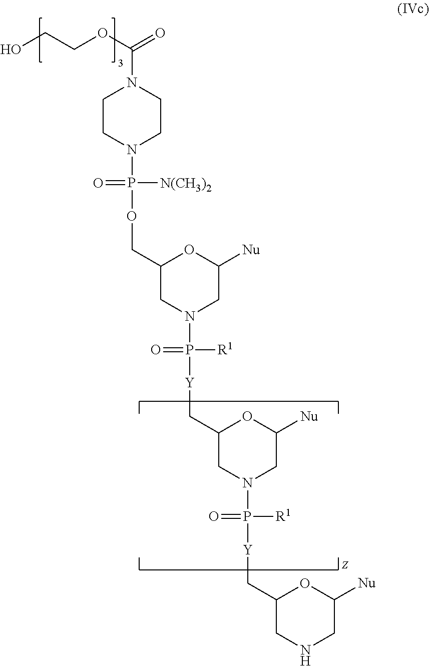

or a pharmaceutically acceptable salt thereof, wherein:

each Nu is a nucleobase which taken together form a targeting sequence;

Z is an integer from 8 to 38;

each Y is O;

each R¹ is independently selected from the group consisting of:

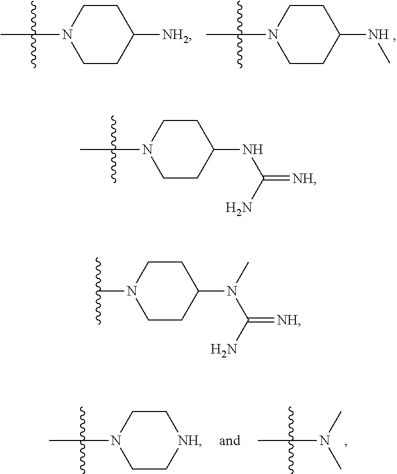

wherein at least one R¹ is —N(CH₃)₂, and
wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ. ID NOS: 4 to 30, 133 to 255, or 296 to 342, is a fragment of at least 10 contiguous nucleotides of a sequence selected from SEQ. ID NOS: 4 to 30, 133 to 255, or 296 to 342, or is variant having at least 80% sequence identity to a sequence selected from SEQ. ID NOS: 4 to 30, 133 to 255, or 296 to 342, where X is selected from uracil (U) or thymine (T).

In some embodiments, the targeting sequence is selected from SEQ ID NOS: 4 to 30, 133 to 255, or 296 to 342, wherein X is selected from uracil (U) or thymine (T). In some embodiments, each R¹ is —N(CH₃)₂.

In certain embodiments, the antisense oligomer is a compound of formula (V):

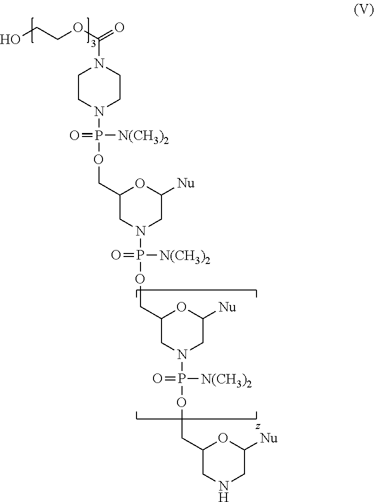

or a pharmaceutically acceptable salt thereof, wherein:

each Nu is a nucleobase which taken together form a targeting sequence; and

Z is an integer from 8 to 38;

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ ID. NOS: 4 to 30, 133 to 255, or 296 to 342, is a fragment of at least 10 contiguous nucleotides of a sequence selected from SEQ ID. NOS: 4 to 30, 133 to 255, or 296 to 342, or is variant having at least 80% sequence identity to a sequence selected from SEQ ID. NOS: 4 to 30, 133 to 255, or 296 to 342, where X is selected from uracil (U) or thymine (T). In some embodiments, the targeting sequence is selected from SEQ ID NOS: 4 to 30, 133 to 255, or 296 to 342, wherein X is selected from uracil (U) or thymine (T).

In certain embodiments, the antisense oligomer is a compound of formula (VI):

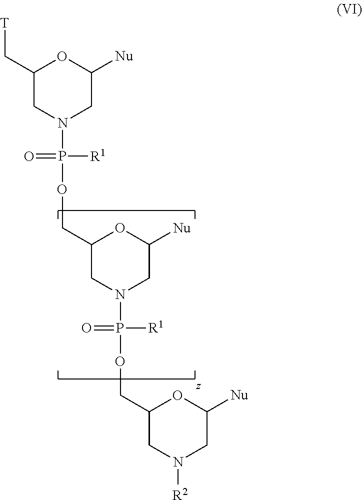

or a pharmaceutically acceptable salt thereof,

where each Nu is a nucleobase which taken together forms a targeting sequence;

Z is an integer from 8 to 38;

T is selected from:

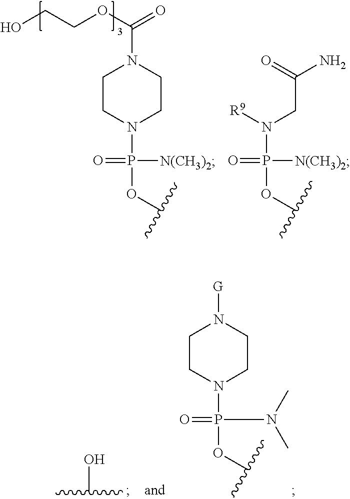

each R¹ is independently selected from the group consisting of:

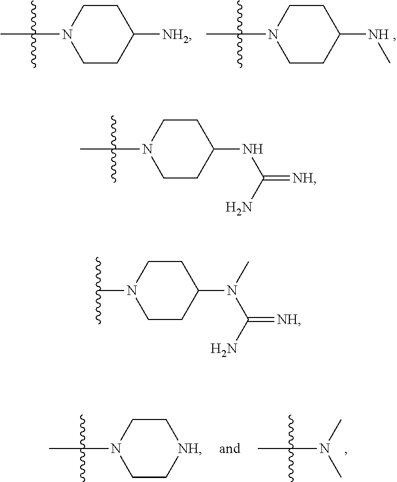

R² is selected from H, G, acyl, trityl, 4-methoxytrityl, benzoyl, and stearoyl,

wherein G is a cell penetrating peptide (“CPP”) and linker moiety selected from —C(O)(CH₂)₅NH-CPP, —C(O)(CH₂)₂NH-CPP, —C(O)(CH₂)₂NHC(O)(CH₂)₅NH-CPP, and —C(O)CH₂NH—CPP, or G is of the formula:

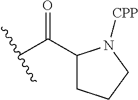

wherein the CPP is attached to the linker moiety by an amide bond at the CPP carboxy terminus, with the proviso that only one instance of G is present,

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ. ID NOS: 4 to 30, 133 to 255, or 296 to 342, is a fragment of at least 10 contiguous nucleotides of a sequence selected from SEQ. ID NOS: 4 to 30, 133 to 255, or 296 to 342, or is variant having at least 80% sequence identity to a sequence selected from SEQ. ID NOS: 4 to 30, 133 to 255, or 296 to 342, where X is selected from uracil (U) or thymine (T).

In certain embodiments, T is of the formula:

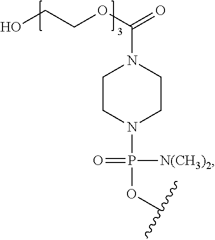

and R² is G. In some embodiments, T is of the formula:

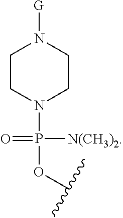

In some embodiments, T is TEG as defined above, R² is G, and R³ is an electron pair or H. In certain embodiments, R² is selected from H, acyl, trityl, 4-methoxytrityl, benzoyl, and stearoyl and T is of

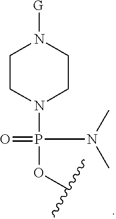

In various embodiments, R² is selected from H, acyl, trityl, 4-methoxytrityl, benzoyl, and stearoyl.

In some embodiments, wherein G is of the formula:

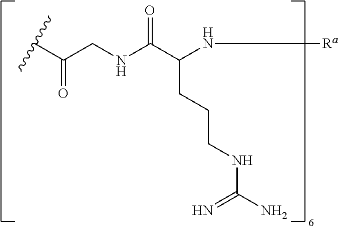

wherein R^{a} is selected from H, acetyl, benzoyl, and stearoyl.

In some embodiments, the CPP is of the formula:

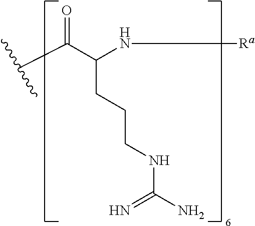

wherein R^{a} is selected from H, acetyl, benzoyl, and stearoyl.

In certain embodiments, the antisense oligomer is a compound of formula (VII):

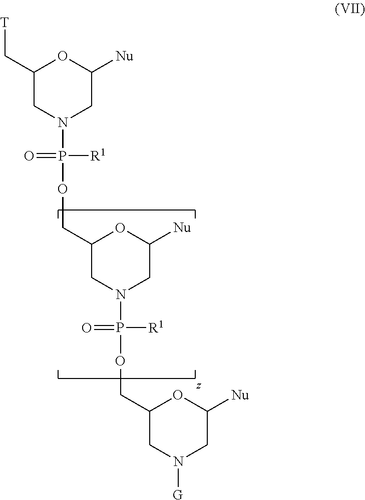

or a pharmaceutically acceptable salt thereof,

where each Nu is a nucleobase which taken together forms a targeting sequence;

Z is an integer from 8 to 38;

T is selected from:

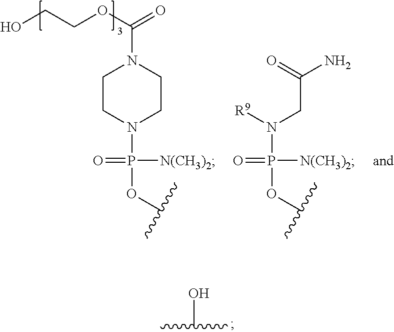

each instance of R¹ is —N(R⁰)₂ wherein each R¹⁰ is independently C₁-C₆ alkyl; and

G is a cell penetrating peptide (“CPP”) and linker moiety selected from —C(O)(CH₂)₅NH-CPP, —C(O)(CH₂)₂NH-CPP, —C(O)(CH₂)₂NHC(O)(CH₂)₅NH-CPP, and —C(O)CH₂NH—CPP, or G is of the formula:

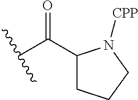

wherein the CPP is attached to the linker moiety by an amide bond at the CPP carboxy terminus,

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ. ID NOS: 4 to 30, 133 to 255, or 296 to 342, is a fragment of at least 10 contiguous nucleotides of a sequence selected from SEQ. ID NOS: 4 to 30, 133 to 255, or 296 to 342, or is variant having at least 80% sequence identity to a sequence selected from SEQ. ID NOS: 4 to 30, 133 to 255, or 296 to 342, where X is selected from uracil (U) or thymine (T).

In some embodiments, at least one instance of R¹ is —N(CH₃)₂. In certain embodiments, each instance of R¹ is —N(CH₃)₂.

In certain embodiments, T is of the formula:

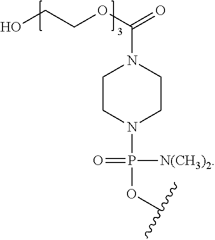

In various embodiments, G is of the formula:

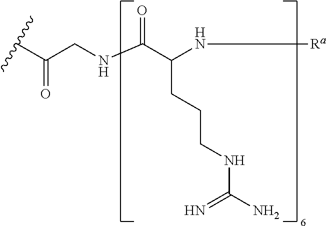

wherein R^{a} is selected from H, acetyl, benzoyl, and stearoyl.

In certain embodiments, the CPP is of the formula:

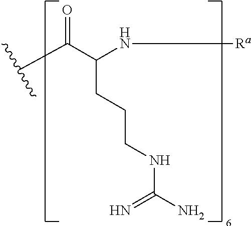

wherein R^{a} is selected from H, acetyl, benzoyl, and stearoyl.

In various aspects, an antisense oligonucleotide of the disclosure includes a compound of formula (VIII):

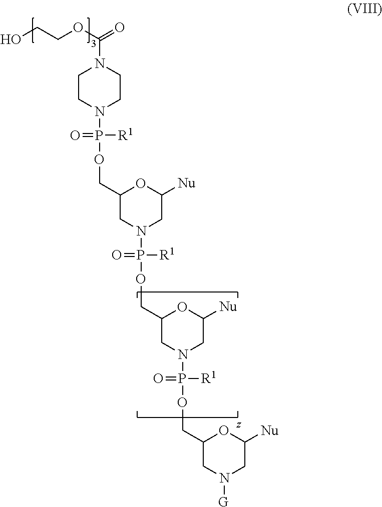

or a pharmaceutically acceptable salt thereof, wherein:

Z is an integer from 8 to 38;

each Nu is a nucleobase which taken together forms a targeting sequence;

each instance of R¹ is —N(R⁰)₂ wherein each R¹⁰ is independently C₁-C₆ alkyl; and

G is a cell penetrating peptide (“CPP”) and linker moiety selected from —C(O)(CH₂)₅NH-CPP, —C(O)(CH₂)₂NH-CPP, —C(O)(CH₂)₂NHC(O)(CH₂)₅NH-CPP, and —C(O)CH₂NH—CPP, or G is of the formula:

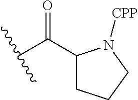

wherein the CPP is attached to the linker moiety by an amide bond at the CPP carboxy terminus,

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ. ID NOS: 4 to 30, 133 to 255, or 296 to 342, is a fragment of at least 10 contiguous nucleotides of a sequence selected from SEQ. ID NOS: 4 to 30, 133 to 255, or 296 to 342, or is variant having at least 80% sequence identity to a sequence selected from SEQ. ID NOS: 4 to 30, 133 to 255, or 296 to 342, where X is selected from uracil (U) or thymine (T).

In some embodiments, at least one instance of R¹ is —N(CH₃)₂. In certain embodiments, each instance of R¹ is —N(CH₃)₂.

In some embodiments, G is of the formula:

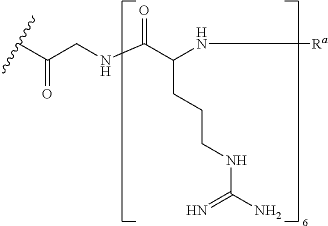

wherein R^{a} is selected from H, acetyl, benzoyl, and stearoyl.

In certain embodiments, the CPP is of the formula:

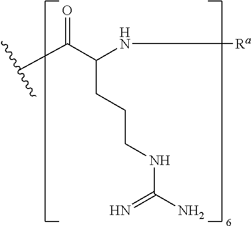

wherein R^{a} is selected from H, acetyl, benzoyl, and stearoyl.

In various aspects, an antisense oligomer of the disclosure can be a compound of formula (IX):

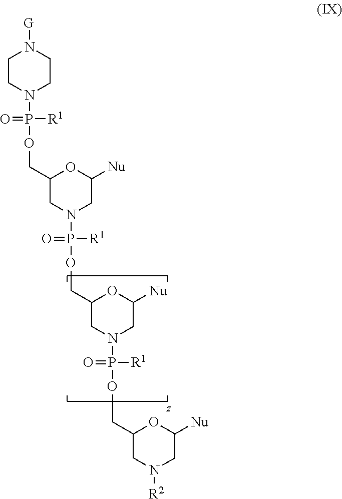

wherein:

Z is an integer from 8 to 38;

each Nu is a nucleobase which taken together forms a targeting sequence;

each instance of R¹ is —N(R⁰)₂R¹¹ wherein each R¹⁰ is independently C₁-C₆ alkyl, and R¹¹ is selected from an electron pair and H; and

R² is selected from H, trityl, 4-methoxytrityl, acyl, benzoyl, and stearoyl,

wherein G is a cell penetrating peptide (“CPP”) and linker moiety selected from —C(O)(CH₂)₅NH-CPP, —C(O)(CH₂)₂NH-CPP, —C(O)(CH₂)₂NHC(O)(CH₂)₅NH-CPP, and —C(O)CH₂NH—CPP, or G is of the formula:

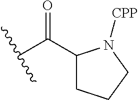

wherein the CPP is attached to the linker moiety by an amide bond at the CPP carboxy terminus,

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ. ID NOS: 4 to 30, 133 to 255, or 296 to 342, is a fragment of at least 10 contiguous nucleotides of a sequence selected from SEQ. ID NOS: 4 to 30, 133 to 255, or 296 to 342, or is variant having at least 80% sequence identity to a sequence selected from SEQ. ID NOS: 4 to 30, 133 to 255, or 296 to 342, where X is selected from uracil (U) or thymine (T).

In some embodiments, at least one instance of R¹ is —N(CH₃)₂. In certain embodiments, each instance of R¹ is —N(CH₃)₂.

In some embodiments, G is of the formula:

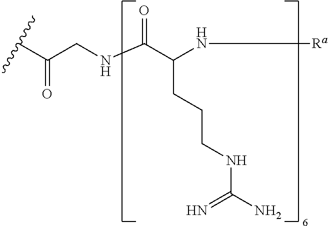

wherein R^{a} is selected from H, acetyl, benzoyl, and stearoyl.

In certain embodiments, the CPP is of the formula:

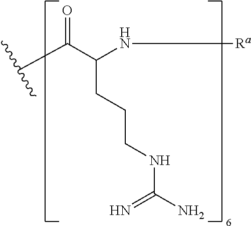

wherein R^{a} is selected from H, acetyl, benzoyl, and stearoyl.

In various aspects, an antisense oligonucleotide of the disclosure includes a compound of formula (X):

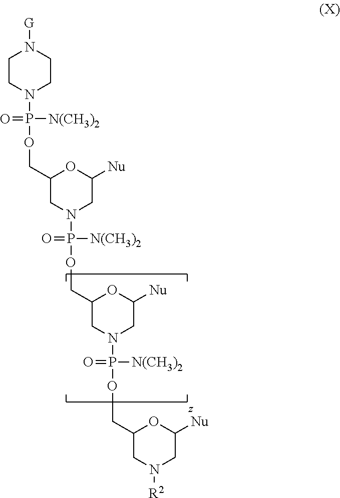

or a pharmaceutically acceptable salt thereof, wherein:

Z is an integer from 8 to 38;

each Nu is a nucleobase which taken together forms a targeting sequence;

R² is selected from H or acyl; and

G is a cell penetrating peptide (“CPP”) and linker moiety selected from —C(O)(CH₂)₅NH-CPP, —C(O)(CH₂)₂NH-CPP, —C(O)(CH₂)₂NHC(O)(CH₂)₅NH-CPP, and —C(O)CH₂NH—CPP, or G is of the formula:

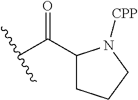

wherein the CPP is attached to the linker moiety by an amide bond at the CPP carboxy terminus,

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4 to 30, 133 to 255, or 296 to 342, is selected from SEQ. ID NOS: 4 to 30, 133 to 255, or 296 to 342, is a fragment of at least 10 contiguous nucleotides of a sequence selected from SEQ. ID NOS: 4 to 30, 133 to 255, or 296 to 342, or is variant having at least 80% sequence identity to a sequence selected from SEQ. ID NOS: 4 to 30, 133 to 255, or 296 to 342, where X is selected from uracil (U) or thymine (T).

In some embodiments, G is of the formula:

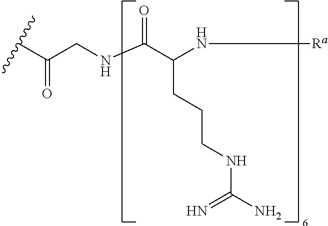

wherein R^{a} is selected from H, acetyl, benzoyl, and stearoyl.

In certain embodiments, the CPP is of the formula:

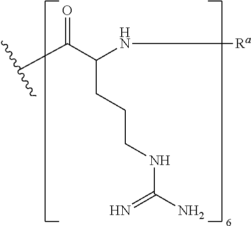

wherein R^{a} is selected from H, acetyl, benzoyl, and stearoyl.

In some embodiments of any of the methods or compositions described herein, Z is an integer from 8 to 28, from 15 to 38, 15 to 28, 8 to 25, from 15 to 25, from 10 to 38, from 10 to 25, from 12 to 38, from 12 to 25, from 14 to 38, or from 14 to 25. In some embodiments of any of the methods or compositions described herein, Z is 12, 13, 14, 15, 16, 17, 18, 19, 20, 21, 22, 23, 24, 25, 26, 27, 28, 29, 30, 31, 32, 33, 34, 35, 36, 37, or 38. In some embodiments of any of the methods or compositions described herein, Z is 12, 13, 14, 15, 16, 17, 18, 19, 20, 21, 22, 23, 24, 25, 26, 27, or 28. In some embodiments of any of the methods or compositions described herein, Z is 15, 16, 17, 18, 19, 20, 21, 22, 23, 24, or 25.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 8 to 28.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 15 to 38.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 15 to 28.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 8 to 25.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 15 to 25.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 10 to 38.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 10 to 25.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 12 to 38.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 12 to 25.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 14 to 38.

In some embodiments, each Z of the modified antisense oligomers of the disclosure, including compounds of formulas (I), (IVa), (IVb), (IVc), (V), (VI), (VII), (VIII), (IX), (X), and (XI), is an integer from 14 to 25.

In some embodiments, each Z of the modified antisense oligomers of the disclosure, including compounds of formulas (I), (IVa), (IVb), (IVc), (V), (VI), (VII), (VIII), (IX), (X), and (XI), is 12, 13, 14, 15, 16, 17, 18, 19, 20, 21, 22, 23, 24, 25, 26, 27, 28, 29, 30, 31, 32, 33, 34, 35, 36, 37, or 38.

In some embodiments, each Z of the modified antisense oligomers of the disclosure, including compounds of formulas (I), (IVa), (IVb), (IVc), (V), (VI), (VII), (VIII), (IX), (X), and (XI), is 12, 13, 14, 15, 16, 17, 18, 19, 20, 21, 22, 23, 24, 25, 26, 27, or 28.

In some embodiments, each Z of the modified antisense oligomers of the disclosure, including compounds of formulas (I), (IVa), (IVb), (IVc), (V), (VI), (VII), (VIII), (IX), (X), and (XI), is 15, 16, 17, 18, 19, 20, 21, 22, 23, 24, or 25.

In some embodiments, each Nu of the antisense oligomers of the disclosure, including compounds of formula (I), (IVa), (IVb), (IVc), (V), (VI), (VII), (VIII), (IX), and (X), is independently selected from the group consisting of adenine, guanine, thymine, uracil, cytosine, hypoxanthine, 2,6-diaminopurine, 5-methyl cytosine, C5-propynyl-modified pyrimidines, and 9-(aminoethoxy)phenoxazine.

In some embodiments, the targeting sequence of the antisense oligomers of the disclosure, including compounds of formula (I), (IVa), (IVb), (IVc), (V), (VI), (VII), (VIII), (IX), and (X), is complementary 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence of the antisense oligomers of the disclosure, including compounds of formula (I), (IVa), (IVb), (IVc), (V), (VI), (VII), (VIII), (IX), and (X), comprises a sequence selected from SEQ. ID NOS: 1-5 or 10-31, is selected from SEQ. ID NOS: 1-5 or 10-31, is a fragment of at least 12 contiguous nucleotides of a sequence selected from SEQ. ID NOS: 1-5 or 10-31, or is variant having at least 90% sequence identity to a sequence selected from SEQ. ID NOS: 1-5 or 10-31, where X is selected from uracil (U) or thymine (T). In certain embodiments, the targeting sequence of the antisense oligomers of the disclosure, including compounds of formula (I), (IVa), (IVb), (IVc), (V), (VI), (VII), (VIII), (IX), and (X), comprises a sequence selected from SEQ. ID NOS:133-255, is selected from SEQ. ID NOS: 133-255, is a fragment of at least 12 contiguous nucleotides of a sequence selected from SEQ. ID NOS: 133-255, or is variant having at least 90% sequence identity to a sequence selected from SEQ. ID NOS: 133-255, where X is selected from uracil (U) or thymine (T).

Additional antisense oligomers/chemistries that can be used in accordance with the present disclosure include those described in the following patents and patent publications, the contents of which are incorporated herein by reference: PCT Publication Nos. WO/2007/002390; WO/2010/120820; and WO/2010/148249; U.S. Pat. No. 7,838,657; and U.S. Application No. 2011/0269820.

The antisense oligonucleotides can be prepared by stepwise solid-phase synthesis, employing methods known in the art and described in the references cited herein.

C. CPPs and Arginine-Rich Peptide Conjugates of PMOs (PPMOs)

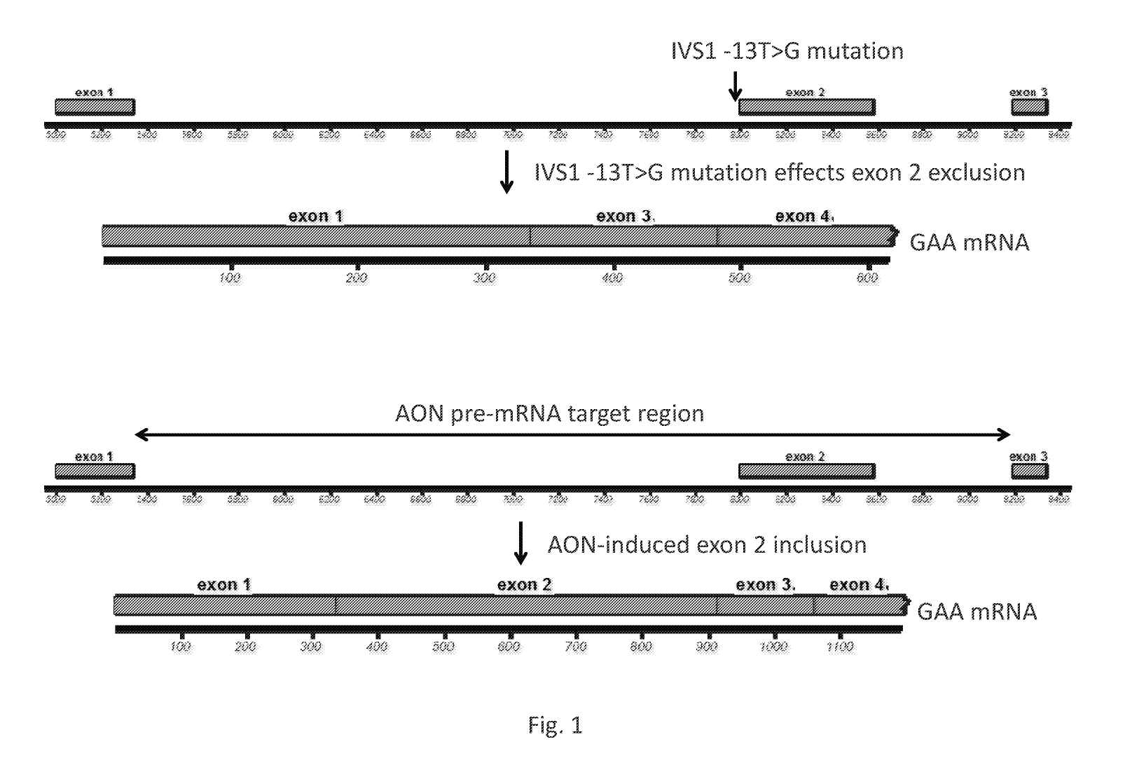

In certain embodiments, the antisense oligonucleotide is conjugated to a cell-penetrating peptide (CPP). In some embodiments, the CPP is an arginine-rich peptide. By “arginine-rich carrier peptide” is meant that the CPP has at least 2, and preferably 2, 3, 4, 5, 6, 7, or 8 arginine residues, each optionally separated by one or more uncharged, hydrophobic residues, and optionally containing about 6-14 amino acid residues. FIGS. 1F-1H show exemplary chemical structures of CPP-PMO conjugates used in the Examples, including 5′ and 3′ PMO conjugates. CPPs are further described in U.S. Application Publication No. 2012/0289457 and International Patent Application Publication Nos. WO 2004/097017 and WO 2009/005793, the disclosures of which are incorporated herein by reference in their entirety.

In some embodiments, the CPP is linked at its C-terminus to the 3′-end or the 5′-end of the oligonucleotide via a 1, 2, 3, 4, or 5 amino acid linker. In particular embodiments, the linkers can include:

—C(O)(CH₂)₅NH-CPP (X linker);

—C(O)(CH₂)₂NH-CPP (B linker);

—C(O)(CH₂)₂NHC(O)(CH₂)₅NH-CPP (XB peptide linker);

and —C(O)CH₂NH—CPP, or

G is of the formula:

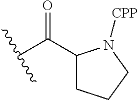

wherein the CPP is attached to the linker moiety by an amide bond at the CPP carboxy terminus.

In various embodiments, the CPP is an arginine-rich peptide. In certain embodiments, the arginine-rich peptide is R₆ (six arginine residues; SEQ ID NO: 31) and the linker is selected from the group described above wherein the R₆ peptide is attached to the linker at the CPP carboxy terminus. In certain embodiments, G is —C(O)CH₂NH—R₆, also referred to as R₆G- (SEQ ID NO: 32, where G is the amino acid glycine), linked to an antisense oligomer of the disclosure at the 5′ or 3′ end of the oligomer.

In some embodiments, G is of the formula:

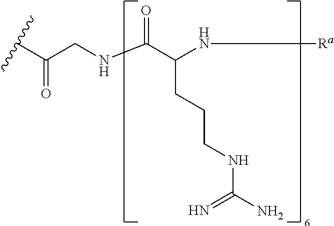

wherein R^{a} is selected from H, acetyl, benzoyl, and stearoyl. In certain embodiments, G is SEQ ID NO: 32)

In various embodiments, the CPP is of the formula:

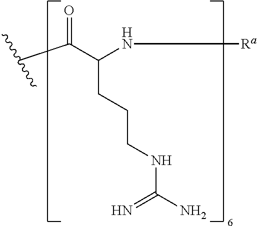

wherein R^{a} is selected from H, acetyl, benzoyl, and stearoyl. In certain embodiments, the CPP is SEQ ID NO: 31.

In some embodiments, an antisense oligomer of the disclosure is a compound of formula (XI) selected from:

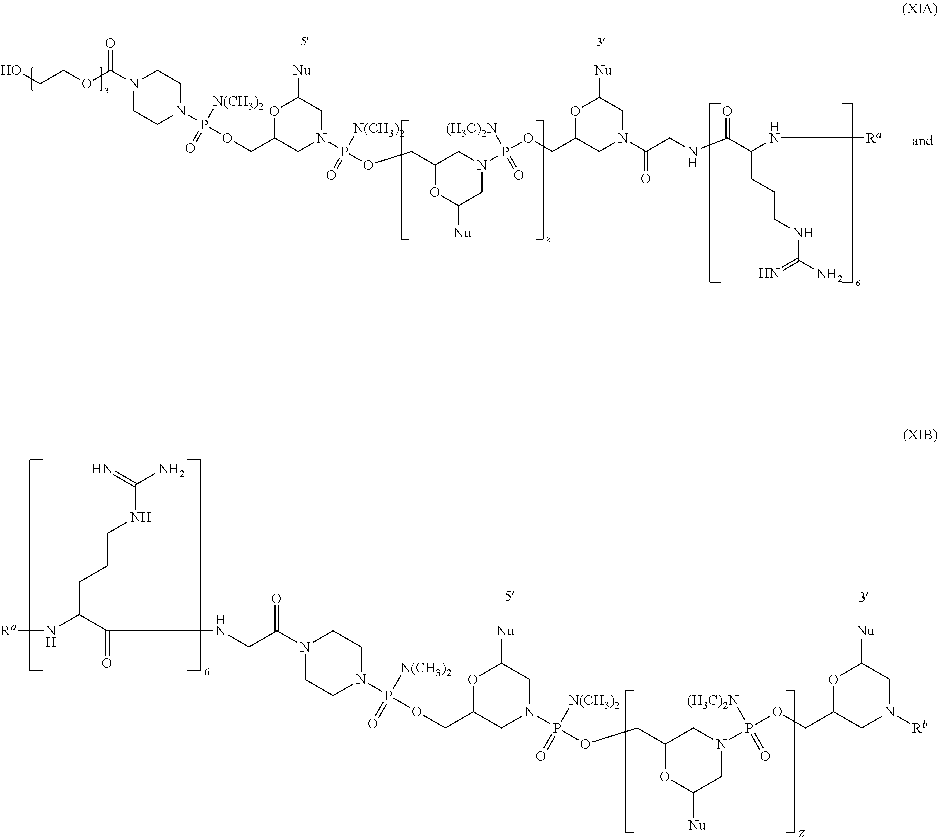

or a pharmaceutically acceptable salt of either of the foregoing,

wherein Z is an integer from 8 to 38, R^{a} is selected from H, acetyl, benzoyl, and stearoyl, R^{b} is selected from H, acetyl, benzoyl, stearoyl, trityl, and 4-methoxytrityl, and each Nu is a purine or pyrimidine base-pairing moiety which taken together form a targeting sequence, and

wherein the targeting sequence is complementary to 10 or more contiguous nucleotides in a target region within intron 1 (SEQ ID. NO. 1), intron 2 (SEQ ID. NO. 2), or exon 2 (SEQ ID. NO. 3) of a pre-mRNA of the human acid alpha-glucosidase (GAA) gene. In certain embodiments, the targeting sequence comprises a sequence selected from SEQ. ID NOS: 4-30 or 133-255, is selected from SEQ. ID NOS: 4-30 or 133-255, is a fragment of at least 10 contiguous nucleotides of a sequence selected from SEQ. ID NOS: 4-30 or 133-255, or is variant having at least 80% sequence identity to a sequence selected from SEQ. ID NOS: 4-30 or 133-255, where X is selected from uracil (U) or thymine (T).

In some embodiments, the targeting sequence of an antisense oligomers of the disclosure, including compounds of formula (I), (IVa), (IVb), (IVc), (V), (VI), (VII), (VIII), (IX), (X), and (XI), is selected from:

a) SEQ ID NO: 4 (GCC CXG GXC XGC XGG CXC CCX GCX G) wherein Z is 23;

b) SEQ ID NO: 5 (CCC XGG XCT GCT GGC TCC CTG CTG G) wherein Z is 23;

c) SEQ ID NO: 6 (CCX GGX CXG CXG GCX CCC XGC XGG X) wherein Z is 23;

d) SEQ ID NO: 7 (CXG GXC XGC XGG CXC CCX GCX GGX G) wherein Z is 23;

e) SEQ ID NO: 8 (XGG XCX GCX GGC XCC CXG CXG GXG A) wherein Z is 23;

f) SEQ ID NO: 9 (GGX CXG CXG GCX CCC XGC XGG XGA G) wherein Z is 23;

g) SEQ ID NO: 10 (GXC XGC XGG CXC CCX GCX GGX GAG C) wherein Z is 23;

h) SEQ ID NO: 11 (XCX GCX GGC XCC CXG CXG GXG AGC X) wherein Z is 23;

i) SEQ ID NO: 12 (GGG CCC XGG XCX GCX GGC XCC CXG C) wherein Z is 23;

j) SEQ ID NO: 13 (GGG GCC CXG GXC XGC XGG CXC CCX G) wherein Z is 23;

k) SEQ ID NO: 14 (CGG GGC CCX GGX CXG CXG GCX CCC X) wherein Z is 23;

l) SEQ ID NO: 15 (CCG GGG CCC XGG XCX GCX GGC XCC C) wherein Z is 23;

m) SEQ ID NO: 16 (CCC GGG GCC CXG GXC XGC XGG CXC C) wherein Z is 25;

n) SEQ ID NO: 17 (XCC CGG GGC CCX GGX CXG CXG GCX C) wherein Z is 25;

o) SEQ ID NO: 18 (AXC CCG GGG CCC XGG XCX GCX GGC X) wherein Z is 23;

p) SEQ ID NO: 19 (CAX CCC GGG GCC CXG GXC XGC XGG C) wherein Z is 23;

q) SEQ ID NO: 20 (XCX GCC CXG GCC GCC GCC CCC GCC CCX) wherein Z is 25;

r) SEQ ID NO: 21 (XGA GGX GCG XGG GXG XCG AXG XCC A) wherein Z is 23;

s) SEQ ID NO: 22 (GAG GXG CGX GGG XGX CGA XGX CCA C) wherein Z is 23;

t) SEQ ID NO: 23 (AGG XGC GXG GGX GXC GAX GXC CAC G) wherein Z is 23;

u) SEQ ID NO: 24 (GCG CGX GGA CAX CGA CAC CCA CGC A) wherein Z is 23;

v) SEQ ID NO: 25 (XGX GAG GGC GCG XGG ACA XCG ACA C) wherein Z is 23;

w) SEQ ID NO: 26 (XXG XGA GGG CGC GXG GAC AXC GAC A) wherein Z is 23;

x) SEQ ID NO: 27 (CX GXG AGG GCG CGX GGA CAX CGA C) wherein Z is 22;

y) SEQ ID NO: 28 (GGC CCX GGX CXG CXG GCX CCC XGC X) wherein Z is 23;

z) SEQ ID NO: 29 (XGG CCG CCG CCC CCG CCC CX) wherein Z is 18; and

aa) SEQ ID NO: 30 (GXG AGG XGC GXG GGX GXC GA) wherein Z is 18,
wherein X is selected from uracil (U) or thymine (T).

In some embodiments, the targeting sequence of an antisense oligomers of the disclosure, including compounds of formula (I), (IVa), (IVb), (IVc), (V), (VI), (VII), (VIII), (IX), (X), and (XI), is selected from:

a) SEQ ID NO: 133 (GCX CAG CAG GGA GGC GGG AG) wherein Z is 18;

b) SEQ ID NO: 134 (GGC XCX CAA AGC AGC XCX GA) wherein Z is 18;

c) SEQ ID NO: 135 (GAC AXC AAC CGC GGC XGG CAC XGC A) wherein Z is 23;

d) SEQ ID NO: 136 (GGG XAA GGX GGC CAG GGX GGG XGX X) wherein Z is 23;

e) SEQ ID NO: 137 (GCC CXG CXG XCX AGA CXG G) wherein Z is 17;

f) SEQ ID NO: 138 (GAG AGG GCC AGA AGG AAG GG) wherein Z is 18;

g) SEQ ID NO: 139 (CCC GCC CCX GCC CXG CC) wherein Z is 15;

h) SEQ ID NO: 140 (XGG CCG CCG CCC CCG CCC) wherein Z is 16;

i) SEQ ID NO: 141 (XGX CCA CGC GCA CCC XCX GC) wherein Z is 18;

j) SEQ ID NO: 142 (GXG AGG XGC GXG GGX GXC GA) wherein Z is 18;

k) SEQ ID NO: 143 (GCA ACA XGC ACC CCA CCC XX) wherein Z is 18;

l) SEQ ID NO: 144 (AGG GCC CAG CAC ACA GXG GX) wherein Z is 18;

m) SEQ ID NO: 145 (XCA CAC CXC CGC XCC CAG CA) wherein Z is 18;

n) SEQ ID NO: 146 (GGC GCX GCC AXX GXC XGC) wherein Z is 16;

o) SEQ ID NO: 147 (GXG XCC CCA CXG CXC CCC GA) wherein Z is 18;

p) SEQ ID NO: 148 (CXG GAG XAC CXG XCA CCG XG) wherein Z is 18;

q) SEQ ID NO: 149 (XGA GCC CCG AGC CCX GCC XX) wherein Z is 18;

r) SEQ ID NO: 150 (XGA CCC ACC XXX XCA XAA AGA XGA A) wherein Z is 23;

s) SEQ ID NO: 151 (CXC XGG CAG CCC XAC XCX ACC XGA C) wherein Z is 23;

t) SEQ ID NO: 152 (CXA GXA XAA AXA CAX CCC AAA XXX XGC) wherein Z is 25;

u) SEQ ID NO: 153 (GGC CCX GGX CXG CXG GCX CCC XGC X) wherein Z is 23;

v) SEQ ID NO: 154 (GCX CCC XGC AGC CCC XGC XXX GCA G) wherein Z is 23;

w) SEQ ID NO: 155 (GCG GGG CAG ACG XCA GGX GX) wherein Z is 18;

x) SEQ ID NO: 156 (CAG CGC GGG GCA GAC GXC AG) wherein Z is 18;

y) SEQ ID NO: 157 (CCG GCA GCG CGG GGC AGA CG) wherein Z is 18;

z) SEQ ID NO: 158 (CCG CCG GCA GCG CGG GGC AG) wherein Z is 18;

aa) SEQ ID NO: 159 (GAX GXX ACC GCC GGC AGC GC) wherein Z is 18;

bb) SEQ ID NO: 160 (CXG GGA XGX XAC CGC CGG CA) wherein Z is 18;

cc) SEQ ID NO: 161 (GCX XCX GGG AXG XXA CCG CC) wherein Z is 18;

dd) SEQ ID NO: 162 (XGG CAA CXC GXA XGX CCX XA) wherein Z is 18;

ee) SEQ ID NO: 163 (AXX CXG GCA ACX CGX AXG XC) wherein Z is 18;

ff) SEQ ID NO: 164 (AAG XGA XXC XGG CAA CXC GX) wherein Z is 18;

gg) SEQ ID NO: 165 (XGG GXG XCA GCG GAA GXG AX) wherein Z is 18;

hh) SEQ ID NO: 166 (GXC CAC XGG GXG XCA GCG GA) wherein Z is 18;

ii) SEQ ID NO: 167 (GCX XGG XCC ACX GGG XGX CA) wherein Z is 18;

jj) SEQ ID NO: 168 (CCC CAC XXC XGC AXA AAG GX) wherein Z is 18;

kk) SEQ ID NO: 169 (GGA GCC CCA CXX CXG CAX AA) wherein Z is 18;

ll) SEQ ID NO: 170 (GCX GGG AGC CCC ACX XCX GC) wherein Z is 18;

mm) SEQ ID NO: 171 (CCA CGC CXG GCX GGG AGC CC) wherein Z is 18;

nn) SEQ ID NO: 172 (XCC GAA GXG CXG GGA XXX CA) wherein Z is 18;

oo) SEQ ID NO: 173 (XCC ACC CCC CXX GGC CXX CC) wherein Z is 18;

pp) SEQ ID NO: 174 (XGA XCC ACC CCC CXX GGC CX) wherein Z is 18;

qq) SEQ ID NO: 175 (XCA AGX GAX CCA CCC CCC XX) wherein Z is 18;

rr) SEQ ID NO: 176 (GAA CXC CXG AGC XCA AGX GA) wherein Z is 18;

ss) SEQ ID NO:177 (XCX CGA ACX CCX GAG CXC AA) wherein Z is 18;

tt) SEQ ID NO: 178 (CCA GGC XGG XCX CGA ACX CC) wherein Z is 18;

uu) SEQ ID NO: 179 (XXX GCC AXG XXA CCC AGG CX) wherein Z is 18;

vv) SEQ ID NO: 180 (ACG GGA XXX XGC CAX GXX AC) wherein Z is 18;

ww) SEQ ID NO: 181 (XAG AGA CGG GAX XXX GCC AX) wherein Z is 18;

xx) SEQ ID NO: 182 (XXX XGX AGA GAC GGG AXX XX) wherein Z is 18;

yy) SEQ ID NO: 183 (XCX GXA XXX XXG XAG AGA CG) wherein Z is 18;

zz) SEQ ID NO: 184 (AXX XXC XGX AXX XXX GXA GA) wherein Z is 18;

aaa) SEQ ID NO: 185 (GCX AAX XXX CXG XAX XXX XG) wherein Z is 18;

bbb) SEQ ID NO: 186 (CCG CCG CCC CCG CCC CXG CC) wherein Z is 18;

ccc) SEQ ID NO: 187 (XGG CCG CCG CCC CCG CCC CX) wherein Z is 18;

ddd) SEQ ID NO: 188 (CXG CCC XGG CCG CCG CCC CC) wherein Z is 18;

eee) SEQ ID NO: 189 (CAC CCX CXG CCC XGG CCG CC) wherein Z is 18;

fff) SEQ ID NO: 190 (GCG CAC CCX CXG CCC XGG CC) wherein Z is 18;

ggg) SEQ ID NO: 191 (XGX CGA XGX CCA CGC GCA CC) wherein Z is 18;

hhh) SEQ ID NO: 192 (XGC GXG GGX GXC GAX GXC CA) wherein Z is 18;

iii) SEQ ID NO: 193 (GCA CCC CAC CCX XGX GAG GX) wherein Z is 18;

jjj) SEQ ID NO: 194 (AAC AXG CAC CCC ACC CXX GX) wherein Z is 18;

kkk) SEQ ID NO: 195 (AGG AGG AGG ACG CCX CCC CC) wherein Z is 18;

lll) SEQ ID NO: 196 (CXC AXC XGC AGA GCC AGG AG) wherein Z is 18;

mmm) SEQ ID NO: 197 (GCX CCC XCA XCX GCA GAG CC) wherein Z is 18;

nnn) SEQ ID NO: 198 (XCG GCX CCC XCA XCX GCA GA) wherein Z is 18;

ooo) SEQ ID NO: 199 (GCC XCG GCX CCC XCA XCX GC) wherein Z is 18;

ppp) SEQ ID NO:200 (XXC XGG GAX GXX ACC GCC GG) wherein Z is 18;

qqq) SEQ ID NO:201 (CXX CXG GGA XGX XAC CGC CG) wherein Z is 18;

rrr) SEQ ID NO:202 (CGC XXC XGG GAX GXX ACC GC) wherein Z is 18;

sss) SEQ ID NO:203 (CCG CXX CXG GGA XGX XAC CG) wherein Z is 18;

ttt) SEQ ID NO:204 (ACC CGC XXC XGG GAX GXX AC) wherein Z is 18;

uuu) SEQ ID NO:205 (XCA AAC CCG CXX CXG GGA XG) wherein Z is 18;

vvv) SEQ ID NO:206 (ACG XXC AAA CCC GCX XCX GG) wherein Z is 18;

www) SEQ ID NO:207 (GGG CXC XCA AAG CAG CXC XG) wherein Z is 18;

xxx) SEQ ID NO:208 (GGG GCX CXC AAA GCA GCX CX) wherein Z is 18;

yyy) SEQ ID NO:209 (ACG GGG CXC XCA AAG CAG CX) wherein Z is 18;

zzz) SEQ ID NO:210 (XCA CGG GGC XCX CAA AGC AG) wherein Z is 18;

aaaa) SEQ ID NO:211 (GCX CXC AAA GCA GCX CXG AG) wherein Z is 18;

bbbb) SEQ ID NO:212 (CXC XCA AAG CAG CXC XGA GA) wherein Z is 18;

cccc) SEQ ID NO:213 (CXC AAA GCA GCX CXG AGA CA) wherein Z is 18;

dddd) SEQ ID NO:214 (CAA AGC AGC XCX GAG ACA XC) wherein Z is 18;

eeee) SEQ ID NO:215 (AAG CAG CXC XGA GAC AXC AA) wherein Z is 18;

ffff) SEQ ID NO:216 (GCC CXG GXC XGC XGG CXC CCX GCX G) wherein Z is 23;

gggg) SEQ ID NO:217 (CCC XGG XCX GCX GGC XCC CXG CXG G) wherein Z is 23;

hhhh) SEQ ID NO:218 (CCX GGX CXG CXG GCX CCC XGC XGG X) wherein Z is 23;

iiii) SEQ ID NO:219 (CXG GXC XGC XGG CXC CCX GCX GGX G) wherein Z is 23;

jjjj) SEQ ID NO:220 (XGG XCX GCX GGC XCC CXG CXG GXG A) wherein Z is 23;

kkkk) SEQ ID NO:221 (GGX CXG CXG GCX CCC XGC XGG XGA G) wherein Z is 23;

llll) SEQ ID NO:222 (GXC XGC XGG CXC CCX GCX GGX GAG C) wherein Z is 23;

mmmm) SEQ ID NO:223 (XCX GCX GGC XCC CXG CXG GXG AGC X) wherein Z is 23;

nnnn) SEQ ID NO:224 (GGG CCC XGG XCX GCX GGC XCC CXG C) wherein Z is 23;

oooo) SEQ ID NO:225 (GGG GCC CXG GXC XGC XGG CXC CCX G) wherein Z is 23;

pppp) SEQ ID NO:226 (CGG GGC CCX GGX CXG CXG GCX CCC X) wherein Z is 23;

qqqq) SEQ ID NO:227 (CCG GGG CCC XGG XCX GCX GGC XCC C) wherein Z is 23;

rrrr) SEQ ID NO:228 (CCC GGG GCC CXG GXC XGC XGG CXC C) wherein Z is 23;

ssss) SEQ ID NO:229 (XCC CGG GGC CCX GGX CXG CXG GCX C) wherein Z is 23;

tttt) SEQ ID NO:230 (AXC CCG GGG CCC XGG XCX GCX GGC X) wherein Z is 23;

uuuu) SEQ ID NO:231 (CAX CCC GGG GCC CXG GXC XGC XGG C) wherein Z is 23;

vvvv) SEQ ID NO:232 (XCX GCC CXG GCC GCC GCC CCC GCC CCX) wherein Z is 23;

wwww) SEQ ID NO:233 (XGA GGX GCG XGG GXG XCG AXG XCC A) wherein Z is 23;

xxxx) SEQ ID NO:234 (GAG GXG CGX GGG XGX CGA XGX CCA C) wherein Z is 23;

yyyy) SEQ ID NO:235 (AGG XGC GXG GGX GXC GAX GXC CAC G) wherein Z is 23;

zzzz) SEQ ID NO:236 (GCG CGX GGA CAX CGA CAC CCA CGC A) wherein Z is 23;

aaaaa) SEQ ID NO:237 (XGX GAG GGC GCG XGG ACA XCG ACA C) wherein Z is 23;

bbbbb) SEQ ID NO:238 (XXG XGA GGG CGC GXG GAC AXC GAC A) wherein Z is 23;

ccccc) SEQ ID NO:239 (CXX GXG AGG GCG CGX GGA CAX CGA C) wherein Z is 23;

ddddd) SEQ ID NO:240 (GAG AGG GCC AGA AGG AAG) wherein Z is 16;

eeeee) SEQ ID NO:241 (GAG GGC CAG AAG GAA GGG) wherein Z is 16;

fffff) SEQ ID NO:242 (GGG CCA GAA GGA AGG GCG) wherein Z is 16;

ggggg) SEQ ID NO:243 (GGG AGA GGG CCA GAA GGA) wherein Z is 16;

hhhhh) SEQ ID NO:244 (AGA GGG CCA GAA GGA AGG GC) wherein Z is 18;

iiiii) SEQ ID NO:245 (GAG GGC CAG AAG GAA GGG CG) wherein Z is 18;

jjjjj) SEQ ID NO:246 (AGG GCC AGA AGG AAG GGC GA) wherein Z is 18;

kkkkk) SEQ ID NO:247 (GGG CCA GAA GGA AGG GCG AG) wherein Z is 18;

lllll) SEQ ID NO:248 (GGC CAG AAG GAA GGG CGA GA) wherein Z is 18;

mmmmm) SEQ ID NO:249 (GCC AGA AGG AAG GGC GAG AA) wherein Z is 18;

nnnnn) SEQ ID NO:250 (GGG AGA GGG CCA GAA GGA AGG G) wherein Z is 20;

ooooo) SEQ ID NO:251 (CXG GGG AGA GGG CCA GAA GGA AGG G) wherein Z is 23;

ppppp) SEQ ID NO:252 (GAG AGG GCC AGA AGG AAG GGC G) wherein Z is 20;

qqqqq) SEQ ID NO:253 (GAG AGG GCC AGA AGG AAG GGC GAG A) wherein Z is 23;

rrrrr) SEQ ID NO:254 (GAA GGA AGG GCG AGA AAA GC) wherein Z is 18; and

sssss) SEQ ID NO:255 (GCA GAA AAG CXC CAG CAG GG) wherein Z is 18,
wherein X is selected from uracil (U) or thymine (T).

In some embodiments, the targeting sequence of an antisense oligomers of the disclosure, including compounds of formula (I), (IVa), (IVb), (IVc), (V), (VI), (VII), (VIII), (IX), (X), and (XI), is selected from:

a) SEQ ID NO:296 (AAG CXC CAG CAG GGG AGX GCA GAG C) wherein Z is 23;

b) SEQ ID NO:297 (AAA AGC XCC AGC AGG GGA GXG CAG A) wherein Z is 23;

c) SEQ ID NO:298 (AGA AAA GCX CCA GCA GGG GAG XGC A) wherein Z is 23;

d) SEQ ID NO:299 (CGA GAA AAG CXC CAG CAG GGG AGX G) wherein Z is 23;

e) SEQ ID NO:300 (GGC GAG AAA AGC XCC AGC AGG GGA G) wherein Z is 23;

f) SEQ ID NO:301 (AGG GCG AGA AAA GCX CCA GCA GGG G) wherein Z is 23;

g) SEQ ID NO:302 (GAA GGG CGA GAA AAG CXC CAG CAG G) wherein Z is 23;

h) SEQ ID NO:303 (AGG AAG GGC GAG AAA AGC XCC AGC A) wherein Z is 23;

i) SEQ ID NO:304 (GAA GGA AGG GCG AGA AAA GCX CCA G) wherein Z is 23;

j) SEQ ID NO:305 (CAG AAG GAA GGG CGA GAA AAG CXC C) wherein Z is 23;

k) SEQ ID NO:306 (GCC AGA AGG AAG GGC GAG AAA AGC X) wherein Z is 23;

l) SEQ ID NO:307 (GGG CCA GAA GGA AGG GCG AGA AAA G) wherein Z is 23;

m) SEQ ID NO:308 (GAG GGC CAG AAG GAA GGG CGA GAA A) wherein Z is 23;

n) SEQ ID NO:309 (GAG AGG GCC AGA AGG AAG GGC GAG A) wherein Z is 23;

o) SEQ ID NO:310 (AGG GCC AGA AGG AAG GGC GA) wherein Z is 18;

p) SEQ ID NO:311 (GAG AGG GCC AGA AGG AAG GG) wherein Z is 18;

q) SEQ ID NO:312 (CXG GGG AGA GGG CCA GAA GGA AGG G) wherein Z is 23;

r) SEQ ID NO:313 (GAG AGG GCC AGA AGG AAG) wherein Z is 18;

s) SEQ ID NO:314 (GGG AGA GGG CCA GAA GGA) wherein Z is 18;

t) SEQ ID NO:315 (GGX CXG CXG GCX CCC XGC XGG XGA G) wherein Z is 23;

u) SEQ ID NO:316 (CAC XCA CGG GGC XCX CAA AGC AGC X) wherein Z is 23;

v) SEQ ID NO:317 (XCX GGG AXG XXA CCG CCG GCA GCG C) wherein Z is 23;

w) SEQ ID NO:318 (XXX GCC AXG XXA CCC AGG CX) wherein Z is 18;

x) SEQ ID NO:319 (ACX CAC GGG GCX CXC AAA GCA GCX C) wherein Z is 23;

y) SEQ ID NO:320 (GCA CXC ACG GGG CXC XCA AAG CAG C) wherein Z is 23;

z) SEQ ID NO:321 (CGG GGC XCX CAA AGC AGC XCX GAG A) wherein Z is 23;

aa) SEQ ID NO:322 (ACG GGG CXC XCA AAG CAG CXC XGA G) wherein Z is 23;

bb) SEQ ID NO:323 (CAC GGG GCX CXC AAA GCA GCX CXG A) wherein Z is 23;

cc) SEQ ID NO:324 (XCA CGG GGC XCX CAA AGC AGC XCX G) wherein Z is 23;

dd) SEQ ID NO:325 (CXC ACG GGG CXC XCA AAG CAG CXC X) wherein Z is 23;

ee) SEQ ID NO:326 (GGC ACX CAC GGG GCX CXC AAA GCA G) wherein Z is 23;

ff) SEQ ID NO:327 (CGG CAC XCA CGG GGC XCX CAA AGC A) wherein Z is 23;

gg) SEQ ID NO:328 (GCG GCA CXC ACG GGG CXC XCA AAG C) wherein Z is 23;

hh) SEQ ID NO:329 (GGC GGC ACX CAC GGG GCX CXC AAA G) wherein Z is 23;

ii) SEQ ID NO:330 (GGG CGG CAC XCA CGG GGC XCX CAA A) wherein Z is 23;

jj) SEQ ID NO:331 (GGG GCG GCA CXC ACG GGG CXC XCA A) wherein Z is 23;

kk) SEQ ID NO:332 (AGG GGC GGC ACX CAC GGG GCX CXC A) wherein Z is 23;

ll) SEQ ID NO:333 (GAG GGG CGG CAC XCA CGG GGC XCX C) wherein Z is 23;

mm) SEQ ID NO: 334 (GGC XCX CAA AGC AGC XCX GA) wherein Z is 18;

nn) SEQ ID NO: 335 (XXC XGG GAX GXX ACC GCC GGC AGC G) wherein Z is 23;

oo) SEQ ID NO: 336 (CXX CXG GGA XGX XAC CGC CGG CAG C) wherein Z is 23;

pp) SEQ ID NO: 337 (GCX XCX GGG AXG XXA CCG CCG GCA G) wherein Z is 23;

qq) SEQ ID NO: 338 (CGC XXC XGG GAX GXX ACC GCC GGC A) wherein Z is 23;

rr) SEQ ID NO: 339 (CCG CXX CXG GGA XGX XAC CGC CGG C) wherein Z is 23;

ss) SEQ ID NO: 340 (CCC GCX XCX GGG AXG XXA CCG CCG G) wherein Z is 23;

tt) SEQ ID NO: 341 (ACC CGC XXC XGG GAX GXX ACC GCC G) wherein Z is 23; and

uu) SEQ ID NO: 342 (AAC CCG CXX CXG GGA XGX XAC CGC C) wherein Z is 23,
wherein X is selected from uracil (U) or thymine (T).

D. The Preparation of PMO-X with Basic Nitrogen Internucleoside Linkers

Morpholino subunits, the modified intersubunit linkages, and oligomers comprising the same can be prepared as described, for example, in U.S. Pat. Nos. 5,185,444, and 7,943,762, which are incorporated by reference in their entireties. The morpholino subunits can be prepared according to the following general Reaction Scheme I.

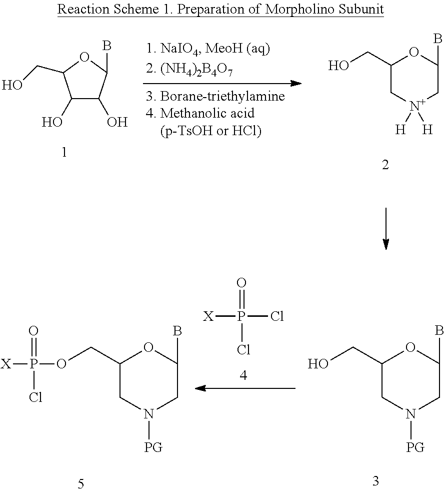

Referring to Reaction Scheme 1, wherein B represents a base pairing moiety and PG represents a protecting group, the morpholino subunits may be prepared from the corresponding ribonucleoside (1) as shown. The morpholino subunit (2) may be optionally protected by reaction with a suitable protecting group precursor, for example trityl chloride. The 3′ protecting group is generally removed during solid-state oligomer synthesis as described in more detail below. The base pairing moiety may be suitably protected for sold phase oligomer synthesis. Suitable protecting groups include benzoyl for adenine and cytosine, phenylacetyl for guanine, and pivaloyloxymethyl for hypoxanthine (I). The pivaloyloxymethyl group can be introduced onto the N1 position of the hypoxanthine heterocyclic base. Although an unprotected hypoxanthine subunit, may be employed, yields in activation reactions are far superior when the base is protected. Other suitable protecting groups include those disclosed in co-pending U.S. application Ser. No. 12/271,040, which is hereby incorporated by reference in its entirety.

Reaction of 3 with the activated phosphorous compound 4, results in morpholino subunints having the desired linkage moiety 5. Compounds of structure 4 can be prepared using any number of methods known to those of skill in the art. For example, such compounds may be prepared by reaction of the corresponding amine and phosphorous oxychloride. In this regard, the amine starting material can be prepared using any method known in the art, for example those methods described in the Examples and in U.S. Pat. No. 7,943,762.

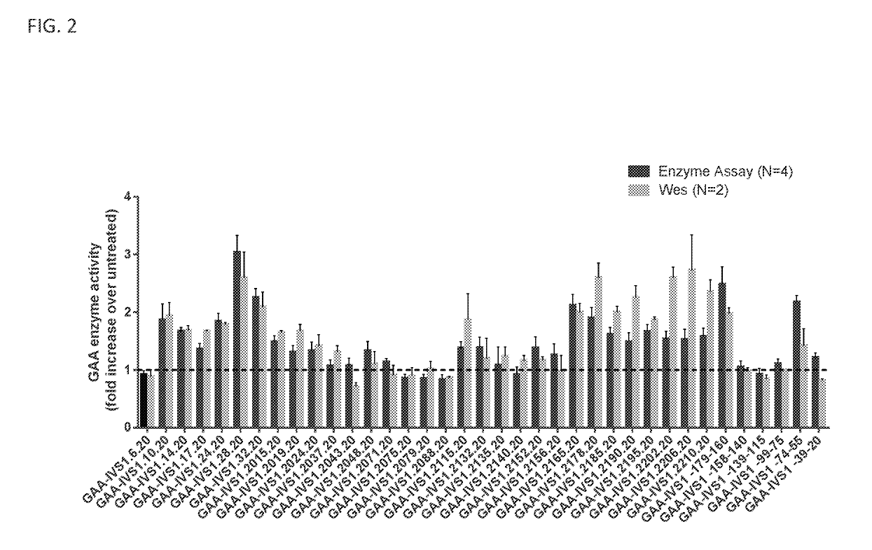

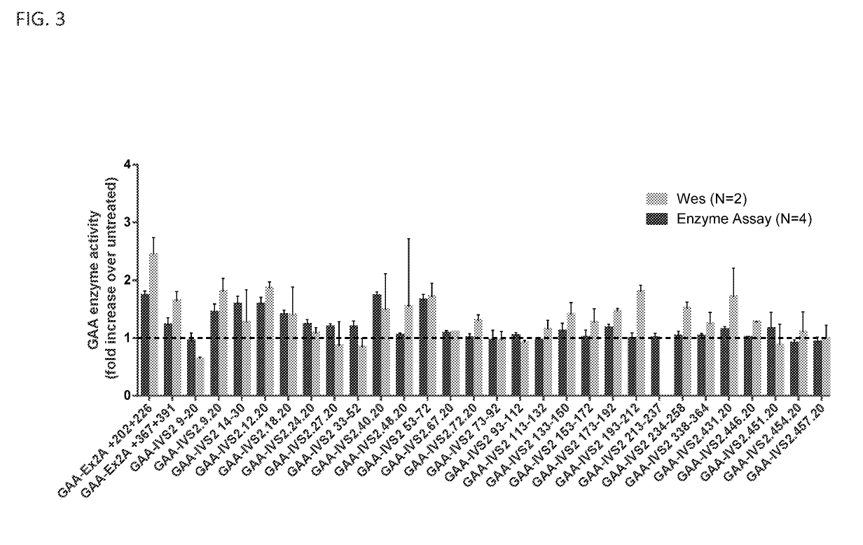

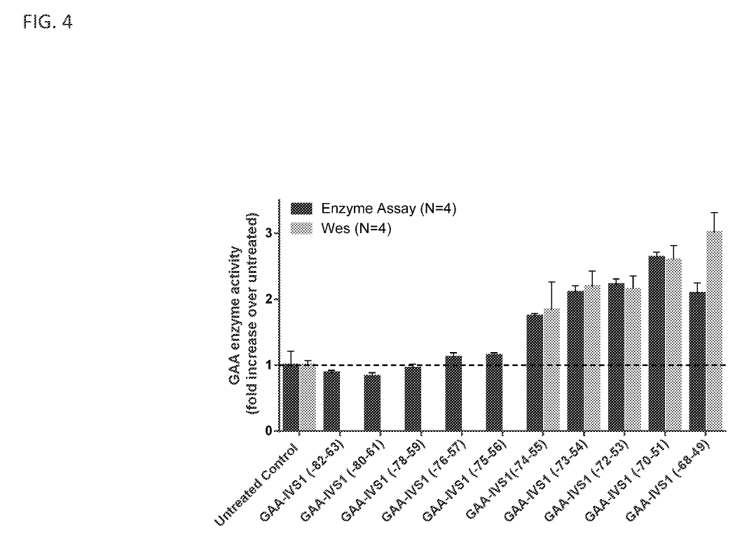

Compounds of structure 5 can be used in solid-phase automated oligomer synthesis for preparation of oligomers comprising the intersubunit linkages. Such methods are well known in the art. Briefly, a compound of structure 5 may be modified at the 5′ end to contain a linker to a solid support. For example, compound 5 may be linked to a solid support by a linker comprising L¹¹ and L¹⁵. An exemplary method is demonstrated in FIGS. 1 and 2. Once supported, the protecting group (e.g., trityl) is removed and the free amine is reacted with an activated phosphorous moiety of a second compound of structure 5. This sequence is repeated until the desired length of oligo is obtained. The protecting group in the terminal 5′ end may either be removed or left on if a 5′-modification is desired. The oligo can be removed from the solid support using any number of methods, for example treatment with DTT followed by ammonium hydroxide as depicted in FIGS. 3 and 4.

The preparation of modified morpholino subunits and morpholino oligomers are described in more detail in the Examples. The morpholino oligomers containing any number of modified linkages may be prepared using methods described herein, methods known in the art and/or described by reference herein. Also described in the examples are global modifications of morpholino oligomers prepared as previously described (see e.g., PCT publication WO2008036127).

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid moieties may be blocked with base labile groups such as, without limitation, methyl, or ethyl, and hydroxy reactive moieties may be blocked with base labile groups such as acetyl in the presence of amines blocked with acid labile groups such as tert-butyl carbamate or with carbamates that are both acid and base stable but hydrolytically removable.

Carboxylic acid and hydroxyl reactive moieties may also be blocked with hydrolytically removable protective groups such as the benzyl group, while amine groups may be blocked with base labile groups such as Fmoc. A particularly useful amine protecting group for the synthesis of compounds of Formula (I) is the trifluoroacetamide. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups are known in the art and include, but are not limited to the following moieties:

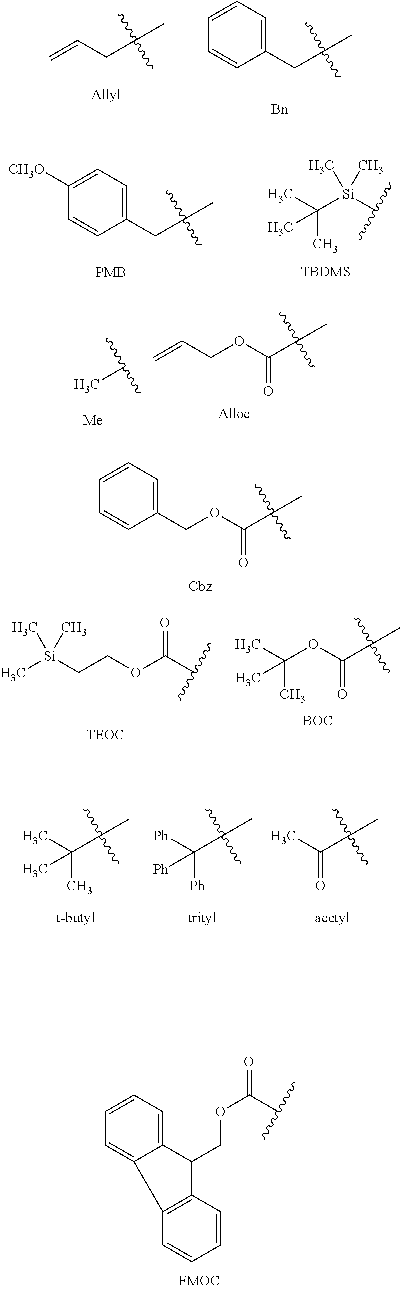

Unless otherwise noted, all chemicals were obtained from Sigma-Aldrich-Fluka. Benzoyl adenosine, benzoyl cytidine, and phenylacetyl guanosine were obtained from Carbosynth Limited, UK.

Synthesis of PMO, PMO+, PPMO, and PMO-X containing further linkage modifications as described herein was done using methods known in the art and described in pending U.S. application Ser. Nos. 12/271,036 and 12/271,040 and PCT publication number WO/2009/064471, which are hereby incorporated by reference in their entirety.

PMO with a 3′ trityl modification are synthesized essentially as described in PCT publication number WO/2009/064471 with the exception that the detritylation step is omitted.

### IV. Formulations

The compounds of the disclosure may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the disclosure encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the disclosure, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligomers of the disclosure are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the disclosure: i.e., salts that retain the desired biological activity of the parent compound and do not impart undesired toxicological effects thereto. For oligomers, examples of pharmaceutically acceptable salts and their uses are further described in U.S. Pat. No. 6,287,860, which is incorporated herein in its entirety.

The present disclosure also includes pharmaceutical compositions and formulations which include the antisense compounds of the disclosure. The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligomers with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, gel capsules, liquid syrups, soft gels, suppositories, and enemas. The compositions of the present disclosure may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present disclosure include, but are not limited to, solutions, emulsions, foams and liposome-containing formulations. The pharmaceutical compositions and formulations of the present disclosure may comprise one or more penetration enhancers, carriers, excipients or other active or inactive ingredients.

Emulsions are typically heterogeneous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an embodiment of the present disclosure. Emulsions and their uses are well known in the art and are further described in U.S. Pat. No. 6,287,860, which is incorporated herein in its entirety.

Formulations of the present disclosure include liposomal formulations. As used in the present disclosure, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the present disclosure may also include surfactants. The use of surfactants in drug products, formulations and in emulsions is well known in the art. Surfactants and their uses are further described in U.S. Pat. No. 6,287,860, which is incorporated herein in its entirety.

In some embodiments, the present disclosure employs various penetration enhancers to effect the efficient delivery of nucleic acids, particularly oligomers. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Formulations for topical administration include those in which the oligomers of the disclosure are in admixture with a topical delivery agent such as lipids, liposomes, fatty acids, fatty acid esters, steroids, chelating agents and surfactants. Lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligomers of the disclosure may be encapsulated within liposomes or may form complexes thereto, in particular to cationic liposomes. Alternatively, oligomers may be complexed to lipids, in particular to cationic lipids. Fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Oral formulations are those in which oligomers of the disclosure are administered in conjunction with one or more penetration enhancers, surfactants and chelators. Surfactants include fatty acids and/or esters or salts thereof, bile acids and/or salts thereof. Bile acids/salts and fatty acids and their uses are further described in U.S. Pat. No. 6,287,860, which is incorporated herein in its entirety. In some embodiments, the present disclosure provides combinations of penetration enhancers, for example, fatty acids/salts in combination with bile acids/salts. An exemplary combination is the sodium salt of lauric acid, capric acid and UDCA. Further penetration enhancers include polyoxyethylene-9-lauryl ether, polyoxyethylene-20-cetyl ether. Oligomers of the disclosure may be delivered orally, in granular form including sprayed dried particles, or complexed to form micro or nanoparticles. Oligomer complexing agents and their uses are further described in U.S. Pat. No. 6,287,860, which is incorporated herein in its entirety. Oral formulations for oligomers and their preparation are described in detail in U.S. application Ser. No. 09/108,673 (filed Jul. 1, 1998), U.S. Ser. No. 09/315,298 (filed May 20, 1999) and U.S. Ser. No. 10/071,822, filed Feb. 8, 2002, each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments of the disclosure provide pharmaceutical compositions containing one or more oligomeric compounds and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxyco-formycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds of the disclosure, such chemotherapeutic agents may be used individually (e.g., 5-FU and oligomer), sequentially (e.g., 5-FU and oligomer for a period of time followed by MTX and oligomer), or in combination with one or more other such chemotherapeutic agents (e.g., 5-FU, MTX and oligomer, or 5-FU, radiotherapy and oligomer). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the disclosure. Combinations of antisense compounds and other non-antisense drugs are also within the scope of this disclosure. Two or more combined compounds may be used together or sequentially.

In another related embodiment, compositions of the disclosure may contain one or more antisense compounds, particularly oligomers, targeted to a first nucleic acid and one or more additional antisense compounds targeted to a second nucleic acid target. Alternatively, compositions of the disclosure may contain two or more antisense compounds targeted to different regions of the same nucleic acid target. Numerous examples of antisense compounds are known in the art. Two or more combined compounds may be used together or sequentially.

### V. Methods of Use

Certain embodiments relate to methods of increasing expression of exon 2-containing GAA mRNA and/or protein using the antisense oligomers of the present disclosure for therapeutic purposes (e.g., treating subjects with GSD-II). Accordingly, in some embodiments, the present disclosure provides methods of treating an individual afflicted with or at risk for developing GSD-II, comprising administering an effective amount of an antisense oligomer of the disclosure to the subject. In some embodiments, the antisense oligomer comprising a nucleotide sequence of sufficient length and complementarity to specifically hybridize to a region within the pre-mRNA of the acid alpha-glucosidase (GAA) gene, wherein binding of the antisense oligomer to the region increases the level of exon 2-containing GAA mRNA in a cell and/or tissue of the subject. Exemplary antisense targeting sequences are shown in Tables 2A-2C.

Also included are antisense oligomers for use in the preparation of a medicament for the treatment of glycogen storage disease type II (GSD-II; Pompe disease), comprising a nucleotide sequence of sufficient length and complementarity to specifically hybridize to a region within the pre-mRNA of the acid alpha-glucosidase (GAA) gene, wherein binding of the antisense oligomer to the region increases the level of exon 2-containing GAA mRNA.

In some embodiments of the method of treating GSD-II or the medicament for the treatment of GSD-II, the antisense oligomer compound comprises:

a non-natural chemical backbone selected from a phosphoramidate or phosphorodiamidate morpholino oligomer (PMO), a peptide nucleic acid (PNA), a locked nucleic acid (LNA), a phosphorothioate oligomer, a tricyclo-DNA oligomer, a tricyclo-phosphorothioate oligomer, a 2′O-Me-modified oligomer, or any combination of the foregoing; and

a targeting sequence complementary to a region within intron 1 (SEQ ID. NO: 1), intron 2 (SEQ ID. NO: 2), or exon 2 (SEQ ID. NO: 3) of a pre-mRNA of the human acid alpha-glucosidase (GAA) gene.

As noted above, “GSD-II” refers to glycogen storage disease type II (GSD-II or Pompe disease), a human autosomal recessive disease that is often characterized by under expression of GAA protein in affected individuals. Included are subjects having infantile GSD-II and those having late onset forms of the disease.

In certain embodiments, a subject has reduced expression and/or activity of GAA protein in one or more tissues (for example, relative to a healthy subject or an earlier point in time), including heart, skeletal muscle, liver, and nervous system tissues. In some embodiments, the subject has increased accumulation of glycogen in one or more tissues (for example, relative to a healthy subject or an earlier point in time), including heart, skeletal muscle, liver, and nervous system tissues. In specific embodiments, the subject has at least one IVS1-13T>G mutation (also referred to as c.336-13T>G), possibly in combination with other mutation(s) that leads to reduced expression of functional GAA protein. A summary of molecular genetic testing used in GSD-II is shown in Table 3 below.

TABLE 3

| | | | Mutation | |
|---|---|---|---|---|
| | | | Detection | |
| Gene | | | Frequency by | Test |
| Symbol | Test Method | Mutations Detected | Test Method | Availability |
| GAA | Sequence | p.Arg854* | ~50%-60% | Clinical |
| | analysis | p.Asp645Glu | ~40%-80% | |
| | | IVS1-13T > G | ~50%-85% | |
| | | Other sequence variants | 83%-93% | |
| | | in the gene | | |
| | Sequence analysis of | Sequence variants in | 83%-93% | |
| | select exons | the select exons | | |
| | Targeted mutation | Sequence variants in | 100% of for | |
| | analysis | targeted sites | variants among | |
| | | | the targeted | |
| | | | mutations | |
| | Deletion/duplication | Exonic and whole-gene | 5%-13% | |
| | analysis | deletions/duplications | | |

Certain embodiments relate to methods of increasing expression of exon 2-containing GAA mRNA or protein in a cell, tissue, and/or subject, as described herein. In some instances, exon-2 containing GAA mRNA or protein is increased by about or at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% relative to a control, for example, a control cell/subject, a control composition without the antisense oligomer, the absence of treatment, and/or an earlier time-point. Also included are methods of maintaining the expression of containing GAA mRNA or protein relative to the levels of a healthy control.

Some embodiments relate to methods of increasing expression of functional/active GAA protein a cell, tissue, and/or subject, as described herein. In certain instances, the level of functional/active GAA protein is increased by about or at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% relative to a control, for example, a control cell/subject, a control composition without the antisense oligomer, the absence of treatment, and/or an earlier time-point. Also included are methods of maintaining the expression of functional/active GAA protein relative to the levels of a healthy control.

Particular embodiments relate to methods of reducing the accumulation of glycogen in one or more cells, tissues, and/or subjects, as described herein. In certain instances, the accumulation of glycogen is reduced by about or at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% relative to a control, for example, a control cell/subject, a control composition without the antisense oligomer, the absence of treatment, and/or an earlier time-point. Also included are methods of maintaining normal or otherwise healthy glycogen levels in a cell, tissue, and/or subject (e.g., asymptomatic levels or levels associated with reduced symptoms of GSD-II).

Also included are methods of reducing one or more symptoms of GSD-II in a subject in need thereof. Particular examples include symptoms of infantile GSD-II such as cardiomegaly, hypotonia, cardiomyopathy, left ventricular outflow obstruction, respiratory distress, motor delay/muscle weakness, and feeding difficulties/failure to thrive. Additional examples include symptoms of late onset GSD-II such as muscle weakness (e.g., skeletal muscle weakness including progressive muscle weakness), impaired cough, recurrent chest infections, hypotonia, delayed motor milestones, difficulty swallowing or chewing, and reduced vital capacity or respiratory insufficiency.

The antisense oligomers of the disclosure can be administered to subjects to treat (prophylactically or therapeutically) GSD-II. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug.

Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Effective delivery of the antisense oligomer to the target nucleic acid is one aspect of treatment. Routes of antisense oligomer delivery include, but are not limited to, various systemic routes, including oral and parenteral routes, e.g., intravenous, subcutaneous, intraperitoneal, and intramuscular, as well as inhalation, transdermal and topical delivery. The appropriate route may be determined by one of skill in the art, as appropriate to the condition of the subject under treatment. Vascular or extravascular circulation, the blood or lymph system, and the cerebrospinal fluid are some non-limiting sites where the RNA may be introduced. Direct CNS delivery may be employed, for instance, intracerebral ventribular or intrathecal administration may be used as routes of administration.

In particular embodiments, the antisense oligomer(s) are administered to the subject by intramuscular injection (IM), i.e., they are administered or delivered intramuscularly. Non-limiting examples of intramuscular injection sites include the deltoid muscle of the arm, the vastus lateralis muscle of the leg, and the ventrogluteal muscles of the hips, and dorsogluteal muscles of the buttocks. In specific embodiments, a PMO, PMO-X, or PPMO is administered by IM.

In certain embodiments, the subject in need thereof as glycogen accumulation in central nervous system tissues. Examples include instances where central nervous system pathology contributes to respiratory deficits in GSD-II (see, e.g., DeRuisseau et al., PNAS USA. 106:9419-24, 2009). Accordingly, the antisense oligomers described herein can be delivered to the nervous system of a subject by any art-recognized method, e.g., where the subject has GSD-II with involvement of the CNS. For example, peripheral blood injection of the antisense oligomers of the disclosure can be used to deliver said reagents to peripheral neurons via diffusive and/or active means. Alternatively, the antisense oligomers can be modified to promote crossing of the blood-brain-barrier (BBB) to achieve delivery of said reagents to neuronal cells of the central nervous system (CNS). Specific recent advancements in antisense oligomer technology and delivery strategies have broadened the scope of antisense oligomer usage for neuronal disorders (see, e.g., Forte, A., et al. 2005. Curr. Drug Targets 6:21-29; Jaeger, L. B., and W. A. Banks. 2005. Methods Mol. Med. 106:237-251; Vinogradov, S. V., et al. 2004. Bioconjug. Chem. 5:50-60; the foregoing are incorporated herein in their entirety by reference). For example, the antisense oligomers of the disclosure can be generated as peptide nucleic acid (PNA) compounds. PNA reagents have each been identified to cross the BBB (Jaeger, L. B., and W. A. Banks. 2005. Methods Mol. Med. 106:237-251). Treatment of a subject with, e.g., a vasoactive agent, has also been described to promote transport across the BBB (Id). Tethering of the antisense oligomers of the disclosure to agents that are actively transported across the BBB may also be used as a delivery mechanism. Administration of antisense agents together with contrast agents such as iohexol (e.g., separately, concurrently, in the same formulation) can also facilitate delivery across the BBB, as described in PCT Publication No. WO/2013/086207, incorporated by reference in its entirety.

In certain embodiments, the antisense oligomers of the disclosure can be delivered by transdermal methods (e.g., via incorporation of the antisense oligomers into, e.g., emulsions, with such antisense oligomers optionally packaged into liposomes). Such transdermal and emulsion/liposome-mediated methods of delivery are described for delivery of antisense oligomers in the art, e.g., in U.S. Pat. No. 6,965,025, the contents of which are incorporated in their entirety by reference herein.

The antisense oligomers described herein may also be delivered via an implantable device. Design of such a device is an art-recognized process, with, e.g., synthetic implant design described in, e.g., U.S. Pat. No. 6,969,400, the contents of which are incorporated in their entirety by reference herein.

Antisense oligomers can be introduced into cells using art-recognized techniques (e.g., transfection, electroporation, fusion, liposomes, colloidal polymeric particles and viral and non-viral vectors as well as other means known in the art). The method of delivery selected will depend at least on the oligomer chemistry, the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, antisense oligomers may be delivered using, e.g., methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The antisense oligomers may be administered in any convenient vehicle or carrier which is physiologically and/or pharmaceutically acceptable. Such a composition may include any of a variety of standard pharmaceutically acceptable carriers employed by those of ordinary skill in the art. Examples include, but are not limited to, saline, phosphate buffered saline (PBS), water, aqueous ethanol, emulsions, such as oil/water emulsions or triglyceride emulsions, tablets and capsules. The choice of suitable physiologically acceptable carrier will vary dependent upon the chosen mode of administration. “Pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compounds (e.g., antisense oligomers) of the present disclosure may generally be utilized as the free acid or free base. Alternatively, the compounds of this disclosure may be used in the form of acid or base addition salts. Acid addition salts of the free amino compounds of the present disclosure may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids.

Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” is intended to encompass any and all acceptable salt forms.

In addition, prodrugs are also included within the context of this disclosure. Prodrugs are any covalently bonded carriers that release a compound in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this disclosure wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the antisense oligomers of the disclosure. Further, in the case of a carboxylic acid (—COOH), esters may be employed, such as methyl esters, ethyl esters, and the like.

In some instances, liposomes may be employed to facilitate uptake of the antisense oligomer into cells (see, e.g., Williams, S. A., Leukemia 10(12):1980-1989, 1996; Lappalainen et al., Antiviral Res. 23:119, 1994; Uhlmann et al., antisense oligomers: a new therapeutic principle, Chemical Reviews, Volume 90, No. 4, 25 pages 544-584, 1990; Gregoriadis, G., Chapter 14, Liposomes, Drug Carriers in Biology and Medicine, pp. 287-341, Academic Press, 1979). Hydrogels may also be used as vehicles for antisense oligomer administration, for example, as described in WO 93/01286. Alternatively, the oligomers may be administered in microspheres or microparticles. (See, e.g., Wu, G. Y. and Wu, C. H., J. Biol. Chem. 262:4429-4432, 30 1987). Alternatively, the use of gas-filled microbubbles complexed with the antisense oligomers can enhance delivery to target tissues, as described in U.S. Pat. No. 6,245,747. Sustained release compositions may also be used. These may include semipermeable polymeric matrices in the form of shaped articles such as films or microcapsules.

In one embodiment, the antisense oligomer is administered to a mammalian subject, e.g., human or domestic animal, exhibiting the symptoms of a lysosomal storage disorder, in a suitable pharmaceutical carrier. In one aspect of the method, the subject is a human subject, e.g., a patient diagnosed as having GSD-II (Pompe disease). In one preferred embodiment, the antisense oligomer is contained in a pharmaceutically acceptable carrier, and is delivered orally. In another preferred embodiment, the oligomer is contained in a pharmaceutically acceptable carrier, and is delivered intravenously (i.v.).

In one embodiment, the antisense compound is administered in an amount and manner effective to result in a peak blood concentration of at least 200-400 nM antisense oligomer. Typically, one or more doses of antisense oligomer are administered, generally at regular intervals, for a period of about one to two weeks. Preferred doses for oral administration are from about 1-1000 mg oligomer per 70 kg. In some cases, doses of greater than 1000 mg oligomer/patient may be necessary. For i.v. administration, preferred doses are from about 0.5 mg to 1000 mg oligomer per 70 kg. The antisense oligomer may be administered at regular intervals for a short time period, e.g., daily for two weeks or less. However, in some cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

An effective in vivo treatment regimen using the antisense oligomers of the disclosure may vary according to the duration, dose, frequency and route of administration, as well as the condition of the subject under treatment (i.e., prophylactic administration versus administration in response to localized or systemic infection). Accordingly, such in vivo therapy will often require monitoring by tests appropriate to the particular type of disorder under treatment, and corresponding adjustments in the dose or treatment regimen, in order to achieve an optimal therapeutic outcome.

Treatment may be monitored, e.g., by general indicators of disease known in the art. The efficacy of an in vivo administered antisense oligomer of the disclosure may be determined from biological samples (tissue, blood, urine etc.) taken from a subject prior to, during and subsequent to administration of the antisense oligomer. Assays of such samples include (1) monitoring the presence or absence of heteroduplex formation with target and non-target sequences, using procedures known to those skilled in the art, e.g., an electrophoretic gel mobility assay; (2) monitoring the amount of a mutant mRNA in relation to a reference normal mRNA or protein as determined by standard techniques such as RT-PCR, Northern blotting, ELISA or Western blotting.

In some embodiments, the antisense oligomer is actively taken up by mammalian cells. In further embodiments, the antisense oligomer may be conjugated to a transport moiety (e.g., transport peptide or CPP) as described herein to facilitate such uptake.

### VI. Dosing

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligomers, and can generally be estimated based on EC50s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligomer is administered in maintenance doses, ranging from 0.01 μg to 100 g per kg of body weight, once or more daily, to once every 20 years.

While the present disclosure has been described with specificity in accordance with certain of its embodiments, the following examples serve only to illustrate the disclosure and are not intended to limit the same. Each of the references, patents, patent applications, GenBank accession numbers, and the like recited in the present application are incorporated herein by reference in its entirety.

### VII. Examples

### Example 1

### Design of Antisense Targeting Sequences

Antisense oligomer targeting sequences were designed for therapeutic splice-switching applications related to the IVS1-13T>G mutation in the human GAA gene. Here, it is expected that splice-switching oligomers will suppress intronic and exonic splice silencer elements (ISS and ESS elements, respectively) and thereby promote exon 2 retention in the mature GAA mRNA. Restoration of normal or near-normal GAA expression would then allow functional enzyme to be synthesized, thereby providing a clinical benefit to GSD-II patients.

Certain antisense targeting sequences were thus designed to mask splice silencer elements, either within exon 2 of the GAA gene or within its flanking introns. Non-limiting examples of potential silencer element targets include hnRNPA1 motifs (TAGGGA), Tra2-β motifs, and 9G8 motifs. In silico secondary structure analysis (mFold) of introns 1 and 2 (IVS1 and IVS2, respectively) mRNAs was also employed to identify long distance interactions that could provide suitable antisense target sequences. The antisense targeting sequences resulting from this analysis are shown in Table 2A (see also SEQ ID NOS:4-30), Table 2B (see also SEQ ID NOS: 133-255), and Table 2C (see also SEQ ID NOS:296-342).

Exemplary oligomers comprising a targeting sequence as set forth in Tables 2A, 2B, or 2C were prepared as PMOs (see, e.g., Table 5 below). These oligomers can, optionally, be conjugated to an arginine-rich peptide (such a conjugate is referred to as a “PPMO”). As described below, these antisense oligomers were introduced into GSD-II patient-derived fibroblasts using a nucleofection protocol as also described in Example 2 below.

### Example 2

### Materials and Methods

GSD-II cells. Patient-derived fibroblasts or lymphocytes from individuals with GSD-II (Coriell cell lines GM00443, GM11661, GM14463 and/or GM14484) are cultured according to standard protocols in Eagle's MEM with 10% FBS. Cells are passaged about 3-5 days before the experiments and are approximately 80% confluent at transfection or nucleofection.

GM00443 fibroblasts are from a 30 year old male. Adult form; onset in third decade; normal size and amount of mRNA for GAA, GAA protein detected by antibody, but only 9 to 26% of normal acid-alpha-1,4 glucosidase activity; passage 3 at CCR; donor subject is heterozygous with one allele carrying a T>G transversion at position −13 of the acceptor site of intron 1 of the GAA gene, resulting in alternatively spliced transcripts with deletion of the first coding exon [exon 2 (IVS1-13T>G)].

GM11661 fibroblasts are from a 38 year old male. Abnormal liver function tests; occasional charley-horse in legs during physical activity; morning headaches; intolerance to greasy foods; abdominal cyst; deficient fibroblast and WBC acid-alpha-1,4 glucosidase activity; donor subject is a compound heterozygote: allele one carries a T>G transversion at position −13 of the acceptor site of intron 1 of the GAA gene (IVS1-13T>G); the resulting alternatively spliced transcript has an in frame deletion of exon 2 which contains the initiation codon; allele two carries a deletion of exon 18.

GM14463 lymphocytes are from a 26 year old female. Clinically affected; adult onset; severe generalized muscle weakness and wasting; severe respiratory insufficiency; muscle biopsy showed acid maltase deficiency; donor subject is a compound heterozygote: one allele has a T>G transversion at position −13 of the acceptor site of intron 1 of the GAA gene (IVS1-13T>G) resulting in alternatively spliced transcripts with deletion of the first coding exon, exon 2; the second allele has a 1 bp deletion at nucleotide 366 in exon 2 (c.366delT) resulting in a frameshift and protein truncation[Gn 124SerfsX18).

GM14484 lymphocytes are from a 61 year old male. Clinically affected; adult onset); donor subject is a compound heterozygote: one allele has a T>G transversion at position −13 of the acceptor site of intron 1 of the GAA gene (IVS1-13T>G) resulting in alternatively spliced transcripts with deletion of the first coding exon, exon 2; the second allele has a C>T transition at nucleotide 172 in exon 2 (c.172C>T) resulting in a stop at codon 58 [Gln58Ter (Q58X)].

Upon arrival, GSD-II patient cells are expanded and aliquots frozen for long-term storage. Cells are then propagated and RT-PCR is performed on total RNA extracted from the cells to confirm exon 2 is missing from the mature GAA-coding transcript.

Nucleofection Protocol.

Antisense PMOs/PPMOs (PMOs conjugated to an arginine-rich peptide) are prepared as 1-2 mM stock solutions in nuclease-free water (not treated with DEPC) from which appropriate dilutions are made for nucleofection. GSD-II cells are trypsinized, counted, centrifuged at 90 g for 10 minutes, and 1-5×10⁵ cells per well are resuspended in nucleofection Solution P2 (Lonza). Antisense PMO solution and cells are then added to each well of a Nucleocuvette 16-well strip, and pulsed with program EN-100. Cells are incubated at room temperature for 10 minutes and transferred to a 12-well plate in duplicate. Total RNA is isolated from treated cells after 48 hours using the GE Illustra 96 Spin kit following the manufacturer's recommended protocol. Recovered RNA is stored at −80° C. prior to analysis.

GAA RT-PCR.

For PCR detection of exon 2-containing mRNAs, primer sequences are chosen from exon 1(forward) to exon 3(reverse). RT-PCR across exons 1-3 will generate a full length amplicon of around 1177 bases. The size difference between the intact amplicon (˜1177 bases) and the ˜600 base transcript that is missing exon 2 (exon 2 is ˜578 bases) means there will be substantial preferential amplification of the shorter product. This will set a high benchmark in assaying the efficacy of antisense oligomers to induce splicing of the full-length transcript or exon2-containing transcript.

Reverse transcriptase PCR is performed to amplify the GAA allele using the SuperScript III One-Step RT-PCR system (Invitrogen). 400 ng total RNA isolated from nucleofected cells is reverse transcribed and amplified with the gene-specific primers.

The amplification solution provided in the One-Step kit is supplemented with Cy5-labeled dCTP (GE) to enable band visualization by fluorescence. Digested samples are run on a pre-cast 10% acrylamide/TBE gel (Invitrogen) and visualized on a Typhoon Trio (GE) using the 633 nm excitation laser and 670 nm BP 30 emission filter with the focal plane at the platen surface. Gels are analyzed with ImageQuant (GE) to determine the intensities of the bands. Intensities from all bands containing exon 2 are added together to represent the full exon 2 transcript levels in the inclusion analysis.

Alternatively, PCR amplification products (without the supplemented Cy5-labeled dCTP) are analyzed on a Caliper LabChip GX bioanalyzer or Agilent 2200 Tape Station for determination of % exon inclusion.

GAA Enzyme Assay & Protein Simple Wes.

Untransformed patient-derived fibroblasts (GM00443) were nucleofected with PMO at various concentrations in Lonza's P3 nucleofector solution and incubated at 37° C. with 5% CO₂ for six days. Cells were washed twice with Hank's Balanced Salt Solution (HBSS), lysed with unbuffered H₂O, frozen/thawed three times, and then shaken at 1000 rpm for 1 minute. The Bio-Rad DC™ Assay Kit was used to quantify total protein concentration. For the enzyme assay, cell lysate was combined with 1.4 mM 4-methylumbelliferyl α-D-glucopyranoside in 0.2 M acetate buffer (pH 3.9 or 6.5), incubated at 37° C. for three hours, and then fluorescence was read at 360 nm excitation and 460 nm emission. A standard curve was generated using 4-methylumbelliferone.

A Western blot on GAA protein was performed using the ProteinSimple® Wes™ system (12-230 kDa Master Kit). Rabbit anti-GAA antibody [clone EPR4716(2)] from Abcam was diluted 1:100 and was duplexed with mouse anti-GAPDH [clone 6c5] from Santa Cruz Biotechnology diluted 1:5. Mouse and rabbit secondary antibodies from ProteinSimple® were combined 1:1 for duplexing. GAA was quantified using ProteinSimple® Compass software as area under the curve for all forms of GAA and normalized to GAPDH.

### Example 3

### Antisense PMO-Induced Dose-Dependent Exon 2 Inclusion in GSD-II Patient-Derived Fibroblasts

GM00443 fibroblasts are treated using the above-described nucleofection procedure and antisense sequences made as PPMOs based on the initial GAA exon 2 inclusion results described above in Example 2. 20 μM PMOs, described in Table 4A below, are nucleofected as previously described, and cells are incubated at 37° C. with 5% CO₂ for 24 hours before total RNA isolation. RT-PCR amplification of RNA with primers FWD124 (SEQ ID NO: 33), FWD645 (SEQ ID NO: 34) and REV780 (SEQ ID NO: 35) of Table 4B are analyzed using a Caliper LabChip to determine percent exon 2 inclusion.

TABLE 4A
Nucleofected PMO targeting compounds

| | | | 5′ | 3′ | |
|---|---|---|---|---|---|
| | Targeting Sequence (TS)* | TS SEQ | Attach- | Attach- | CPP SEQ |
| Name | (5′-3′) | ID NO | ment** | ment** | ID NO |
| GAAEx2A(+201+225) | GCC CTG GTC TGC TGG | 4 | TEG | R₆G- | 32 |
| | CTC CCT GCT G | | | | |
| GAAEx2A(+200+224) | CCC TGG TCT GCT GGC | 5 | TEG | R₆G- | 32 |
| | TCC CTG CTG G | | | | |
| GAAEx2A(+199+223) | CCT GGT CTG CTG GCT | 6 | TEG | R₆G- | 32 |
| | CCC TGC TGG T | | | | |
| GAAEx2A(+198+222) | CTG GTC TGC TGG CTC | 7 | TEG | R₆G- | 32 |
| | CCT GCT GGT G | | | | |
| GAAEx2A(+197+221) | TGG TCT GCT GGC TCC | 8 | TEG | R₆G- | 32 |
| | CTG CTG GTG A | | | | |
| GAAEx2A(+196+220) | GGT CTG CTG GCT CCC | 9 | TEG | R₆G- | 32 |
| | TGC TGG TGA G | | | | |
| GAAEx2A(+195+219) | GTC TGC TGG CTC CCT | 10 | TEG | R₆G- | 32 |
| | GCT GGT GAG C | | | | |
| GAAEx2A(+194+218) | TCT GCT GGC TCC CTG | 11 | TEG | R₆G- | 32 |
| | CTG GTG AGC T | | | | |
| GAAEx2A(+203+227) | GGG CCC TGG TCT GCT | 12 | TEG | R₆G- | 32 |
| | GGC TCC CTG C | | | | |
| GAAEx2A(+204+228) | GGG GCC CTG GTC TGC | 13 | TEG | R₆G- | 32 |
| | TGG CTC CCT G | | | | |
| GAAEx2A(+205+229) | CGG GGC CCT GGT CTG | 14 | TEG | R₆G- | 32 |
| | CTG GCT CCC T | | | | |
| GAAEx2A(+206+230) | CCG GGG CCC TGG TCT | 15 | TEG | R₆G- | 32 |
| | GCT GGC TCC C | | | | |
| GAAEx2A(+207+231) | CCC GGG GCC CTG GTC | 16 | TEG | R₆G- | 32 |
| | TGC TGG CTC C | | | | |
| GAAEx2A(+208+232) | TCC CGG GGC CCT GGT | 17 | TEG | R₆G- | 32 |
| | CTG CTG GCT C | | | | |
| GAAEx2A(+209+233) | ATC CCG GGG CCC TGG | 18 | TEG | R₆G- | 32 |
| | TCT GCT GGC T | | | | |
| GAAEx2A(+210+234) | CAT CCC GGG GCC CTG | 19 | TEG | R₆G- | 32 |
| | GTC TGC TGG C | | | | |
| GAAEx2D(-12-38) | TCT GCC CTG GCC GCC | 20 | TEG | R₆G- | 32 |
| | GCC CCC GCC CCT | | | | |
| GAAEx2D(-54-78) | TGA GGT GCG TGG | 21 | TEG | R₆G- | 32 |
| | GTG TCG ATG TCC A | | | | |
| GAAEx2D(-55-79) | GAG GTG CGT GGG | 22 | TEG | R₆G- | 32 |
| | TGT CGA TGT CCA C | | | | |
| GAAEx2D(-56-80) | AGG TGC GTG GGT GTC | 23 | TEG | R₆G- | 32 |
| | GAT GTC CAC G | | | | |
| GAAEx2D(-59-83) | GCG CGT GGA CAT | 24 | TEG | R₆G- | 32 |
| | CGA CAC CCA CGC A | | | | |
| GAAEx2D(-52-76) | TGT GAG GGC GCG | 25 | TEG | R₆G- | 32 |
| | TGG ACA TCG ACA C | | | | |
| GAAEx2D(-51-75) | TTG TGA GGG CGC GTG | 26 | TEG | R₆G- | 32 |
| | GAC ATC GAC A | | | | |
| GAAEx2D(-50-74) | CTT GTG AGG GCG CGT | 351 | TEG | R₆G- | 32 |
| | GGA CAT CGA C | | | | |
| GAAEx2A(+202+226) | GGC CCT GGT CTG CTG | 28 | TEG | R₆G- | 32 |
| | GCT CCC TGC T | | | | |
| GAA-IVS2.12.20 | TGG CCG CCG CCC CCG | 29 | TEG | R₆G- | 32 |
| | CCC CT | | | | |
| GAA-IVS2(53-72) | GTG AGG TGC GTG | 30 | TEG | R₆G- | 32 |
| | GGT GTC GA | | | | |

*Thymines (T) are optionally uracils (U).
**TEG is defined above, and G is glycine.

TABLE 4B
RT-PCR primer sequences for RNA amplification

| | | SEQ ID |
|---|---|---|
| Name | Sequence (5′-3′) | NO |
| FWD124 | CGTTGTTCAGCGAGGGA | 33 |
| FWD645 | CTCCTCTGAAATGGGCTACAC | 34 |
| REV780 | ACCTCGTAGCGCCTGTTA | 35 |

Thus, the disclosure also includes a method of detecting exon 2 inclusion in a human acid alpha-glucosidase (GAA) gene mRNA, the method comprising:

amplifying the GAA mRNA with at least one polymerase chain reaction primer comprising a base sequence selected from the group consisting of SEQ ID NOS: 33, 34, or 35.

### Example 4

### Preparation of Antisense PMOs

Antisense PMOs designed to target exon 2 of the human GAA pre-mRNA were synthesized and used to treat GSD-II patient-derived fibroblasts. The antisense oligomers of the disclosure included those described in Tables 5 and 6 below.

TABLE 5
Nucleofected PMO targeting compounds

| | | | 5′ | 3′ |
|---|---|---|---|---|
| | Targeting Sequence (TS)* | TS SEQ | Attach- | Attach- |
| Name | (5′-3′) | ID NO | ment** | ment** |
| GAA-IVS1(-39-20) | GCT CAG CAG GGA | 38 | TEG | H |
| | GGC GGG AG | | | |
| GAA-IVS1(-74-55) | GGC TCT CAA AGC | 39 | TEG | H |
| | AGC TCT GA | | | |
| GAA-IVS1(-99-75) | GAC ATC AAC CGC | 40 | TEG | H |
| | GGC TGG CAC TGC A | | | |
| GAA-IVS1(-139-115) | GGG TAA GGT GGC | 41 | TEG | H |
| | CAG GGT GGG TGT T | | | |
| GAA-IVS1(-158-140) | GCC CTG CTG TCT AGA | 42 | TEG | H |
| | CTG G | | | |
| GAA-IVS1(-179-160) | GAG AGG GCC AGA | 43 | TEG | H |
| | AGG AAG GG | | | |
| GAA-IVS2(-9-20) | CCC GCC CCT GCC CTG | 44 | TEG | H |
| | CC | | | |
| GAA-IVS2(-14-30) | TGG CCG CCG CCC CCG | 45 | TEG | H |
| | CCC | | | |
| GAA-IVS2(-33-52) | TGT CCA CGC GCA CCC | 46 | TEG | H |
| | TCT GC | | | |
| GAA-IVS2(-53-72) | GTG AGG TGC GTG | 30 | TEG | H |
| | GGT GTC GA | | | |
| GAA-IVS2(-73-92) | GCA ACA TGC ACC | 47 | TEG | H |
| | CCA CCC TT | | | |
| GAA-IVS2(-93-112) | AGG GCC CAG CAC | 48 | TEG | H |
| | ACA GTG GT | | | |
| GAA-IVS2(-113-132) | TCA CAC CTC CGC TCC | 49 | TEG | H |
| | CAG CA | | | |
| GAA-IVS2(-133-150) | GGC GCT GCC ATT GTC | 50 | TEG | H |
| | TGC | | | |
| GAA-IVS2(-153-172) | GTG TCC CCA CTG CTC | 51 | TEG | H |
| | CCC GA | | | |
| GAA-IVS2(-173-192) | CTG GAG TAC CTG TCA | 52 | TEG | H |
| | CCG TG | | | |
| GAA-IVS2(-193-212) | TGA GCC CCG AGC CCT | 53 | TEG | H |
| | GCC TT | | | |
| GAA-IVS2(-213-237) | TGA CCC ACC TTT TCA | 54 | TEG | H |
| | TAA AGA TGA A | | | |
| GAA-IVS2(-234-258) | CTC TGG CAG CCC TAC | 55 | TEG | H |
| | TCT ACC TGA C | | | |
| GAA-IVS2(-338-364) | CTA GTA TAA ATA CAT | 56 | TEG | H |
| | CCC AAA TTT TGC | | | |
| GAAEx2A(+202+226) | GGC CCT GGT CTG CTG | 57 | TEG | H |
| | GCT CCC TGC T | | | |
| GAAEx2A(+367+391) | GCT CCC TGC AGC CCC | 58 | TEG | H |
| | TGC TTT GCA G | | | |
| GAA-IVS1.6.20 | GCG GGG CAG ACG | 59 | TEG | H |
| | TCA GGT GT | | | |
| GAA-IVS1.10.20 | CAG CGC GGG GCA | 60 | TEG | H |
| | GAC GTC AG | | | |
| GAA-IVS1.14.20 | CCG GCA GCG CGG | 61 | TEG | H |
| | GGC AGA CG | | | |
| GAA-IVS1.17.20 | CCG CCG GCA GCG | 62 | TEG | H |
| | CGG GGC AG | | | |
| GAA-IVS1.24.20 | GAT GTT ACC GCC GGC | 63 | TEG | H |
| | AGC GC | | | |
| GAA-IVS1.28.20 | CTG GGA TGT TAC CGC | 64 | TEG | H |
| | CGG CA | | | |
| GAA-IVS1.32.20 | GCT TCT GGG ATG TTA | 65 | TEG | H |
| | CCG CC | | | |
| GAA-IVS1.2015.20 | TGG CAA CTC GTA TGT | 66 | TEG | H |
| | CCT TA | | | |
| GAA-IVS1.2019.20 | ATT CTG GCA ACT CGT | 67 | TEG | H |
| | ATG TC | | | |
| GAA-IVS1.2024.20 | AAG TGA TTC TGG CAA | 68 | TEG | H |
| | CTC GT | | | |
| GAA-IVS1.2037.20 | TGG GTG TCA GCG | 69 | TEG | H |
| | GAA GTG AT | | | |
| GAA-IVS1.2043.20 | GTC CAC TGG GTG TCA | 70 | TEG | H |
| | GCG GA | | | |
| GAA-IVS1.2048.20 | GCT TGG TCC ACT GGG | 71 | TEG | H |
| | TGT CA | | | |
| GAA-IVS1.2071.20 | CCC CAC TTC TGC ATA | 72 | TEG | H |
| | AAG GT | | | |
| GAA-IVS1.2075.20 | GGA GCC CCA CTT CTG | 73 | TEG | H |
| | CAT AA | | | |
| GAA-IVS1.2079.20 | GCT GGG AGC CCC ACT | 74 | TEG | H |
| | TCT GC | | | |
| GAA-IVS1.2088.20 | CCA CGC CTG GCT GGG | 75 | TEG | H |
| | AGC CC | | | |
| GAA-IVS1.2115.20 | TCC GAA GTG CTG | 76 | TEG | H |
| | GGA TTT CA | | | |
| GAA-IVS1.2132.20 | TCC ACC CCC CTT GGC | 77 | TEG | H |
| | CTT CC | | | |
| GAA-IVS1.2135.20 | TGA TCC ACC CCC CTT | 78 | TEG | H |
| | GGC CT | | | |
| GAA-IVS1.2140.20 | TCA AGT GAT CCA CCC | 79 | TEG | H |
| | CCC TT | | | |
| GAA-IVS1.2152.20 | GAA CTC CTG AGC TCA | 80 | TEG | H |
| | AGT GA | | | |
| GAA-IVS1.2156.20 | TCT CGA ACT CCT GAG | 81 | TEG | H |
| | CTC AA | | | |
| GAA-IVS1.2165.20 | CCA GGC TGG TCT CGA | 82 | TEG | H |
| | ACT CC | | | |
| GAA-IVS1.2178.20 | TTT GCC ATG TTA CCC | 83 | TEG | H |
| | AGG CT | | | |
| GAA-IVS1.2185.20 | ACG GGA TTT TGC CAT | 84 | TEG | H |
| | GTT AC | | | |
| GAA-IVS1.2190.20 | TAG AGA CGG GAT TTT | 85 | TEG | H |
| | GCC AT | | | |
| GAA-IVS1.2195.20 | TTT TGT AGA GAC GGG | 86 | TEG | H |
| | ATT TT | | | |
| GAA-IVS1.2202.20 | TCT GTA TTT TTG TAG | 87 | TEG | H |
| | AGA CG | | | |
| GAA-IVS1.2206.20 | ATT TTC TGT ATT TTT | 88 | TEG | H |
| | GTA GA | | | |
| GAA-IVS1.2210.20 | GCT AAT TTT CTG TAT | 89 | TEG | H |
| | TTT TG | | | |
| GAA-IVS2.9.20 | CCG CCG CCC CCG CCC | 90 | TEG | H |
| | CTG CC | | | |
| GAA-IVS2.12.20 | TGG CCG CCG CCC CCG | 29 | TEG | H |
| | CCC CT | | | |
| GAA-IVS2.18.20 | CTG CCC TGG CCG CCG | 91 | TEG | H |
| | CCC CC | | | |
| GAA-IVS2.24.20 | CAC CCT CTG CCC TGG | 92 | TEG | H |
| | CCG CC | | | |
| GAA-IVS2.27.20 | GCG CAC CCT CTG CCC | 93 | TEG | H |
| | TGG CC | | | |
| GAA-IVS2.40.20 | TGT CGA TGT CCA CGC | 94 | TEG | H |
| | GCA CC | | | |
| GAA-IVS2.48.20 | TGC GTG GGT GTC GAT | 95 | TEG | H |
| | GTC CA | | | |
| GAA-IVS2.67.20 | GCA CCC CAC CCT TGT | 96 | TEG | H |
| | GAG GT | | | |
| GAA-IVS2.72.20 | AAC ATG CAC CCC ACC | 97 | TEG | H |
| | CTT GT | | | |
| GAA-IVS2.431.20 | AGG AGG AGG ACG | 98 | TEG | H |
| | CCT CCC CC | | | |
| GAA-IVS2.446.20 | CTC ATC TGC AGA GCC | 99 | TEG | H |
| | AGG AG | | | |
| GAA-IVS2.451.20 | GCT CCC TCA TCT GCA | 100 | TEG | H |
| | GAG CC | | | |
| GAA-IVS2.454.20 | TCG GCT CCC TCA TCT | 101 | TEG | H |
| | GCA GA | | | |
| GAA-IVS2.457.20 | GCC TCG GCT CCC TCA | 102 | TEG | H |
| | TCT GC | | | |
| GAA-IVS1.30.20 | TTC TGG GAT GTT ACC | 103 | TEG | H |
| | GCC GG | | | |
| GAA-IVS1.31.20 | CTT CTG GGA TGT TAC | 104 | TEG | H |
| | CGC CG | | | |
| GAA-IVS1.33.20 | CGC TTC TGG GAT GTT | 105 | TEG | H |
| | ACC GC | | | |
| GAA-IVS1.34.20 | CCG CTT CTG GGA TGT | 106 | TEG | H |
| | TAC CG | | | |
| GAA-IVS1.36.20 | ACC CGC TTC TGG GAT | 107 | TEG | H |
| | GTT AC | | | |
| GAA-IVS1.40.20 | TCA AAC CCG CTT CTG | 108 | TEG | H |
| | GGA TG | | | |
| GAA-IVS1.44.20 | ACG TTC AAA CCC GCT | 109 | TEG | H |
| | TCT GG | | | |
| GAA-IVS1(-73-54) | GGG CTC TCA AAG | 110 | TEG | H |
| | CAG CTC TG | | | |
| GAA-IVS1(-72-53) | GGG GCT CTC AAA | 111 | TEG | H |
| | GCA GCT CT | | | |
| GAA-IVS1(-70-51) | ACG GGG CTC TCA | 112 | TEG | H |
| | AAG CAG CT | | | |
| GAA-IVS1(-68-49) | TCA CGG GGC TCT CAA | 113 | TEG | H |
| | AGC AG | | | |
| GAA-IVS1(-75-56) | GCT CTC AAA GCA GCT | 114 | TEG | H |
| | CTG AG | | | |
| GAA-IVS1(-76-57) | CTC TCA AAG CAG CTC | 115 | TEG | H |
| | TGA GA | | | |
| GAA-IVS1(-78-59) | CTC AAA GCA GCT CTG | 116 | TEG | H |
| | AGA CA | | | |
| GAA-IVS1(-80-61) | CAA AGC AGC TCT | 117 | TEG | H |
| | GAG ACA TC | | | |
| GAA-IVS1(-82-63) | AAG CAG CTC TGA | 118 | TEG | H |
| | GAC ATC AA | | | |
| GAAEx2A(+201+225) | GCC CTG GTC TGC TGG | 4 | TEG | H |
| | CTC CCT GCT G | | | |
| GAAEx2A(+200+224) | CCC TGG TCT GCT GGC | 5 | TEG | H |
| | TCC CTG CTG G | | | |
| GAAEx2A(+199+223) | CCT GGT CTG CTG GCT | 6 | TEG | H |
| | CCC TGC TGG T | | | |
| GAAEx2A(+198+222) | CTG GTC TGC TGG CTC | 7 | TEG | H |
| | CCT GCT GGT G | | | |
| GAAEx2A(+197+221) | TGG TCT GCT GGC TCC | 8 | TEG | H |
| | CTG CTG GTG A | | | |
| GAAEx2A(+196+220) | GGT CTG CTG GCT CCC | 9 | TEG | H |
| | TGC TGG TGA G | | | |
| GAAEx2A(+195+219) | GTC TGC TGG CTC CCT | 10 | TEG | H |
| | GCT GGT GAG C | | | |
| GAAEx2A(+194+218) | TCT GCT GGC TCC CTG | 11 | TEG | H |
| | CTG GTG AGC T | | | |
| GAAEx2A(+203+227) | GGG CCC TGG TCT GCT | 12 | TEG | H |
| | GGC TCC CTG C | | | |
| GAAEx2A(+204+228) | GGG GCC CTG GTC TGC | 13 | TEG | H |
| | TGG CTC CCT G | | | |
| GAAEx2A(+205+229) | CGG GGC CCT GGT CTG | 14 | TEG | H |
| | CTG GCT CCC T | | | |
| GAAEx2A(+206+230) | CCG GGG CCC TGG TCT | 15 | TEG | H |
| | GCT GGC TCC C | | | |
| GAAEx2A(+207+231) | CCC GGG GCC CTG GTC | 16 | TEG | H |
| | TGC TGG CTC C | | | |
| GAAEx2A(+208+232) | TCC CGG GGC CCT GGT | 17 | TEG | H |
| | CTG CTG GCT C | | | |
| GAAEx2A(+209+233) | ATC CCG GGG CCC TGG | 18 | TEG | H |
| | TCT GCT GGC T | | | |
| GAAEx2A(+210+234) | CAT CCC GGG GCC CTG | 19 | TEG | H |
| | GTC TGC TGG C | | | |
| GAAEx2D(-12-38) | TCT GCC CTG GCC GCC | 20 | TEG | H |
| | GCC CCC GCC CCT | | | |
| GAAEx2D(-54-78) | TGA GGT GCG TGG | 21 | TEG | H |
| | GTG TCG ATG TCC A | | | |
| GAAEx2D(-55-79) | GAG GTG CGT GGG | 22 | TEG | H |
| | TGT CGA TGT CCA C | | | |
| GAAEx2D(-56-80) | AGG TGC GTG GGT GTC | 23 | TEG | H |
| | GAT GTC CAC G | | | |
| GAAEx2D(-59-83) | GCG CGT GGA CAT | 24 | TEG | H |
| | CGA CAC CCA CGC A | | | |
| GAAEx2D(-52-76) | TGT GAG GGC GCG | 25 | TEG | H |
| | TGG ACA TCG ACA C | | | |
| GAAEx2D(-51-75) | TTG TGA GGG CGC GTG | 26 | TEG | H |
| | GAC ATC GAC A | | | |
| GAAEx2D(-50-74) | CTT GTG AGG GCG CGT | 351 | TEG | H |
| | GGA CAT CGA C | | | |
| GAA-IVS1(-177-160) | GAG AGG GCC AGA | 119 | TEG | H |
| | AGG AAG | | | |
| GAA-IVS1(-179-162) | GAG GGC CAG AAG | 120 | TEG | H |
| | GAA GGG | | | |
| GAA-IVS1(-181-164) | GGG CCA GAA GGA | 121 | TEG | H |
| | AGG GCG | | | |
| GAA-IVS1(-175-158) | GGG AGA GGG CCA | 122 | TEG | H |
| | GAA GGA | | | |
| GAA-IVS1(-180-161) | AGA GGG CCA GAA | 123 | TEG | H |
| | GGA AGG GC | | | |
| GAA-IVS1(-181-162) | GAG GGC CAG AAG | 124 | TEG | H |
| | GAA GGG CG | | | |
| GAA-IVS1(-182-163) | AGG GCC AGA AGG | 125 | TEG | H |
| | AAG GGC GA | | | |
| GAA-IVS1(-182-164) | GGG CCA GAA GGA | 126 | TEG | H |
| | AGG GCG AG | | | |
| GAA-IVS1(-184-165) | GGC CAG AAG GAA | 127 | TEG | H |
| | GGG CGA GA | | | |
| GAA-IVS1(-185-166) | GCC AGA AGG AAG | 128 | TEG | H |
| | GGC GAG AA | | | |
| GAA-IVS1(-179-158) | GGG AGA GGG CCA | 129 | TEG | H |
| | GAA GGA AGG G | | | |
| GAA-IVS1(-179-155) | CTG GGG AGA GGG | 130 | TEG | H |
| | CCA GAA GGA AGG G | | | |
| GAA-IVS1(-181-160) | GAG AGG GCC AGA | 131 | TEG | H |
| | AGG AAG GGC G | | | |
| GAA-IVS1(-184-160) | GAG AGG GCC AGA | 132 | TEG | H |
| | AGG AAG GGC GAG A | | | |
| GAA-IVS1(-189-170) | GAA GGA AGG GCG | 36 | TEG | H |
| | AGA AAA GC | | | |
| GAA-IVS1(-209-190) | GCA GAA AAG CTC | 37 | TEG | H |
| | CAG CAG GG | | | |

*Thymines (T) are optionally uracils (U).
**TEG is defined above.

TABLE 6
Nucleofected PMO targeting compounds

| | | | 5′ | 3′ |
|---|---|---|---|---|
| | Targeting Sequence (TS)* | TS SEQ | Attach- | Attach- |
| Name | (5′-3′) | ID NO | ment** | ment** |
| GAA-IVS1.SA.(-210,- | AAG CTC CAG CAG | 256 | TEG | H |
| 186) | GGG AGT GCA GAG C | | | |
| GAA-IVS1.SA.(-208,- | AAA AGC TCC AGC | 257 | TEG | H |
| 184) | AGG GGA GTG CAG A | | | |
| GAA-IVS1.SA.(-206,- | AGA AAA GCT CCA | 258 | TEG | H |
| 182) | GCA GGG GAG TGC A | | | |
| GAA-IVS1.SA.(-204,- | CGA GAA AAG CTC | 259 | TEG | H |
| 180) | CAG CAG GGG AGT G | | | |
| GAA-IVS1.SA.(-202,- | GGC GAG AAA AGC | 260 | TEG | H |
| 178) | TCC AGC AGG GGA G | | | |
| GAA-IVS1.SA.(-200,- | AGG GCG AGA AAA | 261 | TEG | H |
| 176) | GCT CCA GCA GGG G | | | |
| GAA-IVS1.SA.(-198,- | GAA GGG CGA GAA | 262 | TEG | H |
| 174) | AAG CTC CAG CAG G | | | |
| GAA-IVS1.SA.(-196,- | AGG AAG GGC GAG | 263 | TEG | H |
| 172) | AAA AGC TCC AGC A | | | |
| GAA-IVS1.SA.(-194,- | GAA GGA AGG GCG | 264 | TEG | H |
| 170) | AGA AAA GCT CCA G | | | |
| GAA-IVS1.SA.(-192,- | CAG AAG GAA GGG | 265 | TEG | H |
| 168) | CGA GAA AAG CTC C | | | |
| GAA-IVS1.SA.(-190,- | GCC AGA AGG AAG | 266 | TEG | H |
| 166) | GGC GAG AAA AGC T | | | |
| GAA-IVS1.SA.(-188,- | GGG CCA GAA GGA | 267 | TEG | H |
| 164) | AGG GCG AGA AAA G | | | |
| GAA-IVS1.SA.(-186,- | GAG GGC CAG AAG | 268 | TEG | H |
| 162) | GAA GGG CGA GAA A | | | |
| GAA-IVS1(-184-160) | GAG AGG GCC AGA | 269 | TEG | H |
| | AGG AAG GGC GAG A | | | |
| GAA-IVS1(-182-163) | AGG GCC AGA AGG | 270 | TEG | H |
| | AAG GGC GA | | | |
| GAA-IVS1(-179-160) | GAG AGG GCC AGA | 271 | TEG | H |
| | AGG AAG GG | | | |
| GAA-IVS1(-179-155) | CTG GGG AGA GGG | 272 | TEG | H |
| | CCA GAA GGA AGG G | | | |
| GAA-IVS1(-177-160) | GAG AGG GCC AGA | 273 | TEG | H |
| | AGG AAG | | | |
| GAA-IVS1(-175-158) | GGG AGA GGG CCA | 274 | TEG | H |
| | GAA GGA | | | |
| GAAEx2A(+196+220) | GGT CTG CTG GCT CCC | 275 | TEG | H |
| | TGC TGG TGA G | | | |
| GAA-IVS1(-70-46) | CAC TCA CGG GGC TCT | 276 | TEG | H |
| | CAA AGC AGC T | | | |
| GAA-IVS1.24.25 | TCT GGG ATG TTA CCG | 277 | TEG | H |
| | CCG GCA GCG C | | | |
| GAA-IVS1.2178.20 | TTT GCC ATG TTA CCC | 278 | TEG | H |
| | AGG CT | | | |
| GAA-IVS1(-71-47) | ACT CAC GGG GCT CTC | 279 | TEG | H |
| | AAA GCA GCT C | | | |
| GAA-IVS1(-69-45) | GCA CTC ACG GGG CTC | 280 | TEG | H |
| | TCA AAG CAG C | | | |
| GAA-IVS1(-76-52) | CGG GGC TCT CAA | 281 | TEG | H |
| | AGC AGC TCT GAG A | | | |
| GAA-IVS1(-75-51) | ACG GGG CTC TCA | 282 | TEG | H |
| | AAG CAG CTC TGA G | | | |
| GAA-IVS1(-74-50) | CAC GGG GCT CTC | 283 | TEG | H |
| | AAA GCA GCT CTG A | | | |
| GAA-IVS1(-73-49) | TCA CGG GGC TCT CAA | 284 | TEG | H |
| | AGC AGC TCT G | | | |
| GAA-IVS1(-72-48) | CTC ACG GGG CTC TCA | 285 | TEG | H |
| | AAG CAG CTC T | | | |
| GAA-IVS1(-68-44) | GGC ACT CAC GGG | 286 | TEG | H |
| | GCT CTC AAA GCA G | | | |
| GAA-IVS1(-67-43) | CGG CAC TCA CGG | 287 | TEG | H |
| | GGC TCT CAA AGC A | | | |
| GAA-IVS1(-66-42) | GCG GCA CTC ACG | 288 | TEG | H |
| | GGG CTC TCA AAG C | | | |
| GAA-IVS1(-65-41) | GGC GGC ACT CAC | 290 | TEG | H |
| | GGG GCT CTC AAA G | | | |
| GAA-IVS1(-64-40) | GGG CGG CAC TCA | 291 | TEG | H |
| | CGG GGC TCT CAA A | | | |
| GAA-IVS1(-63-39) | GGG GCG GCA CTC | 292 | TEG | H |
| | ACG GGG CTC TCA A | | | |
| GAA-IVS1(-62-38) | AGG GGC GGC ACT | 293 | TEG | H |
| | CAC GGG GCT CTC A | | | |
| GAA-IVS1(-61-37) | GAG GGG CGG CAC | 294 | TEG | H |
| | TCA CGG GGC TCT C | | | |
| GAA-IVS1(-74-55) | GGC TCT CAA AGC | 295 | TEG | H |
| | AGC TCT GA | | | |
| GAA-IVS1.25.25 | TTC TGG GAT GTT ACC | 343 | TEG | H |
| | GCC GGC AGC G | | | |
| GAA-IVS1.26.25 | CTT CTG GGA TGT TAC | 344 | TEG | H |
| | CGC CGG CAG C | | | |
| GAA-IVS1.27.25 | GCT TCT GGG ATG TTA | 345 | TEG | H |
| | CCG CCG GCA G | | | |
| GAA-IVS1.28.25 | CGC TTC TGG GAT GTT | 346 | TEG | H |
| | ACC GCC GGC A | | | |
| GAA-IVS1.29.25 | CCG CTT CTG GGA TGT | 347 | TEG | H |
| | TAC CGC CGG C | | | |
| GAA-IVS1.30.25 | CCC GCT TCT GGG ATG | 348 | TEG | H |
| | TTA CCG CCG G | | | |
| GAA-IVS1.31.25 | ACC CGC TTC TGG GAT | 349 | TEG | H |
| | GTT ACC GCC G | | | |
| GAA-IVS1.32.25 | AAC CCG CTT CTG GGA | 350 | TEG | H |
| | TGT TAC CGC C | | | |

*Thymines (T) are optionally uracils (U).
**TEG is defined above.

### Example 5

### Antisense Oligomers Induce Elevated Expression Levels of Acid Alpha-Glucosidase in GSD-II Patient-Derived Fibroblasts

The antisense oligomers depicted in Table 5 were delivered to GM00443 cells by nucleofection (see above, e.g., Materials and Methods). After six days of incubation at 37° C. with 5% CO₂, cells were lysed and GAA protein expression was measured by immunoassay as described above. As shown in FIGS. 2-4, protein expression of GAA enzyme in cells treated with antisense oligonucleotides of the disclosure was higher than the GAA expression level in untreated cells. These results indicate that oligonucleotides of the disclosure induce elevated protein expression levels of GAA enzyme in GSD-II patient-derived fibroblasts. While not being bound by any theory or mechanism of action, in view of the experimental results described herein, the inventors believe that the oligomers of the disclosure suppress ISS and/or ESS elements and thereby promote exon 2 retention in the mature GAA mRNA.

### Example 6

### Antisense Oligomers Induce Elevated Levels of Enzymatically Active Acid Alpha-Glucosidase in GSD-II Patient-Derived Fibroblasts

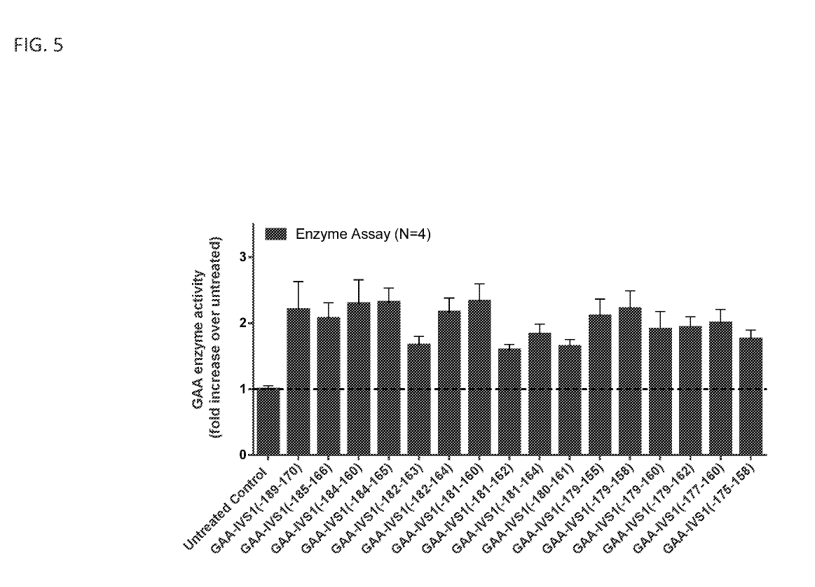

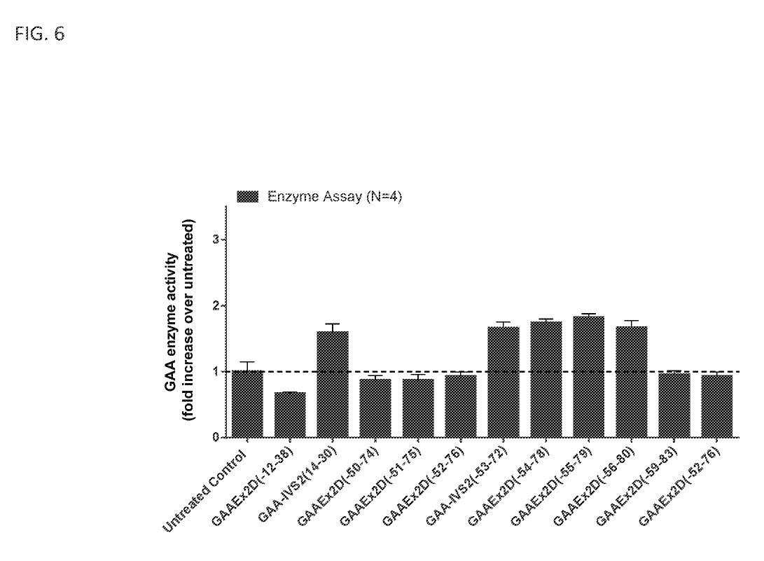

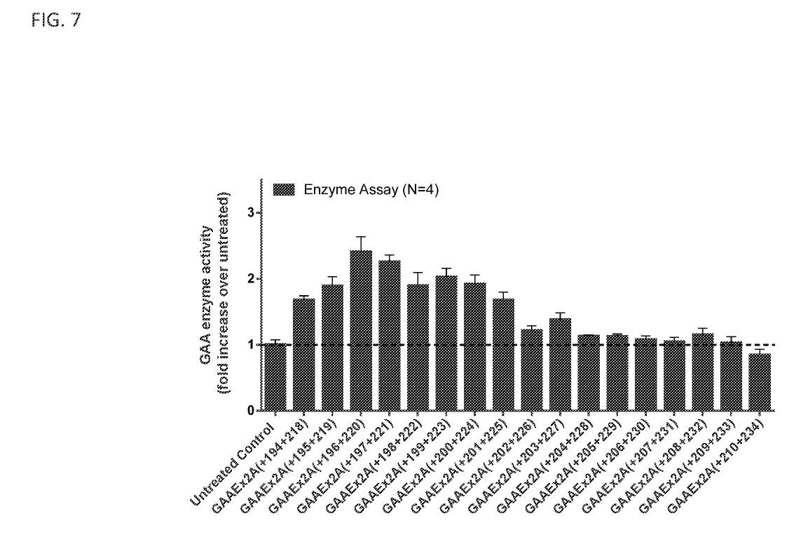

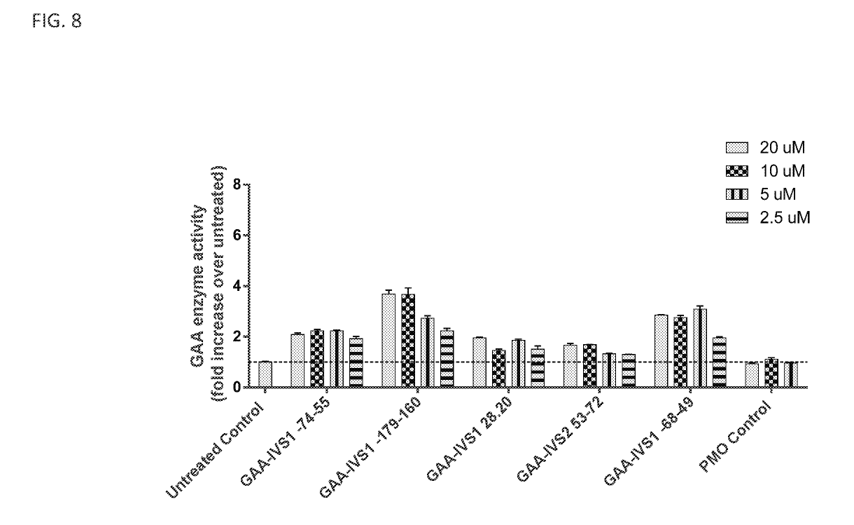

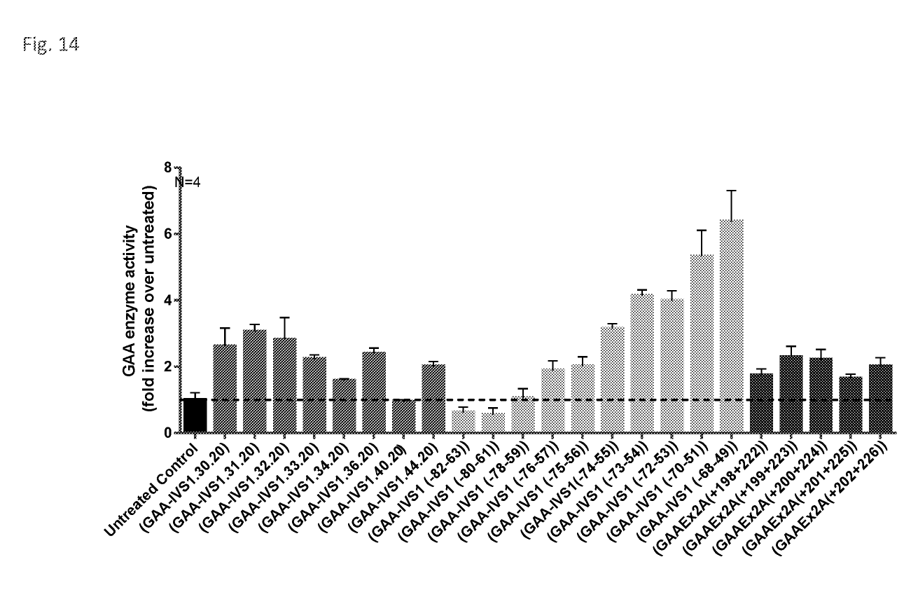

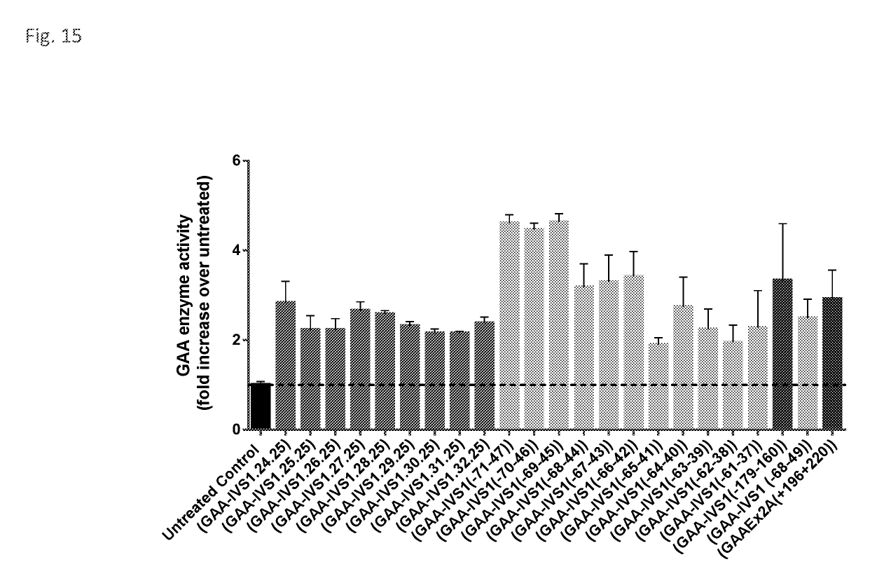

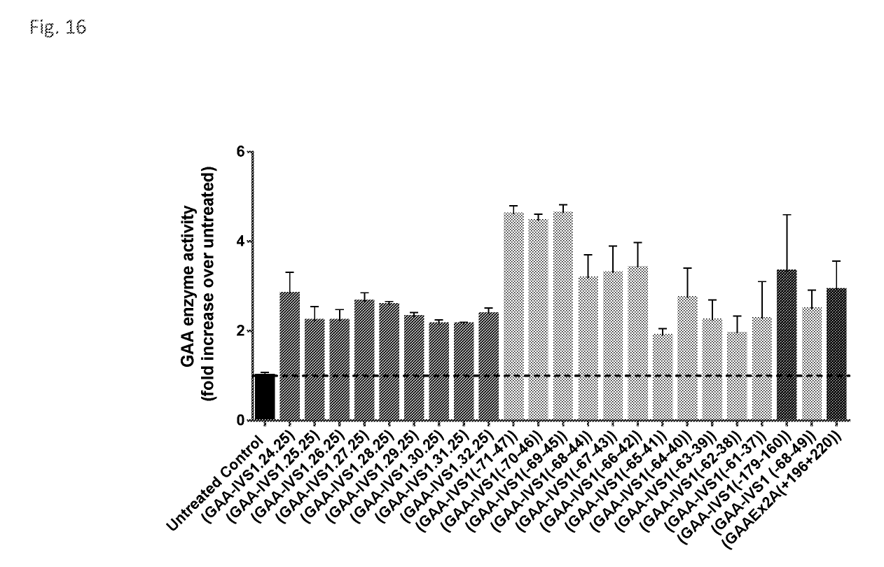

The antisense oligomers depicted in Table 5 were delivered to GM00443 cells by nucleofection (see above, e.g., Materials and Methods). After six days of incubation at 37° C. with 5% CO₂, cells were lysed and GAA activity in the lysates was measured. As shown in FIGS. 2-9, 14, and 15, the GAA enzyme activity level in lysates from cells treated with antisense oligonucleotides of the disclosure was higher than the GAA enzyme activity level in lysates from untreated cells. Selected oligonucleotides IVS1(−74−55), IVS1(−179−160), IVS1 28.20, IVS2(53−72), and IVS1(−68−49) were evaluated in GM00443 cells at multiple doses (2.5 μM, 5 μM, 10 μM, and 20 μM). Following incubation of nucleofected cells for six days, lysates were prepared as above and the GAA enzyme activity was measured in the lysates. As shown in FIG. 8, the lysates of cells treated with each of these compounds at all concentrations tested exhibited increased GAA enzyme activity as compared to the GAA enzyme activity level in lysates from untreated cells, or cells treated with a control oligonucleotide that is not capable of hybridizing to the human GAA pre-mRNA.

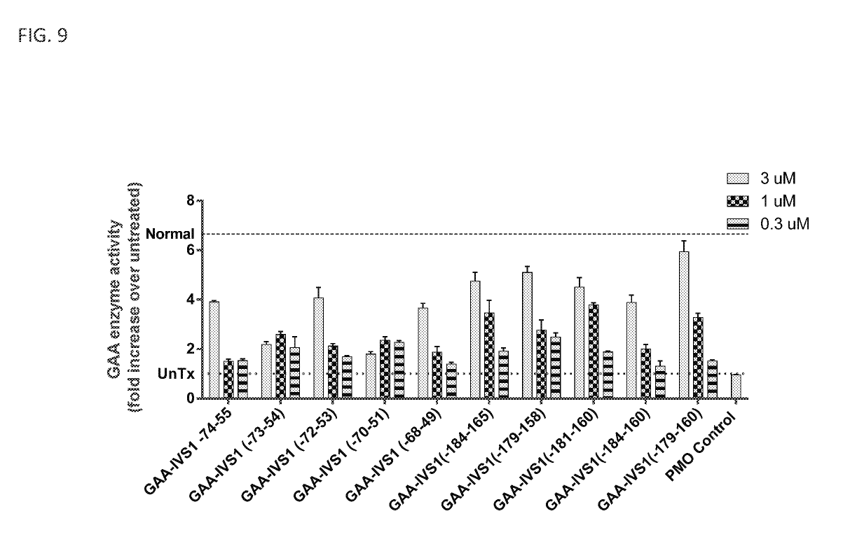

In another experiment, selected oligonucleotides IVS1(−74−55), IVS1(−73−54), IVS1(−72−53), IVS1(−70−51), IVS1(−68−49), IVS1(−184−165), IVS1(−179−158), IVS1(−181−160), IVS1(−184−160), and IVS1(−179−160), were evaluated in GM00443 cells at several doses (0.3 μM, 1 μM, and 3 μM). As shown in FIG. 9, the lysates of cells treated with each of these compounds at all concentrations tested exhibited increased GAA enzyme activity as compared to the GAA enzyme activity level in lysates from untreated cells, or cells treated with a control oligonucleotide that is not capable of hybridizing to the human GAA pre-mRNA. Treatment of cells with several of the PMOs resulted in a GAA enzyme activity that reached a level near normal GAA enzyme activity (see, e.g., IVS1(−179−160) at 3 μM, FIG. 9).

The above results indicate that oligonucleotides of the disclosure induce elevated levels of active GAA enzyme in GSD-II patient-derived fibroblasts. Accordingly, elevated GAA mRNA and protein expression levels following treatment with oligomers of the disclosure allow functional enzyme to be synthesized, and thereby provide a clinical benefit to GSD-II patients treated with the oligomers.

### Example 7

### Antisense Oligomers Induce Elevated Levels of Enzymatically Active Acid Alpha-Glucosidase in GSD-II Patient-Derived Fibroblasts

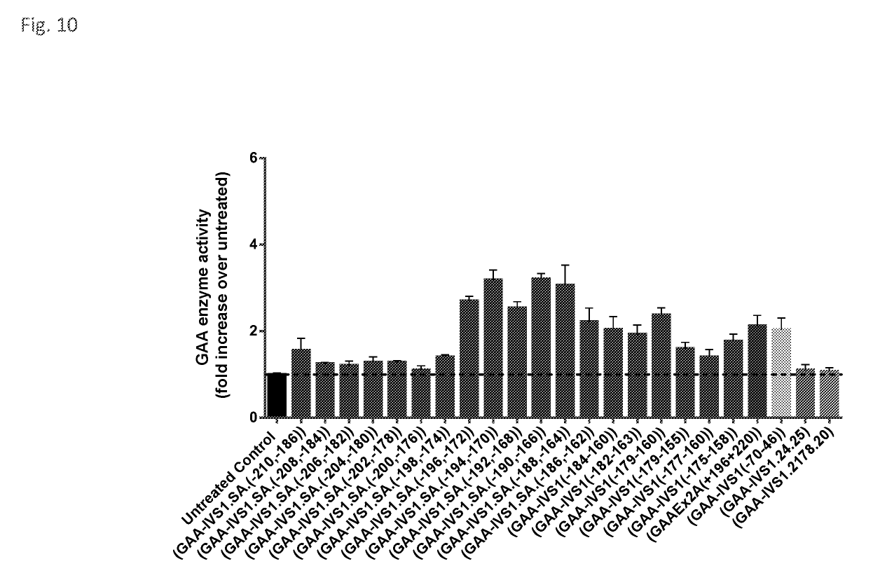

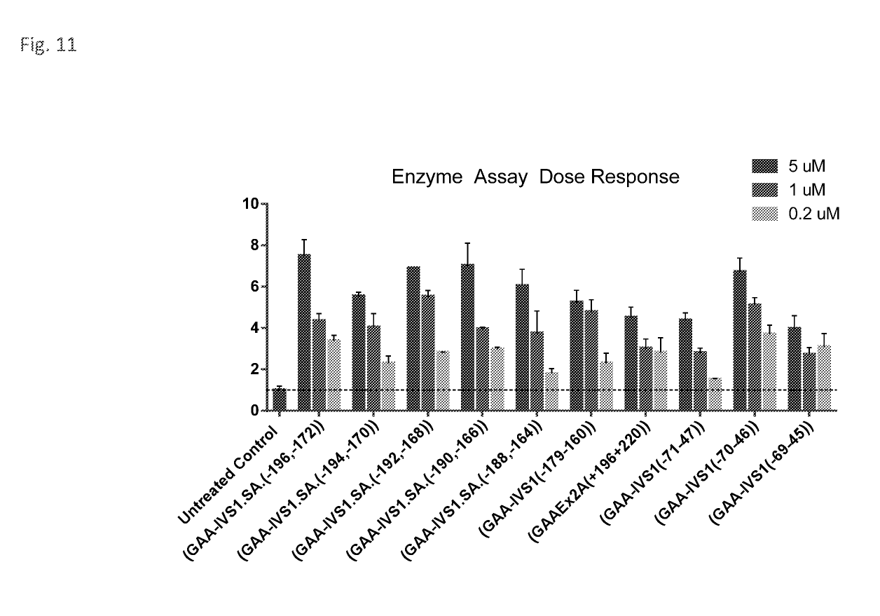

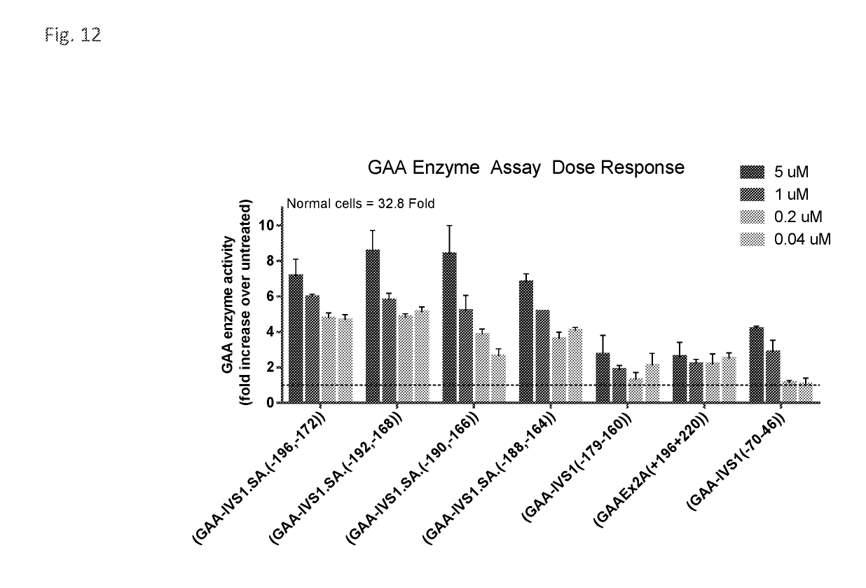

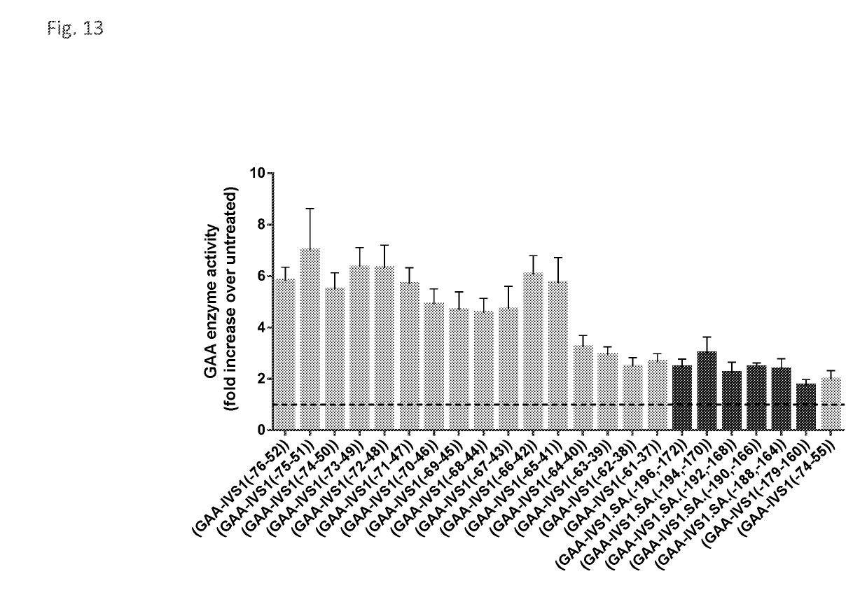

The antisense oligomers depicted in Table 6 were delivered to GM00443 cells by nucleofection (see above, e.g., Materials and Methods). After six days of incubation at 37° C. with 5% CO₂, cells were lysed and GAA activity in the lysates was measured. As shown in FIGS. 10-15, the GAA enzyme activity level in lysates from cells treated with antisense oligonucleotides of the disclosure was higher than the GAA enzyme activity level in lysates from untreated cells. Selected oligonucleotides were evaluated in GM00443 cells at multiple doses (5 μM, 1.0 μM, and 0.2 μM (FIG. 10) or 5 μM, 1.0 μM, 0.2 μM, and 0.04 μM (FIG. 12)). Following incubation of nucleofected cells for six days, lysates were prepared as above and the GAA enzyme activity was measured in the lysates. As shown in FIGS. 10 and 12, the lysates of cells treated with each of these compounds exhibited increased GAA enzyme activity as compared to the GAA enzyme activity level in lysates from untreated cells, or cells treated with a control oligonucleotide that is not capable of hybridizing to the human GAA pre-mRNA. A shown in FIG. 12, for example, treatment of cells with several of the PMOs (e.g., GAA-IVS1.SA(−196−172), GAA-IVS1.SA(−192−168), GAA-IVS1.SA(−190−166) and GAA-IVS1.SA(−188−164) resulted in a GAA enzyme activity that reached a level of GAA enzyme activity between 6-10 fold that of untreated cells.

1. A compound of formula (I): 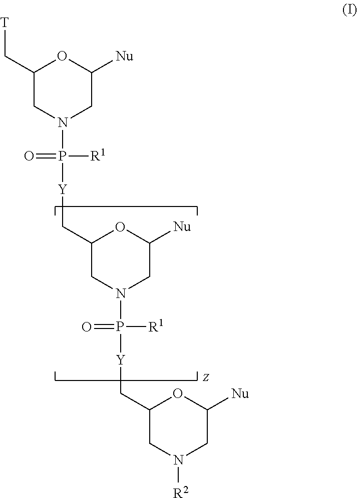
or a pharmaceutically acceptable salt thereof, wherein:
each Nu is a nucleobase which taken together form a targeting sequence;
each Y is independently selected from O and —NR⁴, wherein each R⁴ is independently selected from H, C₁-C₆ alkyl, aralkyl, —C(═NH)NH₂, —C(O)(CH₂)ₙNR⁵C(═NH)NH₂, —C(O)(CH₂)₂NHC(O)(CH₂)₅NR⁵C(═NH)NH₂, and G, wherein R⁵ is selected from H and C₁-C₆ alkyl and n is an integer from 1 to 5;
T is selected from OH and a moiety of the formula: 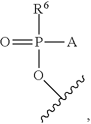
wherein:
A is selected from —OH, —N(R⁷)₂, and R¹ wherein:
each R⁷ is independently selected from H and C₁-C₆ alkyl, and
R⁶ is selected from OH, —N(R⁹)CH₂C(O)NH₂, and a moiety of the formula: 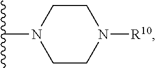
wherein:
R⁹ is selected from H and C₁-C₆ alkyl; and
R¹⁰ is selected from G, —C(O)—R¹OH, acyl, trityl, 4-methoxytrityl, —C(═NH)NH₂, —C(O)(CH₂)ₘNR¹²C(═NH)NH₂, and —C(O)(CH₂)₂NHC(O)(CH₂)₅NR¹²C(═NH)NH₂, wherein:
m is an integer from 1 to 5,
R¹¹ is of the formula —(O-alkyl)_{y}- wherein y is an integer from 3 to 10 and
each of the y alkyl groups is independently selected from C₂-C₆ alkyl; and
R¹² is selected from H and C₁-C₆ alkyl;
each instance of R¹ is independently selected from:
—N(R¹³)₂, wherein each R¹³ is independently selected from H and C₁-C₆ alkyl;
a moiety of formula (II): 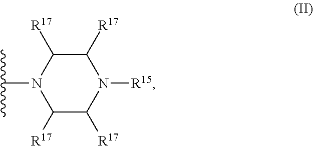
wherein:
R¹⁵ is selected from H, G, C₁-C₆ alkyl, —C(═NH)NH₂, —C(O)(CH₂)_{q}NR¹⁸C(═NH)NH₂, and —C(O)(CH₂)₂NHC(O)(CH₂)₅NR¹⁸C(═NH)NH₂, wherein:
R¹⁸ is selected from H and C₁-C₆ alkyl; and
q is an integer from 1 to 5; and
each R¹⁷ is independently selected from H and methyl; and
a moiety of formula (III): 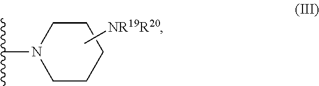
wherein:
R¹⁹ is selected from H, C₁-C₆ alkyl, —C(═NH)NH₂, —C(O)(CH₂)ᵣNR²²C(═NH)NH₂, —C(O)CH(NH₂)(CH₂)₃NHC(═NH)NH₂, —C(O)(CH₂)₂NHC(O)(CH₂)₅NR²²C(═NH)NH₂, —C(O)CH(NH₂)(CH₂)₄NH₂ and G, wherein:
R²² is selected from H and C₁-C₆ alkyl; and
r is an integer from 1 to 5,
R²⁰ is selected from H and C₁-C₆ alkyl; and
R² is selected from H, G, acyl, trityl, 4-methoxytrityl, C₁-C₆ alkyl, —C(═NH)NH₂, —C(O)—R²³, —C(O)(CH₂)ₛNR²⁴C(═NH)NH₂, —C(O)(CH₂)₂NHC(O)(CH₂)₅NR²⁴C(═NH)NH₂, —C(O)CH(NH₂)(CH₂)₃NHC(═NH)NH₂, and a moiety of the formula: 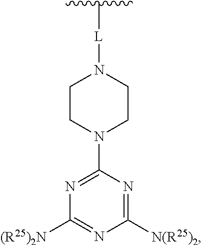
wherein,
R²³ is of the formula —(O-alkyl)ᵥ-OH wherein v is an integer from 3 to 10 and each of the v alkyl groups is independently selected from C₂-C₆ alkyl; and
R²⁴ is selected from H and C₁-C₆ alkyl;
s is an integer from 1 to 5;
L is selected from —C(O)(CH₂)₆C(O)— and —C(O)(CH₂)₂S₂(CH₂)₂C(O)—; and
each R²⁵ is of the formula —(CH₂)₂₀C(O)N(R²⁶)₂ wherein each R²⁶ is of the formula —(CH₂)₆NHC(═NH)NH₂,
wherein G is a cell penetrating peptide (“CPP”) and linker moiety selected from —C(O)(CH₂)₅NH-CPP, —C(O)(CH₂)₂NH-CPP, —C(O)(CH₂)₂NHC(O)(CH₂)₅NH-CPP, and —C(O)CH₂NH—CPP, or G is of the formula: 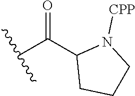
wherein the CPP is attached to the linker moiety by an amide bond at the CPP carboxy terminus, with the proviso that up to one instance of G is present, and
wherein the targeting sequence is:
I.
a) SEQ ID NO: 4 (GCC CXG GXC XGC XGG CXC CCX GCX G) wherein Z is 23;
b) SEQ ID NO: 5 (CCC XGG XCT GCT GGC TCC CTG CTG G) wherein Z is 23;
c) SEQ ID NO: 6 (CCX GGX CXG CXG GCX CCC XGC XGG X) wherein Z is 23;
d) SEQ ID NO: 7 (CXG GXC XGC XGG CXC CCX GCX GGX G) wherein Z is 23;
e) SEQ ID NO: 8 (XGG XCX GCX GGC XCC CXG CXG GXG A) wherein Z is 23;
f) SEQ ID NO: 9 (GGX CXG CXG GCX CCC XGC XGG XGA G) wherein Z is 23;
g) SEQ ID NO: 10 (GXC XGC XGG CXC CCX GCX GGX GAG C) wherein Z is 23;
h) SEQ ID NO: 11 (XCX GCX GGC XCC CXG CXG GXG AGC X) wherein Z is 23;
i) SEQ ID NO: 12 (GGG CCC XGG XCX GCX GGC XCC CXG C) wherein Z is 23;
j) SEQ ID NO: 13 (GGG GCC CXG GXC XGC XGG CXC CCX G) wherein Z is 23;
k) SEQ ID NO: 14 (CGG GGC CCX GGX CXG CXG GCX CCC X) wherein Z is 23;
l) SEQ ID NO: 15 (CCG GGG CCC XGG XCX GCX GGC XCC C) wherein Z is 23;
m) SEQ ID NO: 16 (CCC GGG GCC CXG GXC XGC XGG CXC C) wherein Z is 23;
n) SEQ ID NO: 17 (XCC CGG GGC CCX GGX CXG CXG GCX C) wherein Z is 23;
o) SEQ ID NO: 18 (AXC CCG GGG CCC XGG XCX GCX GGC X) wherein Z is 23;
p) SEQ ID NO: 19 (CAX CCC GGG GCC CXG GXC XGC XGG C) wherein Z is 23;
q) SEQ ID NO: 20 (XCX GCC CXG GCC GCC GCC CCC GCC CCX) wherein Z is 25;
r) SEQ ID NO: 21 (XGA GGX GCG XGG GXG XCG AXG XCC A) wherein Z is 23;
s) SEQ ID NO: 22 (GAG GXG CGX GGG XGX CGA XGX CCA C) wherein Z is 23;
t) SEQ ID NO: 23 (AGG XGC GXG GGX GXC GAX GXC CAC G) wherein Z is 23;
u) SEQ ID NO: 24 (GCG CGX GGA CAX CGA CAC CCA CGC A) wherein Z is 23;
v) SEQ ID NO: 25 (XGX GAG GGC GCG XGG ACA XCG ACA C) wherein Z is 23;
w) SEQ ID NO: 26 (XXG XGA GGG CGC GXG GAC AXC GAC A) wherein Z is 23;
x) SEQ ID NO: 27 (CX GXG AGG GCG CGX GGA CAX CGA C) wherein Z is 22;
y) SEQ ID NO: 28 (GGC CCX GGX CXG CXG GCX CCC XGC X) wherein Z is 23;
z) SEQ ID NO: 29 (XGG CCG CCG CCC CCG CCC CX) wherein Z is 18;
aa) SEQ ID NO: 30 (GXG AGG XGC GXG GGX GXC GA) wherein Z is 18, or
wherein X is selected from uracil (U) or thymine (T);
II.
a) SEQ ID NO: 133 (GCX CAG CAG GGA GGC GGG AG) wherein Z is 18;
b) SEQ ID NO:134 (GGC XCX CAA AGC AGC XCX GA) wherein Z is 18;
c) SEQ ID NO:135 (GAC AXC AAC CGC GGC XGG CAC XGC A) wherein Z is 23;
d) SEQ ID NO: 136 (GGG XAA GGX GGC CAG GGX GGG XGX X) wherein Z is 23;
e) SEQ ID NO: 137 (GCC CXG CXG XCX AGA CXG G) wherein Z is 17;
f) SEQ ID NO:138 (GAG AGG GCC AGA AGG AAG GG) wherein Z is 18;
g) SEQ ID NO:139 (CCC GCC CCX GCC CXG CC) wherein Z is 15;
h) SEQ ID NO: 140 (XGG CCG CCG CCC CCG CCC) wherein Z is 16;
i) SEQ ID NO:141 (XGX CCA CGC GCA CCC XCX GC) wherein Z is 18;
j) SEQ ID NO: 142 (GXG AGG XGC GXG GGX GXC GA) wherein Z is 18;
k) SEQ ID NO:143 (GCA ACA XGC ACC CCA CCC XX) wherein Z is 18;
l) SEQ ID NO:144 (AGG GCC CAG CAC ACA GXG GX) wherein Z is 18;
m) SEQ ID NO:145 (XCA CAC CXC CGC XCC CAG CA) wherein Z is 18;
n) SEQ ID NO:146 (GGC GCX GCC AXX GXC XGC) wherein Z is 16;
o) SEQ ID NO:147 (GXG XCC CCA CXG CXC CCC GA) wherein Z is 18;
p) SEQ ID NO: 148 (CXG GAG XAC CXG XCA CCG XG) wherein Z is 18;
q) SEQ ID NO: 149 (XGA GCC CCG AGC CCX GCC XX) wherein Z is 18;
r) SEQ ID NO:150 (XGA CCC ACC XXX XCA XAA AGA XGA A) wherein Z is 23;
s) SEQ ID NO:151 (CXC XGG CAG CCC XAC XCX ACC XGA C) wherein Z is 23;
t) SEQ ID NO:152 (CXA GXA XAA AXA CAX CCC AAA XXX XGC) wherein Z is 25;
u) SEQ ID NO:153 (GGC CCX GGX CXG CXG GCX CCC XGC X) wherein Z is 23;
v) SEQ ID NO:154 (GCX CCC XGC AGC CCC XGC XXX GCA G) wherein Z is 23;
w) SEQ ID NO:155 (GCG GGG CAG ACG XCA GGX GX) wherein Z is 18;
x) SEQ ID NO:156 (CAG CGC GGG GCA GAC GXC AG) wherein Z is 18;
y) SEQ ID NO:157 (CCG GCA GCG CGG GGC AGA CG) wherein Z is 18;
z) SEQ ID NO:158 (CCG CCG GCA GCG CGG GGC AG) wherein Z is 18;
aa) SEQ ID NO:159 (GAX GXX ACC GCC GGC AGC GC) wherein Z is 18;
bb) SEQ ID NO: 160 (CXG GGA XGX XAC CGC CGG CA) wherein Z is 18;
cc) SEQ ID NO: 161 (GCX XCX GGG AXG XXA CCG CC) wherein Z is 18;
dd) SEQ ID NO: 162 (XGG CAA CXC GXA XGX CCX XA) wherein Z is 18;
ee) SEQ ID NO: 163 (AXX CXG GCA ACX CGX AXG XC) wherein Z is 18;
ff) SEQ ID NO:164 (AAG XGA XXC XGG CAA CXC GX) wherein Z is 18;
gg) SEQ ID NO:165 (XGG GXG XCA GCG GAA GXG AX) wherein Z is 18;
hh) SEQ ID NO:166 (GXC CAC XGG GXG XCA GCG GA) wherein Z is 18;
ii) SEQ ID NO: 167 (GCX XGG XCC ACX GGG XGX CA) wherein Z is 18;
jj) SEQ ID NO:168 (CCC CAC XXC XGC AXA AAG GX) wherein Z is 18;
kk) SEQ ID NO:169 (GGA GCC CCA CXX CXG CAX AA) wherein Z is 18;
ll) SEQ ID NO:170 (GCX GGG AGC CCC ACX XCX GC) wherein Z is 18;
mm) SEQ ID NO:171 (CCA CGC CXG GCX GGG AGC CC) wherein Z is 18;
nn) SEQ ID NO: 172 (XCC GAA GXG CXG GGA XXX CA) wherein Z is 18;
oo) SEQ ID NO:173 (XCC ACC CCC CXX GGC CXX CC) wherein Z is 18;
pp) SEQ ID NO: 174 (XGA XCC ACC CCC CXX GGC CX) wherein Z is 18;
qq) SEQ ID NO:175 (XCA AGX GAX CCA CCC CCC XX) wherein Z is 18;
rr) SEQ ID NO:176 (GAA CXC CXG AGC XCA AGX GA) wherein Z is 18;
ss) SEQ ID NO:177 (XCX CGA ACX CCX GAG CXC AA) wherein Z is 18;
tt) SEQ ID NO:178 (CCA GGC XGG XCX CGA ACX CC) wherein Z is 18;
uu) SEQ ID NO:179 (XXX GCC AXG XXA CCC AGG CX) wherein Z is 18;
vv) SEQ ID NO: 180 (ACG GGA XXX XGC CAX GXX AC) wherein Z is 18;
ww) SEQ ID NO: 181 (XAG AGA CGG GAX XXX GCC AX) wherein Z is 18;
xx) SEQ ID NO:182 (XXX XGX AGA GAC GGG AXX XX) wherein Z is 18;
yy) SEQ ID NO:183 (XCX GXA XXX XXG XAG AGA CG) wherein Z is 18;
zz) SEQ ID NO:184 (AXX XXC XGX AXX XXX GXA GA) wherein Z is 18;
aaa) SEQ ID NO: 185 (GCX AAX XXX CXG XAX XXX XG) wherein Z is 18;
bbb) SEQ ID NO:186 (CCG CCG CCC CCG CCC CXG CC) wherein Z is 18;
ccc) SEQ ID NO:187 (XGG CCG CCG CCC CCG CCC CX) wherein Z is 18;
ddd) SEQ ID NO:188 (CXG CCC XGG CCG CCG CCC CC) wherein Z is 18;
eee) SEQ ID NO:189 (CAC CCX CXG CCC XGG CCG CC) wherein Z is 18;
fff) SEQ ID NO:190 (GCG CAC CCX CXG CCC XGG CC) wherein Z is 18;
ggg) SEQ ID NO:191 (XGX CGA XGX CCA CGC GCA CC) wherein Z is 18;
hhh) SEQ ID NO:192 (XGC GXG GGX GXC GAX GXC CA) wherein Z is 18;
iii) SEQ ID NO:193 (GCA CCC CAC CCX XGX GAG GX) wherein Z is 18;
jjj) SEQ ID NO:194 (AAC AXG CAC CCC ACC CXX GX) wherein Z is 18;
kkk) SEQ ID NO: 195 (AGG AGG AGG ACG CCX CCC CC) wherein Z is 18;
lll) SEQ ID NO:196 (CXC AXC XGC AGA GCC AGG AG) wherein Z is 18;
mmm) SEQ ID NO:197 (GCX CCC XCA XCX GCA GAG CC) wherein Z is 18;
nnn) SEQ ID NO:198 (XCG GCX CCC XCA XCX GCA GA) wherein Z is 18;
ooo) SEQ ID NO:199 (GCC XCG GCX CCC XCA XCX GC) wherein Z is 18;
ppp) SEQ ID NO:200 (XXC XGG GAX GXX ACC GCC GG) wherein Z is 18;
qqq) SEQ ID NO:201 (CXX CXG GGA XGX XAC CGC CG) wherein Z is 18;
rrr) SEQ ID NO:202 (CGC XXC XGG GAX GXX ACC GC) wherein Z is 18;
sss) SEQ ID NO:203 (CCG CXX CXG GGA XGX XAC CG) wherein Z is 18;
ttt) SEQ ID NO:204 (ACC CGC XXC XGG GAX GXX AC) wherein Z is 18;
uuu) SEQ ID NO:205 (XCA AAC CCG CXX CXG GGA XG) wherein Z is 18;
vvv) SEQ ID NO:206 (ACG XXC AAA CCC GCX XCX GG) wherein Z is 18;
www) SEQ ID NO:207 (GGG CXC XCA AAG CAG CXC XG) wherein Z is 18;
xxx) SEQ ID NO:208 (GGG GCX CXC AAA GCA GCX CX) wherein Z is 18;
yyy) SEQ ID NO:209 (ACG GGG CXC XCA AAG CAG CX) wherein Z is 18;
zzz) SEQ ID NO:210 (XCA CGG GGC XCX CAA AGC AG) wherein Z is 18;
aaaa) SEQ ID NO:211 (GCX CXC AAA GCA GCX CXG AG) wherein Z is 18;
bbbb) SEQ ID NO:212 (CXC XCA AAG CAG CXC XGA GA) wherein Z is 18;
cccc) SEQ ID NO:213 (CXC AAA GCA GCX CXG AGA CA) wherein Z is 18;
dddd) SEQ ID NO:214 (CAA AGC AGC XCX GAG ACA XC) wherein Z is 18;
eeee) SEQ ID NO:215 (AAG CAG CXC XGA GAC AXC AA) wherein Z is 18;
ffff) SEQ ID NO:216 (GCC CXG GXC XGC XGG CXC CCX GCX G) wherein Z is 23;
gggg) SEQ ID NO:217 (CCC XGG XCX GCX GGC XCC CXG CXG G) wherein Z is 23;
hhhh) SEQ ID NO:218 (CCX GGX CXG CXG GCX CCC XGC XGG X) wherein Z is 23;
iiii) SEQ ID NO:219 (CXG GXC XGC XGG CXC CCX GCX GGX G) wherein Z is 23;
jjjj) SEQ ID NO:220 (XGG XCX GCX GGC XCC CXG CXG GXG A) wherein Z is 23;
kkkk) SEQ ID NO:221 (GGX CXG CXG GCX CCC XGC XGG XGA G) wherein Z is 23;
llll) SEQ ID NO:222 (GXC XGC XGG CXC CCX GCX GGX GAG C) wherein Z is 23;
mmmm) SEQ ID NO:223 (XCX GCX GGC XCC CXG CXG GXG AGC X) wherein Z is 23;
nnnn) SEQ ID NO:224 (GGG CCC XGG XCX GCX GGC XCC CXG C) wherein Z is 23;
oooo) SEQ ID NO:225 (GGG GCC CXG GXC XGC XGG CXC CCX G) wherein Z is 23;
pppp) SEQ ID NO:226 (CGG GGC CCX GGX CXG CXG GCX CCC X) wherein Z is 23;
qqqq) SEQ ID NO:227 (CCG GGG CCC XGG XCX GCX GGC XCC C) wherein Z is 23;
rrrr) SEQ ID NO:228 (CCC GGG GCC CXG GXC XGC XGG CXC C) wherein Z is 23;
ssss) SEQ ID NO:229 (XCC CGG GGC CCX GGX CXG CXG GCX C) wherein Z is 23;
tttt) SEQ ID NO:230 (AXC CCG GGG CCC XGG XCX GCX GGC X) wherein Z is 23;
uuuu) SEQ ID NO:231 (CAX CCC GGG GCC CXG GXC XGC XGG C) wherein Z is 23;
vvvv) SEQ ID NO:232 (XCX GCC CXG GCC GCC GCC CCC GCC CCX) wherein Z is 23;
wwww) SEQ ID NO:233 (XGA GGX GCG XGG GXG XCG AXG XCC A) wherein Z is 23;
xxxx) SEQ ID NO:234 (GAG GXG CGX GGG XGX CGA XGX CCA C) wherein Z is 23;
yyyy) SEQ ID NO:235 (AGG XGC GXG GGX GXC GAX GXC CAC G) wherein Z is 23;
zzzz) SEQ ID NO:236 (GCG CGX GGA CAX CGA CAC CCA CGC A) wherein Z is 23;
aaaaa) SEQ ID NO:237 (XGX GAG GGC GCG XGG ACA XCG ACA C) wherein Z is 23;
bbbbb) SEQ ID NO:238 (XXG XGA GGG CGC GXG GAC AXC GAC A) wherein Z is 23;
cccc) SEQ ID NO:239 (CXX GXG AGG GCG CGX GGA CAX CGA C) wherein Z is 23;
ddddd) SEQ ID NO:240 (GAG AGG GCC AGA AGG AAG) wherein Z is 16;
eeeee) SEQ ID NO:241 (GAG GGC CAG AAG GAA GGG) wherein Z is 16;
fffff) SEQ ID NO:242 (GGG CCA GAA GGA AGG GCG) wherein Z is 16;
ggggg) SEQ ID NO:243 (GGG AGA GGG CCA GAA GGA) wherein Z is 16;
hhhhh) SEQ ID NO:244 (AGA GGG CCA GAA GGA AGG GC) wherein Z is 18;
iiiii) SEQ ID NO:245 (GAG GGC CAG AAG GAA GGG CG) wherein Z is 18;
jjjjj) SEQ ID NO:246 (AGG GCC AGA AGG AAG GGC GA) wherein Z is 18;
kkkkk) SEQ ID NO:247 (GGG CCA GAA GGA AGG GCG AG) wherein Z is 18;
lllll) SEQ ID NO:248 (GGC CAG AAG GAA GGG CGA GA) wherein Z is 18;
mmmmm) SEQ ID NO:249 (GCC AGA AGG AAG GGC GAG AA) wherein Z is 18;
nnnnn) SEQ ID NO:250 (GGG AGA GGG CCA GAA GGA AGG G) wherein Z is 20;
ooooo) SEQ ID NO:251 (CXG GGG AGA GGG CCA GAA GGA AGG G) wherein Z is 23;
ppppp) SEQ ID NO:252 (GAG AGG GCC AGA AGG AAG GGC G) wherein Z is 20;
qqqqq) SEQ ID NO:253 (GAG AGG GCC AGA AGG AAG GGC GAG A) wherein Z is 23;
rrrrr) SEQ ID NO:254 (GAA GGA AGG GCG AGA AAA GC) wherein Z is 18; or
sssss) SEQ ID NO:255 (GCA GAA AAG CXC CAG CAG GG) wherein Z is 18,
wherein X is selected from uracil (U) or thymine (T); or
III.
a) SEQ ID NO:296 (AAG CXC CAG CAG GGG AGX GCA GAG C) wherein Z is 23;
b) SEQ ID NO:297 (AAA AGC XCC AGC AGG GGA GXG CAG A) wherein Z is 23;
c) SEQ ID NO:298 (AGA AAA GCX CCA GCA GGG GAG XGC A) wherein Z is 23;
d) SEQ ID NO:299 (CGA GAA AAG CXC CAG CAG GGG AGX G) wherein Z is 23;
e) SEQ ID NO:300 (GGC GAG AAA AGC XCC AGC AGG GGA G) wherein Z is 23;
f) SEQ ID NO:301 (AGG GCG AGA AAA GCX CCA GCA GGG G) wherein Z is 23;
g) SEQ ID NO:302 (GAA GGG CGA GAA AAG CXC CAG CAG G) wherein Z is 23;
h) SEQ ID NO:303 (AGG AAG GGC GAG AAA AGC XCC AGC A) wherein Z is 23;
i) SEQ ID NO:304 (GAA GGA AGG GCG AGA AAA GCX CCA G) wherein Z is 23;
j) SEQ ID NO:305 (CAG AAG GAA GGG CGA GAA AAG CXC C) wherein Z is 23;
k) SEQ ID NO:306 (GCC AGA AGG AAG GGC GAG AAA AGC X) wherein Z is 23;
l) SEQ ID NO:307 (GGG CCA GAA GGA AGG GCG AGA AAA G) wherein Z is 23;
m) SEQ ID NO:308 (GAG GGC CAG AAG GAA GGG CGA GAA A) wherein Z is 23;
n) SEQ ID NO:309 (GAG AGG GCC AGA AGG AAG GGC GAG A) wherein Z is 23;
o) SEQ ID NO:310 (AGG GCC AGA AGG AAG GGC GA) wherein Z is 18;
p) SEQ ID NO:311 (GAG AGG GCC AGA AGG AAG GG) wherein Z is 18;
q) SEQ ID NO:312 (CXG GGG AGA GGG CCA GAA GGA AGG G) wherein Z is 23;
r) SEQ ID NO:313 (GAG AGG GCC AGA AGG AAG) wherein Z is 18;
s) SEQ ID NO:314 (GGG AGA GGG CCA GAA GGA) wherein Z is 18;
t) SEQ ID NO:315 (GGX CXG CXG GCX CCC XGC XGG XGA G) wherein Z is 23;
u) SEQ ID NO:316 (CAC XCA CGG GGC XCX CAA AGC AGC X) wherein Z is 23;
v) SEQ ID NO:317 (XCX GGG AXG XXA CCG CCG GCA GCG C) wherein Z is 23;
w) SEQ ID NO:318 (XXX GCC AXG XXA CCC AGG CX) wherein Z is 18;
x) SEQ ID NO:319 (ACX CAC GGG GCX CXC AAA GCA GCX C) wherein Z is 23;
y) SEQ ID NO:320 (GCA CXC ACG GGG CXC XCA AAG CAG C) wherein Z is 23;
z) SEQ ID NO:321 (CGG GGC XCX CAA AGC AGC XCX GAG A) wherein Z is 23;
aa) SEQ ID NO:322 (ACG GGG CXC XCA AAG CAG CXC XGA G) wherein Z is 23;
bb) SEQ ID NO:323 (CAC GGG GCX CXC AAA GCA GCX CXG A) wherein Z is 23;
cc) SEQ ID NO:324 (XCA CGG GGC XCX CAA AGC AGC XCX G) wherein Z is 23;
dd) SEQ ID NO:325 (CXC ACG GGG CXC XCA AAG CAG CXC X) wherein Z is 23;
ee) SEQ ID NO:326 (GGC ACX CAC GGG GCX CXC AAA GCA G) wherein Z is 23;
ff) SEQ ID NO:327 (CGG CAC XCA CGG GGC XCX CAA AGC A) wherein Z is 23;
gg) SEQ ID NO:328 (GCG GCA CXC ACG GGG CXC XCA AAG C) wherein Z is 23;
hh) SEQ ID NO:329 (GGC GGC ACX CAC GGG GCX CXC AAA G) wherein Z is 23;
ii) SEQ ID NO:330 (GGG CGG CAC XCA CGG GGC XCX CAA A) wherein Z is 23;
jj) SEQ ID NO:331 (GGG GCG GCA CXC ACG GGG CXC XCA A) wherein Z is 23;
kk) SEQ ID NO:332 (AGG GGC GGC ACX CAC GGG GCX CXC A) wherein Z is 23;
ll) SEQ ID NO:333 (GAG GGG CGG CAC XCA CGG GGC XCX C) wherein Z is 23;
mm) SEQ ID NO: 334 (GGC XCX CAA AGC AGC XCX GA) wherein Z is 18;
nn) SEQ ID NO: 335 (XXC XGG GAX GXX ACC GCC GGC AGC G) wherein Z is 23;
oo) SEQ ID NO: 336 (CXX CXG GGA XGX XAC CGC CGG CAG C) wherein Z is 23;
pp) SEQ ID NO: 337 (GCX XCX GGG AXG XXA CCG CCG GCA G) wherein Z is 23;
qq) SEQ ID NO: 338 (CGC XXC XGG GAX GXX ACC GCC GGC A) wherein Z is 23;
rr) SEQ ID NO: 339 (CCG CXX CXG GGA XGX XAC CGC CGG C) wherein Z is 23;
ss) SEQ ID NO: 340 (CCC GCX XCX GGG AXG XXA CCG CCG G) wherein Z is 23;
tt) SEQ ID NO: 341 (ACC CGC XXC XGG GAX GXX ACC GCC G) wherein Z is 23;
uu) SEQ ID NO: 342 (AAC CCG CXX CXG GGA XGX XAC CGC C) wherein Z is 23,
wherein X is selected from uracil (U) or thymine (T).

2. The compound of claim 1, wherein at least one R¹ is selected from:

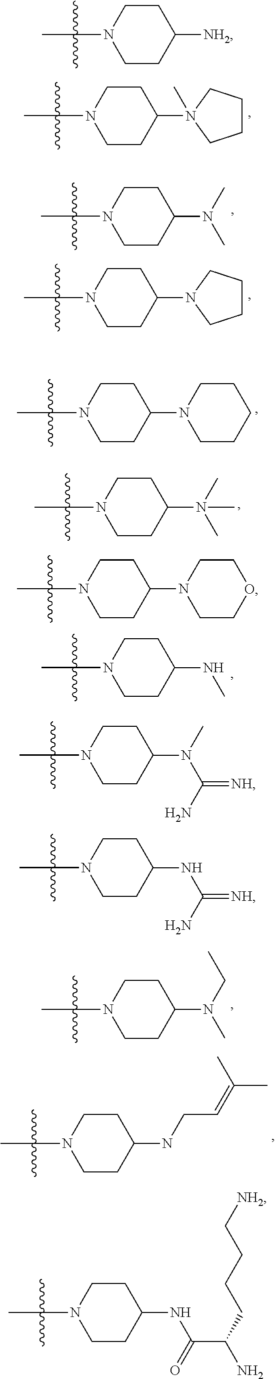

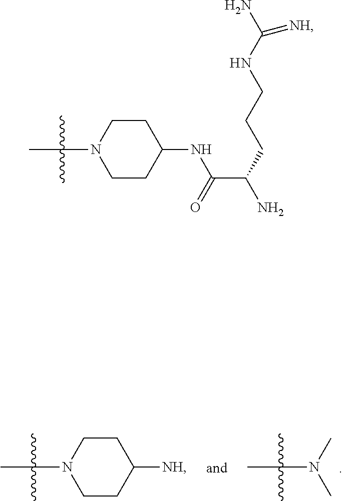

3. The compound of claim 1, wherein each R¹ is —N(CH₃)₂.

4. The compound of claim 1, wherein 50-90% of the R¹ groups are —N(CH₂)₃.

5. The compound of claim 4, wherein 66% of the R¹ groups are —N(CH₂)₃.

6. The compound of claim 1, wherein T is selected from: 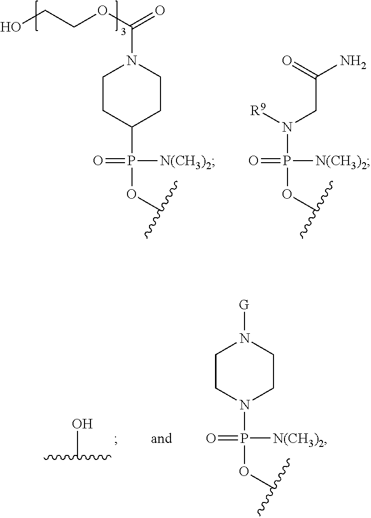
and
Y is O at each occurrence.

7. The compound of claim 1, wherein R² is selected from H, G, acyl, trityl, 4-methoxytrityl, benzoyl, and stearoyl.

8. The compound of claim 1, wherein T is selected from: 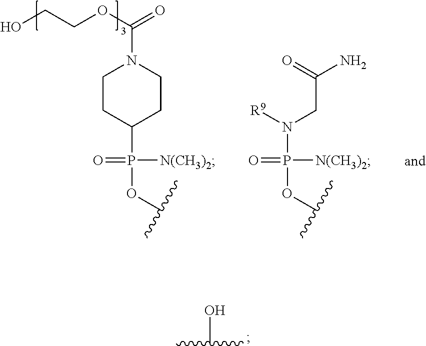
Y is O at each occurrence, and R² is G.

9. The compound of claim 1, wherein T is of the formula: 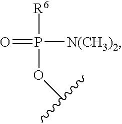
R⁶ is of the formula: 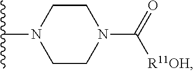
Y is O at each occurrence and R² is G.

10. The compound of claim 1, wherein T is of the formula: 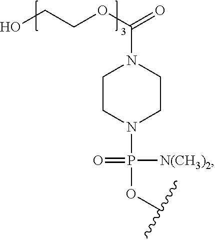
Y is O at each occurrence and R² is G.

11. The compound of claim 1, wherein T is of the formula: 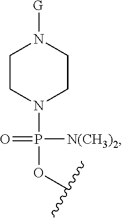
and
Y is O at each occurrence and R² is selected from H, acyl, trityl, 4-methoxytrityl, benzoyl, and stearoyl.

12. (canceled)

13. The compound of claim 1, wherein G is of the formula: 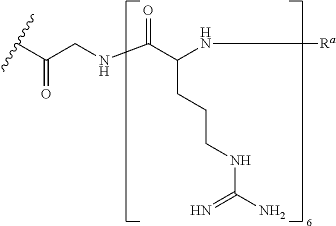
wherein R^{a} is selected from H, acetyl, benzoyl, and stearoyl.

14. The compound of claim 1, wherein the CPP is of the formula: 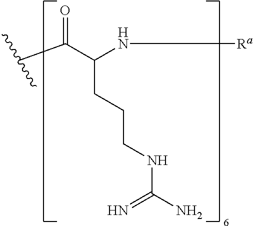
wherein R^{a} is selected from H, acetyl, benzoyl, and stearoyl.

15. The compound of claim 1, wherein:
T is of the formula: 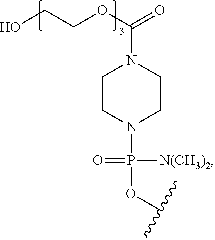
Y is O at each occurrence, each R¹ is —N(CH₃)₂, and R² is H.

16. A compound of formula (IVb): 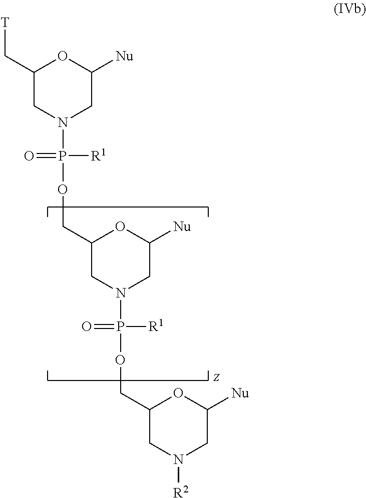
or a pharmaceutically acceptable salt thereof, where:
each Nu is a nucleobase which taken together forms a targeting sequence;
T is selected from a moiety of the formula: 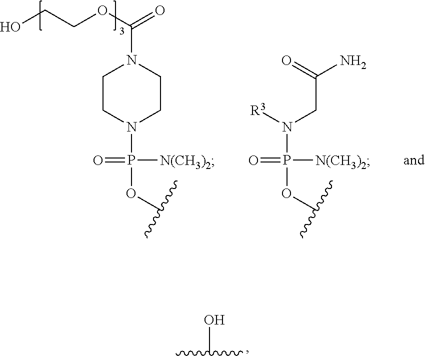
wherein R³ is selected from H and C₁-C₆ alkyl;
each instance of R¹ is independently —N(R⁴)₂, wherein each R⁴ is independently selected from H and C₁-C₆ alkyl; and
R² is selected from H, acyl, trityl, 4-methoxytrityl, and C₁-C₆ alkyl,
wherein the targeting sequence is:
I.
a) SEQ ID NO: 4 (GCC CXG GXC XGC XGG CXC CCX GCX G) wherein Z is 23;
b) SEQ ID NO: 5 (CCC XGG XCT GCT GGC TCC CTG CTG G) wherein Z is 23;
c) SEQ ID NO: 6 (CCX GGX CXG CXG GCX CCC XGC XGG X) wherein Z is 23;
d) SEQ ID NO: 7 (CXG GXC XGC XGG CXC CCX GCX GGX G) wherein Z is 23;
e) SEQ ID NO: 8 (XGG XCX GCX GGC XCC CXG CXG GXG A) wherein Z is 23;
f) SEQ ID NO: 9 (GGX CXG CXG GCX CCC XGC XGG XGA G) wherein Z is 23;
g) SEQ ID NO: 10 (GXC XGC XGG CXC CCX GCX GGX GAG C) wherein Z is 23;
h) SEQ ID NO: 11 (XCX GCX GGC XCC CXG CXG GXG AGC X) wherein Z is 23;
i) SEQ ID NO: 12 (GGG CCC XGG XCX GCX GGC XCC CXG C) wherein Z is 23;
j) SEQ ID NO: 13 (GGG GCC CXG GXC XGC XGG CXC CCX G) wherein Z is 23;
k) SEQ ID NO: 14 (CGG GGC CCX GGX CXG CXG GCX CCC X) wherein Z is 23;
l) SEQ ID NO: 15 (CCG GGG CCC XGG XCX GCX GGC XCC C) wherein Z is 23;
m) SEQ ID NO: 16 (CCC GGG GCC CXG GXC XGC XGG CXC C) wherein Z is 23;
n) SEQ ID NO: 17 (XCC CGG GGC CCX GGX CXG CXG GCX C) wherein Z is 23;
o) SEQ ID NO: 18 (AXC CCG GGG CCC XGG XCX GCX GGC X) wherein Z is 23;
p) SEQ ID NO: 19 (CAX CCC GGG GCC CXG GXC XGC XGG C) wherein Z is 23;
q) SEQ ID NO: 20 (XCX GCC CXG GCC GCC GCC CCC GCC CCX) wherein Z is 25;
r) SEQ ID NO: 21 (XGA GGX GCG XGG GXG XCG AXG XCC A) wherein Z is 23;
s) SEQ ID NO: 22 (GAG GXG CGX GGG XGX CGA XGX CCA C) wherein Z is 23;
t) SEQ ID NO: 23 (AGG XGC GXG GGX GXC GAX GXC CAC G) wherein Z is 23;
u) SEQ ID NO: 24 (GCG CGX GGA CAX CGA CAC CCA CGC A) wherein Z is 23;
v) SEQ ID NO: 25 (XGX GAG GGC GCG XGG ACA XCG ACA C) wherein Z is 23;
w) SEQ ID NO: 26 (XXG XGA GGG CGC GXG GAC AXC GAC A) wherein Z is 23;
x) SEQ ID NO: 27 (CX GXG AGG GCG CGX GGA CAX CGA C) wherein Z is 22;
y) SEQ ID NO: 28 (GGC CCX GGX CXG CXG GCX CCC XGC X) wherein Z is 23;
z) SEQ ID NO: 29 (XGG CCG CCG CCC CCG CCC CX) wherein Z is 18;
aa) SEQ ID NO: 30 (GXG AGG XGC GXG GGX GXC GA) wherein Z is 18, or
wherein X is selected from uracil (U) or thymine (T);
II.
a) SEQ ID NO: 133 (GCX CAG CAG GGA GGC GGG AG) wherein Z is 18;
b) SEQ ID NO:134 (GGC XCX CAA AGC AGC XCX GA) wherein Z is 18;
c) SEQ ID NO: 135 (GAC AXC AAC CGC GGC XGG CAC XGC A) wherein Z is 23;
d) SEQ ID NO: 136 (GGG XAA GGX GGC CAG GGX GGG XGX X) wherein Z is 23;
e) SEQ ID NO: 137 (GCC CXG CXG XCX AGA CXG G) wherein Z is 17;
f) SEQ ID NO:138 (GAG AGG GCC AGA AGG AAG GG) wherein Z is 18;
g) SEQ ID NO:139 (CCC GCC CCX GCC CXG CC) wherein Z is 15;
h) SEQ ID NO: 140 (XGG CCG CCG CCC CCG CCC) wherein Z is 16;
i) SEQ ID NO:141 (XGX CCA CGC GCA CCC XCX GC) wherein Z is 18;
j) SEQ ID NO:142 (GXG AGG XGC GXG GGX GXC GA) wherein Z is 18;
k) SEQ ID NO:143 (GCA ACA XGC ACC CCA CCC XX) wherein Z is 18;
l) SEQ ID NO:144 (AGG GCC CAG CAC ACA GXG GX) wherein Z is 18;
m) SEQ ID NO: 145 (XCA CAC CXC CGC XCC CAG CA) wherein Z is 18;
n) SEQ ID NO: 146 (GGC GCX GCC AXX GXC XGC) wherein Z is 16;
o) SEQ ID NO:147 (GXG XCC CCA CXG CXC CCC GA) wherein Z is 18;
p) SEQ ID NO:148 (CXG GAG XAC CXG XCA CCG XG) wherein Z is 18;
q) SEQ ID NO: 149 (XGA GCC CCG AGC CCX GCC XX) wherein Z is 18;
r) SEQ ID NO:150 (XGA CCC ACC XXX XCA XAA AGA XGA A) wherein Z is 23;
s) SEQ ID NO:151 (CXC XGG CAG CCC XAC XCX ACC XGA C) wherein Z is 23;
t) SEQ ID NO:152 (CXA GXA XAA AXA CAX CCC AAA XXX XGC) wherein Z is 25;
u) SEQ ID NO:153 (GGC CCX GGX CXG CXG GCX CCC XGC X) wherein Z is 23;
v) SEQ ID NO:154 (GCX CCC XGC AGC CCC XGC XXX GCA G) wherein Z is 23;
w) SEQ ID NO: 155 (GCG GGG CAG ACG XCA GGX GX) wherein Z is 18;
x) SEQ ID NO:156 (CAG CGC GGG GCA GAC GXC AG) wherein Z is 18;
y) SEQ ID NO:157 (CCG GCA GCG CGG GGC AGA CG) wherein Z is 18;
z) SEQ ID NO:158 (CCG CCG GCA GCG CGG GGC AG) wherein Z is 18;
aa) SEQ ID NO:159 (GAX GXX ACC GCC GGC AGC GC) wherein Z is 18;
bb) SEQ ID NO:160 (CXG GGA XGX XAC CGC CGG CA) wherein Z is 18;
cc) SEQ ID NO: 161 (GCX XCX GGG AXG XXA CCG CC) wherein Z is 18;
dd) SEQ ID NO: 162 (XGG CAA CXC GXA XGX CCX XA) wherein Z is 18;
ee) SEQ ID NO: 163 (AXX CXG GCA ACX CGX AXG XC) wherein Z is 18;
ff) SEQ ID NO: 164 (AAG XGA XXC XGG CAA CXC GX) wherein Z is 18;
gg) SEQ ID NO: 165 (XGG GXG XCA GCG GAA GXG AX) wherein Z is 18;
hh) SEQ ID NO: 166 (GXC CAC XGG GXG XCA GCG GA) wherein Z is 18;
ii) SEQ ID NO: 167 (GCX XGG XCC ACX GGG XGX CA) wherein Z is 18;
jj) SEQ ID NO:168 (CCC CAC XXC XGC AXA AAG GX) wherein Z is 18;
kk) SEQ ID NO:169 (GGA GCC CCA CXX CXG CAX AA) wherein Z is 18;
ll) SEQ ID NO:170 (GCX GGG AGC CCC ACX XCX GC) wherein Z is 18;
mm) SEQ ID NO:171 (CCA CGC CXG GCX GGG AGC CC) wherein Z is 18;
nn) SEQ ID NO: 172 (XCC GAA GXG CXG GGA XXX CA) wherein Z is 18;
oo) SEQ ID NO:173 (XCC ACC CCC CXX GGC CXX CC) wherein Z is 18;
pp) SEQ ID NO:174 (XGA XCC ACC CCC CXX GGC CX) wherein Z is 18;
qq) SEQ ID NO:175 (XCA AGX GAX CCA CCC CCC XX) wherein Z is 18;
rr) SEQ ID NO: 176 (GAA CXC CXG AGC XCA AGX GA) wherein Z is 18;
ss) SEQ ID NO:177 (XCX CGA ACX CCX GAG CXC AA) wherein Z is 18;
tt) SEQ ID NO:178 (CCA GGC XGG XCX CGA ACX CC) wherein Z is 18;
uu) SEQ ID NO:179 (XXX GCC AXG XXA CCC AGG CX) wherein Z is 18;
vv) SEQ ID NO: 180 (ACG GGA XXX XGC CAX GXX AC) wherein Z is 18;
ww) SEQ ID NO: 181 (XAG AGA CGG GAX XXX GCC AX) wherein Z is 18;
xx) SEQ ID NO:182 (XXX XGX AGA GAC GGG AXX XX) wherein Z is 18;
yy) SEQ ID NO: 183 (XCX GXA XXX XXG XAG AGA CG) wherein Z is 18;
zz) SEQ ID NO:184 (AXX XXC XGX AXX XXX GXA GA) wherein Z is 18;
aaa) SEQ ID NO: 185 (GCX AAX XXX CXG XAX XXX XG) wherein Z is 18;
bbb) SEQ ID NO: 186 (CCG CCG CCC CCG CCC CXG CC) wherein Z is 18;
ccc) SEQ ID NO: 187 (XGG CCG CCG CCC CCG CCC CX) wherein Z is 18;
ddd) SEQ ID NO:188 (CXG CCC XGG CCG CCG CCC CC) wherein Z is 18;
eee) SEQ ID NO:189 (CAC CCX CXG CCC XGG CCG CC) wherein Z is 18;
fff) SEQ ID NO:190 (GCG CAC CCX CXG CCC XGG CC) wherein Z is 18;
ggg) SEQ ID NO:191 (XGX CGA XGX CCA CGC GCA CC) wherein Z is 18;
hhh) SEQ ID NO:192 (XGC GXG GGX GXC GAX GXC CA) wherein Z is 18;
iii) SEQ ID NO:193 (GCA CCC CAC CCX XGX GAG GX) wherein Z is 18;
jjj) SEQ ID NO:194 (AAC AXG CAC CCC ACC CXX GX) wherein Z is 18;
kkk) SEQ ID NO: 195 (AGG AGG AGG ACG CCX CCC CC) wherein Z is 18;
lll) SEQ ID NO:196 (CXC AXC XGC AGA GCC AGG AG) wherein Z is 18;
mmm) SEQ ID NO:197 (GCX CCC XCA XCX GCA GAG CC) wherein Z is 18;
nnn) SEQ ID NO:198 (XCG GCX CCC XCA XCX GCA GA) wherein Z is 18;
ooo) SEQ ID NO:199 (GCC XCG GCX CCC XCA XCX GC) wherein Z is 18;
ppp) SEQ ID NO:200 (XXC XGG GAX GXX ACC GCC GG) wherein Z is 18;
qqq) SEQ ID NO:201 (CXX CXG GGA XGX XAC CGC CG) wherein Z is 18;
rrr) SEQ ID NO:202 (CGC XXC XGG GAX GXX ACC GC) wherein Z is 18;
sss) SEQ ID NO:203 (CCG CXX CXG GGA XGX XAC CG) wherein Z is 18;
ttt) SEQ ID NO:204 (ACC CGC XXC XGG GAX GXX AC) wherein Z is 18;
uuu) SEQ ID NO:205 (XCA AAC CCG CXX CXG GGA XG) wherein Z is 18;
vvv) SEQ ID NO:206 (ACG XXC AAA CCC GCX XCX GG) wherein Z is 18;
www) SEQ ID NO:207 (GGG CXC XCA AAG CAG CXC XG) wherein Z is 18;
xxx) SEQ ID NO:208 (GGG GCX CXC AAA GCA GCX CX) wherein Z is 18;
yyy) SEQ ID NO:209 (ACG GGG CXC XCA AAG CAG CX) wherein Z is 18;
zzz) SEQ ID NO:210 (XCA CGG GGC XCX CAA AGC AG) wherein Z is 18;
aaaa) SEQ ID NO:211 (GCX CXC AAA GCA GCX CXG AG) wherein Z is 18;
bbbb) SEQ ID NO:212 (CXC XCA AAG CAG CXC XGA GA) wherein Z is 18;
cccc) SEQ ID NO:213 (CXC AAA GCA GCX CXG AGA CA) wherein Z is 18;
dddd) SEQ ID NO:214 (CAA AGC AGC XCX GAG ACA XC) wherein Z is 18;
eeee) SEQ ID NO:215 (AAG CAG CXC XGA GAC AXC AA) wherein Z is 18;
ffff) SEQ ID NO:216 (GCC CXG GXC XGC XGG CXC CCX GCX G) wherein Z is 23;
gggg) SEQ ID NO:217 (CCC XGG XCX GCX GGC XCC CXG CXG G) wherein Z is 23;
hhhh) SEQ ID NO:218 (CCX GGX CXG CXG GCX CCC XGC XGG X) wherein Z is 23;
iiii) SEQ ID NO:219 (CXG GXC XGC XGG CXC CCX GCX GGX G) wherein Z is 23;
jjjj) SEQ ID NO:220 (XGG XCX GCX GGC XCC CXG CXG GXG A) wherein Z is 23;
kkkk) SEQ ID NO:221 (GGX CXG CXG GCX CCC XGC XGG XGA G) wherein Z is 23;
llll) SEQ ID NO:222 (GXC XGC XGG CXC CCX GCX GGX GAG C) wherein Z is 23;
mmmm) SEQ ID NO:223 (XCX GCX GGC XCC CXG CXG GXG AGC X) wherein Z is 23;
nnnn) SEQ ID NO:224 (GGG CCC XGG XCX GCX GGC XCC CXG C) wherein Z is 23;
oooo) SEQ ID NO:225 (GGG GCC CXG GXC XGC XGG CXC CCX G) wherein Z is 23;
pppp) SEQ ID NO:226 (CGG GGC CCX GGX CXG CXG GCX CCC X) wherein Z is 23;
qqqq) SEQ ID NO:227 (CCG GGG CCC XGG XCX GCX GGC XCC C) wherein Z is 23;
rrrr) SEQ ID NO:228 (CCC GGG GCC CXG GXC XGC XGG CXC C) wherein Z is 23;
ssss) SEQ ID NO:229 (XCC CGG GGC CCX GGX CXG CXG GCX C) wherein Z is 23;
tttt) SEQ ID NO:230 (AXC CCG GGG CCC XGG XCX GCX GGC X) wherein Z is 23;
uuuu) SEQ ID NO:231 (CAX CCC GGG GCC CXG GXC XGC XGG C) wherein Z is 23;
vvvv) SEQ ID NO:232 (XCX GCC CXG GCC GCC GCC CCC GCC CCX) wherein Z is 23;
wwww) SEQ ID NO:233 (XGA GGX GCG XGG GXG XCG AXG XCC A) wherein Z is 23;
xxxx) SEQ ID NO:234 (GAG GXG CGX GGG XGX CGA XGX CCA C) wherein Z is 23;
yyyy) SEQ ID NO:235 (AGG XGC GXG GGX GXC GAX GXC CAC G) wherein Z is 23;
zzzz) SEQ ID NO:236 (GCG CGX GGA CAX CGA CAC CCA CGC A) wherein Z is 23;
aaaaa) SEQ ID NO:237 (XGX GAG GGC GCG XGG ACA XCG ACA C) wherein Z is 23;
bbbbb) SEQ ID NO:238 (XXG XGA GGG CGC GXG GAC AXC GAC A) wherein Z is 23;
ccccc) SEQ ID NO:239 (CXX GXG AGG GCG CGX GGA CAX CGA C) wherein Z is 23;
ddddd) SEQ ID NO:240 (GAG AGG GCC AGA AGG AAG) wherein Z is 16;
eeeee) SEQ ID NO:241 (GAG GGC CAG AAG GAA GGG) wherein Z is 16;
fffff) SEQ ID NO:242 (GGG CCA GAA GGA AGG GCG) wherein Z is 16;
ggggg) SEQ ID NO:243 (GGG AGA GGG CCA GAA GGA) wherein Z is 16;
hhhhh) SEQ ID NO:244 (AGA GGG CCA GAA GGA AGG GC) wherein Z is 18;
iiiii) SEQ ID NO:245 (GAG GGC CAG AAG GAA GGG CG) wherein Z is 18;
jjjjj) SEQ ID NO:246 (AGG GCC AGA AGG AAG GGC GA) wherein Z is 18;
kkkkk) SEQ ID NO:247 (GGG CCA GAA GGA AGG GCG AG) wherein Z is 18;
lllll) SEQ ID NO:248 (GGC CAG AAG GAA GGG CGA GA) wherein Z is 18;
mmmmm) SEQ ID NO:249 (GCC AGA AGG AAG GGC GAG AA) wherein Z is 18;
nnnnn) SEQ ID NO:250 (GGG AGA GGG CCA GAA GGA AGG G) wherein Z is 20;
ooooo) SEQ ID NO:251 (CXG GGG AGA GGG CCA GAA GGA AGG G) wherein Z is 23;
ppppp) SEQ ID NO:252 (GAG AGG GCC AGA AGG AAG GGC G) wherein Z is 20;
qqqqq) SEQ ID NO:253 (GAG AGG GCC AGA AGG AAG GGC GAG A) wherein Z is 23;
rrrrr) SEQ ID NO:254 (GAA GGA AGG GCG AGA AAA GC) wherein Z is 18; or
sssss) SEQ ID NO:255 (GCA GAA AAG CXC CAG CAG GG) wherein Z is 18, wherein X is selected from uracil (U) or thymine (T); or
III.
a) SEQ ID NO:296 (AAG CXC CAG CAG GGG AGX GCA GAG C) wherein Z is 23;
b) SEQ ID NO:297 (AAA AGC XCC AGC AGG GGA GXG CAG A) wherein Z is 23;
c) SEQ ID NO:298 (AGA AAA GCX CCA GCA GGG GAG XGC A) wherein Z is 23;
d) SEQ ID NO:299 (CGA GAA AAG CXC CAG CAG GGG AGX G) wherein Z is 23;
e) SEQ ID NO:300 (GGC GAG AAA AGC XCC AGC AGG GGA G) wherein Z is 23;
f) SEQ ID NO:301 (AGG GCG AGA AAA GCX CCA GCA GGG G) wherein Z is 23;
g) SEQ ID NO:302 (GAA GGG CGA GAA AAG CXC CAG CAG G) wherein Z is 23;
h) SEQ ID NO:303 (AGG AAG GGC GAG AAA AGC XCC AGC A) wherein Z is 23;
i) SEQ ID NO:304 (GAA GGA AGG GCG AGA AAA GCX CCA G) wherein Z is 23;
j) SEQ ID NO:305 (CAG AAG GAA GGG CGA GAA AAG CXC C) wherein Z is 23;
k) SEQ ID NO:306 (GCC AGA AGG AAG GGC GAG AAA AGC X) wherein Z is 23;
l) SEQ ID NO:307 (GGG CCA GAA GGA AGG GCG AGA AAA G) wherein Z is 23;
m) SEQ ID NO:308 (GAG GGC CAG AAG GAA GGG CGA GAA A) wherein Z is 23;
n) SEQ ID NO:309 (GAG AGG GCC AGA AGG AAG GGC GAG A) wherein Z is 23;
o) SEQ ID NO:310 (AGG GCC AGA AGG AAG GGC GA) wherein Z is 18;
p) SEQ ID NO:311 (GAG AGG GCC AGA AGG AAG GG) wherein Z is 18;
q) SEQ ID NO:312 (CXG GGG AGA GGG CCA GAA GGA AGG G) wherein Z is 23;
r) SEQ ID NO:313 (GAG AGG GCC AGA AGG AAG) wherein Z is 18;
s) SEQ ID NO:314 (GGG AGA GGG CCA GAA GGA) wherein Z is 18;
t) SEQ ID NO:315 (GGX CXG CXG GCX CCC XGC XGG XGA G) wherein Z is 23;
u) SEQ ID NO:316 (CAC XCA CGG GGC XCX CAA AGC AGC X) wherein Z is 23;
v) SEQ ID NO:317 (XCX GGG AXG XXA CCG CCG GCA GCG C) wherein Z is 23;
w) SEQ ID NO:318 (XXX GCC AXG XXA CCC AGG CX) wherein Z is 18;
x) SEQ ID NO:319 (ACX CAC GGG GCX CXC AAA GCA GCX C) wherein Z is 23;
y) SEQ ID NO:320 (GCA CXC ACG GGG CXC XCA AAG CAG C) wherein Z is 23;
z) SEQ ID NO:321 (CGG GGC XCX CAA AGC AGC XCX GAG A) wherein Z is 23;
aa) SEQ ID NO:322 (ACG GGG CXC XCA AAG CAG CXC XGA G) wherein Z is 23;
bb) SEQ ID NO:323 (CAC GGG GCX CXC AAA GCA GCX CXG A) wherein Z is 23;
cc) SEQ ID NO:324 (XCA CGG GGC XCX CAA AGC AGC XCX G) wherein Z is 23;
dd) SEQ ID NO:325 (CXC ACG GGG CXC XCA AAG CAG CXC X) wherein Z is 23;
ee) SEQ ID NO:326 (GGC ACX CAC GGG GCX CXC AAA GCA G) wherein Z is 23;
ff) SEQ ID NO:327 (CGG CAC XCA CGG GGC XCX CAA AGC A) wherein Z is 23;
gg) SEQ ID NO:328 (GCG GCA CXC ACG GGG CXC XCA AAG C) wherein Z is 23;
hh) SEQ ID NO:329 (GGC GGC ACX CAC GGG GCX CXC AAA G) wherein Z is 23;
ii) SEQ ID NO:330 (GGG CGG CAC XCA CGG GGC XCX CAA A) wherein Z is 23;
jj) SEQ ID NO:331 (GGG GCG GCA CXC ACG GGG CXC XCA A) wherein Z is 23;
kk) SEQ ID NO:332 (AGG GGC GGC ACX CAC GGG GCX CXC A) wherein Z is 23;
ll) SEQ ID NO:333 (GAG GGG CGG CAC XCA CGG GGC XCX C) wherein Z is 23;
mm) SEQ ID NO: 334 (GGC XCX CAA AGC AGC XCX GA) wherein Z is 18;
nn) SEQ ID NO: 335 (XXC XGG GAX GXX ACC GCC GGC AGC G) wherein Z is 23;
oo) SEQ ID NO: 336 (CXX CXG GGA XGX XAC CGC CGG CAG C) wherein Z is 23;
pp) SEQ ID NO: 337 (GCX XCX GGG AXG XXA CCG CCG GCA G) wherein Z is 23;
qq) SEQ ID NO: 338 (CGC XXC XGG GAX GXX ACC GCC GGC A) wherein Z is 23;
rr) SEQ ID NO: 339 (CCG CXX CXG GGA XGX XAC CGC CGG C) wherein Z is 23;
ss) SEQ ID NO: 340 (CCC GCX XCX GGG AXG XXA CCG CCG G) wherein Z is 23;
tt) SEQ ID NO: 341 (ACC CGC XXC XGG GAX GXX ACC GCC G) wherein Z is 23;
uu) SEQ ID NO: 342 (AAC CCG CXX CXG GGA XGX XAC CGC C) wherein Z is 23,
wherein X is selected from uracil (U) or thymine (T).

17. The compound of claim 16, wherein at least one instance of R¹ is —N(CH₃)₂.

18. The compound of claim 16, wherein each instance of R¹ is —N(CH₃)₂.

19. The compound of claim 16, wherein T is of the formula:

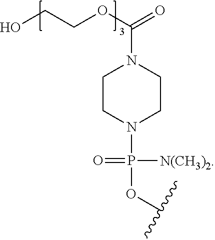

20. A compound of formula (V): 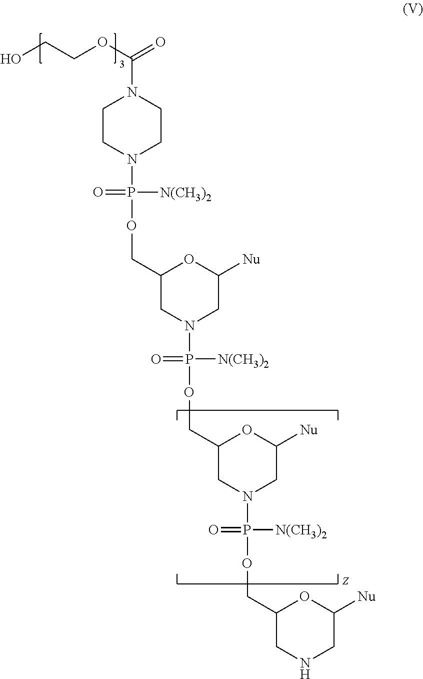
or a pharmaceutically acceptable salt thereof, wherein:
each Nu is a nucleobase which taken together form a targeting sequence; and
Z is an integer from 8 to 38,
wherein the targeting sequence is
I.
a) SEQ ID NO: 4 (GCC CXG GXC XGC XGG CXC CCX GCX G) wherein Z is 23;
b) SEQ ID NO: 5 (CCC XGG XCT GCT GGC TCC CTG CTG G) wherein Z is 23;
c) SEQ ID NO: 6 (CCX GGX CXG CXG GCX CCC XGC XGG X) wherein Z is 23;
d) SEQ ID NO: 7 (CXG GXC XGC XGG CXC CCX GCX GGX G) wherein Z is 23;
e) SEQ ID NO: 8 (XGG XCX GCX GGC XCC CXG CXG GXG A) wherein Z is 23;
f) SEQ ID NO: 9 (GGX CXG CXG GCX CCC XGC XGG XGA G) wherein Z is 23;
g) SEQ ID NO: 10 (GXC XGC XGG CXC CCX GCX GGX GAG C) wherein Z is 23;
h) SEQ ID NO: 11 (XCX GCX GGC XCC CXG CXG GXG AGC X) wherein Z is 23;
i) SEQ ID NO: 12 (GGG CCC XGG XCX GCX GGC XCC CXG C) wherein Z is 23;
j) SEQ ID NO: 13 (GGG GCC CXG GXC XGC XGG CXC CCX G) wherein Z is 23;
k) SEQ ID NO: 14 (CGG GGC CCX GGX CXG CXG GCX CCC X) wherein Z is 23;
l) SEQ ID NO: 15 (CCG GGG CCC XGG XCX GCX GGC XCC C) wherein Z is 23;
m) SEQ ID NO: 16 (CCC GGG GCC CXG GXC XGC XGG CXC C) wherein Z is 23;
n) SEQ ID NO: 17 (XCC CGG GGC CCX GGX CXG CXG GCX C) wherein Z is 23;
o) SEQ ID NO: 18 (AXC CCG GGG CCC XGG XCX GCX GGC X) wherein Z is 23;
p) SEQ ID NO: 19 (CAX CCC GGG GCC CXG GXC XGC XGG C) wherein Z is 23;
q) SEQ ID NO: 20 (XCX GCC CXG GCC GCC GCC CCC GCC CCX) wherein Z is 25;
r) SEQ ID NO: 21 (XGA GGX GCG XGG GXG XCG AXG XCC A) wherein Z is 23;
s) SEQ ID NO: 22 (GAG GXG CGX GGG XGX CGA XGX CCA C) wherein Z is 23;
t) SEQ ID NO: 23 (AGG XGC GXG GGX GXC GAX GXC CAC G) wherein Z is 23;
u) SEQ ID NO: 24 (GCG CGX GGA CAX CGA CAC CCA CGC A) wherein Z is 23;
v) SEQ ID NO: 25 (XGX GAG GGC GCG XGG ACA XCG ACA C) wherein Z is 23;
w) SEQ ID NO: 26 (XXG XGA GGG CGC GXG GAC AXC GAC A) wherein Z is 23;
x) SEQ ID NO: 27 (CX GXG AGG GCG CGX GGA CAX CGA C) wherein Z is 22;
y) SEQ ID NO: 28 (GGC CCX GGX CXG CXG GCX CCC XGC X) wherein Z is 23;
z) SEQ ID NO: 29 (XGG CCG CCG CCC CCG CCC CX) wherein Z is 18;
aa) SEQ ID NO: 30 (GXG AGG XGC GXG GGX GXC GA) wherein Z is 18, or
wherein X is selected from uracil (U) or thymine (T);
II.
a) SEQ ID NO: 133 (GCX CAG CAG GGA GGC GGG AG) wherein Z is 18;
b) SEQ ID NO:134 (GGC XCX CAA AGC AGC XCX GA) wherein Z is 18;
c) SEQ ID NO: 135 (GAC AXC AAC CGC GGC XGG CAC XGC A) wherein Z is 23;
d) SEQ ID NO: 136 (GGG XAA GGX GGC CAG GGX GGG XGX X) wherein Z is 23;
e) SEQ ID NO: 137 (GCC CXG CXG XCX AGA CXG G) wherein Z is 17;
f) SEQ ID NO:138 (GAG AGG GCC AGA AGG AAG GG) wherein Z is 18;
g) SEQ ID NO:139 (CCC GCC CCX GCC CXG CC) wherein Z is 15;
h) SEQ ID NO: 140 (XGG CCG CCG CCC CCG CCC) wherein Z is 16;
i) SEQ ID NO:141 (XGX CCA CGC GCA CCC XCX GC) wherein Z is 18;
j) SEQ ID NO:142 (GXG AGG XGC GXG GGX GXC GA) wherein Z is 18;
k) SEQ ID NO:143 (GCA ACA XGC ACC CCA CCC XX) wherein Z is 18;
l) SEQ ID NO:144 (AGG GCC CAG CAC ACA GXG GX) wherein Z is 18;
m) SEQ ID NO: 145 (XCA CAC CXC CGC XCC CAG CA) wherein Z is 18;
n) SEQ ID NO: 146 (GGC GCX GCC AXX GXC XGC) wherein Z is 16;
o) SEQ ID NO:147 (GXG XCC CCA CXG CXC CCC GA) wherein Z is 18;
p) SEQ ID NO:148 (CXG GAG XAC CXG XCA CCG XG) wherein Z is 18;
q) SEQ ID NO: 149 (XGA GCC CCG AGC CCX GCC XX) wherein Z is 18;
r) SEQ ID NO:150 (XGA CCC ACC XXX XCA XAA AGA XGA A) wherein Z is 23;
s) SEQ ID NO:151 (CXC XGG CAG CCC XAC XCX ACC XGA C) wherein Z is 23;
t) SEQ ID NO:152 (CXA GXA XAA AXA CAX CCC AAA XXX XGC) wherein Z is 25;
u) SEQ ID NO:153 (GGC CCX GGX CXG CXG GCX CCC XGC X) wherein Z is 23;
v) SEQ ID NO:154 (GCX CCC XGC AGC CCC XGC XXX GCA G) wherein Z is 23;
w) SEQ ID NO: 155 (GCG GGG CAG ACG XCA GGX GX) wherein Z is 18;
x) SEQ ID NO:156 (CAG CGC GGG GCA GAC GXC AG) wherein Z is 18;
y) SEQ ID NO:157 (CCG GCA GCG CGG GGC AGA CG) wherein Z is 18;
z) SEQ ID NO:158 (CCG CCG GCA GCG CGG GGC AG) wherein Z is 18;
aa) SEQ ID NO:159 (GAX GXX ACC GCC GGC AGC GC) wherein Z is 18;
bb) SEQ ID NO:160 (CXG GGA XGX XAC CGC CGG CA) wherein Z is 18;
cc) SEQ ID NO: 161 (GCX XCX GGG AXG XXA CCG CC) wherein Z is 18;
dd) SEQ ID NO: 162 (XGG CAA CXC GXA XGX CCX XA) wherein Z is 18;
ee) SEQ ID NO: 163 (AXX CXG GCA ACX CGX AXG XC) wherein Z is 18;
ff) SEQ ID NO: 164 (AAG XGA XXC XGG CAA CXC GX) wherein Z is 18;
gg) SEQ ID NO: 165 (XGG GXG XCA GCG GAA GXG AX) wherein Z is 18;
hh) SEQ ID NO: 166 (GXC CAC XGG GXG XCA GCG GA) wherein Z is 18;
ii) SEQ ID NO: 167 (GCX XGG XCC ACX GGG XGX CA) wherein Z is 18;
jj) SEQ ID NO:168 (CCC CAC XXC XGC AXA AAG GX) wherein Z is 18;
kk) SEQ ID NO:169 (GGA GCC CCA CXX CXG CAX AA) wherein Z is 18;
ll) SEQ ID NO:170 (GCX GGG AGC CCC ACX XCX GC) wherein Z is 18;
mm) SEQ ID NO:171 (CCA CGC CXG GCX GGG AGC CC) wherein Z is 18;
nn) SEQ ID NO: 172 (XCC GAA GXG CXG GGA XXX CA) wherein Z is 18;
oo) SEQ ID NO:173 (XCC ACC CCC CXX GGC CXX CC) wherein Z is 18;
pp) SEQ ID NO:174 (XGA XCC ACC CCC CXX GGC CX) wherein Z is 18;
qq) SEQ ID NO:175 (XCA AGX GAX CCA CCC CCC XX) wherein Z is 18;
rr) SEQ ID NO: 176 (GAA CXC CXG AGC XCA AGX GA) wherein Z is 18;
ss) SEQ ID NO:177 (XCX CGA ACX CCX GAG CXC AA) wherein Z is 18;
tt) SEQ ID NO:178 (CCA GGC XGG XCX CGA ACX CC) wherein Z is 18;
uu) SEQ ID NO:179 (XXX GCC AXG XXA CCC AGG CX) wherein Z is 18;
vv) SEQ ID NO: 180 (ACG GGA XXX XGC CAX GXX AC) wherein Z is 18;
ww) SEQ ID NO: 181 (XAG AGA CGG GAX XXX GCC AX) wherein Z is 18;
xx) SEQ ID NO:182 (XXX XGX AGA GAC GGG AXX XX) wherein Z is 18;
yy) SEQ ID NO: 183 (XCX GXA XXX XXG XAG AGA CG) wherein Z is 18;
zz) SEQ ID NO:184 (AXX XXC XGX AXX XXX GXA GA) wherein Z is 18;
aaa) SEQ ID NO: 185 (GCX AAX XXX CXG XAX XXX XG) wherein Z is 18;
bbb) SEQ ID NO: 186 (CCG CCG CCC CCG CCC CXG CC) wherein Z is 18;
ccc) SEQ ID NO: 187 (XGG CCG CCG CCC CCG CCC CX) wherein Z is 18;
ddd) SEQ ID NO:188 (CXG CCC XGG CCG CCG CCC CC) wherein Z is 18;
eee) SEQ ID NO:189 (CAC CCX CXG CCC XGG CCG CC) wherein Z is 18;
fff) SEQ ID NO:190 (GCG CAC CCX CXG CCC XGG CC) wherein Z is 18;
ggg) SEQ ID NO:191 (XGX CGA XGX CCA CGC GCA CC) wherein Z is 18;
hhh) SEQ ID NO:192 (XGC GXG GGX GXC GAX GXC CA) wherein Z is 18;
iii) SEQ ID NO:193 (GCA CCC CAC CCX XGX GAG GX) wherein Z is 18;
jjj) SEQ ID NO:194 (AAC AXG CAC CCC ACC CXX GX) wherein Z is 18;
kkk) SEQ ID NO: 195 (AGG AGG AGG ACG CCX CCC CC) wherein Z is 18;
lll) SEQ ID NO:196 (CXC AXC XGC AGA GCC AGG AG) wherein Z is 18;
mmm) SEQ ID NO:197 (GCX CCC XCA XCX GCA GAG CC) wherein Z is 18;
nnn) SEQ ID NO:198 (XCG GCX CCC XCA XCX GCA GA) wherein Z is 18;
ooo) SEQ ID NO:199 (GCC XCG GCX CCC XCA XCX GC) wherein Z is 18;
ppp) SEQ ID NO:200 (XXC XGG GAX GXX ACC GCC GG) wherein Z is 18;
qqq) SEQ ID NO:201 (CXX CXG GGA XGX XAC CGC CG) wherein Z is 18;
rrr) SEQ ID NO:202 (CGC XXC XGG GAX GXX ACC GC) wherein Z is 18;
sss) SEQ ID NO:203 (CCG CXX CXG GGA XGX XAC CG) wherein Z is 18;
ttt) SEQ ID NO:204 (ACC CGC XXC XGG GAX GXX AC) wherein Z is 18;
uuu) SEQ ID NO:205 (XCA AAC CCG CXX CXG GGA XG) wherein Z is 18;
vvv) SEQ ID NO:206 (ACG XXC AAA CCC GCX XCX GG) wherein Z is 18;
www) SEQ ID NO:207 (GGG CXC XCA AAG CAG CXC XG) wherein Z is 18;
xxx) SEQ ID NO:208 (GGG GCX CXC AAA GCA GCX CX) wherein Z is 18;
yyy) SEQ ID NO:209 (ACG GGG CXC XCA AAG CAG CX) wherein Z is 18;
zzz) SEQ ID NO:210 (XCA CGG GGC XCX CAA AGC AG) wherein Z is 18;
aaaa) SEQ ID NO:211 (GCX CXC AAA GCA GCX CXG AG) wherein Z is 18;
bbbb) SEQ ID NO:212 (CXC XCA AAG CAG CXC XGA GA) wherein Z is 18;
cccc) SEQ ID NO:213 (CXC AAA GCA GCX CXG AGA CA) wherein Z is 18;
dddd) SEQ ID NO:214 (CAA AGC AGC XCX GAG ACA XC) wherein Z is 18;
eeee) SEQ ID NO:215 (AAG CAG CXC XGA GAC AXC AA) wherein Z is 18;
ffff) SEQ ID NO:216 (GCC CXG GXC XGC XGG CXC CCX GCX G) wherein Z is 23;
gggg) SEQ ID NO:217 (CCC XGG XCX GCX GGC XCC CXG CXG G) wherein Z is 23;
hhhh) SEQ ID NO:218 (CCX GGX CXG CXG GCX CCC XGC XGG X) wherein Z is 23;
iiii) SEQ ID NO:219 (CXG GXC XGC XGG CXC CCX GCX GGX G) wherein Z is 23;
jjjj) SEQ ID NO:220 (XGG XCX GCX GGC XCC CXG CXG GXG A) wherein Z is 23;
kkkk) SEQ ID NO:221 (GGX CXG CXG GCX CCC XGC XGG XGA G) wherein Z is 23;
llll) SEQ ID NO:222 (GXC XGC XGG CXC CCX GCX GGX GAG C) wherein Z is 23;
mmmm) SEQ ID NO:223 (XCX GCX GGC XCC CXG CXG GXG AGC X) wherein Z is 23;
nnnn) SEQ ID NO:224 (GGG CCC XGG XCX GCX GGC XCC CXG C) wherein Z is 23;
oooo) SEQ ID NO:225 (GGG GCC CXG GXC XGC XGG CXC CCX G) wherein Z is 23;
pppp) SEQ ID NO:226 (CGG GGC CCX GGX CXG CXG GCX CCC X) wherein Z is 23;
qqqq) SEQ ID NO:227 (CCG GGG CCC XGG XCX GCX GGC XCC C) wherein Z is 23;
rrrr) SEQ ID NO:228 (CCC GGG GCC CXG GXC XGC XGG CXC C) wherein Z is 23;
ssss) SEQ ID NO:229 (XCC CGG GGC CCX GGX CXG CXG GCX C) wherein Z is 23;
tttt) SEQ ID NO:230 (AXC CCG GGG CCC XGG XCX GCX GGC X) wherein Z is 23;
uuuu) SEQ ID NO:231 (CAX CCC GGG GCC CXG GXC XGC XGG C) wherein Z is 23;
vvvv) SEQ ID NO:232 (XCX GCC CXG GCC GCC GCC CCC GCC CCX) wherein Z is 23;
wwww) SEQ ID NO:233 (XGA GGX GCG XGG GXG XCG AXG XCC A) wherein Z is 23;
xxxx) SEQ ID NO:234 (GAG GXG CGX GGG XGX CGA XGX CCA C) wherein Z is 23;
yyyy) SEQ ID NO:235 (AGG XGC GXG GGX GXC GAX GXC CAC G) wherein Z is 23;
zzzz) SEQ ID NO:236 (GCG CGX GGA CAX CGA CAC CCA CGC A) wherein Z is 23;
aaaaa) SEQ ID NO:237 (XGX GAG GGC GCG XGG ACA XCG ACA C) wherein Z is 23;
bbbbb) SEQ ID NO:238 (XXG XGA GGG CGC GXG GAC AXC GAC A) wherein Z is 23;
ccccc) SEQ ID NO:239 (CXX GXG AGG GCG CGX GGA CAX CGA C) wherein Z is 23;
ddddd) SEQ ID NO:240 (GAG AGG GCC AGA AGG AAG) wherein Z is 16;
eeeee) SEQ ID NO:241 (GAG GGC CAG AAG GAA GGG) wherein Z is 16;
fffff) SEQ ID NO:242 (GGG CCA GAA GGA AGG GCG) wherein Z is 16;
ggggg) SEQ ID NO:243 (GGG AGA GGG CCA GAA GGA) wherein Z is 16;
hhhhh) SEQ ID NO:244 (AGA GGG CCA GAA GGA AGG GC) wherein Z is 18;
iiiii) SEQ ID NO:245 (GAG GGC CAG AAG GAA GGG CG) wherein Z is 18;
jjjjj) SEQ ID NO:246 (AGG GCC AGA AGG AAG GGC GA) wherein Z is 18;
kkkkk) SEQ ID NO:247 (GGG CCA GAA GGA AGG GCG AG) wherein Z is 18;
lllll) SEQ ID NO:248 (GGC CAG AAG GAA GGG CGA GA) wherein Z is 18;
mmmmm) SEQ ID NO:249 (GCC AGA AGG AAG GGC GAG AA) wherein Z is 18;
nnnnn) SEQ ID NO:250 (GGG AGA GGG CCA GAA GGA AGG G) wherein Z is 20;
ooooo) SEQ ID NO:251 (CXG GGG AGA GGG CCA GAA GGA AGG G) wherein Z is 23;
ppppp) SEQ ID NO:252 (GAG AGG GCC AGA AGG AAG GGC G) wherein Z is 20;
qqqqq) SEQ ID NO:253 (GAG AGG GCC AGA AGG AAG GGC GAG A) wherein Z is 23;
rrrrr) SEQ ID NO:254 (GAA GGA AGG GCG AGA AAA GC) wherein Z is 18; or
sssss) SEQ ID NO:255 (GCA GAA AAG CXC CAG CAG GG) wherein Z is 18,
wherein X is selected from uracil (U) or thymine (T); or
III.
a) SEQ ID NO:296 (AAG CXC CAG CAG GGG AGX GCA GAG C) wherein Z is 23;
b) SEQ ID NO:297 (AAA AGC XCC AGC AGG GGA GXG CAG A) wherein Z is 23;
c) SEQ ID NO:298 (AGA AAA GCX CCA GCA GGG GAG XGC A) wherein Z is 23;
d) SEQ ID NO:299 (CGA GAA AAG CXC CAG CAG GGG AGX G) wherein Z is 23;
e) SEQ ID NO:300 (GGC GAG AAA AGC XCC AGC AGG GGA G) wherein Z is 23;
f) SEQ ID NO:301 (AGG GCG AGA AAA GCX CCA GCA GGG G) wherein Z is 23;
g) SEQ ID NO:302 (GAA GGG CGA GAA AAG CXC CAG CAG G) wherein Z is 23;
h) SEQ ID NO:303 (AGG AAG GGC GAG AAA AGC XCC AGC A) wherein Z is 23;
i) SEQ ID NO:304 (GAA GGA AGG GCG AGA AAA GCX CCA G) wherein Z is 23;
j) SEQ ID NO:305 (CAG AAG GAA GGG CGA GAA AAG CXC C) wherein Z is 23;
k) SEQ ID NO:306 (GCC AGA AGG AAG GGC GAG AAA AGC X) wherein Z is 23;
l) SEQ ID NO:307 (GGG CCA GAA GGA AGG GCG AGA AAA G) wherein Z is 23;
m) SEQ ID NO:308 (GAG GGC CAG AAG GAA GGG CGA GAA A) wherein Z is 23;
n) SEQ ID NO:309 (GAG AGG GCC AGA AGG AAG GGC GAG A) wherein Z is 23;
o) SEQ ID NO:310 (AGG GCC AGA AGG AAG GGC GA) wherein Z is 18;
p) SEQ ID NO:311 (GAG AGG GCC AGA AGG AAG GG) wherein Z is 18;
q) SEQ ID NO:312 (CXG GGG AGA GGG CCA GAA GGA AGG G) wherein Z is 23;
r) SEQ ID NO:313 (GAG AGG GCC AGA AGG AAG) wherein Z is 18;
s) SEQ ID NO:314 (GGG AGA GGG CCA GAA GGA) wherein Z is 18;
t) SEQ ID NO:315 (GGX CXG CXG GCX CCC XGC XGG XGA G) wherein Z is 23;
u) SEQ ID NO:316 (CAC XCA CGG GGC XCX CAA AGC AGC X) wherein Z is 23;
v) SEQ ID NO:317 (XCX GGG AXG XXA CCG CCG GCA GCG C) wherein Z is 23;
w) SEQ ID NO:318 (XXX GCC AXG XXA CCC AGG CX) wherein Z is 18;
x) SEQ ID NO:319 (ACX CAC GGG GCX CXC AAA GCA GCX C) wherein Z is 23;
y) SEQ ID NO:320 (GCA CXC ACG GGG CXC XCA AAG CAG C) wherein Z is 23;
z) SEQ ID NO:321 (CGG GGC XCX CAA AGC AGC XCX GAG A) wherein Z is 23;
aa) SEQ ID NO:322 (ACG GGG CXC XCA AAG CAG CXC XGA G) wherein Z is 23;
bb) SEQ ID NO:323 (CAC GGG GCX CXC AAA GCA GCX CXG A) wherein Z is 23;
cc) SEQ ID NO:324 (XCA CGG GGC XCX CAA AGC AGC XCX G) wherein Z is 23;
dd) SEQ ID NO:325 (CXC ACG GGG CXC XCA AAG CAG CXC X) wherein Z is 23;
ee) SEQ ID NO:326 (GGC ACX CAC GGG GCX CXC AAA GCA G) wherein Z is 23;
ff) SEQ ID NO:327 (CGG CAC XCA CGG GGC XCX CAA AGC A) wherein Z is 23;
gg) SEQ ID NO:328 (GCG GCA CXC ACG GGG CXC XCA AAG C) wherein Z is 23;
hh) SEQ ID NO:329 (GGC GGC ACX CAC GGG GCX CXC AAA G) wherein Z is 23;
ii) SEQ ID NO:330 (GGG CGG CAC XCA CGG GGC XCX CAA A) wherein Z is 23;
jj) SEQ ID NO:331 (GGG GCG GCA CXC ACG GGG CXC XCA A) wherein Z is 23;
kk) SEQ ID NO:332 (AGG GGC GGC ACX CAC GGG GCX CXC A) wherein Z is 23;
ll) SEQ ID NO:333 (GAG GGG CGG CAC XCA CGG GGC XCX C) wherein Z is 23;
mm) SEQ ID NO: 334 (GGC XCX CAA AGC AGC XCX GA) wherein Z is 18;
nn) SEQ ID NO: 335 (XXC XGG GAX GXX ACC GCC GGC AGC G) wherein Z is 23;
oo) SEQ ID NO: 336 (CXX CXG GGA XGX XAC CGC CGG CAG C) wherein Z is 23;
pp) SEQ ID NO: 337 (GCX XCX GGG AXG XXA CCG CCG GCA G) wherein Z is 23;
qq) SEQ ID NO: 338 (CGC XXC XGG GAX GXX ACC GCC GGC A) wherein Z is 23;
rr) SEQ ID NO: 339 (CCG CXX CXG GGA XGX XAC CGC CGG C) wherein Z is 23;
ss) SEQ ID NO: 340 (CCC GCX XCX GGG AXG XXA CCG CCG G) wherein Z is 23;
tt) SEQ ID NO: 341 (ACC CGC XXC XGG GAX GXX ACC GCC G) wherein Z is 23;
uu) SEQ ID NO: 342 (AAC CCG CXX CXG GGA XGX XAC CGC C) wherein Z is 23,
wherein X is selected from uracil (U) or thymine (T).

21-40. (canceled)

41. A method of treating glycogen storage disease type II in a subject in need thereof, the method comprising administering to the subject an effective amount of a compound of formula (I): 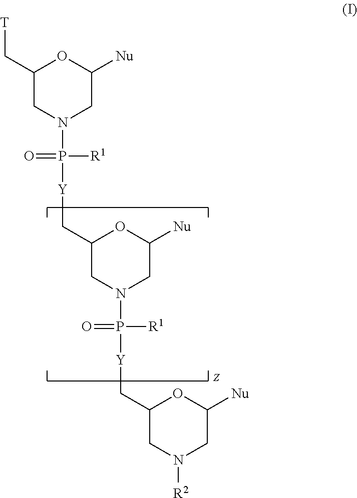
or a pharmaceutically acceptable salt thereof, wherein:
each Nu is a nucleobase which taken together form a targeting sequence;
each Y is independently selected from O and —NR⁴, wherein each R⁴ is independently selected from H, C₁-C₆ alkyl, aralkyl, —C(═NH)NH₂, —C(O)(CH₂)NR⁵C(═NH)NH₂, —C(O)(CH₂)₂NHC(O)(CH₂)₅NR⁵C(═NH)NH₂, and G, wherein R⁵ is selected from H and C₁-C₆ alkyl and n is an integer from 1 to 5;
T is selected from OH and a moiety of the formula: 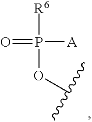
wherein:
A is selected from —OH, —N(R⁷)₂, and R¹ wherein:
each R⁷ is independently selected from H and C₁-C₆ alkyl, and
R⁶ is selected from OH, —N(R⁹)CH₂C(O)NH₂, and a moiety of the formula: 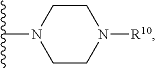
wherein:
R⁹ is selected from H and C₁-C₆ alkyl; and
R¹⁰ is selected from G, —C(O)—R¹¹OH, acyl, trityl, 4-methoxytrityl, —C(═NH)NH₂, —C(O)(CH₂)ₘNR¹²C(═NH)NH₂, and —C(O)(CH₂)₂NHC(O)(CH₂)₅NR¹²C(═NH)NH₂, wherein:
m is an integer from 1 to 5,
R¹¹ is of the formula —(O-alkyl)_{y}- wherein y is an integer from 3 to 10 and
each of the y alkyl groups is independently selected from C₂-C₆ alkyl; and
R¹² is selected from H and C₁-C₆ alkyl;
each instance of R¹ is independently selected from:
—N(R¹³)₂, wherein each R¹³ is independently selected from H and C₁-C₆ alkyl;
a moiety of formula (II): 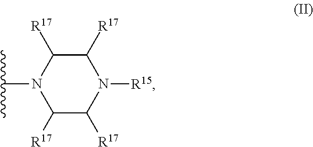
wherein:
R¹⁵ is selected from H, G, C₁-C₆ alkyl, —C(═NH)NH₂, —C(O)(CH₂)_{q}NR¹⁸C(═NH)NH₂, and —C(O)(CH₂)₂NHC(O)(CH₂)₅NR¹⁸C(═NH)NH₂, wherein:
R¹⁸ is selected from H and C₁-C₆ alkyl; and
q is an integer from 1 to 5; and
each R¹⁷ is independently selected from H and methyl; and
a moiety of formula (III): 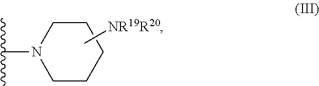
wherein:
R¹⁹ is selected from H, C₁-C₆ alkyl, —C(═NH)NH₂, —C(O)(CH₂)ᵣNR²²C(═NH)NH₂, —C(O)CH(NH₂)(CH₂)₃NHC(═NH)NH₂, —C(O)(CH₂)₂NHC(O)(CH₂)₅NR²²C(═NH)NH₂, —C(O)CH(NH₂)(CH₂)₄NH₂ and G, wherein:
R²² is selected from H and C₁-C₆ alkyl; and
r is an integer from 1 to 5,
R²⁰ is selected from H and C₁-C₆ alkyl;
R² is selected from H, G, acyl, trityl, 4-methoxytrityl, C₁-C₆ alkyl, —C(═NH)NH₂, —C(O)—R²³, —C(O)(CH₂)ₛNR²⁴C(═NH)NH₂, —C(O)(CH₂)₂NHC(O)(CH₂)₅NR²⁴C(═NH)NH₂, —C(O)CH(NH₂)(CH₂)₃NHC(═NH)NH₂, and a moiety of the formula: 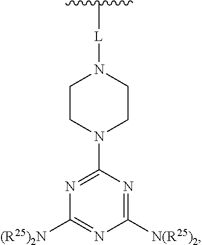
wherein,
R²³ is of the formula —(O-alkyl)ᵥ-OH wherein v is an integer from 3 to 10 and each of the v alkyl groups is independently selected from C₂-C₆ alkyl; and
R²⁴ is selected from H and C₁-C₆ alkyl;
s is an integer from 1 to 5;
L is selected from —C(O)(CH₂)₆C(O)— and —C(O)(CH₂)₂S₂(CH₂)₂C(O)—; and
each R²⁵ is of the formula —(CH₂)₂₀C(O)N(R²⁶)₂ wherein each R²⁶ is of the formula —(CH₂)₆NHC(═NH)NH₂,
wherein G is a cell penetrating peptide (“CPP”) and linker moiety selected from —C(O)(CH₂)₅NH-CPP, —C(O)(CH₂)₂NH-CPP, —C(O)(CH₂)₂NHC(O)(CH₂)₅NH-CPP, and —C(O)CH₂NH—CPP, or G is of the formula: 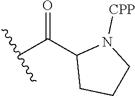
wherein the CPP is attached to the linker moiety by an amide bond at the CPP carboxy terminus, with the proviso that up to one instance of G is present, and
wherein the targeting sequence is:
I.
a) SEQ ID NO: 4 (GCC CXG GXC XGC XGG CXC CCX GCX G) wherein Z is 23;
b) SEQ ID NO: 5 (CCC XGG XCT GCT GGC TCC CTG CTG G) wherein Z is 23;
c) SEQ ID NO: 6 (CCX GGX CXG CXG GCX CCC XGC XGG X) wherein Z is 23;
d) SEQ ID NO: 7 (CXG GXC XGC XGG CXC CCX GCX GGX G) wherein Z is 23;
e) SEQ ID NO: 8 (XGG XCX GCX GGC XCC CXG CXG GXG A) wherein Z is 23;
f) SEQ ID NO: 9 (GGX CXG CXG GCX CCC XGC XGG XGA G) wherein Z is 23;
g) SEQ ID NO: 10 (GXC XGC XGG CXC CCX GCX GGX GAG C) wherein Z is 23;
h) SEQ ID NO: 11 (XCX GCX GGC XCC CXG CXG GXG AGC X) wherein Z is 23;
i) SEQ ID NO: 12 (GGG CCC XGG XCX GCX GGC XCC CXG C) wherein Z is 23;
j) SEQ ID NO: 13 (GGG GCC CXG GXC XGC XGG CXC CCX G) wherein Z is 23;
k) SEQ ID NO: 14 (CGG GGC CCX GGX CXG CXG GCX CCC X) wherein Z is 23;
l) SEQ ID NO: 15 (CCG GGG CCC XGG XCX GCX GGC XCC C) wherein Z is 23;
m) SEQ ID NO: 16 (CCC GGG GCC CXG GXC XGC XGG CXC C) wherein Z is 23;
n) SEQ ID NO: 17 (XCC CGG GGC CCX GGX CXG CXG GCX C) wherein Z is 23;
o) SEQ ID NO: 18 (AXC CCG GGG CCC XGG XCX GCX GGC X) wherein Z is 23;
p) SEQ ID NO: 19 (CAX CCC GGG GCC CXG GXC XGC XGG C) wherein Z is 23;
q) SEQ ID NO: 20 (XCX GCC CXG GCC GCC GCC CCC GCC CCX) wherein Z is 25;
r) SEQ ID NO: 21 (XGA GGX GCG XGG GXG XCG AXG XCC A) wherein Z is 23;
s) SEQ ID NO: 22 (GAG GXG CGX GGG XGX CGA XGX CCA C) wherein Z is 23;
t) SEQ ID NO: 23 (AGG XGC GXG GGX GXC GAX GXC CAC G) wherein Z is 23;
u) SEQ ID NO: 24 (GCG CGX GGA CAX CGA CAC CCA CGC A) wherein Z is 23;
v) SEQ ID NO: 25 (XGX GAG GGC GCG XGG ACA XCG ACA C) wherein Z is 23;
w) SEQ ID NO: 26 (XXG XGA GGG CGC GXG GAC AXC GAC A) wherein Z is 23;
x) SEQ ID NO: 27 (CX GXG AGG GCG CGX GGA CAX CGA C) wherein Z is 22;
y) SEQ ID NO: 28 (GGC CCX GGX CXG CXG GCX CCC XGC X) wherein Z is 23;
z) SEQ ID NO: 29 (XGG CCG CCG CCC CCG CCC CX) wherein Z is 18;
aa) SEQ ID NO: 30 (GXG AGG XGC GXG GGX GXC GA) wherein Z is 18, or
wherein X is selected from uracil (U) or thymine (T);
II.
a) SEQ ID NO: 133 (GCX CAG CAG GGA GGC GGG AG) wherein Z is 18;
b) SEQ ID NO:134 (GGC XCX CAA AGC AGC XCX GA) wherein Z is 18;
c) SEQ ID NO:135 (GAC AXC AAC CGC GGC XGG CAC XGC A) wherein Z is 23;
d) SEQ ID NO: 136 (GGG XAA GGX GGC CAG GGX GGG XGX X) wherein Z is 23;
e) SEQ ID NO: 137 (GCC CXG CXG XCX AGA CXG G) wherein Z is 17;
f) SEQ ID NO:138 (GAG AGG GCC AGA AGG AAG GG) wherein Z is 18;
g) SEQ ID NO:139 (CCC GCC CCX GCC CXG CC) wherein Z is 15;
h) SEQ ID NO: 140 (XGG CCG CCG CCC CCG CCC) wherein Z is 16;
i) SEQ ID NO:141 (XGX CCA CGC GCA CCC XCX GC) wherein Z is 18;
j) SEQ ID NO: 142 (GXG AGG XGC GXG GGX GXC GA) wherein Z is 18;
k) SEQ ID NO:143 (GCA ACA XGC ACC CCA CCC XX) wherein Z is 18;
l) SEQ ID NO:144 (AGG GCC CAG CAC ACA GXG GX) wherein Z is 18;
m) SEQ ID NO:145 (XCA CAC CXC CGC XCC CAG CA) wherein Z is 18;
n) SEQ ID NO:146 (GGC GCX GCC AXX GXC XGC) wherein Z is 16;
o) SEQ ID NO:147 (GXG XCC CCA CXG CXC CCC GA) wherein Z is 18;
p) SEQ ID NO: 148 (CXG GAG XAC CXG XCA CCG XG) wherein Z is 18;
q) SEQ ID NO: 149 (XGA GCC CCG AGC CCX GCC XX) wherein Z is 18;
r) SEQ ID NO:150 (XGA CCC ACC XXX XCA XAA AGA XGA A) wherein Z is 23;
s) SEQ ID NO:151 (CXC XGG CAG CCC XAC XCX ACC XGA C) wherein Z is 23;
t) SEQ ID NO:152 (CXA GXA XAA AXA CAX CCC AAA XXX XGC) wherein Z is 25;
u) SEQ ID NO:153 (GGC CCX GGX CXG CXG GCX CCC XGC X) wherein Z is 23;
v) SEQ ID NO:154 (GCX CCC XGC AGC CCC XGC XXX GCA G) wherein Z is 23;
w) SEQ ID NO:155 (GCG GGG CAG ACG XCA GGX GX) wherein Z is 18;
x) SEQ ID NO:156 (CAG CGC GGG GCA GAC GXC AG) wherein Z is 18;
y) SEQ ID NO:157 (CCG GCA GCG CGG GGC AGA CG) wherein Z is 18;
z) SEQ ID NO:158 (CCG CCG GCA GCG CGG GGC AG) wherein Z is 18;
aa) SEQ ID NO:159 (GAX GXX ACC GCC GGC AGC GC) wherein Z is 18;
bb) SEQ ID NO: 160 (CXG GGA XGX XAC CGC CGG CA) wherein Z is 18;
cc) SEQ ID NO: 161 (GCX XCX GGG AXG XXA CCG CC) wherein Z is 18;
dd) SEQ ID NO: 162 (XGG CAA CXC GXA XGX CCX XA) wherein Z is 18;
ee) SEQ ID NO: 163 (AXX CXG GCA ACX CGX AXG XC) wherein Z is 18;
ff) SEQ ID NO:164 (AAG XGA XXC XGG CAA CXC GX) wherein Z is 18;
gg) SEQ ID NO:165 (XGG GXG XCA GCG GAA GXG AX) wherein Z is 18;
hh) SEQ ID NO:166 (GXC CAC XGG GXG XCA GCG GA) wherein Z is 18;
ii) SEQ ID NO: 167 (GCX XGG XCC ACX GGG XGX CA) wherein Z is 18;
jj) SEQ ID NO:168 (CCC CAC XXC XGC AXA AAG GX) wherein Z is 18;
kk) SEQ ID NO:169 (GGA GCC CCA CXX CXG CAX AA) wherein Z is 18;
ll) SEQ ID NO:170 (GCX GGG AGC CCC ACX XCX GC) wherein Z is 18;
mm) SEQ ID NO:171 (CCA CGC CXG GCX GGG AGC CC) wherein Z is 18;
nn) SEQ ID NO: 172 (XCC GAA GXG CXG GGA XXX CA) wherein Z is 18;
oo) SEQ ID NO:173 (XCC ACC CCC CXX GGC CXX CC) wherein Z is 18;
pp) SEQ ID NO: 174 (XGA XCC ACC CCC CXX GGC CX) wherein Z is 18;
qq) SEQ ID NO:175 (XCA AGX GAX CCA CCC CCC XX) wherein Z is 18;
rr) SEQ ID NO:176 (GAA CXC CXG AGC XCA AGX GA) wherein Z is 18;
ss) SEQ ID NO:177 (XCX CGA ACX CCX GAG CXC AA) wherein Z is 18;
tt) SEQ ID NO:178 (CCA GGC XGG XCX CGA ACX CC) wherein Z is 18;
uu) SEQ ID NO:179 (XXX GCC AXG XXA CCC AGG CX) wherein Z is 18;
vv) SEQ ID NO: 180 (ACG GGA XXX XGC CAX GXX AC) wherein Z is 18;
ww) SEQ ID NO: 181 (XAG AGA CGG GAX XXX GCC AX) wherein Z is 18;
xx) SEQ ID NO:182 (XXX XGX AGA GAC GGG AXX XX) wherein Z is 18;
yy) SEQ ID NO:183 (XCX GXA XXX XXG XAG AGA CG) wherein Z is 18;
zz) SEQ ID NO:184 (AXX XXC XGX AXX XXX GXA GA) wherein Z is 18;
aaa) SEQ ID NO: 185 (GCX AAX XXX CXG XAX XXX XG) wherein Z is 18;
bbb) SEQ ID NO:186 (CCG CCG CCC CCG CCC CXG CC) wherein Z is 18;
ccc) SEQ ID NO:187 (XGG CCG CCG CCC CCG CCC CX) wherein Z is 18;
ddd) SEQ ID NO:188 (CXG CCC XGG CCG CCG CCC CC) wherein Z is 18;
eee) SEQ ID NO:189 (CAC CCX CXG CCC XGG CCG CC) wherein Z is 18;
fff) SEQ ID NO:190 (GCG CAC CCX CXG CCC XGG CC) wherein Z is 18;
ggg) SEQ ID NO:191 (XGX CGA XGX CCA CGC GCA CC) wherein Z is 18;
hhh) SEQ ID NO:192 (XGC GXG GGX GXC GAX GXC CA) wherein Z is 18;
iii) SEQ ID NO:193 (GCA CCC CAC CCX XGX GAG GX) wherein Z is 18;
jjj) SEQ ID NO:194 (AAC AXG CAC CCC ACC CXX GX) wherein Z is 18;
kkk) SEQ ID NO: 195 (AGG AGG AGG ACG CCX CCC CC) wherein Z is 18;
lll) SEQ ID NO:196 (CXC AXC XGC AGA GCC AGG AG) wherein Z is 18;
mmm) SEQ ID NO:197 (GCX CCC XCA XCX GCA GAG CC) wherein Z is 18;
nnn) SEQ ID NO:198 (XCG GCX CCC XCA XCX GCA GA) wherein Z is 18;
ooo) SEQ ID NO:199 (GCC XCG GCX CCC XCA XCX GC) wherein Z is 18;
ppp) SEQ ID NO:200 (XXC XGG GAX GXX ACC GCC GG) wherein Z is 18;
qqq) SEQ ID NO:201 (CXX CXG GGA XGX XAC CGC CG) wherein Z is 18;
rrr) SEQ ID NO:202 (CGC XXC XGG GAX GXX ACC GC) wherein Z is 18;
sss) SEQ ID NO:203 (CCG CXX CXG GGA XGX XAC CG) wherein Z is 18;
ttt) SEQ ID NO:204 (ACC CGC XXC XGG GAX GXX AC) wherein Z is 18;
uuu) SEQ ID NO:205 (XCA AAC CCG CXX CXG GGA XG) wherein Z is 18;
vvv) SEQ ID NO:206 (ACG XXC AAA CCC GCX XCX GG) wherein Z is 18;
www) SEQ ID NO:207 (GGG CXC XCA AAG CAG CXC XG) wherein Z is 18;
xxx) SEQ ID NO:208 (GGG GCX CXC AAA GCA GCX CX) wherein Z is 18;
yyy) SEQ ID NO:209 (ACG GGG CXC XCA AAG CAG CX) wherein Z is 18;
zzz) SEQ ID NO:210 (XCA CGG GGC XCX CAA AGC AG) wherein Z is 18;
aaaa) SEQ ID NO:211 (GCX CXC AAA GCA GCX CXG AG) wherein Z is 18;
bbbb) SEQ ID NO:212 (CXC XCA AAG CAG CXC XGA GA) wherein Z is 18;
cccc) SEQ ID NO:213 (CXC AAA GCA GCX CXG AGA CA) wherein Z is 18;
dddd) SEQ ID NO:214 (CAA AGC AGC XCX GAG ACA XC) wherein Z is 18;
eeee) SEQ ID NO:215 (AAG CAG CXC XGA GAC AXC AA) wherein Z is 18;
ffff) SEQ ID NO:216 (GCC CXG GXC XGC XGG CXC CCX GCX G) wherein Z is 23;
gggg) SEQ ID NO:217 (CCC XGG XCX GCX GGC XCC CXG CXG G) wherein Z is 23;
hhhh) SEQ ID NO:218 (CCX GGX CXG CXG GCX CCC XGC XGG X) wherein Z is 23;
iiii) SEQ ID NO:219 (CXG GXC XGC XGG CXC CCX GCX GGX G) wherein Z is 23;
jjjj) SEQ ID NO:220 (XGG XCX GCX GGC XCC CXG CXG GXG A) wherein Z is 23;
kkkk) SEQ ID NO:221 (GGX CXG CXG GCX CCC XGC XGG XGA G) wherein Z is 23;
llll) SEQ ID NO:222 (GXC XGC XGG CXC CCX GCX GGX GAG C) wherein Z is 23;
mmmm) SEQ ID NO:223 (XCX GCX GGC XCC CXG CXG GXG AGC X) wherein Z is 23;
nnnn) SEQ ID NO:224 (GGG CCC XGG XCX GCX GGC XCC CXG C) wherein Z is 23;
oooo) SEQ ID NO:225 (GGG GCC CXG GXC XGC XGG CXC CCX G) wherein Z is 23;
pppp) SEQ ID NO:226 (CGG GGC CCX GGX CXG CXG GCX CCC X) wherein Z is 23;
qqqq) SEQ ID NO:227 (CCG GGG CCC XGG XCX GCX GGC XCC C) wherein Z is 23;
rrrr) SEQ ID NO:228 (CCC GGG GCC CXG GXC XGC XGG CXC C) wherein Z is 23;
ssss) SEQ ID NO:229 (XCC CGG GGC CCX GGX CXG CXG GCX C) wherein Z is 23;
tttt) SEQ ID NO:230 (AXC CCG GGG CCC XGG XCX GCX GGC X) wherein Z is 23;
uuuu) SEQ ID NO:231 (CAX CCC GGG GCC CXG GXC XGC XGG C) wherein Z is 23;
vvvv) SEQ ID NO:232 (XCX GCC CXG GCC GCC GCC CCC GCC CCX) wherein Z is 23;
wwww) SEQ ID NO:233 (XGA GGX GCG XGG GXG XCG AXG XCC A) wherein Z is 23;
xxxx) SEQ ID NO:234 (GAG GXG CGX GGG XGX CGA XGX CCA C) wherein Z is 23;
yyyy) SEQ ID NO:235 (AGG XGC GXG GGX GXC GAX GXC CAC G) wherein Z is 23;
zzzz) SEQ ID NO:236 (GCG CGX GGA CAX CGA CAC CCA CGC A) wherein Z is 23;
aaaaa) SEQ ID NO:237 (XGX GAG GGC GCG XGG ACA XCG ACA C) wherein Z is 23;
bbbbb) SEQ ID NO:238 (XXG XGA GGG CGC GXG GAC AXC GAC A) wherein Z is 23;
ccccc) SEQ ID NO:239 (CXX GXG AGG GCG CGX GGA CAX CGA C) wherein Z is 23;
ddddd) SEQ ID NO:240 (GAG AGG GCC AGA AGG AAG) wherein Z is 16;
eeeee) SEQ ID NO:241 (GAG GGC CAG AAG GAA GGG) wherein Z is 16;
fffff) SEQ ID NO:242 (GGG CCA GAA GGA AGG GCG) wherein Z is 16;
ggggg) SEQ ID NO:243 (GGG AGA GGG CCA GAA GGA) wherein Z is 16;
hhhhh) SEQ ID NO:244 (AGA GGG CCA GAA GGA AGG GC) wherein Z is 18;
iiiii) SEQ ID NO:245 (GAG GGC CAG AAG GAA GGG CG) wherein Z is 18;
jjjjj) SEQ ID NO:246 (AGG GCC AGA AGG AAG GGC GA) wherein Z is 18;
kkkkk) SEQ ID NO:247 (GGG CCA GAA GGA AGG GCG AG) wherein Z is 18;
lllll) SEQ ID NO:248 (GGC CAG AAG GAA GGG CGA GA) wherein Z is 18;
mmmmm) SEQ ID NO:249 (GCC AGA AGG AAG GGC GAG AA) wherein Z is 18;
nnnnn) SEQ ID NO:250 (GGG AGA GGG CCA GAA GGA AGG G) wherein Z is 20;
ooooo) SEQ ID NO:251 (CXG GGG AGA GGG CCA GAA GGA AGG G) wherein Z is 23;
ppppp) SEQ ID NO:252 (GAG AGG GCC AGA AGG AAG GGC G) wherein Z is 20;
qqqqq) SEQ ID NO:253 (GAG AGG GCC AGA AGG AAG GGC GAG A) wherein Z is 23;
rrrrr) SEQ ID NO:254 (GAA GGA AGG GCG AGA AAA GC) wherein Z is 18; or
sssss) SEQ ID NO:255 (GCA GAA AAG CXC CAG CAG GG) wherein Z is 18,
wherein X is selected from uracil (U) or thymine (T); or
III.
a) SEQ ID NO:296 (AAG CXC CAG CAG GGG AGX GCA GAG C) wherein Z is 23;
b) SEQ ID NO:297 (AAA AGC XCC AGC AGG GGA GXG CAG A) wherein Z is 23;
c) SEQ ID NO:298 (AGA AAA GCX CCA GCA GGG GAG XGC A) wherein Z is 23;
d) SEQ ID NO:299 (CGA GAA AAG CXC CAG CAG GGG AGX G) wherein Z is 23;
e) SEQ ID NO:300 (GGC GAG AAA AGC XCC AGC AGG GGA G) wherein Z is 23;
f) SEQ ID NO:301 (AGG GCG AGA AAA GCX CCA GCA GGG G) wherein Z is 23;
g) SEQ ID NO:302 (GAA GGG CGA GAA AAG CXC CAG CAG G) wherein Z is 23;
h) SEQ ID NO:303 (AGG AAG GGC GAG AAA AGC XCC AGC A) wherein Z is 23;
i) SEQ ID NO:304 (GAA GGA AGG GCG AGA AAA GCX CCA G) wherein Z is 23;
j) SEQ ID NO:305 (CAG AAG GAA GGG CGA GAA AAG CXC C) wherein Z is 23;
k) SEQ ID NO:306 (GCC AGA AGG AAG GGC GAG AAA AGC X) wherein Z is 23;
l) SEQ ID NO:307 (GGG CCA GAA GGA AGG GCG AGA AAA G) wherein Z is 23;
m) SEQ ID NO:308 (GAG GGC CAG AAG GAA GGG CGA GAA A) wherein Z is 23;
n) SEQ ID NO:309 (GAG AGG GCC AGA AGG AAG GGC GAG A) wherein Z is 23;
o) SEQ ID NO:310 (AGG GCC AGA AGG AAG GGC GA) wherein Z is 18;
p) SEQ ID NO:311 (GAG AGG GCC AGA AGG AAG GG) wherein Z is 18;
q) SEQ ID NO:312 (CXG GGG AGA GGG CCA GAA GGA AGG G) wherein Z is 23;
r) SEQ ID NO:313 (GAG AGG GCC AGA AGG AAG) wherein Z is 18;
s) SEQ ID NO:314 (GGG AGA GGG CCA GAA GGA) wherein Z is 18;
t) SEQ ID NO:315 (GGX CXG CXG GCX CCC XGC XGG XGA G) wherein Z is 23;
u) SEQ ID NO:316 (CAC XCA CGG GGC XCX CAA AGC AGC X) wherein Z is 23;
v) SEQ ID NO:317 (XCX GGG AXG XXA CCG CCG GCA GCG C) wherein Z is 23;
w) SEQ ID NO:318 (XXX GCC AXG XXA CCC AGG CX) wherein Z is 18;
x) SEQ ID NO:319 (ACX CAC GGG GCX CXC AAA GCA GCX C) wherein Z is 23;
y) SEQ ID NO:320 (GCA CXC ACG GGG CXC XCA AAG CAG C) wherein Z is 23;
z) SEQ ID NO:321 (CGG GGC XCX CAA AGC AGC XCX GAG A) wherein Z is 23;
aa) SEQ ID NO:322 (ACG GGG CXC XCA AAG CAG CXC XGA G) wherein Z is 23;
bb) SEQ ID NO:323 (CAC GGG GCX CXC AAA GCA GCX CXG A) wherein Z is 23;
cc) SEQ ID NO:324 (XCA CGG GGC XCX CAA AGC AGC XCX G) wherein Z is 23;
dd) SEQ ID NO:325 (CXC ACG GGG CXC XCA AAG CAG CXC X) wherein Z is 23;
ee) SEQ ID NO:326 (GGC ACX CAC GGG GCX CXC AAA GCA G) wherein Z is 23;
ff) SEQ ID NO:327 (CGG CAC XCA CGG GGC XCX CAA AGC A) wherein Z is 23;
gg) SEQ ID NO:328 (GCG GCA CXC ACG GGG CXC XCA AAG C) wherein Z is 23;
hh) SEQ ID NO:329 (GGC GGC ACX CAC GGG GCX CXC AAA G) wherein Z is 23;
ii) SEQ ID NO:330 (GGG CGG CAC XCA CGG GGC XCX CAA A) wherein Z is 23;
jj) SEQ ID NO:331 (GGG GCG GCA CXC ACG GGG CXC XCA A) wherein Z is 23;
kk) SEQ ID NO:332 (AGG GGC GGC ACX CAC GGG GCX CXC A) wherein Z is 23;
ll) SEQ ID NO:333 (GAG GGG CGG CAC XCA CGG GGC XCX C) wherein Z is 23;
mm) SEQ ID NO: 334 (GGC XCX CAA AGC AGC XCX GA) wherein Z is 18;
nn) SEQ ID NO: 335 (XXC XGG GAX GXX ACC GCC GGC AGC G) wherein Z is 23;
oo) SEQ ID NO: 336 (CXX CXG GGA XGX XAC CGC CGG CAG C) wherein Z is 23;
pp) SEQ ID NO: 337 (GCX XCX GGG AXG XXA CCG CCG GCA G) wherein Z is 23;
qq) SEQ ID NO: 338 (CGC XXC XGG GAX GXX ACC GCC GGC A) wherein Z is 23;
rr) SEQ ID NO: 339 (CCG CXX CXG GGA XGX XAC CGC CGG C) wherein Z is 23;
ss) SEQ ID NO: 340 (CCC GCX XCX GGG AXG XXA CCG CCG G) wherein Z is 23;
tt) SEQ ID NO: 341 (ACC CGC XXC XGG GAX GXX ACC GCC G) wherein Z is 23;
uu) SEQ ID NO: 342 (AAC CCG CXX CXG GGA XGX XAC CGC C) wherein Z is 23, or
wherein X is selected from uracil (U) or thymine (T).

42-59. (canceled)
